(19) United States
(12) Patent Application Publication
(10) Pub. No.: US 2004/0157891 A1
(43) Pub. Date: Aug. 12, 2004

(54) Inhibitor of cox

(75) Inventors: Hirofumi Yamamoto, Takarazuka-shi (JP); Junya Ishida, Nishinomiya-shi (JP); Daisuke Tanabe, Nishinomiya-shi (JP); Shigeki Satoh, Tsukuba-shi (JP); Yuki Sawada, Ushiku-shi (JP); Takehiko Ohkawa, Ishige-machi (JP); Kenichiro Imamura, Tsukuba-shi (JP); Katsuya Nakamura, Nishinomiya-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: FUJISAWA PHARMACEUTICAL CO., LTD., Osaka-shi (JP)

(21) Appl. No.: 10/758,253

(22) Filed: Jan. 16, 2004

(30) Foreign Application Priority Data
Jan. 17, 2003 (AU) 2003900207
Mar. 31, 2003 (AU) 2003901873

(51) Int. Cl. A61K 31/4439; A61K 31/421; C07D 413/02

(52) U.S. Cl. 514/340; 514/374; 546/271.4; 548/235

(57) ABSTRACT

A compound of the formula (I):

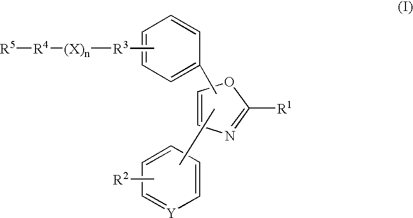

wherein

R¹ is cycloalkyl, etc;

R² is (lower)alkoxy, etc;

R³ is (lower)alkylene, etc;

R⁴ is (lower)alkylene, etc;

R⁵ is hydroxy, etc;

X is “O”, “S”, “SO”, or “SO₂”;

Y is “CH” or “N”;

n is 0 or 1;

or pharmaceutically acceptable salts thereof, which are useful as a medicament.

### BACKGROUND OF THE INVENTION

1. Field of the Invention

This invention relates to new compounds and pharmaceutically acceptable salts thereof having pharmacological activity.

2. Description of the Related Art

Cyclooxygenase catalyzes early stage reaction of arachidonate cascade, which is very important for a living body. For example, this cascade synthesizes prostaglandins as autacoids. So, antagonists or agonists of cyclooxygenase can be expected as medicines for treatment and/or prevention of inflammatory conditions, and so on.

As this cyclooxygenase, the presence of two isoenzymes, cyclooxygenase-I (COX-I) and cyclooxygenase-II (COX-II), is known (Proc. Nat. Acad. Sci. USA, 88, pp.2692-2696 (1991)). COX-I is always expressed over whole body and participates the maintenance of biological function at various tissues. On the other hand, COX-II is not always expressed and is induced by tumor promoter, growth factor, cytokine, and the like.

Among the antagonist of COX, traditional non steroidal anti-inflammatory compounds (NSAIDs) have inhibiting activities of both COX-I and COX-II (J. Biol. Chem., 268, pp.6610-6614(1993), etc). So, the therapeutic use thereof can cause undesired effects on the gastrointestinal tract, such as bleeding, erosions, gastric and intestinal ulcers, etc.

It was reported that selective inhibition of COX-II shows anti-inflammatory and analgesic activities comparable with conventional NSAIDs but with a lower incidence of some gastrointestinal undesired effects (Pro. Nat. Acad. Sci. USA, 91, pp.3228-3232(1994)). Accordingly, various selective COX-II inhibitors have been prepared.

However, it was also reported that those “selective COX-II inhibitors” show some side-effects on kidney and/or insufficient efficacy on acute pains. Therefore, some compounds such as SC-560, mofezolac, etc., which have certain selective inhibiting activity against COX-I, have been researched.

WO98/57910 also shows some compounds having such selective activity. However, their selectivity of inhibiting COX-I does not seem to be enough to use them as a clinically acceptable and satisfactory analgesic agent due to their gastrointestinal disorders.

And, WO02/055502 shows some pyridine derivatives having cyclooxygenase inhibiting activity, particularly cyclooxygenase-I inhibiting activity. WO99/51580 shows some triazole derivatives having an inhibiting activity of cytokine production, and WO03/040110 shows some triazole derivatives having cyclooxygenase inhibiting activity, particularly cyclooxygenase-I inhibiting activity.

Further, WO92/21664, WO92/21665 and U.S. Pat. No. 4,051,250 show oxazole derivatives having anti/inframmatory activity.

However, the compounds described in WO92/21664 and WO92/21665 have necessarily hydroxylamino group in their structure and the compouns described in U.S. Pat. No. 4,051,250 have alkyl thio group substituted by carboxy, ester, —CONH₂ or CN group.

### BRIEF SUMMARY OF THE INVENTION

As a result of studies on the synthesis of new compounds and their pharmaceutical activity, the inventors of this invention have found that the new compounds of this invention have superior activity of inhibiting COX (especially, COX-I inhibiting activity). So, this invention relates to new compounds, which have pharmaceutical activity such as COX inhibiting activity, to a medicament and a pharmaceutical composition containing the new compounds.

Accordingly, one object of this invention is to provide the new compounds, a method for producing the same, a medicament and a pharmaceutical composition, which have a COX inhibiting activity (especially, COX-I inhibiting activity).

Another object of this invention is to provide a method for treatment and/or prevention of the diseases or conditions associated with COX and the new compounds for use as medicament in the treatment and/or prevention of the diseases or conditions associated with COX.

A further object of this invention is to provide a use of the new compounds for treating or preventing the diseases or conditions, and a use of the compounds for manufacturing a medicament for treating or preventing the diseases or conditions.

A further object of this invention is to provide an analgesic agent comprising the new compounds which is usable for treating and/or preventing the diseases or conditions.

A further object of this invention is to provide the commercial package comprising the pharmaceutical composition containing the new compound.

The new compounds of this invention can be represented by the following general formula (I):

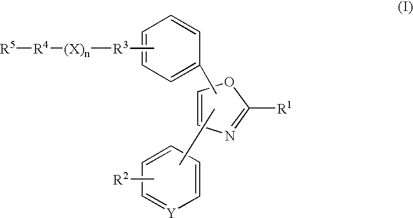

wherein

R¹ is hydrogen, (lower)alkyl, (lower)alkyl substituted with substituent(s) (i) described later, (lower)alkenyl, (lower) alkynyl, cycloalkyl, aryl, saturated heterocyclyl, heteroaryl, (lower)alkoxy, (lower)alkoxy substituted with substituent(s) (i) described later, (lower)alkenyloxy, (lower)alkynyloxy, cycloalkyloxy, aryloxy, heteroaryloxy, (saturated heterocyclyl)oxy, amino, [(lower)alkyl]amino, di[(lower)alkyl]amino, di[(lower)alkyl]amino substituted with substituent(s) (i) described later on (lower)alkyl, [(lower)acyl]amino, cycloalkylamino, arylamino, (saturated heterocyclyl)amino, heteroarylamino, carbamoyl, carbamoyl substituted with substituent(s) (ii) described later, (lower)acyl, cycloalkylcarbonyl, arylcarbonyl, (saturated heterocyclyl)carbonyl, heteroarylcarbonyl, [(lower)alkoxy]carbonyl, [(lower)alkyl]thio, [(lower)alkyl]thio substituted with substituent(s) (i) described later, [(lower)alkyl]sulfinyl, [(lower)alkyl]sulfonyl, cyano, carboxy, hydroxy, mercapto or halogen;

R² is (lower)alkyl, saturated heterocyclyl, (lower)alkoxy or cyano;

R³ is (lower)alkylene, (lower)alkenylene, or covalent bond;

R⁴ is (lower)alkylene, (lower)alkenylene, or covalent bond;

R⁵is hydrogen, (lower)alkyl, aryl, heteroaryl, (lower)alkoxy, [(lower)acyl]oxy, [(lower)alkyl]sulfonyloxy, [tri(lower)alkyl]silyloxy, amino, [(lower)alkyl]amino, di[(lower)alkyl]amino, [(lower)acyl]amino, [(lower)alkoxy]carbonylamino, [(lower)alkyl]sulfonylamino, heteroarylthiocarbonylamino, carbamoylamino, carbamoylamino substituted with substituent(s) (ii) described later on carbamoyl, aryloxycarbonylamino (which may be substituted with substituent(s) (iii) described later on aryl), [(lower)alkoxy]carbonyl, hydroxy, cyano or azido;

X is “O”, “S”, “SO”, or “SO₂”;

Y is “CH” or “N”;

n is 0 or 1;

substituent(s) (i) is(are) selected from the group consisting of (lower)alkyl, cycloalkyl, aryl, heteroaryl, (lower)alkoxy, [(lower)acyl]oxy, aryl[(lower)alkyl]oxy, [(lower)alkyl]sulfonyloxy, amino, [(lower)alkyl]amino, di[(lower)alkyl]amino, [(lower)acyl]amino, carbamoylamino, [(lower)alkylcarbamoyl]amino, [di(lower)alkylcarbamoyl]amino, [(lower)alkoxycarbonyl]amino, [(lower)alkoxy]carbonyl, [(lower)alkyl]thio, arylthio, heteroarylthio, carboxy, hydroxy, hydroxyimino and halogen;

substituent(s) (ii) is(are) selected from the group consisting of (lower)alkyl, (lower)alkyl substituted with hydroxy, (lower)alkyl substituted with carbamoyl, (lower)alkyl substituted with (lower)alkoxy, (lower)alkoxy, amino, [(lower)alkyl]amino and di[(lower)alkyl]amino;

substituent(s) (iii) is(are) selected from the group consisting of (lower)alkyl, (lower)alkoxy, nitro and cyano;

or pharmaceutically acceptable salts thereof.

In the above and subsequent description of this specification, suitable examples of the various definitions to be included within the scope of the invention are explained in detail in the following.

The term “lower” is intended to mean a group having 1 to 6 carbon atom(s), unless otherwise provided.

So, the “(lower)alkyl” means a straight or branched chain aliphatic hydrocarbon, such as methyl, ethyl, propyl, isopropyl, butyl, isobutyl, tert-butyl, pentyl, isoamyl, hexyl, and the like, and it is preferably (C1-C4)alkyl, more preferably (C1-C2)alkyl, most preferably methyl.

The “(lower)alkenyl” means a straight or branched chain aliphatic hydrocarbon having more than one double bond between two carbon atoms, such as ethenyl, propenyl, isopropenyl, butenyl, isobutenyl, pentenyl, hexenyl, and the like, and it is preferably (C2-C4)alkenyl, more preferably (C2-C3)alkenyl.

The “(lower)alkynyl” means a straight or branched chain aliphatic hydrocarbon having more than one triple bond between two carbon atoms, such as ethynyl, propynyl, butynyl, pentynyl, hexynyl, and the like, and it is preferably (C2-C4)alkynyl, more preferably (C2-C3)alkynyl.

The “cycloalkyl” means (C3-C10)cycloalkyl group, such as cyclopropyl, cyclobutyl, cyclopentyl, cyclohexyl, cycloheptyl, norbornyl, adamantyl, and the like, and it is preferably (C3-C6)cycloalkyl, more preferably (C3-C5)cycloalkyl, most preferably cyclopropyl.

The “aryl” means an aromatic hydrocarbon group, such as phenyl, naphtyl, indenyl, or the like, and it is preferably (C6-C10)aryl, more preferably phenyl.

The “saturated heterocyclyl” means 5- or 6-membered saturated heterocyclyl group which contains at least one hetero atom such as nitrogen, oxygen, or sulfur atom. And the “saturated heterocyclyl” may be substituted with general substituent such as (lower)alkyl. The “saturated heterocyclyl” may include 5-membered saturated heterocyclyl group such as pyrrolidinyl, methylpyrrolidinyl, imidazolidinyl, pyrazolidyl, tetrahydrofuranyl, tetrahydrothiophenyl, oxazolidyl, isoxazolidyl, thiazolidyl, isothiazolidyl, or the like; and 6-membered saturated heterocyclyl group such as piperidyl, piperazinyl, tetrahydropyranyl, pentamethylene sulfide, morpholinyl, or the like.

The “heteroaryl” means 5-, 6-membered or condensed polycyclic aromatic heterocyclyl group which contains at least one hetero atom such as nitrogen, oxygen, sulfur atom. The “heteroaryl” may include 5-membered heteroaryl group such as pyrrolyl, imidazolyl, pyrazolyl, tetrazolyl, thienyl, furyl, oxazolyl, isoxazolyl, thiazolyl, isothiazolyl, or the like; 6-membered heteroaryl group such as pyridyl, pyrazinyl, pyrimidinyl, pyridazinyl, or the like; and condensed polycyclic heteroaryl group such as indolyl, isoindolyl, isoindole-1,3-dione-2-yl, quinolyl, isoquinolyl, benzofuranyl, chromenyl, benzothienyl, tetrahydroimidazo[1,2-a]pyrazine, or the like; and is preferably condensed polycyclic aromatic heterocyclic group, more preferably isoindole-1,3-dione-2-yl.

The “(lower) alkoxy” means a straight or branched chain aliphatic hydrocarbon oxy group, such as methoxy, ethoxy, propoxy, isopropoxy, butoxy, isobutoxy, tert-butoxy, pentoxy, hexoxy, and the like, and it is preferably (C1-C4)alkoxy, more preferably (C1-C2)alkoxy, most preferably methoxy.

The “(lower) alkenyloxy” and “(lower) alkynyloxy” mean oxy group subsutituted with the above (lower)alkenyl and (lower)alkynyl, respectively. And “cycloalkyloxy”, “aryloxy”, “heteroaryloxy” and “(saturated heterocyclyl)oxy” mean oxy group subsutituted with the above cycloalkyl, aryl, heteroary and saturated heterocyclyl, respectively.

The “[(lower)alkyl]amino”, “di[(lower)alkyl]amino”, “cycloalkylamino”, “arylamino”, “(saturated heterocyclyl)amino” and “heteroarylamino” mean amino group subsutituted with the above one (lower)alkyl, two (lower)alkyls, cycloalkyl, aryl, saturated heterocyclyl and heteroaryl, respectively.

The “(lower)acyl” means a formyl and a (lower)alkyl carbonyl group, such as acetyl, propionyl, butyryl, isobutyryl, valeryl, isovaleryl, pivaloyl, hexanoyl, and the like, and it is preferably (C1-C4)acyl (including formyl), more preferably (C1-C2)acyl, most preferably acetyl.

The “[(lower)acyl]amino” means an amino group substituted with (lower) acyl group mentioned above, such as formylamino, acetylamino, propionylamino, butyrylamino, isobutyrylamino, valerylamino, isovalerylamino, pivaloylamino, hexanoylamino, and the like, and it is preferably [(C1-C4)acyl]amino, more preferably [(C1-C2)acyl]amino, most preferably acetylamino.

The “cycloalkylcarbonyl”, “arylcarbonyl”, “(saturated heterocyclyl)carbonyl”, “heteroarylcarbonyl” and “[(lower)alkoxy]carbonyl” mean carbonyl group substituted with the above cycloalkyl, aryl, saturated heterocyclyl, heteroaryl and (lower)alkoxy, respectively.

The “[(lower)alkyl]thio”, “[(lower)alkyl]sulfinyl” and “[(lower)alkyl]sulfonyl” mean thio group, sulfinyl group and sulfonyl group substituted with the above (lower)alkyl, respectively.

The “(lower)alkylene” means a straight or branched chain aliphatic hydrocarbon divalent group, such as methylene, ethylene, propylene, methylethylene, butylene, methylpropylene, dimethylpropylene, pentylene, hexylene, and the like, and it is preferably (C1-C4)alkylene, more preferably (C1-C3)alkylene, most preferably (C1-C2)alkylene.

The “(lower)alkenylene” means a straight or branched chain aliphatic hydrocarbon divalent group having more than one double bond between two carbon atom, such as ethenylene, propenylene, methylethenylene, butenylene, methylpropenylene, dimethylpropenylene, pentenylene, hexenylene, and the like, and it is preferably (C2-C4)alkenylne, more preferably (C2-C3)alkenylne.

The “[(lower)acyl]oxy” means an oxy group substituted with (lower)acyl group mentioned above, such as formyloxy, acetyloxy, propionyloxy, butyryloxy, isobutyryloxy, valeryloxy, isovaleryloxy, pivaloyloxy, hexanoyloxy, and the like, and it is preferably (C1-C4)acyloxy, more preferably (C1-C2)acyloxy, most preferably acetyloxy.

The “[(lower)alkyl]sulfonyloxy” means a sulfonyloxy group substituted with (lower)alkyl group mentioned above, such as methanesulfonyloxy, ethanesulfonyloxy, propanesulfonyloxy, butanesulfonyloxy, hexanesulfonyloxy, and the like, and it is preferably [(C1-C4)alkyl]sulfonyloxy, more preferably [(C1-C2)alkyl]sulfonyloxy, most preferably methanesulfonyloxy.

The “[tri(lower)alkyl]silyloxy” means silyloxy group substituted with three (lower)alkyls mentioned above on silicon atom. The three(lower)alkyls may be the same or defferent each other. Such “[tri(lower)alkyl]silyloxy” includes trimethylsilyloxy and tert-butyldimethylsilyloxy, and it is preferably [(C1-C4)alkyl]silyloxy.

The “[(lower)alkoxy]carbonyl” means a [(lower)alkyl]—OCO— group, such as methoxycarbonyl, ethoxycarbonyl, propoxycarbonyl, isopropoxycarbonyl, butoxycarbonyl, isobutoxycarbonyl, tert-butoxycarbonyl, pentoxycarbonyl, hexoxycarbonyl, and the like, and it is preferably [(C1-C4)alkoxy]carbonyl, more preferably etoxycarbonyl.

The “[(lower)alkoxy]carbonylamino” means an amino group substituted with [(lower)alkoxy]carbonyl group mentioned above, such as methoxycarbonylamino, ethoxycarbonylamino, propoxycarbonylamino, isopropoxycarbonylamino, butoxycarbonylamino, isobutoxycarbonylamino, tert-butoxycarbonylamino, pentoxycarbonylamino, hexoxycarbonylamino, and the like, and it is preferably [(C1-C4)alkoxy]carbonylamino, more preferably tert-butoxycarbonylamino.

The “[(lower)alkyl]sulfonylamino” means a sulfonylamino group substituted on the sulfonyl group with (lower)alkyl group mentioned above, such as methanesulfonylamino, ethanesulfonylamino, propanesulfonylamino, butanesulfonylamino, hexanesulfonylamino, and the like, and it is preferably [(C1-C4)alkyl]sulfonylamino, more preferably [(C1-C2)alkyl]sulfonylamino, most preferably methanesulfonylamino.

The “heteroarylthiocarbonylamino” means an amino group substituted with heteroarylthiocarbonyl group, such as (5-membered heteroaryl)thiocarbonylamino such as pyrrolylthiocarbonylamino, imidazolylthiocarbonylamino, pyrazolylthiocarbonylamino, tetrazolylthiocarbonylamino, or the like; (6-membered heteroaryl)thiocarbonylamino; and (condensed polycyclic heteroaryl)thiocarbonylamino.

The “aryloxycarbonylamino” means an amino group substituted with aryloxycarbonyl group such as phenyloxycarbonylamino.

The “aryl[(lower)alkyl]oxy” means a (lower)alkoxy group substituted with aryl group mentioned above, such as benzyloxy, phenethyloxy, phenylpropyloxy, phenylbutyloxy, naphthylmethyloxy, or the like, and it is preferably aryl[(C1-C4)alkyl]oxy, more preferably aryl[(C1-C2)alkyl]oxy, more preferably phenyl[(C1-C2)alkyl]oxy, most preferably benzyloxy.

The “[(lower)alkylcarbamoyl]amino” means carbamoylamino group substituted with a (lower)alkyl group mentioned above on the nitorogen atom in the carbamoyl, such as methylcarbamoylamino, ethylcarbamoylamino, isopropylcarbamoylamino, tert-butylcarbamoylamino, and the like, and it is preferably [(C1-C4)alkylcarbamoyl]amino, more preferably [(C1-C2)alkylcarbamoyl]amino.

The “[di(lower)alkylcarbamoyl]amino” means carbamoylamino group substituted with two (lower)alkyl groups mentioned above on the nitorogen atom in the carbamoyl, such as dimethylcarbamoylamino, ethylmethylcarbamoylamino, diethylcarbamoylamino, and the like, and it is preferably [di(C1-C4)alkylcarbamoyl]amino, more preferably [di(C1-C2)alkylcarbamoyl]amino.

The “[(lower)alkoxycarbonyl]amino” means an amino group substituted with [(lower)alkoxy]carbonyl group mentioned above, such as methoxycarbonylamino, ethoxycarbonylamino, isopropoxycarbonylamino, tert-butoxycarbonylamino, and the like, and it is preferably [(C1-C4)alkoxy]carbonylamino.

The “arylthio” and “heteroarylthio” mean thio group subsutituted with the above aryl and heteroaryl, respectively.

The “halogen” may include a fluorine atom, a chlorine atom, a bromine atom and an iodine atom, and is preferably a fluorine atom or a chlorine atom, more preferably a fluorine atom.

The (lower)alkyl, (lower)alkoxy, di[(lower)alkyl]amino and [(lower)alkyl]thio in the definition of R¹ may be substituted with substituent(s) (i). The carbamoyl in the definition of R¹ and carbamoylamino in the definition of R⁵ may be substituted with substituent(s) (ii). And the aryloxycarbonylamino in the definition of R⁵ may be substituted with substituent(s) (iii).

And the “(lower)alkyl substituted with hydroxy” may include hydroxymethyl, hydroxyethyl, hydroxypropyl, 1-hydroxyisopropyl, 1-hydroxyisobutyl, 1-hydroxyisoamyl, and the like, and is preferably hydroxy(C1-C4)alkyl, more preferably hydroxy(C1-C2)alkyl.

The “(lower)alkyl substituted with carbamoyl” may include carbamoylmethyl, carbamoylethyl, carbamoylpropyl, carbamoylisopropyl, carbamoylisobutyl, carbamoylisoamyl, and the like, and is preferably carbamoyl(C1-C4)alkyl, more preferably carbamoyl(C1-C2)alkyl.

The “(lower)alkyl substituted with (lower)alkoxy” may include methoxymethyl, and the like, and is preferably (C1-C2)alkyl substituted with (C1-C2)alkoxy, more preferably methoxyethyl.

The “(lower)alkyl substituted with halogen” may include fluoromethyl, chloromethyl, difluoromethyl, dichloromethyl, dibromomethyl, trifluoromethyl, trichloromethyl, fluoroethyl, chloroethyl, 2,2,2-trifluoroethyl, 2,2,2-trichloroethyl, 2,2,3,3,3-pentafluoroethyl, fluoropropyl, fluorobutyl, fluorohexyl, and the like, and is preferably (C1-C4)alkyl substituted with fluorine(s), more preferably (C1-C2)alkyl substituted with fluorine(s), more preferably methyl substituted with fluorine(s), most preferably difluoromethyl or trifluoromethyl.

In case of the number of “substituent(s) (i) to (iii)” are plural, they may be same or different each other. For example, R¹ may be hydroxy(phenyl)methyl.

The compounds of formula (I) may contain one or more asymmetric centers and thus they can exist as enantiomers or diastereoisomers. This invention includes both mixtures and separate individual isomers.

The compounds of the formula (I) may also exist in tautomeric forms and the invention includes both mixtures and separate individual tautomers. For example, when R¹ is hydroxy, the compounds of the formula (I) may be tautomeric forms as follows.

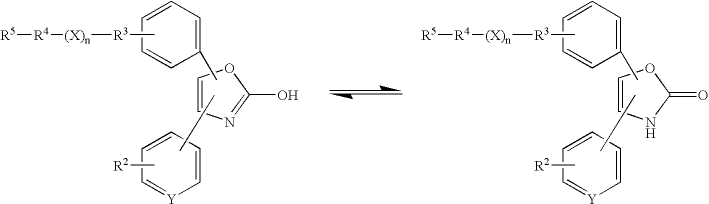

In the scope of this invention, these tautameric forms are included.

The compounds of the formula (I) and its salts can be in a form of a solvate, which is included within the scope of the present invention. The solvate preferably include a hydrate.

Also included in the scope of invention are radiolabelled derivatives of compounds of formula (I) which are suitable for biological studies.

The new compounds of this invention can be converted to salt according to a conventional method. Suitable salts of the compounds (I) are pharmaceutically acceptable conventional non-toxic salts and include a metal salt such as an alkali metal salt (e.g., sodium salt, potassium salt, etc.) and an alkaline earth metal salt (e.g., calcium salt, magnesium salt, etc.), an ammonium salt, an organic base salt (e.g., trimethylamine salt, triethylamine salt, pyridine salt, picoline salt, dicyclohexylamine salt, etc.), an organic acid salt (e.g., acetate, maleate, tartrate, methanesulfonate, benzenesulfonate, formate, toluenesulfonate, trifluoroacetate, etc.), an inorganic acid salt (e.g., hydrochloride, hydrobromide, sulfate, phosphate, etc.), a salt with an amino acid (e.g., arginine, aspartic acid, glutamic acid, etc.), or the like.

### DETAILED DESCRIPTION OF THE INVENTION

The compound of the formula (I) of the present invention can be prepared according to the following processes A-1 to A-3.

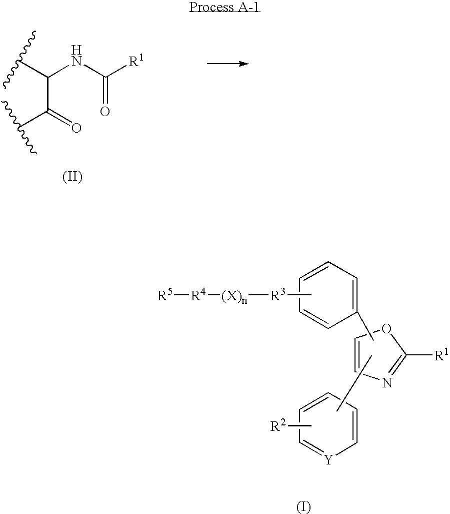

In the above formula, R¹ to R⁵, X, Y and “n” represent the same meanings as defined above. And Compound (II) may have either of following structure.

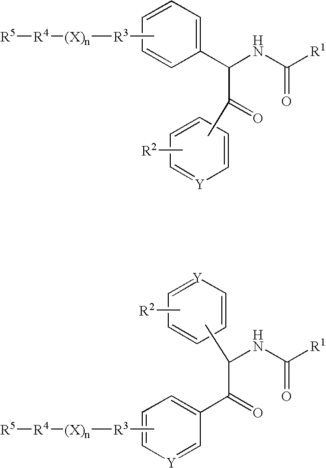

Hereinafter, this condition is the same with Compound (III), (IV), (VI) and (VII).

Process A-1 is the process for preparing Compound (I) from Compound (II) by forming oxazole ring.

Compound (II) may be purchased if it is commercial, or synthesized according to Process B mentioned after or other general methods obvious to the person skilled in the organic chemistry from commercial compounds.

As this process, two methods are mainly employable, which are one using phosphorus oxychloride (POCl₃) as condensation agent (A-1(1)) and the other using triphenylphosphine (A-1(2)).

Process A-1(1) is generally carried out by adding phosphorus oxychloride to the solution of Compound (II). The temperature at that time to be employable depends on the starting material, the solvent, etc., but it is usually room temperature. And after adding, the temperature is preferably raised to reflux.

The solvent employable in Process A-1(1) is not particularly limited so long as it is inactive in this reaction and dissolves moderately Compound (II) and phosphorus oxychloride. It may preferably include liquid hydrocarbon such as benzene, toluene.

The reaction time after adding phosphorus oxychloride depends on the starting material, the solvent, etc., but it is usually from 12 hrs to 3 days.

Process A-1(2) is generally carried out by adding the solution of triphenylphosphine, iodine and base (triethylamine, etc.) to the solution of Compound (II). The temperature at that time depends on the starting material, the solvent, etc., but it is usually room temperature.

The solvent employable in Process A-1(2) is not particularly limited so long as it is inactive in this reaction and can dissolve substrates moderately, and may preferably include halogenated hydrocarbon such as dichloromethane, chloroform, carbon tetrachloride.

The reaction time after adding triphenylphosphine depends on the starting material, the solvent, etc., but it is usually from 12 hrs to 3 days.

After the reaction, the mixture is partitioned between water and organic solvent insoluble with water such as ethyl acetate, chloroform, etc., and the organic layer is separated. The organic layer is washed by water, hydrochloric acid, saturated sodium hydrogencarbonate solution, brine, etc., dried over anhydrous magnesium sulfate or sodium sulfate, and evaporated in vacuo. The target compound is purified by the conventional method such as silica gel column chromatography, etc.

Which to be selected A-1(1) or A-1(2) in this process is mainly dependent on the property of R¹ group. So, either method of which yield is higher may be employed.

Compound (I) can be also synthesized by following Process A-2.

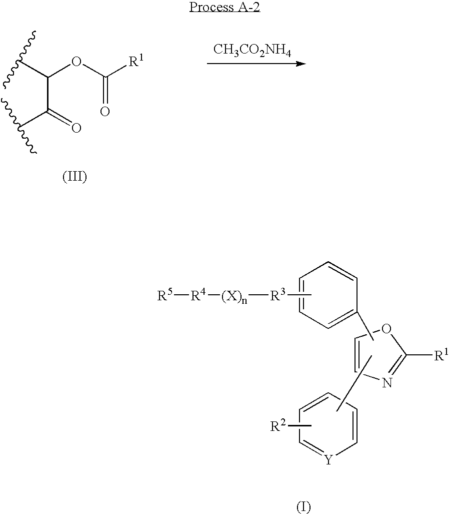

In the above formula, R¹ to R⁵, X, Y and “n” represent the same meanings as defined above.

Process A-2 is the process for preparing Compound (I) from Compound (III) by forming oxazole ring besides Process A-1.

Compound (III) may be purchased if it is commercial, or synthesized according to Process C mentioned after or other general methods obvious to the person skilled in the organic chemistry from commercial compounds.

This process is generally carried out by adding ammonium acetate to the acetic acid solution of Compound (III). The temperature at that time depends on the starting material, the solvent, etc., but it is usually room temperature. And after adding ammonium acetate, the temperature is preferably raised to reflux.

The reaction time after adding ammonium acetate depends on the starting material, the solvent, etc., but it is usually from 30 min to 12 hrs, preferably from 1 hr to 5 hrs.

After the reaction, the solvent is removed in vacuo, and acetic acid is azeotropically removed with toluene, etc. The residue is partitioned between water and organic solvent insoluble with water such as ethyl acetate, chloroform, etc., and the organic layer is separated. The organic layer is washed by water, saturated sodium hydrogencarbonate solution, brine, etc., dried over anhydrous magnesium sulfate or sodium sulfate, and evaporated in vacuo. The target compound is purified by the conventional method such as silica gel column chromatography, etc.

Compound (I) can be transformed to the other Compound (I) by functional group trans formation, which is obvious to the person skilled in the organic chemistry. For example, first, Process A-1 or A-2 are carried out by using the compound which does not have reactive group as R¹ and the like, then, the R¹ and the like are transformed to reactive group. Some of such functional group trans formation reactions are illustrated as following Process A-3.

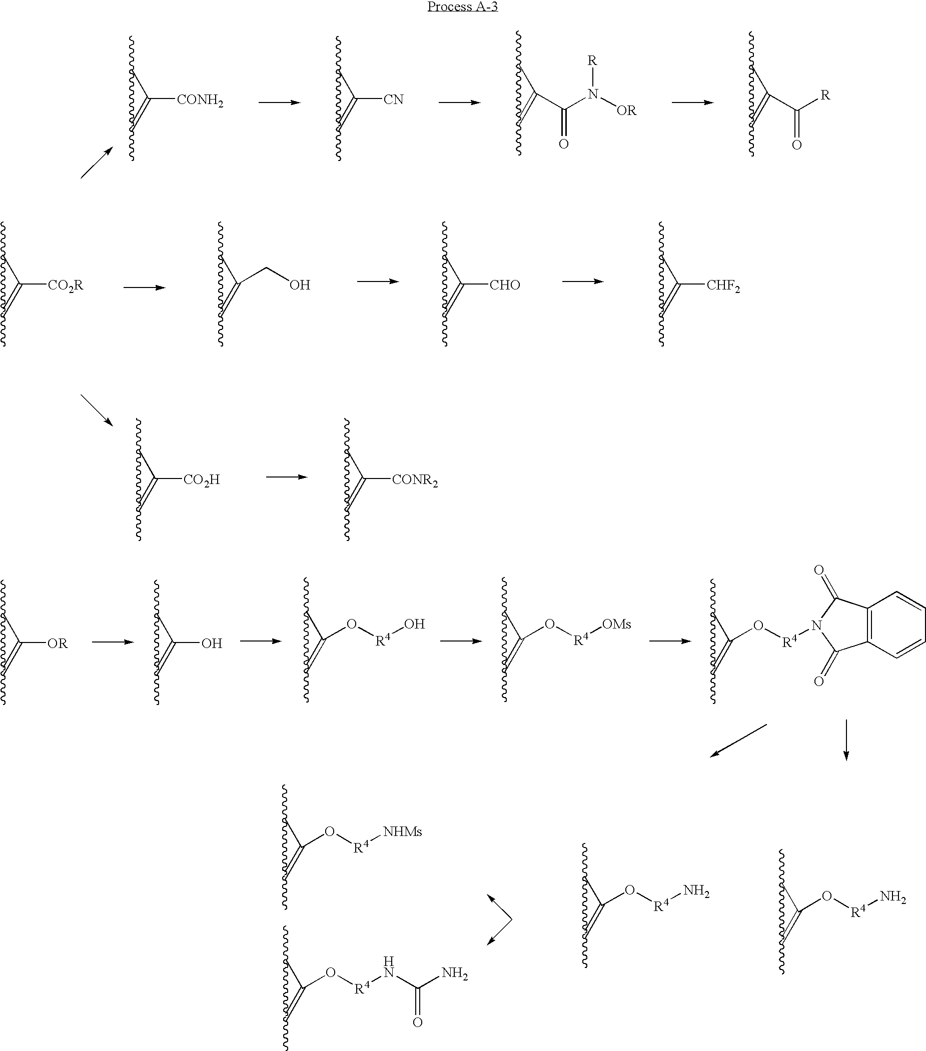

In the above formulae, R represents H, lower alkyl or aryl, which is not specified, and plural R may be same or different each other. “Ms” represents methanesulfonyl group. And R⁴ represents the same meanings as defined above.

Compound (II), which is the starting compound of Process A-1, can be synthesized by following Process B.

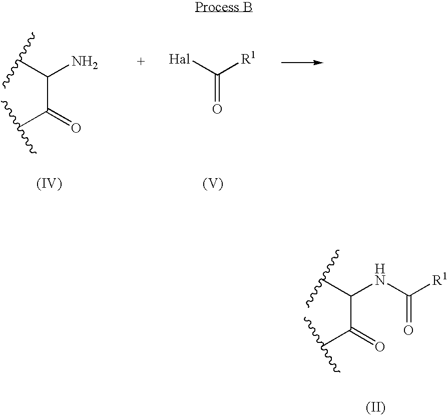

In the above formula, R¹ represents the same meanings as defined above. And “Hal” represents halogen atom, especially, chlorine or bromine atom.

Process B is the process for preparing the Compound (II) by condensing Compound (IV) and (V).

Compound (IV) and (V) may be purchased if it is commercial, or synthesized according to general methods obvious to the person skilled in the organic chemistry from commercial compounds. But, in advance, Compound (V) can be synthesized from corresponding acid and pivaloyl chloride or oxallyl chloride, or the like, in one-pot. And corresponding acid anhydride may be also used as Compound (V).

This process is generally carried out by adding Compound (V) to the solution of Compound (IV). To accelerate the reaction, base such as pyridine may be added. The temperature at that time depends on the starting material, the solvent, etc., but it is usually 0° C. to room temperature. And after adding, the temperature may be raised to reflux.

The solvent employable in Process B is not particularly limited so long as it is inactive in this reaction and dissolves moderately substrates, and may include preferably halogenated hydrocarbon such as dichloromethane, chloroform; liquid hydrocarbon such as benzene, toluene; ethers such as diisopropyl ether, tetrahydrofuran, dioxane.

The reaction time after the adding depends on the starting material, the solvent, etc., but it is usually from 1 hr to 3 days.

After the reaction, the mixture is partitioned between water and organic solvent insoluble with water such as ethyl acetate, chloroform, etc., and the organic layer is separated. The organic layer is washed by water, hydrochloric acid, saturated sodium hydrogencarbonate solution, brine, etc., dried over anhydrous magnesium sulfate or sodium sulfate, and evaporated in vacuo. The target compound is purified by the conventional method such as silica gel column chromatography, etc. However, the target compound may be used in next step (Process A-1) without purification.

Compound (III), which is the starting compound of Process A-2, can be synthesized by following Process C.

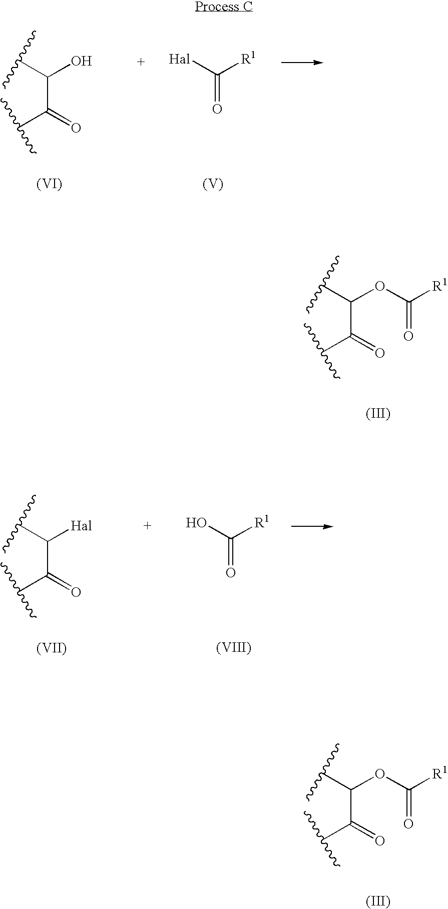

In the above formulae, R¹ and “Hal” represent the same meanings as defined above.

Process C is the process for preparing the Compound (III) in the presence of base.

Compound (V) to (VIII) may be purchased if it is commercial, or synthesized according to general methods obvious to the person skilled in the organic chemistry from commercial compounds, because their structure are comparatively simple.

The above two processes may be generally carried out by almost same condition, that is, by mixing base and Compound (V) and (VI) or Compound (VII) and (VIII) in solvent. The temperature at that time varies depending on the starting material, the solvent, etc., but it is usually room temperature.

The solvent employable in Process C is not particularly limited so long as it is inactive in this reaction and dissolves moderately substrates, and may include preferably halogenated hydrocarbon such as dichloromethane, chloroform; ketone such as acetone, 2-butanone.

The base employable in this process for making basic condition is not particularly limited so long as it accelerates this reaction and may include alkali metal carbonates such as lithium carbonate, sodium carbonate, potassium carbonate; alkaline earth metal carbonates such as magnesium carbonate, calcium carbonate; cesium carbonate; pyridine.

The reaction time depends on the starting material, the solvent, etc., but it is usually from 12 hrs to 2 days.

After the reaction, the mixture is partitioned between water and organic solvent insoluble with water such as ethyl acetate, chloroform, etc., and the organic layer is separated. The organic layer is washed by water, hydrochloric acid, saturated sodium hydrogencarbonate solution, brine, etc., dried over anhydrous magnesium sulfate or sodium sulfate, and evaporated in vacuo. The target compound is purified by the conventional method such as silica gel column chromatography, etc. However, the target compound may be used in next step (Process A-2) without purification.

Compound (IV), (VI) and (VII) have comparably simple structure. So, these compounds can be synthesized according to general methods obvious to the person skilled in the organic chemistry from commercial compounds. For example, these compounds can be synthesized by referring following Process D.

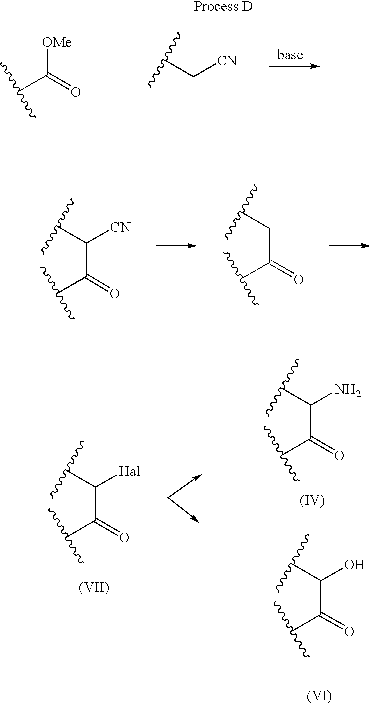

Above Processes A to D, all starting materials and product compounds may be salts. The compounds of above processes can be converted to salt according to a conventional method.

And above Processes A to D, compounds, which have reactive group, may be protected at the group on cue, and be deprotected on cue. In these reactions (protecting or deprotecting steps), concerning the kind of protective group and the condition of the reaction, ┌PROTECTIVE GROUPS IN ORGANIC SYNTHESIS Second Edition┘ T. W. Green and P. G. M. Wuts, John Wiley & Sons, INC. (the contents of which are hereby incorporated by reference) may be referred.

The patents, patent applications and publications cited herein are incorporated by reference.

For therapeutic purpose, the compound (I) and a pharmaceutically acceptable salt thereof of the present invention can be used in a form of pharmaceutical preparation containing said compounds as an active ingredient, in admixture with a pharmaceutically acceptable carrier such as an organic or inorganic solid or liquid excipient suitable for oral, parenteral or external administration. The pharmaceutical preparations may be capsules, tablets, dragees, granules, inhalant, suppositories, solution, lotion, suspension, emulsion, ointment, gel, cream, or the like. If desired, there may be included in these preparations, auxiliary substances, stabilizing agents, wetting or emulsifying agents, buffers and other commonly used additives.

For therapeutic purpose, the analgesic agent of the present invention can be used in a form of pharmaceutical preparation suitable for oral, parenteral or external administration. The pharmaceutical preparations may be capsules, tablets, dragees, granules, inhalant, suppositories, solution, lotion, suspension, emulsion, ointment, gel, or the like.

Particularly, the analgesic agent of this invention is useful for treating or preventing acute or chronic pains associated with acute or chronic inflammations in human beings or animals by using administered systemically or topically.

While the dosage of therapeutically effective amount of the compound (I) will depends on the age and condition of each individual patient, an average single dose of about 0.01 mg, 0.1 mg, 1 mg, 10 mg, 50 mg, 100 mg, 250 mg, 500 mg and 1000 mg of the compound (I) may be effective for treating the above-mentioned diseases. In general, amounts between 0.01 mg/body and about 1,000 mg/body may be administered per day.

The compound (I) includes a compound of the formula (Ia) and (Ib), and is preferably compound of the formula (Ia):

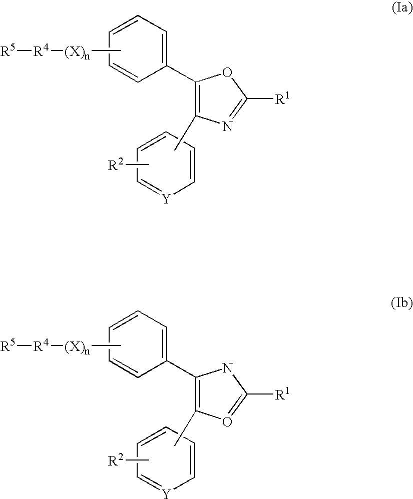

Additionally, the compound (I) includes compound of the formula (Ic) and (Id), and is preferably a compound of the formula (Ic):

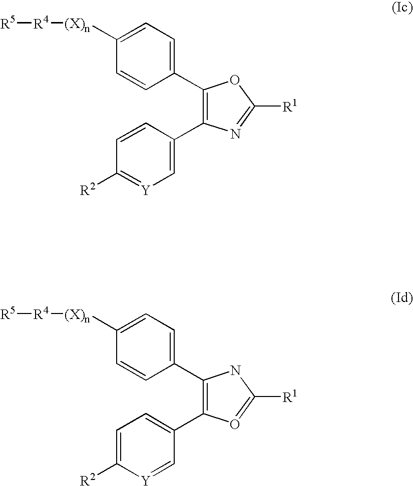

[in the above formulae, R¹ to R⁵, X, Y and n represent the same meanings as defined above.]

And in the each definition of the compound formula(I), preferably,

(1) R¹ is hydrogen, (lower)alkyl, (lower)alkyl substituted with substituent(s) (i), cycloalkyl, heteroaryl, (lower)alkoxy, (lower)alkoxy substituted with substituent(s) (i), (lower)alkynyloxy, cycloalkyloxy, heteroaryloxy, di[(lower)alkyl]amino, di[(lower)alkyl]amino substituted with substituent(s) (i) on (lower)alkyl, [(lower)acyl]amino, heteroarylamino, carbamoyl, carbamoyl substituted with substituent(s) (ii), (lower)acyl, cycloalkylcarbonyl, arylcarbonyl, heteroarylcarbonyl, [(lower)alkoxy]carbonyl, [(lower)alkyl]thio, [(lower)alkyl]thio substituted with substituent(s) (i), [(lower)alkyl]sulfinyl, [(lower)alkyl]sulfonyl, cyano, carboxy or halogen,

(2) R¹ is (C1-C4)alkyl, (C1-C4)alkyl substituted with substituent(s) (i), cycloalkyl or heteroaryl,

(3) R¹ is (lower)alkyl substituted with halogen(s), or cycloalkyl,

(4) R¹ is (C1-C4)alkyl, or cycloalkyl,

(5) R¹ is (C1-C4)alkyl substituted with substituent(s) (i),

(6) R¹ is (C1-C4)alkoxy, (C1-C4)alkoxy substituted with substituent(s) (i), (C1-C4)alkynyloxy, (C3-C6)cycloalkyloxy or heteroaryloxy,

(7) R¹ is (C1-C4)alkoxy substituted with substituent(s) (i),

(8) R¹ is di[(C1-C4)alkyl]amino, di[(C1-C4)alkyl]amino substituted with substituent(s) (i) on (lower)alkyl, [(lower)acyl]amino or heteroarylamino,

(9) R¹ is di[(C1-C4)alkyl]amino substituted with substituent(s) (i),

(10) R¹ is carbamoyl substituted with substituent(s) (ii),

(11) R¹ is (lower)acyl,

(12) R¹ is [(lower)alkyl]thio substituted with substituent(s) (i),

(13) R² is (lower)alkoxy, or cyano,

(14) R² is (lower)alkoxy,

(15) R² is (C1-C4)alkoxy,

(16) R³ is (lower)alkylene, or covalent bond,

(17) R³ is (lower)alkylene,

(18) R³ is (C1-C4)alkylene,

(19) R³ is covalent bond,

(20) R⁴ is (lower)alkylene, or covalent bond,

(21) R⁴ is (lower)alkylene,

(22) R⁴ is (C1-C4)alkylene,

(23) R⁴ is covalent bond,

(24) R⁵ is hydrogen, aryl, heteroaryl, [(lower)acyl]oxy, [(lower)alkyl]sulfonyloxy, [tri(lower)alkyl]silyloxy, amino, [(lower)acyl]amino, [(lower)alkoxy]carbonylamino, carbamoylamino, carbamoylamino substituted with substituent(s) (ii) on carbamoyl, [(lower)alkyl]sulfonylamino, [(lower)alkoxy]carbonyl, aryloxycarbonylamino (which may be substituted with substituent(s) (iii) on aryl), hydroxy, cyano or azido,

(25) R⁵ is hydrogen,

(26) R⁵ is aryl or heteroaryl,

(27) R⁵ is [(C1-C4)alkyl]sulfonyloxy or [tri(C1-C4)alkyl]silyloxy,

(28) R⁵ is amino,

(29) R⁵ is carbamoylamino or carbamoylamino substituted with substituent(s) (ii) on carbamoyl,

(30) R⁵ is carbamoylamino substituted with substituent(s) (ii) on carbamoyl,

(31) R⁵ is aryloxycarbonylamino (which may be substituted with substituent(s) (iii) on aryl),

(32) R⁵ is [(lower)alkyl]sulfonylamino, carbamoylamino or hydroxy,

(33) R⁵ is hydroxy,

(34) X is “O”, or “S”,

(35) X is “O”,

(36) X is “SO”, or “SO₂”,

(37) Y is “CH”,

(38) Y is “N”,

(39) n is 0,

(40) n is 1,

(41) substituent(s) (i) is(are) selected from the group consisting of (lower)alkyl, cycloalkyl, (lower)alkoxy, aryl [(lower)alkyl]oxy, [(lower)acyl]oxy, [(lower)alkyl]sulfonyloxy, di[(lower)alkyl]amino, [di(lower)alkylcarbamoyl]amino, heteroarylthio, hydroxy, hydroxyimino and halogen,

(42) substituent(s) (i) is(are) selected from the group consisting of (lower)alkyl and cycloalkyl,

(43) substituent(s) (i) is(are) selected from the group consisting cycloalkyl and hydroxyimino,

(44) substituent(s) (i) is(are) selected from the group consisting of (lower)alkoxy, aryl[(lower)alkyl]oxy, [(lower)acyl]oxy, [(lower)alkyl]sulfonyloxy,

(45) substituent(s) (i) is(are) selected from the group consisting of di[(lower)alkyl]amino and [di(lower)alkylcarbamoyl]amino,

(46) substituent(s) (i) is heteroarylthio,

(47) substituent(s) (i) is(are) selected from the group consisting of hydroxy and halogen,

(48) substituents (ii) is(are) selected from the group consisting of (lower)alkyl and (lower)alkoxy,

(49) substituents (ii) is(are) selected from the group consisting of (lower)alkyl substituted with hydroxy, (lower)alkyl substituted with carbamoyl and (lower)alkyl substituted with (lower)alkoxy,

(50) substituents (ii) is(are) selected from the group consisting of amino and di[(lower)alkyl]amino,

(51) substituent(s) (iii) is(are) selected from the group consisting of nitro and cyano,

(52) provided that R⁵ is not hydrogen, when both of R³ and R⁴ are covalent bond and n is 0.

Preferred compounds of formula (I) may include

2-{4-[2-(Difluoromethyl)-4-(4-methoxyphenyl)-1,3-oxazol-5-yl]phenoxy}ethanol,

2-{4-[2-(Difluoromethyl)-4-(6-methoxy-3-pyridinyl)-1,3-oxazol-5-yl]phenoxy}ethanol,

N-(2-{4-[4-(6-Methoxy-3-pyridinyl)-2-(trifluoromethyl)-1,3-oxazo 1-5-yl]phenoxy)ethyl)methanesulfonamide,

N-(2-{4-[4-(6-Methoxy-3-pyridinyl)-2-(trifluoromethyl)-1,3-oxazo 1-5-yl]phenoxy}ethyl)urea,

2-{4-[2-Cyclopropyl-4-(6-methoxy-3-pyridinyl)-1,3-oxazol-5-yl]phenoxy)ethanol and

N-(2-{4-[2-Cyclopropyl-4-(6-methoxy-3-pyridinyl)-1,3-oxazol-5-yl]phenoxy}ethyl)methanesulfonamide.

### EXAMPLES

The following Examples are given only for the purpose of illustrating the present invention in more detail.

### Example 1-1

### Ethyl {[1,2-bis(4-methoxyphenyl)-2-oxoethyl]amino}-(oxo)acetate

To a suspension of 2-amino-1,2-bis(4-methoxyphenyl)ethanone hydrochloride (1.0 g, 3.25 mmol) in benzene (10 mL) was added ethyl chlorooxoacetate (532 mg, 3.90 mmol) at room temperature and the mixture was heated to reflux with stirring for 2 days.

After cooling, the reaction mixture was partitioned between water and ethyl acetate. The organic layer was separated; washed with 1 mol/L hydrochloric acid, water, saturated sodium bicarbonate solution and brine, dried over magnesium sulfate and evaporated in vacuo to give the title compound (1.25 g, 103.6%) as an oil.

¹H-NMR (300 MHz, CDCl₃): δ 1.37(3H, t, J=7.5 Hz), 3.75(3H, s), 3.83(3H, s), 4.34(2H, q, J=7.5 Hz), 6.42(1H, d, J=7.5 Hz), 6.83(2H, d, J=8 Hz), 6.87(2H, d, J=8 Hz), 7.34(2H, d, J=8 Hz), 7.95(2H, d, J=8 Hz), 8.49(1H, d, J=7.5 Hz). MS (ES+): 372.14.

### Example 1-2

### Ethyl 4,5-bis(4-methoxyphenyl)-1,3-oxazole-2-carboxylate

To a solution of ethyl {[1,2-bis(4-methoxyphenyl)-2-oxoethyl]amino}(oxo)acetate obtained by Example 1-1 (1.25 g, 3.37 mmol) in benzene (15 mL) was added phosphorus oxychloride (1.55 g, 10.1 mmol) at room temperature and the mixture was heated to reflux with stirring for 18 hrs.

After cooling, the reaction mixture was partitioned between water and ethyl acetate. The organic layer was separated, washed with 1 mol/L hydrochloric acid, water, saturated sodium bicarbonate solution and brine, dried over magnesium sulfate and evaporated in vacuo. The residue was purified by silica gel column chromatography (n-hexane:ethyl acetate=4:1) to give the title compound (909 mg, 76.4%) as a pale yellow powder.

MP: 95-97° C. ¹H-NMR (300 MHz, CDCl₃): δ 1.46(3H, t, J=7.5 Hz), 3.84(3H, s), 3.85(3H, s), 4.51(2H, q, J=7.5 Hz), 6.91(4H, d-like, J=8 Hz), 7.58-7.62(4H, m). MS (ES+): 354.10.

### Example 2

### 4,5-Bis(4-methoxyphenyl)-1,3-oxazole-2-carboxamide

A mixture of ethyl 4,5-bis(4-methoxyphenyl)-1,3-oxazole-2-carboxylate obtained by Example 1-2 (400 mg, 1.13 mmol) and sodium methoxide (183 mg, 3.40 mmol) in formamide (4 mL) was stirred at 100° C. for 2 hrs.

After cooling to room temperature, the reaction mixture was poured into water and extracted with ethyl acetate. The organic layer was washed with water, saturated sodium bicarbonate solution and brine, dried over magnesium sulfate, and evaporated in vacuo. The residue was triturated with isopropyl ether to give the title compound (264 mg, 71.9%) as a pale yellow powder.

MP: 133-135° C. ¹H-NMR (300 MHz, CDCl₃): δ 3.79(3H, s), 3.81(3H, s), 7.00(2H, d, J=8 Hz), 7.05(2H, d, J=8 Hz), 7.52(2H, d, J=8 Hz), 7.55(2H, d, J=8 Hz), 7.93(1H, br-s), 8.30(1H, br-s). MS (ES+): 325.10.

### Example 3

### 4,5-Bis(4-methoxyphenyl)-1,3-oxazole-2-carbonitrile

A mixture of 4,5-bis(4-methoxyphenyl)-1,3-oxazole-2-carboxamide obtained by Example 2 (239 mg, 0.737 mmol) and phosphorus oxychloride (339 mg, 2.21 mmol) in N,N-dimethylformamide (2 mL) was stirred at room temperature for 1 hr.

The reaction mixture was poured into water and extracted with ethyl acetate. The organic layer was washed with water, saturated sodium bicarbonate solution and brine, dried over magnesium sulfate, and evaporated in vacuo. The residue was crystallized from a mixture of ethyl acetate and n-hexane to give the title compound (175 mg, 77.5%) as pale yellow crystals.

MP: 110-112° C. ¹H-NMR (300 MHz, CDCl₃): δ 3.85(3H, s), 3.86(3H, s), 6.94(4H, d, J=9 Hz), 7.55(4H, d, J=9 Hz). IR (KBr): 2240 cm⁻¹.

### Example 4

### N-Methoxy-4,5-bis(4-methoxyphenyl)-N-methyl-1,3-oxazole-2-carboxamide

To a solution of N,O-dimethylhydroxyamine hydrochloride (414 mg, 4.24 mmol) in tetrahydrofuran (8 mL) was added triethylaluminum (15% solution in hexane) dropwise at 0° C. under nitrogen and the mixture was stirred at room temperature for 1 hr. A solution of ethyl 4,5-bis(4-methoxyphenyl)-1,3-oxazole-2-carboxylate obtained by Example 1-2 (500 mg, 1.41 mmol) in tetrahydrofuran (10 mL) was added dropwise to the mixture at 0° C. and the reaction mixture was stirred at 0° C. for 18 hrs.

The mixture was poured into 1 mol/L hydrochloric acid and extracted with ethyl acetate. The organic layer was washed with 1 mol/L hydrochloric acid, water and brine, dried over magnesium sulfate, and evaporated in vacuo. The residue was purified by silica gel column chromatography (n-hexane:ethyl acetate=4:1) to give the title compound (475 mg, 91.1%) as an amorphous powder.

¹H-NMR (300 MHz, CDCl₃): δ 3.53(3H, br peak), 3.85(6H, s), 3.95(3H, s), 6.86-6.95(4H, m), 7.60(4H, s). MS (ES+): 369.53(M+H), 737.39(2M+H), 759.77(2M+Na).

### Example 5

### 1-[4,5-Bis(4-methoxyphenyl)-1,3-oxazol-2-yl]ethanone

To a solution of N-methoxy-4,5-bis(4-methoxyphenyl)-N-methyl-1,3-oxazole-2-carboxamide obtained by Example 4 (120 mg, 0.326 mmol) in tetrahydrofuran (3 mL) was added 1N solution of methylmagnesium bromide in tetrahydrofuran (1.0 mL, 0.95 mmol) dropwise at 0° C. under nitrogen and the mixture was stirred at the same temperature for 2 hrs.

The reaction mixture was poured into water and extracted with ethyl acetate. The organic layer was washed with 1 mol/L hydrochloric acid, water, saturated sodium bicarbonate solution and brine, dried over magnesium sulfate, and evaporated in vacuo. The residue was crystallized from a mixture of ethyl acetate and hexane to give the title compound (63 mg, 59.8%) as pale yellow crystals.

MP: 139-140° C. ¹H-NMR (300 MHz, CDCl₃): δ 2.72(3H, s), 3.85(3H, s), 3.86(3H, s), 6.90(2H, d, J=8 Hz), 6.94(2H, d, J=8 Hz), 7.58(2H, d, J=8 Hz), 7.63(2H, d, J=8 Hz). MS (ES+): 324.40(M+H), 647.68(2M+H).

### Example 6

### [4,5-Bis(4-methoxyphenyl)-1,3-oxazol-2-yl](phenyl)methanone

The title compound (193 mg, 61.5%) as yellow crystals was obtained from N-methoxy-4,5-bis(4-methoxyphenyl)-N-methyl-1,3-oxazole-2-carboxamide obtained by Example 4 (300 mg, 0.814 mmol) and 3N solution of phenylmagnesium bromide in diethyl ether (0.82 mL, 2.46 mmol) in a manner similar to that of Example 5.

MP: 164-166° C. ¹H-NMR (300 MHz, CDCl₃): δ 3.86(3H, s), 3.87(3H, s), 6.93(2H, d, J=8 Hz), 6.96(2H, d, J=8 Hz), 7.49-7.57(2H, m), 7.60-7.71(5H, m), 7.53-7.59(2H, m). MS (ES+): 386.30.

### Example 7-1

### 2-(4-Methoxyphenyl)-3-(6-methoxy-3-pyridinyl)-3-oxopropanenitrile

To a stirred suspension of potassium tert-butoxide (3.69 g, 32.9 mmol) in tert-butanol (40 mL) was added methyl 6-methoxynicotinate (5.0 g, 29.9 mmol) followed by dropwise addition of (4-methoxyphenyl)acetonitrile (4.4 g, 29.9 mmol) in tert-butanol (10 mL) at room temperature. The resulting mixture was heated at 120° C. for 1.5 hrs.

The mixture was allowed to cool and water was added to the mixture (160 mL). The mixture was extracted with ether (100 mL) and the aqueous phase was separated. The aqueous layer was neutralized with hydrogen chloride (37%) and then extracted with ethyl acetate (100 mL). The organic layer was separated, washed with water (100 mL) and brine (100 mL), and dried over anhydrous magnesium sulfate. The solvent was removed in vacuo to give the title compound (6.49 g, 77%) as a brown viscous oil.

¹H-NMR (300 MHz, CDCl₃): δ 3.80(3H, s), 3.99(3H, s), 5.44(1H, s), 6.78(1H, d, J=8.8 Hz), 6.92(2H, d, J=8.8 Hz), 7.35(2H, d, J=8.8 Hz), 8.12(1H, dd, J=8.8, 2.6 Hz), 8.78(1H, d, J=2.6 Hz).

### Example 7-2

### 1-(6-Hydroxy-3-pyridinyl)-2-(4-methoxyphenyl)ethanone

To a stirred solution of 2-(4-methoxyphenyl)-3-(6-methoxy-3-pyridinyl)-3-oxopropanenitrile obtained by Example 7-1 (4.19 g, 14.8 mmol) in 1,4-dioxane (20 mL) was added hydrogen chloride (37%, 40 mL), and the resulting mixture was heated at 80° C. for 20 hrs.

The mixture was allowed to cool and the solvent was removed in vacuo. The residual solid was suspended in water (50 mL) and the suspension was neutralized with saturated sodium bicarbonate solution. The precipitate was filtered and washed with water to afford the title compound (3.17 g, 88%) as a brown solid.

MP: 177-181° C. ¹H-NMR (300 MHz, DMSO-d₆): δ 3.72(3H, s), 4.10(2H, s), 6.37(1H, d, J=9.6 Hz), 6.87(2H, d, J=8.8 Hz), 7.15(2H, d, J=8.8 Hz), 7.87(1H, dd, J=9.6, 2.6 Hz), 8.35(1H, d, J=2.6 Hz).

### Example 7-3

### 1-(6-Chloro-3-pyridinyl)-2-(4-methoxyphenyl)ethanone

A suspension of 1-(6-hydroxy-3-pyridinyl)-2-(4 -methoxyphenyl)ethanone obtained by Example 7-2 (3.80 g, 15.6 mmol) in phosphorous oxychloride (12 mL) was heated at 80° C. for 1 hr.

The mixture was concentrated in vacuo and the residue was poured into ice-water (40 mL). The mixture was neutralized with saturated sodium bicarbonate solution and stirred in ice bath for 1 hr. The precipitate was filtered and washed with water to give the title compound (3.77 g, 92%) as a pale brown solid.

MP: 77-81° C. ¹H-NMR (300MHz, CDCl₃): δ 3.79(3H, s), 4.21(2H, s), 6.87(2H, d, J=8.8 Hz), 7.16(2H, d, J=8.8 Hz), 7.42(1H, d, J=8.4 Hz), 8.20(1H, dd, J=8.8, 2.6 Hz), 8.98(1H, d, J=2.6 Hz). MS (ES+): 262.00(M+1).

### Example 7-4

### 2-(4-Methoxyphenyl)-1-(6-methoxy-3-pyridinyl)ethanone

To a stirred suspension of 1-(6-chloro-3-pyridinyl)-2-(4-methoxyphenyl)ethanone obtained by Example 7-3 (3.66 g, 14 mmol) in methanol (40 mL) was added 5.19M solution of sodium methoxide in methanol (3.0 mL, 15.4 mmol) at room temperature and the resulting mixture was refluxed for 1.5 hrs. Additional 5.19M solution of sodium methoxide in methanol (1.48 mL, 7.7 mmol) was added and the mixture was refluxed for 1.5 hrs. The mixture was allowed to cool, methanol (10 mL) was added to this mixture, and the mixture was neutralized with hydrogen chloride (37%). To the suspension was added water (10 mL) and the mixture was stirred in ice bath for 1 hr. The precipitate was filtered and washed with water (10 mL) three times to afford the title compound (2.96 g, 82%) as an off-white solid.

MP: 101-102° C. ¹H-NMR (300 MHz, CDCl₃): δ 3.78(3H, s), 3.99(3H, s), 4.16(2H, s), 6.77(1H, d, J=8.8 Hz), 6.86(2H, d, J=8.4 Hz), 7.18(2H, d, J=8.4 Hz), 8.16(1H, dd, J=8.8, 2.6 Hz), 8.85(1H, d, J=2.6 Hz). MS (ES+): 258.09(M+1).

### Example 7-5

### 2-Azido-2-(4-methoxyphenyl)-1-(6-methoxy-3-pyridinyl)ethanone

To a solution of 2-(4-methoxyphenyl)-1-(6-methoxy-3-pyridinyl)ethanone obtained by Example 7-4 (3.0 g, 11.7 mmol) in dichloromethane (30 mL) were added pyridinium tribromide (4.1 g, 12.8 mmol) and hydrogen bromide (33% solution in acetic acid, 3 mL) at room temperature under nitrogen, and the mixture was stirred at the same temperature for 40 min.

The reaction mixture was evaporated in vacuo and acetic acid was azeotropically removed with toluene. The residue was partitioned between water and ethyl acetate. The organic layer was separated, washed with water and brine, dried over magnesium sulfate, and evaporated in vacuo.

The residue was dissolved in N,N-dimethylformamide (15 mL). To the solution was added sodium azide (758 mg, 11.7 mmol) at 0° C. and the mixture was stirred at room temperature for 1 hr.

The reaction mixture was poured into water and extracted with ethyl acetate. The organic layer was washed with 1 mol/L hydrochloric acid, water, saturated sodium bicarbonate solution and brine, dried over magnesium sulfate, and evaporated in vacuo. The residue was purified by silica gel column chromatography (n-hexane:ethyl acetate=4:1) to give the title compound (1.5 g, 43.1%) as an oil.

¹H-NMR (300 MHz, DMSO-d₆): δ 3.77(3H, s), 3.92(3H, s), 5.55(1H, s), 6.70(1H, d, J=8 Hz), 6.90(2H, d, J=8 Hz), 7.20-7.40(3H, m), 8.06(1H, dd, J=8.2 Hz), 8.64(1H, d, J=2 Hz). MS (ES+): 299.06.

### Example 7-6

### 2-Amino-2-(4-methoxyphenyl)-1-(6-methoxy-3-pyridinyl)ethanone hydrochloride

A mixture of 2-azido-2-(4-methoxyphenyl)-1-(6-methoxy-3-pyridinyl)ethanone obtained by Example 7-5 (1.5 g, 5.03 mmol), hydrochloric acid (37%, 0.42 mL) and 10% palladium on carbon (300 mg) in methanol (40 mL) was stirred at room temperature under hydrogen for 30 min.

The reaction mixture was filtered through Celite and evaporated in vacuo. The residue was triturated with diethyl ether to give the title compound (1.46 g, 94.0%) as a pale yellow powder.

¹H-NMR (300 MHz, DMSO-d₆): δ 3.73(3H, s), 3.91(3H, s), 6.21-6.34(1H, m), 6.92(1H, d, J=8 Hz), 6.99(2H, d, J=8 Hz), 7.49(2H, d, J=8 Hz), 8.25(1H, dd, J=8.2 Hz), 8.82-8.99(3H, m).

### Example 7-7

### 2-Methoxy-N-[1-(4-methoxyphenyl)-2-(6-methoxy-3-pyridinyl)-2-oxo ethyl]acetamide

To a solution of 2-amino-2-(4-methoxyphenyl)-1-(6-methoxy-3-pyridinyl)ethanone hydrochloride obtained by Example 7-6 (150 mg, 0. 489 mmol) and pyridine (115 mg, 1.46 mmol) in dichloromethane (3 mL) was added methoxyacetyl chloride (74.6 mg, 0.632 mmol) under nitrogen at room temperature, and the mixture was stirred at the same temperature for 2 hrs.

The mixture was pored into 1 mol/L hydrochloric acid and extracted with chloroform. The organic layer was washed with 1 mol/L hydrochloric acid and water, dried over magnesium sulfate, and evaporated in vacuo. The residue was purified by preparative thin layer chromatography (toluene:ethyl acetate=3:1) to give the title compound (100 mg, 59.6%) as an oil.

¹H-NMR (300 MHz, CDCl₃): δ 3.44(3H, s), 3.76(3H, s), 3.92(2H, s), 3.96(3H, s), 6.43(1H, d, J=8 Hz), 6.74(1H, d, J=8 Hz), 6.85(2H, d, J=8 Hz), 7.31(2H, d, J=8 Hz), 7.82(1H, d, J=8 Hz), 8.12(1H, dd, J=8.2 Hz), 8.80 (1H, d, J=2 Hz).

### Example 7-8

### 2-Methoxy-5-[2-(methoxymethyl)-4-(4-methoxyphenyl)-1,3-oxazol-5-yl]pyridine

The title compound (32 mg, 33.8%) was obtained as an amorphous from 2-methoxy-N-[1-(4-methoxyphenyl)-2-(6-methoxy-3-pyridinyl)-2-oxoethyl]acetamide obtained by Example 7-7 (100 mg, 0.29 mmol) in a manner similar to that of Example 1-2.

¹H-NMR (300 MHz, CDCl₃): δ 3.52(3H, s), 3.84(3H, s), 3.97(3H, s), 4.60(2H, s), 6.75(1H, d, J=8 Hz), 6.91(2H, d, J=8 Hz), 7.55(2H, d, J=8 Hz), 7.76(1H, dd, J=8.2 Hz), 8.41(1H, d, J=2 Hz). MS (ES+): 327.07.

### Example 8-1

### 2-{[1-(4-Methoxyphenyl)-2-(6-methoxy-3-pyridinyl)-2-oxoethyl]amino}-2-oxoethyl acetate

The title compound (673 mg, 38%) was obtained from 2-amino-2-(4-methoxyphenyl)-1-(6-methoxy-3-pyridinyl)ethanone hydrochloride obtained by Example 7-6 (1.47 g, 4.76 mmol) and acetoxyacetyl chloride (731 mg, 6.19 mmol) in a manner similar to that of Example 7-7.

¹H-NMR (300 MHz, CDCl₃): δ 2.22(3H, s), 3.76(3H, s), 3.96(3H, s), 4.54(1H, d, J=15 Hz), 4.62(1H, d, J=15 Hz), 6.40(1H, d, J=8 Hz), 6.74(1H, d, J=8 Hz), 6.85(2H, d, J=8 Hz), 7.31(2H, d, J=8 Hz), 7.59(1H, d, J=8 Hz), 8.11(1H, dd, J=8.2 Hz), 8.80(1H, d, J=2 Hz). MS (ES+): 373.06.

### Example 8-2

### [4-(4-Methoxyphenyl)-5-(6-methoxy-3-pyridinyl)-1,3-oxazol-2-yl]methanol

To a solution of 2-{[1-(4-methoxyphenyl)-2-(6-methoxy-3-pyridinyl)-2-oxoethyl]amino}-2-oxoethyl acetate obtained by Example 8-1 (670 mg, 1.8 mmol) in toluene (12 mL) was added phosphorus oxychloride (828 mg, 5.4 mmol) at room temperature, and the mixture was heated to reflux with stirring for 15 hrs.

After cooling, the reaction mixture was partitioned between water and ethyl acetate. The organic layer was separated, washed with 1 mol/L hydrochloric acid, water, saturated sodium bicarbonate solution and brine, dried over magnesium sulfate, and evaporated in vacuo.

The residue was dissolved in methanol. To a solution was added potassium carbonate (49.7 mg) at room temperature and the mixture was stirred at the same temperature for 1 hr.

The reaction mixture was evaporated in vacuo, and the residue was partitioned between water and ethyl acetate. The organic layer was separated, washed with 1 mol/L hydrochloric acid, water, saturated sodium bicarbonate solution and brine, dried over magnesium sulfate, and evaporated in vacuo. The residue was purified by preparative thin layer chromatography (n-hexane:ethyl acetate=1:1) to give the title compound (26 mg, 4.6%) as an amorphous solid.

¹H-NMR (300 MHz, CDCl₃): δ 2.58(1H, t, J=7H), 3.84(3H, s), 3.97(3H, s), 4.81(2H, d, J=7 Hz), 6.75(1H, d, J=8 Hz), 6.91(2H, d, J=8 Hz), 7.53(2H, d, J=8 Hz), 7.74(1H, dd, J=8.2 Hz), 8.40(1H, d, J=2 Hz). MS (ES+): 313.10.

### Example 9-1

### 1-[4-(Benzyloxy)phenyl]-2-bromo-2-(4-methoxyphenyl)ethanone

The title compound (20.65 g, 99.9%) was obtained as an oil from 1-[4-(benzyloxy)phenyl]-2-(4-methoxyphenyl)ethanone (16.7 g, 50.2 mmol) in a manner similar to that of Example 78-3 described later.

¹H-NMR (300 MHz, CDCl₃): δ 3.80(3H, s), 5.12(2H, s), 6.36(1H, s), 6.89(2H, d, J=8 Hz), 6.99(2H, d, J=8 Hz), 7.31-7.50(7H, m), 7.96(2H, d, J=8 Hz).

### Example 9-2

### 2-[2-[4-(Benzyloxy)phenyl]-1-(4-methoxyphenyl)-2-oxoethyl]-1H-isoindole-1,3(2H)-dione

To a solution of 1-[4-(benzyloxy)phenyl]-2-bromo-2-(4-methoxyphenyl)ethanone obtained by Example 9-1 (20.65 g, 50.2 mmol) in N,N-dimethylformamide (200 mL) was added potassium phthalimide (9.3 g, 50.2 mmol) at 0° C., and the mixture was stirred at the same temperature for 2 hrs.

The reaction mixture was poured into water and extracted with ethyl acetate. The organic layer was washed with 1 mol/L hydrochloric acid, water, saturated sodium bicarbonate solution and brine, dried over magnesium sulfate, and evaporated in vacuo. The residue was triturated with ethanol to give the title compound (20.47 g, 85.4%) as a powder.

¹H-NMR (300 MHz, CDCl₃): δ 3.77(3H, s), 5.07(2H, s), 6.70(1H, s), 6.85(2H, d, J=8 Hz), 6.91(2H, d, J=8 Hz), 7.30-7.47(7H, m), 7.65-7.73(2H, m), 7.78-7.88(4H, m).

### Example 9-3

### 2-Amino-1-[4-(benzyloxy)phenyl]-2-(4-methoxyphenyl)ethanone hydrochloride

To a suspension of 2-[2-[4-(benzyloxy)phenyl]-1-(4-methoxyphenyl)-2-oxoethyl]-1H-is oindole-1,3(2H)-dione obtained by Example 9-2 (20.47 g, 42.9 mmol) in ethanol (200 mL) was added hydrazine monohydrate (8.58 g, 171 mmol) at room temperature, and the mixture was heated to reflux with stirring for 30 min.

After cooling, hydrochloric acid (37%, 24 mL) was added to the mixture and the precipitate was filtered off. The filtrate was concentrated in vacuo and the residue was triturated with ethyl acetate to give the title compound (10.62 g, 64.5%) as a powder.

¹H-NMR (300 MHz, DMSO-d₆): δ 3.72(3H, s), 5.18(2H, s), 6.24(1H, br peak), 6.96(2H, d, J=8 Hz), 7.10(2H, d, J=8 Hz), 7.24-7.50(7H, m), 8.00(2H, d, J=8 Hz), 8.77(2H, br peak). MS (ES+): 348.16.

### Example 9-4

N-[2-[4-(Benzyloxy)phenyl]-1-(4-methoxyphenyl)-2-oxoethyl]-2,2-difluoroacetamide

To a mixture of difluoroacetic acid (981 mg, 10.2 mmol) and triethylamine (1.77 g, 17.5 mmol) in tetrahydrofuran (50 mL) was added pivaloyl chloride (1.23 g, 10.2 mmol) dropwise at 0° C. under nitrogen, and the mixture was stirred at the same temperature for 1 hr. 2-Amino-1-[4-(benzyloxy)phenyl]-2-(4-methoxyphenyl)ethanone hydrochloride obtained by Example 9-3 (2.8 g, 7.29 mmol) was added portionwise to the mixture at 0° C. and the reaction mixture was stirred at the same temperature for 2 hrs.

The reaction mixture was evaporated in vacuo, and partitioned between water and ethyl acetate. The organic layer was separated, washed with water, saturated sodium bicarbonate solution and brine, successively, dried over magnesium sulfate. After evaporation of solvent, the residue was purified by silica gel column chromatography (n-hexane:ethyl acetate=3:1) to give the title compound (2.0 g, 64.5%) as an oil.

¹H NMR (300 MHz, CDCl₃): δ 3.76(3H, s), 5.09(2H, s), 5.89(1H, t, J=53 Hz), 6.40(1H,br-s), 6.84(2H, d, J=8 Hz), 6.95(2H, d, J=8 Hz), 7.26-7.43(7H, m), 7.84-7.98(3H, m).

### Example 9-5

### 5-[4-(Benzyloxy)phenyl]-2-(difluoromethyl)-4-(4-methoxyphenyl)-1,3-oxazole

To a mixture of triphenylphosphine (6.88 g, 26.2 mmol), iodine (6.66 g, 26.2 mmol) and triethylamine (5.31 g, 52.5 mmol) in dichloromethane (100 mL) were added a solution of N-[2-[4-(benzyloxy)phenyl]-1-(4-methoxyphenyl)-2-oxoethyl]-2,2-difluoroacetamide obtained by Example 9-4 (5.58 g, 13.1 mmol) in dichloromethane (10 mL) at room temperature under nitrogen, and the mixture was stirred at the same temperature for 2 days.

The reaction mixture was evaporated in vacuo, and partitioned between water and ethyl acetate. The organic layer was separated, washed with 1 mol/L hydrochloric acid, water, saturated sodium bicarbonate solution and brine, successively, dried over magnesium sulfate. After evaporation of solvent, the residue was purified by silica gel column chromatography (n-hexane:ethyl acetate=3:1) and triturated with petroleum ether to give the title compound (3.43 g, 64.2%) as a powder.

¹H-NMR (300 MHz, CDCl₃): δ 3.84(3H, s), 5.10(2H, s), 6.70(H, t, J=53 Hz), 6.91(2H, d, J=8 Hz), 6.98(2H, d, J=8 Hz), 7.29-7.46(5H, m), 7.50-7.60(4H, m).

### Example 10

### 4-[2-(Difluoromethyl)-4-(4-methoxyphenyl)-1,3-oxazol-5-yl]phenol

The title compound (2.75 g, 103.2%) was obtained as a powder from 5-[4-(benzyloxy)phenyl]-2-(difluoromethyl)-4-(4-methoxyphenyl)-1,3-oxazole obtained by Example 9-5 (3.42 g, 8.39 mmol) in a manner similar to that of Example 65 described later.

¹H-NMR (300 MHz, CDCl₃): δ 3.84(3H, s), 5.27(1H, s), 6.70(1H, t, J=53 Hz), 6.85(2H, d, J=8 Hz), 6.92(2H, d, J=8 Hz), 7.51(2H, d, J=8 Hz), 7.56(2H, d, J=8 Hz) MS (ES−): 316.19.

### Example 11

### Ethyl {4-[2-(difluoromethyl)-4-(4-methoxyphenyl)-1,3-oxazol-5-yl]phenoxy}acetate

To a suspension of sodium hydride (60% in oil, 410 mg, 10.2 mmol) in N,N-dimethylformamide (5 mL) was added a solution of 4-[2-(difluoromethyl)-4-(4-methoxyphenyl)-1,3-oxazol-5-yl]phenol obtained by Example 10 (2.5 g, 9.85 mmol) in N,N-dimethylformamide (20 mL) dropwise at 0° C. under nitrogen, and the mixture was stirred at the same temperature for 1 hr. Then ethyl bromoacetate (1.64 g, 9.85 mmol) was added and stirred at the same temperature for 3 hrs.

The reaction mixture was poured into water and extracted with ethyl acetate. The organic layer was washed with 1 mol/L hydrochloric acid, water, saturated sodium bicarbonate solution and brine, dried over magnesium sulfate, and evaporated in vacuo. The residue was crystalized from a mixture of water and ethanol to give the title compound (2.66 g, 83.7%) as crystals.

¹H-NMR (300 MHz, CDCl₃): δ 1.31(3H, t, J=7.5Hz), 3.85(3H, s), 4.28(2H, q, J=7.5 Hz), 4.66(2H, s), 6.69(1H, t, J=53 Hz), 6.88-6.95(4H, m), 7.54(2H, d, J=8 Hz), 7.58(2H, d, J=8 Hz) MS (ES+): 404.13.

### Example 12

### 2-{4-[2-(Difluoromethyl)-4-(4-methoxyphenyl)-1,3-oxazol-5-yl]phenoxy}ethanol

To a solution of ethyl {4-[2-(difluoromethyl)-4-(4-methoxyphenyl)-1,3-oxazol-5-yl]phenoxy}acetate obtained by Example 11 (4.3 g, 10.7 mmol) in a mixture of diethyl ether (40 mL) and tetrahydrofuran (10 mL) was added lithium aluminum hydride (405 mg, 10.7 mmol) portionwise at 0° C. under nitrogen, and the mixture was stirred at the same temperature for 3 hrs.

To the reaction mixture was added water dropwise at 0° C. The precipitate was removed by vacuum filtration and the filtrate was evaporated in vacuo. The residue was partitioned between water and ethyl acetate. The organic layer was separated, washed with 1 mol/L hydrochloric acid, water, saturated sodium bicarbonate solution and brine, dried over magnesium sulfate, and evaporated in vacuo. The residue was purified by silica gel column chromatography (n-hexane:ethyl acetate=2:1) and crystallized from a mixture of ethyl acetate and n-hexane to give the title compound (3.1 g, 80.5%) as white crystals.

MP: 114-116° C. ¹H-NMR (300 MHz, CDCl₃): δ 2.02(1H, t, J=7Hz), 3.85(3H, s), 3.98(2H, td, J=5.7 Hz), 4.12(2H, t, J=5 Hz), 6.70(1H, t, J=52 Hz), 6.91(2H, d, J=8 Hz),6.94(2H, d, J=8 Hz), 7.52-7.60 (4H, m). MS (ES+): 362.13.

### Example 13

### 3-{4-[2-(Difluoromethyl)-4-(4-methoxyphenyl)-1,3-oxazol-5-yl]phenoxy}-1-propanol

To a solution of 4-[2-(difluoromethyl)-4-(4-methoxyphenyl)-1,3-oxazol-5-yl]phenol obtained by Example 10 (40 mg, 0.126 mmol) in N,N-dimethylformamide (1 mL) were added 3-bromo-1-propanol (26.3 mg, 0.189 mmol) and potassium carbonate (52.3 mg, 0.378 mmol) at room temperature, and the mixture was stirred at the same temperature for 18 hrs.

The reaction mixture was poured into water and extracted with ethyl acetate. The organic layer was washed with 1 mol/L hydrochloric acid, water, saturated sodium bicarbonate solution and brine, dried over magnesium sulfate, and evaporated in vacuo. The residue was purified by preparative thin layer chromatography (n-hexane:ethyl acetate=1:1) to give the title compound (25 mg, 52.8%) as an oil.

¹H-NMR (300 MHz, CDCl₃): δ 1.64(1H, br peak), 2.01-2.14(2H, m), 3.84(3H, s), 3.88(2H, t, J=5 Hz), 4.16(2H, t, J=5 Hz), 6.69(1H, t, J=53 Hz), 6.88-6.95(4H, m), 7.50-7.60(4H, m). MS (ES+): 376.07.

### Example 14

### {4-[2-(Difluoromethyl)-4-(4-methoxyphenyl)-1,3-oxazol-5-yl]phenoxy}acetonitrile

The title compound (241 mg, 71.5%) was obtained as a powder from 4-[2-(difluoromethyl)-4-(4-methoxyphenyl)-1,3-oxazol-5-yl]phenol obtained by Example 10 (300 mg, 0.946 mmol) and iodoacetonitrile (316 mg, 1.89 mmol) in a manner similar to that of Example 13.

¹H-NMR (300 MHz, CDCl₃): δ 3.85(3H, s), 4.82(2H, s), 6.71(1H, t, J=53 Hz), 6.94(2H, d, J=8 Hz), 7.00(2H, d, J=8 Hz), 7.55(2H, d, J=8 Hz), 7.64(2H, d, J=8 Hz).

### Example 15

### N-(2-{4-[2-(Difluoromethyl)-4-(4-methoxyphenyl)-1,3-oxazol-5-yl]phenoxy}ethyl)acetamide

To a solution of {4-[2-(difluoromethyl)-4-(4-methoxyphenyl)-1,3-oxazol-5-yl]phenoxy}acetonitrile obtained by Example 14 (97 mg, 0.272 mmol) in tetrahydrofuran (2 mL) was added lithium aluminum hydride (12.4 mg, 0.327 mmol) at 0° C. under nitrogen, and the mixture was stirred at the same temperature for 3 hrs. To the reaction mixture was added water dropwise at 0° C.

The precipitate was removed by vacuum filtration and the filtrate was evaporated in vacuo. The residue was partitioned between water and ethyl acetate. The organic layer was separated, washed with water and brine, dried over magnesium sulfate, and evaporated in vacuo.

The residue was dissolved in dichloromethane (2 mL). To the solution were added pyridine (64. 6 mg, 0.817 mmol) and acetyl chloride (25.6 mg, 0.327 mmol) at 0° C., and the mixture was stirred at the same temperature for 2 hrs.

The reaction mixture was evaporated in vacuo, and the residue was partitioned between water and ethyl acetate. The organic layer was separated, washed with 1 mol/L hydrochloric acid, water, saturated sodium bicarbonate solution and brine, dried over magnesium sulfate, and evaporated in vacuo. The residue was purified by preparative thin layer chromatography (ethyl acetate:chloroform:n-hexane=: 12:7:1) to give the title compound (28 mg, 25.6%) as a powder.

1H-NMR (300 MHz, CDCl₃): δ 2.03(3H, s), 3.68(2H, q, J=5 Hz), 3.85(3H, s), 4.08(2H, t, J=5 Hz), 5.93(1H, br peak), 6.70(1H, t, J=53 Hz), 6.86-6.96(4H, m), 7.51-7.60(4H, m). MS (ES+): 403.10.

### Example 16

### tert-Butyl 2-{4-[2-(difluoromethyl)-4-(4-methoxyphenyl)-1,3-oxazol-5-yl]phenoxy}ethylcarbamate

To a solution of {4-[2-(difluoromethyl)-4-(4-methoxyphenyl)-1,3-oxazol-5-yl]phenoxy}acetonitrile obtained by Example 14 (245 mg, 0.688 mmol) in tetrahydrofuran (2 mL) was added lithium aluminum hydride (31.3 mg, 0.825 mmol) at 0° C. under nitrogen, and the mixture was stirred at the same temperature for 3 hrs.

To the reaction mixture was added water dropwise at 0° C. The precipitate was removed by vacuum filtration and the filtrate was evaporated in vacuo. The residue was partitioned between water and ethyl acetate. The organic layer was separated, washed with water and brine, dried over magnesium sulfate, and evaporated in vacuo.

The residue was dissolved in dichloromethane (2 mL). To a solution were added triethylamine (83.5 mg, 0.115 mmol) and di-tert-butyl dicarbonate (180 mg, 0.115 mmol) 0° C., and the mixture was stirred at the same temperature for 2 hrs.

The reaction mixture was evaporated in vacuo, and the residue was partitioned between water and ethyl acetate. The organic layer was separated, washed with 1 mol/L hydrochloric acid, water, saturated sodium bicarbonate solution and brine, dried over magnesium sulfate, and evaporated in vacuo. The residue was purified by preparative thin layer chromatography (ethyl acetate:n-hexane=1:1) to give the title compound (94 mg, 29.7%) as an oil.

¹H-NMR (300 MHz, CDCl₃): δ 1.46(9H, s), 3.56(2H, q, J=5 Hz), 3.85(3H, s), 4.06(2H, t, J=5 Hz), 4.99(1H, br peak), 6.70(1H, t, J=53 Hz), 6.88-6.95(4H, m), 7.51-7.59(4H, m). MS (ES+): 461.15.

### Example 17

### 2-{4-[2-(Difluoromethyl)-4-(4-methoxyphenyl)-1,3-oxazol-5-yl]phenoxy}ethanamine hydrochloride

4N Hydrogen chloride solution in ethyl acetate (0.5 mL) was added to a solution of 1-tert-butyl 2-{4-[2-(difluoromethyl)-4-(4-methoxyphenyl)-1,3-oxazol-5-yl]phenoxy}ethylcarbamate obtained by Example 16 (92 mg, 0.2 mmol) in ethyl acetate (1 mL) at room temperature. The mixture was stirred at the same temperature for 3 hrs.

After evaporation of solvent, the residue was triturated with ether to give the title compound (52 mg, 65.6%) as an amorphous powder.

¹H-NMR (300 MHz, DMSO-d₆): δ 3.24(2H, br peak), 3.79(3H, s), 4.23(2H, t, J=5 Hz), 7.00(2H, d, J=8 Hz), 7.10(2H, d, J=8 Hz), 7.31(1H, t, J=53 Hz), 7.50(2H, d, J=8 Hz), 7.55(2H, d, J=8 Hz), 8.09(3H, br peak). MS (ES+): 361.13.

### Example 18

### N-(2-{4-[2-(Difluoromethyl)-4-(4-methoxyphenyl)-1,3-oxazol-5-yl]phenoxy}ethyl)urea

To a solution of 2-{4-[2-(difluoromethyl)-4-(4-methoxyphenyl)-1,3-oxazol-5-yl]phenoxy}ethanamine (136 mg, 0.377 mmol) in dichloromethane (3 mL) was added trimethylsilyl isocyanate (87 mg, 0.755 mmol) at room temperature, and the mixture was stirred at the same temperature for 24 hrs.

The reaction mixture was poured into water and extracted with chloroform. The organic layer was washed with 1 mol/L hydrochloric acid, water, saturated sodium bicarbonate solution and brine, dried over magnesium sulfate, and evaporated in vacuo. The residue was purified by preparative thin layer chromatography (chloroform:methanol=10:1) to give the title compound (95 mg, 62.4%) as an amorphous powder.

MP: 146-149° C. ¹H-NMR (300 MHz, DMSO-d₆): δ 3.25-3.40(2H, m), 3.80(3H, s), 4.00(2H, t, J=7 Hz), 5.54(2H, s), 6.17(1H, t, J=7 Hz), 7.00(2H, d, J=8 Hz), 7.06(2H, d, J=8 Hz), 7.29(1H, t, J=53 Hz), 7.47-7.55(4H, m).

### Example 19-1

### Ethyl {[2-[4-(benzyloxy)phenyl]-1-(4-methoxyphenyl)-2-oxoethyl]amino}(oxo)acetate

The title compound (1.9 g, 88.1%) was obtained as an oil from 2-amino-1-[4-(benzyloxy)phenyl]-2-(4-methoxyphenyl)ethanone hydrochloride (1.85 g, 4.82 mmol) and ethyl chlorooxoacetate (888 mg, 6.51 mmol) in a manner similar to that of Example 1-1.

¹H-NMR (300 MHz, CDCl₃): δ 1.37(3H, t, J=7.5Hz), 3.75(3H, s), 4.34(2H, q, J=7.5 Hz), 5.08(2H, s), 6.42(1H, d, J=7.5 Hz), 6.82(2H, d, J=8 Hz), 6.94(2H, d, J=8 Hz), 7.29-7.45(7H, m), 7.94(2H, d, J=8 Hz), 8.48(1H, d, J=7.5 Hz). MS (ES+): 448.14.

### Example 19-2

### Ethyl 5-[4-(benzyloxy)phenyl]-4-(4-methoxyphenyl)-1,3-oxazole-2-carboxylate

The title compound (1.06 g, 58.3%) was obtained as an oil from ethyl {[2-[4-(benzyloxy)phenyl]-1-(4-methoxyphenyl)-2-oxoethyl]amino)(oxo)acetate obtained by Example 19-1 (1.9 g, 4.25 mmol) in a manner similar to that of Example 1-2.

¹H-NMR (300 MHz, CDCl₃): δ 1.45(3H, t, J=7.5Hz), 3.84(3H, s), 4.50(2H, q, J=7.5 Hz), 5.10(2H, s), 6.91(2H, d, J=8 Hz), 6.98(2H, d, J=8 Hz), 7.30-7.46(5H, m), 7.55-7.65(4H, m). MS (ES+): 430.14.

### Example 20

### 5-[4-(Benzyloxy)phenyl]-4-(4-methoxyphenyl)-1,3-oxazole-2-carboxamide

The title compound (980 mg, 99.2%) was obtained as a powder from ethyl 5-[4-(benzyloxy)phenyl]-4-(4-methoxyphenyl)-1,3-oxazole-2-carboxylate obtained by Example 19-2 (1.06 g, 2.47 mmol) in a manner similar to that of Example 2.

¹H-NMR (300 MHz, CDCl₃): δ 3.85(3H, s), 5.09(2H, s), 5.76(1H, br peak), 6.90-7.04(5H, m), 7.30-7.46(5H, m), 7.56(2H, d, J=8 Hz), 7.61(2H, d, J=8 Hz). MS (ES+): 401.12.

### Example 21

### 5-(4-Hydroxyphenyl)-4-(4-methoxyphenyl)-1,3-oxazole-2-carboxamide

The title compound (298 mg, 91.6%) was obtained as a powder from 5-[4-(benzyloxy)phenyl]-4-(4-methoxyphenyl)-1,3-oxazole-2-carboxamide obtained by Example 20 (420 mg, 1.05 mmol) in a manner similar to that of Example 65 described later.

¹H-NMR (300 MHz, DMSO-d₆): δ 3.80(3H, s), 6.84(2H, d, J=8 Hz), 7.00(2H, d, J=8 Hz), 7.43(2H, d, J=8 Hz), 7.53(2H, d, J=8 Hz), 7.90(1H, s), 8.26(1H, s), 9.98(1H, s). MS (ES−): 309.20.

### Example 22

### 5-[4-(2-Hydroxyethoxy)phenyl]-4-(4-methoxyphenyl)-1,3-oxazole-2-carboxamide

The title compound (200 mg, 58.8%) was obtained as a powder from 5-(4-hydroxyphenyl)-4-(4-methoxyphenyl)-1,3-oxazole-2-carboxamide obtained by Example 21 (298 mg, 0.96 mmol) and chloroethanol (193 mg, 2.4 mmol) in a manner similar to that of Example 87 described later.

¹H-NMR (300 MHz, CDCl₃): δ 2.01(1H, t, J=7Hz), 3.85(3H, s), 4.03(2H, dd, J=7.5 Hz), 4.12(2H, t, J=5 Hz), 5.14(1H, br-s), 6.87-6.95(4H, m), 6.98(1H, br peak), 7.55(2H, d, J=8 Hz), 7.60(2H, d, J=8 Hz). MS (ES+): 355.20.

### Example 23

### 5-[4-(2-Hydroxyethoxy)phenyl]-4-(4-methoxyphenyl)-1,3-oxazole-2-carbonitrile

To a solution of 5-[4-(2-hydroxyethoxy)phenyl]-4-(4-methoxyphenyl)-1,3-oxazole-2-carboxamide obtained by Example 22 (55.4 mg, 0.156 mmol) and pyridine (61.8 mg, 0.782 mmol) in dichloromethane (2 mL) was added trifluoroacetic anhydride (75.5 mg, 0.36 mmol) under nitrogen at room temperature, and the mixture was stirred at the same temperature for 1 hr.

The mixture was evaporated in vacuo, and the residue was partitioned between water and ethyl acetate. The organic layer was separated, washed with 1 mol/L hydrochloric acid and water, dried over magnesium sulfate, and evaporated in vacuo.

The residue was dissolved in methanol (5 mL) and the solution was allowed to stand at room temperature for 18 hrs.

After evaporation of solvent, the residue was purified by preparative thin layer chromatography (n-hexane:ethyl acetate=1:1), and triturated with a mixture of petroleum ether and diethyl ether to give the title compound (26 mg, 49.4%) as a powder.

¹H-NMR (300 MHz, CDCl₃): δ 2.00(1H, t, J=7 Hz), 3.85(3H, s), 4.00(2H, dd, J=7.5 Hz), 4.14(2H, t, J=5 Hz), 6.93(2H, d, J=8 Hz), 6.95(2H, d, J=8 Hz), 7.51-7.60(4H, m). MS (ES+): 337.15.

### Example 24

### 2-{4-[2-(Aminocarbonyl)-4-(4-methoxyphenyl)-1,3-oxazol-5-yl]phenoxy}ethyl acetate

The title compound (102 mg, 85.2%) was obtained as an oil from 5-[4-(2-hydroxyethoxy)phenyl]-4-(4-methoxyphenyl)-1,3-oxazole-2-carboxamide obtained by Example 22 (107 mg, 0.302 mmol) in a manner similar to that of Example 39-1 described later.

¹H-NMR (300 MHz, CDCl₃): δ 2.11(3H, S), 3.85(3H, s), 4.20(2H, t, J=5 Hz), 4.44(2H, t, J=5 Hz), 5.66(1H, br s), 6.91(2H, d, J=8 Hz), 6.93(2H, d, J=8 Hz), 6.99(1H, br s), 7.55(2H, d, J=8 Hz), 7.60(2H, d, J=8 Hz). MS (ES+): 397.12.

### Example 25

### 2-{4-[2-Cyano-4-(4-methoxyphenyl)-1,3-oxazol-5-yl]phenoxy}ethyl acetate

The title compound (80 mg, 83.8%) was obtained as an oil from 2-{4-[2-(aminocarbonyl)-4-(4-methoxyphenyl)-1,3-oxazol-5-yl]phenoxy}ethyl acetate obtained by Example 24 (100 mg, 0.252 mmol) in a manner similar to that of Example 3.

¹H-NMR (300 MHz, CDCl₃): δ 2.11(3H, s), 3.85(3H, s), 4.23(2H, t, J=5 Hz), 4.45(2H, t, J=5 Hz), 6.89-6.99(4H, m), 7.50-7.60(4H, m).

### Example 26

### 5-[4-(Cyanomethoxy)phenyl]-4-(4-methoxyphenyl)-1,3-oxazole-2-carboxamide

The title compound (383 mg, 86.6%) was obtained as an oil from 5-(4-hydroxyphenyl)-4-(4-methoxyphenyl)-1,3-oxazole-2-carboxamide obtained by Example 21 (393 mg, 1.27 mmol) and iodoacetonitrile (423 mg, 2.53 mmol) in a manner similar to that of Example 13.

¹H-NMR (300 MHz, CDCl₃): δ 3.86(3H, s), 4.81(2H, s), 5.65(1H, br-s), 6.91-7.04(5H, m), 7.55(2H, d, J=8 Hz), 7.68(2H, d, J=8 Hz). MS (ES+): 350.11.

### Example 27

### 5-[4-(2-Aminoethoxy)phenyl]-4-(4-methoxyphenyl)-1,3-oxazole-2-carboxamide

To a mixture of 5-[4-(cyanomethoxy)phenyl]-4-(4-methoxyphenyl)-1,3-oxazole-2-carboxamide obtained by Example 26 (150 mg, 0.429 mmol) and cobalt(II) chloride hexahydrate (30.6 mg, 0.129 mmol) in methanol (3 mL) was added sodium borohydride (162 mg, 4.29 mmol) portionwise in water bath under nitrogen, and the mixture was stirred in water bath for 15 min. 1N Sodium hydroxide solution (0.5 mL) was added to the mixture and the reaction mixture was stirred for 30 min.

The reaction mixture was filtered through Celite and evaporated in vacuo. The residue was partitioned between water and chloroform. The organic layer was separated, dried over magnesium sulfate, and evaporated in vacuo. The residue was purified by preparative thin layer chromatography (chloroform:methanol=10:1) to give the title compound (77 mg, 50.7%) as a powder.

¹H-NMR (300 MHz, CDCl₃): δ 3.11(2H, t, J=5 Hz), 3.85(3H, s), 4.02(2H, t, J=5 Hz), 5.61 (1H, br-s), 6.90(2H, d, J=8 Hz), 6.93(2H, d, J=8 Hz), 6.99(1H, br-s), 7.56(2H, d, J=8 Hz), 7.60(2H, d, J=8 Hz).

### Example 28

### 5-{4-[2-(Acetylamino)ethoxy]phenyl}-4-(4-methoxyphenyl)-1,3-oxazole-2-carboxamide

The title compound (47 mg, 60%) was obtained as an oil from 5-[4-(2-aminoethoxy)phenyl]-4-(4-methoxyphenyl)-1,3-oxazole-2-carboxamide obtained by Example 27 (70 mg, 0.198 mmol) in a manner similar to that of Example 39-1 described later.

¹H-NMR (300 MHz, CDCl₃): δ 2.03(3H, s), 3.70(2H, q, J=5 Hz), 3.85(3H, s), 4.07(2H, t, J=5 Hz), 5.96(1H, br-s), 6.10(1H, br-s), 6.89(2H, d, J=8 Hz), 6.95(2H, d, J=8 Hz), 7.11(1H, br-s), 7.54(2H, d, J=8 Hz), 7.60(2H, d, J=8 Hz). MS (ES+): 396.13.

### Example 29

### N-(2-{4-[2-Cyano-4-(4-methoxyphenyl)-1,3-oxazol-5-yl]phenoxy}ethyl)acetamide

The title compound (26 mg, 56.2%) was obtained as a powder from 5-{4-[2-(acetylamino)ethoxy]phenyl}-4-(4-methoxyphenyl)-1,3-oxazole-2-carboxamide obtained by Example 28 (48.5 mg, 0.123 mmol) in a manner similar to that of Example 23.

¹H-NMR (300 MHz, CDCl₃): δ 2.05(3H, s), 3.69(2H, q, J=5 Hz), 3.85(3H, s), 4.09(2H, t, J=5 Hz), 5.91(1H, br peak), 6.88-6.96(4H, m), 7.50-7.60(4H, m). MS (ES+): 378.10.

### Example 30-1

### (2E)- and (2Z)-2-[4-(Benzyloxy)phenyl]-3-(6-methoxy-3-pyridinyl)-2-propenoic acid

The title compound was obtained in a manner similar to that of Example 91-3 described later.

¹H-NMR (300 MHz, DMSO-d₆): δ 3.81(15/8H, s), 3.87(9/8H, s), 5.13(10/8H, s), 5.15(6/8H, s), 6.64(5/8H, d, J=8 Hz), 6.86(3/8H, d, J=8 Hz), 6.93(3/8H, s), 7.03-7.12(2H, m), 7.18(5/8H, dd, J=8.2 Hz), 7.32-7.50(7H, m), 7.70(5/8H, s), 7.80(3/8H, dd, J=8.2 Hz), 8.04(5/8H, d, J=2 Hz), 8.28(3/8H, d, J=2 Hz).

### Example 30-2

### 1-[4-(Benzyloxy)phenyl]-2-(6-methoxy-3-pyridinyl)ethanone

The title compound was obtained from (2E)- and (2Z)-2-[4-(benzyloxy)phenyl]-3-(6-methoxy-3-pyridinyl)-2-propenoic acid obtained by Example 30-1 in a manner similar to that of Example 91-4 described later.

¹H-NMR (300 MHz, CDCl₃): δ 3.92(3H, s), 4.16(2H, s), 5.14(2H, s), 6.72(1H, d, J=8 Hz), 7.02(2H, d, J=8 Hz), 7.30-7.45(5H, m), 7.49(1H, dd, J=8.2 Hz), 7.99(2H, d, J=8 Hz), 8.02(1H, d, J=2 Hz). MS (ES+): 334.15.

### Example 30-3

### 1-[4-(Benzyloxy)phenyl]-2-bromo-2-(6-methoxy-3-pyridinyl)ethanone

The title compound (21.2 g, 78.1%) was obtained as a powder from 1-[4-(benzyloxy)phenyl]-2-(6-methoxy-3-pyridinyl)ethanone obtained by Example 30-2 (22 g, 66 mmol) and pyridinium tribromide (23.2 g, 72.6 mmol) in a manner similar to that of Example 68-1 described later.

¹H-NMR (300 MHz, CDCl₃): δ 3.95(3H, s), 5.14(2H, s), 6.26(1H, s), 6.80(1H, d, J=8 Hz), 7.02(2H, d, J=8 Hz), 7.30-7.46(5H, m), 7.92(1H, dd, J=8.2 Hz), 8.01(2H, d, J=8 Hz), 8.21(1H, d, J=2 Hz). MS (ES+): 411.98, 413.95.

### Example 30-4

### 2-[2-[4-(Benzyloxy)phenyl]-1-(6-methoxy-3-pyridinyl)-2-oxoethyl]-1H-isoindole-1,3(2H)-dione

The title compound (20.0 g, 81.2%) was obtained as a powder from 1-[4-(benzyloxy)phenyl]-2-bromo-2-(6-methoxy-3-pyridinyl)ethanone obtained by Example 30-3 (21.2 g, 51.5 mmol) and potassium phthalimide (9.54 g, 51.3 mmol) in a manner similar to that of Example 9-2.

¹H-NMR (300 MHz, CDCl₃): δ 3.91(3H, s), 5.07(2H, s), 6.65-6.72(2H, m), 6.93(2H, d, J=8 Hz), 7.27-7.41(5H, m), 7.66-7.78(3H, m), 7.78-7.88(4H, m), 8.26(1H, d, J=2 Hz).

### Example 30-5

### 2-Amino-1-[4-(benzyloxy)phenyl]-2-(6-methoxy-3-pyridinyl)ethanone hydrochloride

The title compound (2.67 g, 110%) was obtained from 2-[2-[4-(benzyloxy)phenyl]-1-(6-methoxy-3-pyridinyl)-2-oxoethyl]-1H-isoindole-1,3(2H)-dione obtained by Example 30-4 (3.0 g, 6.27 mmol) in a manner similar to that of Example 9-3.

¹H-NMR (300 MHz, DMSO-d₆): δ 3.82(3H, s), 5.18(2H, s), 6.32(1H, br peak), 6.85(1H, d, J=8 Hz), 7.10(2H, d, J=8 Hz), 7.26-7.50(5H, m), 7.71(1H, dd, J=8.2 Hz), 8.02(2H, d, J=8Hz), 8.40(1H, d, J=2 Hz), 8.91(2H, br peak).

### Example 30-6

### N-[2-[4-(Benzyloxy)phenyl]-1-(6-methoxy-3-pyridinyl)-2-oxoethyl]-2,2-difluoroacetamide

To a solution of difluoroacetic acid (799 mg, 8.33 mmol) in tetrahydrofuran (8 mL) were added oxalyl chloride (1.06 g, 8.33 mmol) and N,N-dimethylformamide (1 drop) at 0° C. under nitrogen, and the mixture was stirred at room temperature for 1 hr. The mixture was added to a mixture of 2-amino-1-[4-(benzyloxy)phenyl]-2-(6-methoxy-3-pyridinyl)ethanone hydrochloride obtained by Example 30-5 (2.67 g, 6.94 mmol) and triethylamine (2.11 g, 20.8 mmol) in dichloromethane (25 mL) at 0° C., and the reaction mixture was stirred at the same temperature for 2 hrs.

The reaction mixture was evaporated in vacuo, and partitioned between water and ethyl acetate. The organic layer was separated, washed with water, saturated sodium bicarbonate solution and brine, successively, dried over magnesium sulfate. After evaporation of solvent, the residue was purified by silica gel column chromatography (n-hexane:ethyl acetate=3:1) to give the title compound (1.25 g, 42.6%) as a powder.

¹H-NMR (300 MHz, CDCl₃): δ 3.89(3H, s), 5.10(2H, s), 5.89(1H, t, J=53 Hz), 6.40(1H, d, J=8 Hz), 6.68(1H, d, J=8 Hz), 6.96(2H, d, J=8 Hz), 7.31-7.42(5H, m), 7.53(1H, dd, J=8.2 Hz), 7.89-8.00(3H, m), 8.25(1H, d, J=2 Hz).

### Example 30-7

### 5-[5-[4-(Benzyloxy)phenyl]-2-(difluoromethyl)-1,3-oxazol-4-yl]-2-methoxypyridine

The title compound (840 mg, 70.2%) was obtained as a powder from N-[2-[4-(benzyloxy)phenyl]-1-(6-methoxy-3-pyridinyl)-2-oxoethyl]-2,2-difluoroacetamide obtained by Example 30-6 (1.25 g, 2.93 mmol) in a manner similar to that of Example 9-5.

¹H-NMR (300 MHz, CDCl₃): δ 3.97(3H, s), 5.10(2H, s), 6.70(1H, t, J=53 Hz), 6.77(1H, d, J=8 Hz), 7.00(2H, d, J=8 Hz), 7.30-7.48(5H, m), 7.54(2H, d, J=8 Hz), 7.82(1H, dd, J=8.2 Hz), 8.44(1H, d, J=2 Hz).

### Example 31

### 4-[2-(Difluoromethyl)-4-(6-methoxy-3-pyridinyl)-1,3-oxazol-5-yl]phenol

5-[5-[4-(Benzyloxy)phenyl]-2-(difluoromethyl)-1,3-oxazol-4-yl]-2-methoxypyridine obtained by Example 30-7 (830 mg, 2.03 mmol) and dry 20% palladium hydroxide on carbon (240 mg) in ethanol (8 mL) and cyclohexene (4 mL) was stirred at reflux condition for 2 hrs, and cooled to room temperature.

After filtration, the reaction mixture was evaporated in vacuo to give the title compound (630 mg, 97.8%) as a powder.

¹H-NMR (300 MHz, DMSO-d₆): δ 3.89(3H, s), 6.86(2H, d, J=9 Hz), 6.91(1H, d, J=9 Hz), 7.30(1H, t, J=53 Hz), 7.84(1H, dd, J=9.2 Hz), 8.36(1H, d, J=2 Hz).

### Example 32

### Ethyl {4-[2-(difluoromethyl)-4-(6-methoxy-3-pyridinyl)-1,3-oxazol-5-yl]phenoxy}acetate

The title compound (830 mg, 105%) was obtained as a powder from 4-[2-(difluoromethyl)-4-(6-methoxy-3-pyridinyl)-1,3-oxazol-5-yl]phenol obtained by Example 31 (620 mg, 1.95 mmol) and ethyl bromoacetate (390 mg, 2.34 mmol) in a manner similar to that of Example 11.

¹H-NMR (300 MHz, CDCl₃): δ 1.32(3H, t, J=7 Hz), 3.97(3H, s), 4.28(2H, q, J=7 Hz), 4.66(2H, s), 6.69(1H, t, J=53 Hz), 6.78(1H, d, J=8 Hz), 6.94(2H, d, J=8 Hz), 7.55(2H, d, J=8 Hz), 7.80(1H, dd, J=8.2 Hz), 8.42(1H, d, J=2 Hz). MS (ES+): 405.11.

### Example 33

### 2-{4-[2-(Difluoromethyl)-4-(6-methoxy-3-pyridinyl)-1,3-oxazol-5-yl]phenoxy}ethanol

The title compound (630 mg, 82.2%) was obtained as crystals from ethyl {4-[2-(difluoromethyl)-4-(6-methoxy-3-pyridinyl)-1,3-oxazol-5-yl]phenoxy}acetate (855 mg, 2.11 mmol) obtained by Example 32 in a manner similar to that of Example 12.

MP: 126-128° C. ¹H-NMR (300 MHz, CDCl₃): δ 2.01(1H, t, J=6 Hz), 3.98(3H, s), 4.00(2H, dd, J=6.5 Hz), 4.13(2H, t, J=5 Hz), 6.70(1H, t, J=53 Hz), 6.77(1H, d, J=8 Hz), 6.95(2H, d, J=8 Hz), 7.55(2H, d, J=8 Hz), 7.82(1H, dd, J=8.2 Hz), 8.43(1H, d, J=2 Hz). MS (ES+): 363.14.

### Example 34

### 2-{4-[2-(Difluoromethyl)-4-(6-methoxy-3-pyridinyl)-1,3-oxazol-5-yl]phenoxy}ethyl methanesulfonate

To a solution of 2-{4-[2-(difluoromethyl)-4-(6-methoxy-3-pyridinyl)-1,3-oxazol-5-yl]phenoxy}ethanol obtained by Example 33 (203 mg, 0.56 mmol) and triethylamine (85 mg, 0.84 mmol) in dichloromethane (4 mL) was added methanesulfonyl chloride (86.3 mg, 0.84 mmol) at 0° C. under nitrogen, and the mixture was stirred at the same temperature for 2 hrs.

The reaction mixture was evaporated in vacuo, and the residue was partitioned between water and chloroform. The organic layer was separated, washed with 1 mol/L hydrochloric acid, water, saturated sodium bicarbonate solution and brine, dried over magnesium sulfate, and evaporated in vacuo to give the title compound (247 mg, 100.1%) as an oil.

¹H-NMR (300 MHz, CDCl₃): δ 3.11(3H, s), 3.97(3H, s), 4.29(2H, t, J=5 Hz), 4.60(2H, t, J=5 Hz), 6.70(1H, t, J=53 Hz), 6.78(1H, d, J=8 Hz), 6.94(2H, d, J=8 Hz), 7.55(2H, d, J=8 Hz), 7.82(1H, dd, J=8.2 Hz), 8.41(1H, d, J=2 Hz).

### Example 35

### 2-(2-{4-[2-(Difluoromethyl)-4-(6-methoxy-3-pyridinyl)-1,3-oxazol-5-yl]phenoxy}ethyl)-1H-isoindole-1,3(2H)-dione

To a solution of 2-{4-[2-(difluoromethyl)-4-(6-methoxy-3-pyridinyl)-1,3-oxazol-5-yl]phenoxy}ethyl methanesulfonate obtained by Example 34 (247 mg, 0.561 mmol) in N,N-dimethylformamide (5 mL) was added potassium phthalimide (156 mg, 0.841 mmol) at room temperature, and the mixture was stirred at 60° C. for 18 hrs.

The reaction mixture was poured into water and extracted with ethyl acetate. The organic layer was washed with 1 mol/L hydrochloric acid, water, saturated sodium bicarbonate solution and brine, dried over magnesium sulfate, and evaporated in vacuo to give the title compound (260 mg, 94.3%) as an oil.

¹H-NMR (300 MHz, CDCl₃): δ 3.96(3H, s), 4.13 (1H, t, J=7 Hz), 4.27(1H, t, J=7 Hz), 6.69(1H, t, J=53 Hz), 6.76(1H, d, J=8 Hz), 6.91(2H, d, J=8 Hz), 7.79(2H, d, J=8 Hz), 7.70-7.81(3H, m), 7.84-7.91(2H, m), 8.39(1H, d, J=2 Hz).

### Example 36

### 2-{4-[2-(Difluoromethyl)-4-(6-methoxy-3-pyridinyl)-1,3-oxazol-5-yl]phenoxy}ethylamine

To a solution of 2-(2-{4-[2-(difluoromethyl)-4-(6-methoxy-3-pyridinyl)-1,3-oxazol-5-yl]phenoxy}ethyl)-1H-isoindole-1,3(2H)-dione obtained by Example 35 (260 mg, 0.529 mmol) in acetonitrile (5 mL) was added hydrazine monohydrate (212 mg, 4.23 mmol) at room temperature, and the mixture was stirred at 60° C. for 5 hrs.

After cooling, the precipitate was filtered off. The filtrate was concentrated in vacuo to give the title compound (184 mg, 96.2%) as an oil.

¹H-NMR (300 MHz, CDCl₃): δ 3.11(2H, t, J=5 Hz), 3.97(3H, s), 4.03(2H, t, J=5 Hz), 6.70(1H, t, J=53 Hz), 6.78(1H, d, J=8 Hz), 6.94(2H, d, J=8 Hz), 7.54(2H, d, J=8 Hz), 7.82(1H, dd, J=8.2 Hz), 8.43(1H, d, J=2 Hz). MS (ES+): 362.13.

### Example 37

### N-(2-{4-[2-(Difluoromethyl)-4-(6-methoxy-3-pyridinyl)-1,3-oxazol-5-yl]phenoxy}ethyl)urea

The title compound (46 mg, 47.8%) was obtained as a powder from 2-{4-[2-(difluoromethyl)-4-(6-methoxy-3-pyridinyl)-1,3-oxazol-5-yl]phenoxy}ethylamine obtained by Example 36 (86 mg, 0.238 mmol) in a manner similar to that of Example 18.

¹H-NMR (300 MHz, DMSO-d₆): δ 3.28-3.40(2H, m), 3.89(3H, s), 4.00(2H, t, J=5 Hz), 5.55(2H, s), 6.18(1H, t, J=5 Hz), 6.92(1H, d, J=9 Hz), 7.09(2H, d, J=9 Hz), 7.33(1H, t, J=53 Hz), 7.52(2H, d, J=9 Hz), 7.83(1H dd, J=9.2 Hz), 8.37(1H, d, J=2 Hz). MS (ES+): 405.13.

### Example 38

### N-(2-{4-[2-(Difluoromethyl)-4-(6-methoxy-3-pyridinyl)-1,3-oxazol-5-yl]phenoxy}ethyl)methanesulfonamide

To a solution of 2-{4-[2-(difluoromethyl)-4-(6-methoxy-3-pyridinyl)-1,3-oxazol-5-yl]phenoxy}ethylamine obtained by Example 36 (80 mg, 0.221 mmol) and triethylamine (27 mg, 0.266 mmol) in dichloromethane (2 mL) was added methanesulfonyl chloride (30.4 mg, 0.266 mmol) at 0° C. under nitrogen, and the mixture was stirred at the same temperature for 2 hrs.

The reaction mixture was evaporated in vacuo, and the residue was partitioned between water and ethyl acetate. The organic layer was separated, washed with 1 mol/L hydrochloric acid, water, saturated sodium bicarbonate solution and brine, dried over magnesium sulfate, and evaporated in vacuo. The residue was purified by preparative thin layer chromatography (n-hexane:ethyl acetate=2:1) to give the title compound (52 mg, 53.4%) as an oil.

H-NMR (300 MHz, CDCl₃): δ 3.04(3H, s), 3.58(2H, q, J=7 Hz), 3.97(3H, s), 4.15(2H, t, J=7 Hz), 4.76(1H, t, J=7 Hz), 6.70(1H, t, J=53 Hz), 6.78 (1H, d, J=8 Hz), 6.92(2H, d, J=8 Hz), 7.55(2H, d, J=8 Hz), 7.81(1H, dd, J=8.2 Hz), 8.41(1H, d, J=2 Hz). MS (ES+): 440.11.

### Example 39-1

### N-[2-[4-(Benzyloxy)phenyl]-1-(4-methoxyphenyl)-2-oxoethyl]-2,2,2-trifluoroacetamide

To a suspension of 2-amino-1-[4-(benzyloxy)phenyl]-2-(4-methoxyphenyl)ethanone hydrochloride (1.56 g, 4.14 mmol) in dichloromethane (16 mL) were added triethylamine (503 mg, 4.97 mmol) and trifluoroacetic anhydride (1.04 g, 4.97 mmol) at 0° C. under nitrogen, and the mixture was stirred at room temperature for 2 hrs.

The reaction mixture was evaporated in vacuo, and partitioned between water and ethyl acetate. The organic layer was separated, washed with water, saturated sodium bicarbonate solution and brine, successively, dried over magnesium sulfate. After evaporation of solvent, the residue was triturated with hexane to give the title compound (1.20 g, 65.3%) as a powder.

¹H-NMR (300 MHz, CDCl₃): δ 3.76(3H, s), 5.09(2H, s), 6.35(1H, d, J=7 Hz), 6.84(2H, d, J=8 Hz), 6.94(2H, d, J=8 Hz), 7.26-7.44(7H, m), 7.87-8.00(3H, m). MS (ES−): 442.26.

### Example 39-2

### 5-[4-(Benzyloxy)phenyl]-4-(4-methoxyphenyl)-2-(trifluoromethyl)-1,3-oxazole

The title compound (860 mg, 74.7%) was obtained as a powder from N-[2-[4-(benzyloxy)phenyl]-1-(4-methoxyphenyl)-2-oxoethyl]-2,2,2-trifluoroacetamide obtained by Example 39-1 (1.2 g, 2.71 mmol) in a manner similar to that of Example 9-5.

¹H-NMR (300 MHz, CDCl₃): δ 3.85(3H, s), 5.11(2H, s), 6.80(2H, d, J=8 Hz), 6.98(2H, d, J=8 Hz), 7.26-7.46(5H, m), 7.51-7.60(4H, m).

### Example 40

### 4-[4-(4-Methoxyphenyl)-2-(trifluoromethyl)-1,3-oxazol-5-yl]phenol

The title compound (655 mg, 96.6%) was obtained as a powder from 5-[4-(benzyloxy)phenyl]-4-(4-methoxyphenyl)-2-(trifluoromethyl)-1,3-oxazole obtained by Example 39-2 (60 mg, 2.02 mmol) in a manner similar to that of Example 65 described later.

¹H-NMR (300 MHz, DMSO-d₆): δ 3.79(3H, s), 6.85(2H, d, J=8 Hz), 7.00(2H, d, J=8 Hz), 7.42(2H, d, J=8 Hz), 7.52(2H, d, J=8 Hz). MS (ES−): 334.20.

### Example 41

### 2-{4-[4-(4-Methoxyphenyl)-2-(trifluoromethyl)-1,3-oxazol-5-yl]phenoxy}ethanol

The title compound (742 mg, 98.6%) was obtained as a powder from 4-[4-(4-methoxyphenyl)-2-(trifluoromethyl)-1,3-oxazol-5-yl]phenol obtained by Example 40 (665 mg, 1.95 mmol) and 2-chloroethanol (958 mg, 11.9 mmol) in a manner similar to that of Example 87 described later.

MP: 98-100° C. ¹H-NMR (300 MHz, CDCl₃): δ 2.00(1H, t, J=7 Hz), 3.85(3H, s), 4.00(2H, dt, J=7,5 Hz), 4.13(1H, t, J=5 Hz), 6.91(2H, d, J=8 Hz), 7.05(2H, d, J=8 Hz), 7.51-7.61(4H, m).

### Example 42

### 2-{4-[4-(4-Methoxyphenyl)-2-(trifluoromethyl)-1,3-oxazol-5-yl]phenoxy}ethyl methanesulfonate

The title compound (895 mg, 100%) was obtained as an oil from 2-{4-[4-(4-methoxyphenyl)-2-(trifluoromethyl)-1,3-oxazol-5-yl]phenoxy}ethanol obtained by Example 41 (742 mg, 1.96 mmol) in a manner similar to that of Example 34.

¹H-NMR (300 MHz, CDCl₃): δ 3.12(3H, s), 3.87(3H, s), 4.30(2H, t, J=5 Hz), 4.60(2H, t, J=5 Hz), 6.87-6.99(4H, m), 7.53-7.63(4H, m).

### Example 43

### 2-(2-{4-[4-(4-Methoxyphenyl)-2-(trifluoromethyl)-1,3-oxazol-5-yl ]phenoxy}ethyl)-1H-isoindole-1,3(2H)-dione

The title compound (1.03 g, 103%) was obtained as a powder from 2-{4-[4-(4-methoxyphenyl)-2-(trifluoromethyl)-1,3-oxazol-5-yl]phenoxy}ethyl methanesulfonate obtained by Example 42 (895 mg, 1.96 mmol) and potassium phthalimide (544 mg, 2.93 mmol) in a manner similar to that of Example 35.

¹H-NMR (300 MHz, CDCl₃): δ 3.84(3H, s), 4.11(2H, t, J=5 Hz), 4.26(2H, t, J=5 Hz), 6.83-6.95(4H, m), 7.45-7.58(4H, m), 7.68-7.80(2H, m), 7.80-7.93(2H, m).

### Example 44

### 2-{4-[4-(4-Methoxyphenyl)-2-(trifluoromethyl)-1,3-oxazol-5-yl]phenoxy}ethanamine

2-(2-{4-[4-(4-Methoxyphenyl)-2-(trifluoro-methyl)-1,3-oxazol-5-yl]phenoxy}ethyl)-1H-isoindole-1,3(2H)-dione obtained by Example 43 (1.03 g, 2.03 mmol) was dissolved in a solution of 40% methylamine in methanol (5 mL) at room temperature and the mixture was stirred at the same temperature for 1 day.

The reaction mixture was evaporated in vacuo, and the residue was partitioned between water and diethyl ether. The water layer was adjusted to pH10 with saturated sodium bicarbonate solution and extracted with chloroform. The organic layer was dried over magnesium sulfate and evaporated in vacuo. The residue was purified by silica gel column chromatography (chloroform:methanol=40:1) to give the title compound (575 mg, 75%) as an oil.

¹H-NMR (300 MHz, CDCl₃): δ 3.09-3.20(2H, m), 3.85(3H, s), 4.05(2H, t, J=5 Hz), 6.90(2H, d, J=8 Hz), 6.94(2H, d, J=8 Hz), 7.54(2H, d, J=8 Hz), 7.56(2H, d, J=8 Hz). MS (ES+): 379.12.

### Example 45

### N-(2-{4-[4-(4-Methoxyphenyl)-2-(trifluoromethyl)-1,3-oxazol-5-yl]phenoxy}ethyl)urea

The title compound (58 mg, 52.1%) was obtained as a powder from 2-{4-[4-(4-methoxyphenyl)-2-(trifluoromethyl)-1,3-oxazol-5-yl]phenoxy}ethylamine obtained by Example 44 (100 mg, 0.264 mmol) in a manner similar to that of Example 18.

¹H-NMR (300 MHz, DMSO-d₆): δ 3.25-3.40(2H, m), 3.79(3H, s), 4.00(2H, t, J=5 Hz), 5.55(2H, s), 6.19(1H, t, J=5 Hz), 7.00(2H, d, J=8 Hz), 7.06(2H, d, J=8 Hz), 7.51(2H, d, J=8 Hz), 7.55(2H, d, J=8 Hz).

### Example 46

### N-(2-{4-[4-(4-Methoxyphenyl)-2-(trifluoromethyl)-1,3-oxazol-5-yl]phenoxy}ethyl)methanesulfonamide

The title compound (64.9 mg, 53.8%) was obtained as a powder from 2-{4-[4-(4-methoxyphenyl)-2-(trifluoromethyl)-1,3-oxazol-5-yl]phenoxy}ethylamine obtained by Example 44 (100 mg, 0.264 mmol) in a manner similar to that of Example 38.

¹H-NMR (300 MHz, CDCl₃): δ 3.03(3H, s), 3.53-3.61(2H, m), 3.84(3H, s), 4.15(2H, t, J=5 Hz), 4.70-4.80(1H, m), 6.85-6.95(4H, m), 7.51-7.61(4H, m). MS (ES−): 455.18.

### Example 47

### 2-{4-[4-(4-Methoxyphenyl)-2-(trifluoromethyl)-1,3-oxazol-5-yl]phenoxy}ethylamine hydrochloride

To a solution of 2-{4-[4-(4-methoxyphenyl)-2-(trifluoromethyl)-1,3-oxazol-5-yl]phenoxy}ethylamine obtained by Example 44 (288 mg, 0.761 mmol) in methanol (5 mL) was added 10% hydrogen chloride in methanol (1 mL) at room temperature. The reaction mixture was stirred at the same temperature for 30 min.

The solution was evaporated in vacuo and the residue was washed with diethyl ether to give the title compound (302 mg, 95.6%) as a yellow amorphous powder.

¹H-NMR (300 MHz, DMSO-d₆): δ 3.18-3.30(2H, m), 3.80(3H, s), 4.24(2H, t, J=5 Hz), 7.01(2H, d, J=8 Hz), 7.11(2H, d, J=8 Hz), 7.51(2H, d, J=8 Hz), 7.58 (2H, d, J=8 Hz), 8.14(3H, br peak).

### Example 48-1

### N-[2-[4-(Benzyloxy)phenyl]-1-(6-methoxy-3-pyridinyl)-2-oxoethyl]-2,2,2-trifluoroacetamide

The title compound (824 mg, 42%) was obtained as a powder from 2-amino-1-[4-(benzyloxy)phenyl]-2-(6-methoxy-3-pyridinyl)ethanone hydrochloride obtained by Example 30-5 (1.7 g, 4.42 mmol) and trifluoroacetic anhydride (1.21 g, 5.74 mmol) in a manner similar to that of Example 39-1.

¹H-NMR (300 MHz, CDCl₃): δ 3.89(3H, s), 5.10(2H, s), 6.31-6.48(1H, m), 6.68(1H, d, J=8 Hz), 6.96(2H, d, J=8 Hz), 7.26-7.45(5H, m), 7.53(1H, dd, J=8.2 Hz), 7.91(2H, d, J=8 Hz), 8.26(1H, d, J=2 Hz).

### Example 48-2

### 5-[5-[4-(Benzyloxy)phenyl]-2-(trifluoromethyl)-1,3-oxazol-4-yl]-2-methoxypyridine

The title compound (607 mg, 79.1%) was obtained as a powder from N-[2-[4-(benzyloxy)phenyl]-1-(6-methoxy-3-pyridinyl)-2-oxoethyl]-2,2,2-trifluoroacetamide obtained by Example 48-1 (800 mg, 1.8 mmol) in a manner similar to that of Example 9-5.

¹H-NMR (300 MHz, CDCl₃): δ 3.97(3H, s), 5.11(2H, s), 6.78(1H, d, J=8 Hz), 7.00(2H, d, J=8 Hz), 7.30-7.49(5H, m), 7.54(2H, d, J=8 Hz), 7.84(1H, dd, J=8.2 Hz), 8.44(1H, d, J=2 Hz). MS (ES+): 427.12.

### Example 49

### 4-[4-(6-Methoxy-3-pyridinyl)-2-(trifluoromethyl)-1,3-oxazol-5-yl]phenol

The title compound (423 mg, 88.4%) was obtained as a powder from 5-[5-[4-(benzyloxy)phenyl]-2-(trifluoromethyl)-1,3-oxazol-4-yl]-2-methoxypyridine obtained by Example 48-2 (607 mg, 1.42 mmol) in a manner similar to that of Example 31.

¹H-NMR (300 MHz, CDCl₃): δ 3.97(3H, s), 6.81(1H, d, J=8 Hz), 6.88(2H, d, J=8 Hz), 7.49(2H, d, J=8 Hz), 7.89(1H, dd, J=8.2 Hz), 8.43(1H, d, J=2 Hz). MS (ES−): 335.12.

### Example 50

### 2-{4-[4-(6-Methoxy-3-pyridinyl)-2-(trifluoromethyl)-1,3-oxazol-5-yl]phenoxy}ethanol

The title compound (305 mg, 65.8%) was obtained as a powder from 4-[4-(6-methoxy-3-pyridinyl)-2-(trifluoromethyl)-1,3-oxazol-5-yl]phenol obtained by Example 49 (410 mg, 1.22 mmol) and 2-chloroethanol (584 mg, 7.32 mmol) in a manner similar to that of Example 87 described later.

¹H-NMR (300 MHz, CDCl₃): δ 1.99(1H, t, J=7 Hz), 3.97(3H, s), 3.99(2H, dt, J=7,5 Hz), 4.12(1H, t, J=5 Hz), 6.79(1H, d, J=8 Hz), 6.96(2H, d, J=8 Hz), 7.55(2H, d, J=8 Hz), 7.84(1H, dd, J=8.2 Hz), 8.44(1H, d, J=2 Hz). MS (ES+): 381.08.

### Example 51

### 2-{4-[4-(6-Methoxy-3-pyridinyl)-2-(trifluoromethyl)-1,3-oxazol-5-yl]phenoxy}ethyl methanesulfonate

The title compound (355 mg, 99.8%) was obtained as an oil from 2-{4-[4-(6-methoxy-3-pyridinyl)-2-(trifluoromethyl)-1,3-oxazol-5-yl]phenoxy}ethanol obtained by Example 50 (295 mg, 0.776 mmol) in a manner similar to that of Example 34.

¹H-NMR (300 MHz, CDCl₃): δ 3.11(3H, s), 3.97(3H, s), 4.29(2H, t, J=5 Hz), 4.60(2H, t, J=5 Hz), 6.80(1H, d, J=8 Hz), 6.95(2H, d, J=8 Hz), 7.55(2H, d, J=8 Hz), 7.84(1H, dd, J=8.2 Hz), 8.41(1H, d, J=2 Hz). MS (ES+): 459.03.

### Example 52

### 2-(2-{4-[4-(6-Methoxy-3-pyridinyl)-2-(trifluoromethyl)-1,3-oxazol-5-yl]phenoxy}ethyl)-1H-isoindole-1,3(2H)-dione

The title compound (395 mg, 100%) was obtained as a powder from 2-{4-[4-(6-methoxy-3-pyridinyl)-2-(trifluoromethyl)-1,3-oxazol-5-yl]phenoxy}ethyl methanesulfonate obtained by Example 51 (355 mg, 0.774 mmol) and potassium phthalimide (125 mg, 1.16 mmol) in a manner similar to that of Example 35.

¹H-NMR (300 MHz, CDCl₃): δ 3.97(3H, s), 4.14(2H, t, J=5 Hz), 4.28(2H, t, J=5 Hz), 6.77(1H, d, J=9 Hz), 6.92(2H, d, J=9 Hz), 7.50(2H, d, J=9 Hz), 7.69-7.91(5H, m), 8.39(1H, d, J=2 Hz).

### Example 53

### 2-{4-[4-(6-Methoxy-3-pyridinyl)-2-(trifluoromethyl)-1,3-oxazol-5-yl]phenoxy}ethylamine

The title compound (153 mg, 53.4%) was obtained as an oil from 2-(2-{4-[4-(6-methoxy-3-pyridinyl)-2-(trifluoromethyl)-1,3-oxazol-5-yl]phenoxy}ethyl)-1H-isoindole-1,3(2H)-dione obtained by Example 52 (385 mg, 0.756 mmol) in a manner similar to that of Example 36.

¹H-NMR (300 MHz, CDCl₃): δ 3.11(2H, t, J=5 Hz), 3.97(3H, s), 4.03(2H, t, J=5 Hz), 6.79(1H, d, J=8 Hz), 6.95(2H, d, J=8 Hz), 7.54(2H, d, J=8 Hz), 7.84(1H, dd, J=8.2 Hz), 8.44(1H, d, J=2 Hz).

### Example 54

### N-(2-{4-[4-(6-Methoxy-3-pyridinyl)-2-(trifluoromethyl)-1,3-oxazol-5-yl]phenoxy}ethyl)methanesulfonamide

The title compound (53 mg, 61.3%) was obtained as an oil from 2-{4-[4-(6-methoxy-3-pyridinyl)-2-(trifluoromethyl)-1,3-oxazol-5-yl]phenoxy}ethylamine obtained by Example 53 (71.7 mg, 0.189 mmol) in a manner similar to that of Example 38.

¹H-NMR (300 MHz, CDCl₃): δ 3.04(3H, s), 3.59(2H, dd, J=6,5 Hz), 3.97(3H, s), 4.15(2H, t, J=5 Hz), 4.75(1H, t, J=6 Hz), 6.80(1H, d, J=8 Hz), 6.93(2H, d, J=8 Hz), 7.55(2H, d, J=8 Hz), 7.84(1H, dd, J=8.2 Hz), 8.42(1H, d, J=2 Hz).

### Example 55

### N-(2-{4-[4-(6-Methoxy-3-pyridinyl)-2-(trifluoromethyl)-1,3-oxazol-5-yl]phenoxy}ethyl)urea

The title compound (52 mg, 59.6%) was obtained as a powder from 2-{4-[4-(6-methoxy-3-pyridinyl)-2-(trifluoromethyl)-1,3-oxazol-5-yl]phenoxy}ethylamine obtained by Example 53 (79.3 mg, 0.201 mmol) in a manner similar to that of Example 18.

¹H-NMR (300 MHz, CDCl₃: CD₃OD=10:1): δ 3.58(2H, t, J=5 Hz), 3.97(3H, s), 4.07(2H, t, J=5 Hz), 6.81(2H, d, J=8 Hz), 6.95(2H, d, J=8 Hz), 7.53(2H, d, J=8 Hz), 7.85(1H, dd, J=8.2 Hz), 8.40(1H, d, J=2 Hz). MS (ES+): 423.15.

### Example 56-1

### N-[2-[4-(Benzyloxy)phenyl]-1-(4-methoxyphenyl)-2-oxoethyl]-2-methylpropanamide

The title compound (688 mg, 63.3%) was obtained as a powder from 2-amino-1-[4-(benzyloxy)phenyl]-2-(4-methoxyphenyl)ethanone hydrochloride obtained by Example 9-3 (1.0 g, 2.61 mmol) and isobutyryl chloride (333 mg, 3.13 mmol) in a manner similar to that of Example 7-7.

¹H-NMR (300 MHz, CDCl₃): δ 1.12(3H, d, J=7.5 Hz), 1.16(3H, d, J=7.5 Hz), 2.34-2.51(1H, m), 3.75(3H, s), 5.08(2H, s), 6.44(1H, d, J=7 Hz), 6.81(2H, d, J=8 Hz), 6.93(2H, d, J=8 Hz), 6.98(1H, d, J=7 Hz), 7.26-7.41(7H, m), 7.94(2H, d, J=8 Hz). MS (ES+): 418.16.

### Example 56-2

### 5-[4-(Benzyloxy)phenyl]-2-isopropyl-4-(4-methoxyphenyl)-1,3-oxazole

The title compound (422 mg, 74.7%) was obtained as an oil from N-[2-[4-(benzyloxy)phenyl]-1-(4-methoxyphenyl)-2-oxoethyl]-2-methylpropanamide obtained by Example 56-1 (590 mg, 1.41 mmol) in a manner similar to that of Example 1-2.

¹H-NMR (300 MHz, CDCl₃): δ 1.41(6H, d, J=7 Hz), 3.06-3.24(1H, m), 3.83(3H, s), 5.09(2H, s), 6.89(2H, d, J=9 Hz), 6.95(2H, d, J=9 Hz), 7.29-7.45(5H, m), 7.45(2H, d, J=9 Hz), 7.55(2H, d, J=9 Hz). MS (ES+): 400.25.

### Example 57

### 4-[2-Isopropyl-4-(4-methoxyphenyl)-1,3-oxazol-5-yl]phenol

The title compound (222 mg, 67.9%) was obtained as a powder from 5-[4-(benzyloxy)phenyl]-2-isopropyl-4-(4-methoxyphenyl)-1,3-oxazole obtained by Example 56-2 (422 mg, 1.06 mmol) in a manner similar to that of Example 31.

¹H-NMR (300 MHz, CDCl₃): δ 1.41(6H, d, J=7 Hz), 3.08-3.24(1H, m), 3.83(3H, s), 6.81(2H, d, J=9 Hz), 6.88(2H, d, J=9 Hz), 7.44(2H, d, J=9 Hz), 7.54(2H, d, J=9 Hz). MS (ES+): 310.24.

### Example 58

### 2-{4-[2-Isopropyl-4-(4-methoxyphenyl)-1,3-oxazol-5-yl]phenoxy}ethanol

The title compound (163 mg, 66.4%) was obtained as a powder from 4-[2-isopropyl-4-(4-methoxyphenyl)-1,3-oxazol-5-yl]phenol obtained by Example 57 (215 mg, 0.695 mmol) and 2-chloroethanol (336 mg, 4.17 mmol) in a manner similar to that of Example 87 described later.

¹H-NMR (300 MHz, CDCl₃): δ 1.42(6H, d, J=7 Hz), 2.05(1H, t, J=6 Hz), 3.04-3.25(1H, m), 3.83(3H, s), 3.94-4.01(2H, m), 4.10(2H, t, J=5 Hz), 6.85-6.94(4H, m), 7.50(2H, d, J=9 Hz), 7.55(2H, d, J=9 Hz).

### Example 59

### 2-{4-[2-Isopropyl-4-(4-methoxyphenyl)-1,3-oxazol-5-yl]phenoxy}ethyl methanesulfonate

The title compound (132 mg, 101%) was obtained as an oil from 2-{4-[2-isopropyl-4-(4-methoxyphenyl)-1,3-oxazol-5-yl]phenoxy}ethanol obtained by Example 58 (107 mg, 0.303 mmol) in a manner similar to that of Example 34.

¹H-NMR (300 MHz, CDCl₃): δ 1.42(6H, d, J=7 Hz), 3.10(3H, s), 3.11-3.25(1H, m), 3.83(3H, s), 4.24-4.30(2H, m), 4.55-4.61(2H, m), 6.84-6.92(4H, m), 7.50(2H, d, J=9 Hz), 7.55(2H, d, J=9 Hz). MS (ES+): 432.15.

### Example 60

### 2-(2-{4-[2-Isopropyl-4-(4-methoxyphenyl)-1,3-oxazol-5-yl]phenoxy}ethyl)-1H-isoindole-1,3(2H)-dione

The title compound (150 mg, 103%) was obtained as an oil from 2-{4-[2-isopropyl-4-(4-methoxyphenyl)-1,3-oxazol-5-yl]phenoxy}ethyl methanesulfonate obtained by Example 59 (130 mg, 0.301 mmol) and potassium phthalimide (83.7 mg, 0.452 mmol) in a manner similar to that of Example 35.

¹H-NMR (300 MHz, CDCl₃): δ 1.40(6H, d, J=7 Hz), 3.06-3.18(1H, m), 3.81(3H, s), 4.11(2H, t, J=5 Hz), 4.24(2H, t, J=5 Hz), 6.80-6.91(4H, m), 7.45(2H, d, J=9 Hz), 7.52(2H, d, J=9 Hz), 7.70-7.79(2H, m), 7.83-7.90(2H, m).

### Example 61

### 2-{4-[2-Isopropyl-4-(4-methoxyphenyl)-1,3-oxazol-5-yl]phenoxy}ethylamine

The title compound (106 mg, 96.8%) was obtained as an oil from 2-(2-{4-[2-isopropyl-4-(4-methoxyphenyl)-1,3-oxazol-5-yl]phenoxy}ethyl)-1H-isoindole-1,3(2H)-dione obtained by Example 60 (150 mg, 0.311 mmol) in a manner similar to that of Example 36.

hu 1H-NMR (300 MHz, CDCl₃): δ 1.41(6H, d, J=7 Hz), 3.06-3.21(1H, m), 3.83(3H, s), 4.00(2H, t, J=5 Hz), 6.81-6.93(4H, m), 7.47(2H, d, J=9 Hz), 7.54(2H, d, J=9 Hz).

### Example 62

### N-(2-{4-[2-Isopropyl-4-(4-methoxyphenyl)-1.3-oxazol-5-yl]phenoxy}ethyl)methanesulfonamide

The title compound (23 mg, 43.8%) was obtained as a powder from 2-{4-[2-isopropyl-4-(4-methoxyphenyl)-1,3-oxazol-5-yl]phenoxy}ethylamine obtained by Example 61 (43 mg, 0.122 mmol) in a manner similar to that of Example 38.

¹H-NMR (300 MHz, CDCl₃): δ 1.42(6H, d, J=7 Hz), 3.04(3H, s), 3.08-3.22(1H, m), 3.56(2H, q, J=5 Hz), 3.83(3H, s), 4.12(2H, t, J=5 Hz), 4.75(1H, br peak), 6.85(2H, d, J=9 Hz), 6.90(2H, d, J=9 Hz), 7.50(2H, d, J=9 Hz), 7.54(2H, d, J=9 Hz). MS (ES+): 431.13.

### Example 63

### N-(2-{4-[2-Isopropyl-4-(4-methoxyphenyl)-1,3-oxazol-5-yl]phenoxy}ethyl)urea

The title compound (23 mg, 32.5%) was obtained as an oil from 2-{4-[2-isopropyl-4-(4-methoxyphenyl)-1,3-oxazol-5-yl]phenoxy}ethylamine obtained by Example 62 (63 mg, 0.179 mmol) in a manner similar to that of Example 18.

¹H-NMR (300 MHz, CDCl₃) δ 1.41(6H, d, J=7 Hz), 3.08-3.21(1H, m), 3.61(2H, q, J=5 Hz), 3.83(3H, s), 4.05(2H, t, J=5 Hz), 4.40(2H, br-s), 4.95(1H, br peak), 6.85(2H, d, J=9 Hz), 6.89(2H, d, J=9 Hz), 7.49(2H, d, J=9 Hz), 7.54(2H, d, J=9 Hz). MS (ES+): 396.20.

### Example 64-1

### 1,2-Bis(4-methoxyphenyl)-2-oxoethyl (benzyloxy)acetate

To a solution of anisoin (500 mg, 1.84 mmol) and pyridine (581 mg, 7.34 mmol) in dichloromethane (10 mL) was added benzyloxyacetyl chloride (424 mg, 2.30 mmol) under nitrogen at room temperature, and the mixture was stirred at the same temperature for 22 hrs.

The mixture was poured into 1 mol/L hydrochloric acid and extracted with chloroform. The organic layer was washed with 1 mol/L hydrochloric acid and water, dried over magnesium sulfate, and evaporated in vacuo to give the title compound (775 mg, 100.4%) as an oil.

¹H-NMR (300 MHz, CDCl₃): δ 3.78(3H, s), 3.83(3H, s), 4.21(1H, d, J=17 Hz), 4.32(1H, d, J=17 Hz), 4.68(2H, s), 6.82-6.92(5H, m), 7.21-7.42(7H, m), 7.91(2H, d, J=8 Hz).

### Example 64-2

### 2-[(Benzyloxy)methyl]-4,5-bis(4-methoxyphenyl)-1,3-oxazole

To a solution of 1,2-bis(4-methoxyphenyl)-2-oxoethyl (benzyloxy)acetate obtained by Example 64-1 (775 mg, 1.84 mmol) in acetic acid (14 mL) was added ammonium acetate (1.42 g, 18.4 mmol) at room temperature, and the mixture was heated to reflux with stirring for 1 hr.

After cooling, the reaction mixture was evaporated in vacuo and acetic acid was azeotropically removed with toluene. The residue was partitioned between water and ethyl acetate. The organic layer was separated, washed with water, saturated sodium bicarbonate solution and brine, successively, dried over magnesium sulfate. After evaporation of solvent, the residue was purified by silica gel column chromatography (n-hexane ethyl acetate=4:1) and triturated with ethanol to give the title compound (300 mg, 40.5%) as a pale yellow powder.

¹H-NMR (300 MHz, CDCl₃): δ 3.84(6H, s), 4.67(2H, s), 4.70(2H, s), 6.84-6.94(4H, m), 7.26-7.44(5H, m), 7.51(2H, d, J=8 Hz), 7.56(2H, d, J=8 Hz). MS (ES+): 402.12.

### Example 65

### [4,5-Bis(4-methoxyphenyl)-1,3-oxazol-2-yl]methanol

A mixture of 2-[(benzyloxy)methyl]-4,5-bis(4-methoxyphenyl)-1,3-oxazole obtained by Example 64-2 (88 mg, 0.219 mmol) and 10% palladium on carbon (20 mg) in a mixture of methanol (2 mL) and tetrahydrofuran (2 mL) was stirred at room temperature under hydrogen for 6 hrs.

The reaction mixture was filtered through Celite and evaporated in vacuo. The residue was purified by preparative thin layer chromatography (n-hexane:ethyl acetate=1:1), and triturated with a mixture of hexane and diethyl ether to give the title compound (44 mg, 65.4%) as a pale yellow powder.

¹H-NMR (300 MHz, CDCl₃): δ 2.36(1H, t, J=7 Hz), 3.84(6H, s), 4.79(2H, d, J=7 Hz), 6.85-6.94(4H, m), 7.51(2H, d, J=8 Hz), 7.56(2H, d, J=8 Hz). MS (ES+): 312.13.

### Example 66-1

### 1,2-Bis(4-methoxyphenyl)-2-oxoethyl ethyl malonate

The title compound (644 mg, 90.8%) was obtained as an oil from anisoin (500 mg, 1.84 mmol) and ethyl 3-chloro-3-oxopropionate (346 mg, 2.30 mmol) in a manner similar to that of Example 64-1.

¹H-NMR (300 MHz, DMSO-d₆): δ 1.26(3H, t, J=7.5 Hz), 3.53(2H, s), 3.79(3H, s), 3.83(3H, s), 4.20(2H, q, J=7.5 Hz), 6.81-6.93(5H, m), 7.38(2H, d, J=8 Hz), 7.91(2H, d, J=8 Hz).

### Example 66-2

### Ethyl[4,5-bis(4-methoxyphenyl)-1,3-oxazol-2-yl]acetate

The title compound (186 mg, 30.4%) was obtained an oil from 1,2-bis(4-methoxyphenyl)-2-oxoethyl ethyl malonate obtained by Example 66-1 (644 mg, 1.67 mmol) and ammonium acetate (1.28 g, 16.7 mmol) in a manner similar to that of Example 64-2.

¹H-NMR (300 MHz, CDCl₃): δ 1.31(3H, t, J=7.5 Hz), 3.84(6H, s), 3.92(2H, s), 4.25(2H, q, J=7.5 Hz), 6.90(4H, d, J=8 Hz), 7.45-7.65(4H, m). MS (ES+): 368.14.

### Example 67

### [4,5-Bis(4-methoxyphenyl)-1,3-oxazol-2-yl]acetic acid

To a solution of ethyl [4,5-bis(4-methoxyphenyl)-1,3-oxazol-2-yl]acetate obtained by Example 66-2 (70 mg, 0.191 mmol) in ethanol (2 mL) was added 1 mol/L sodium hydroxide solution (0.25 mL) at room temperature, and the mixture was stirred at the same temperature for 3 hrs.

The reaction mixture was evaporated in vacuo and dissolved in water. The water solution was washed with ether, adjusted to pH1 with 6N hydrochloric acid, and extracted with ethyl acetate. The organic layer was dried over magnesium sulfate and evaporated in vacuo. The residue was triturated with diethyl ether to give the title compound (31 mg, 47.9%) as an amorphous powder.

¹H-NMR (300 MHz, DMSO-d₆): δ 3.63(2H, br-s), 3.77(3H, s), 3.79(3H, s), 6.95(2H, d, J=8 Hz), 6.99(2H, d, J=8 Hz), 7.43(2H, d, J=8 Hz), 7.49(2H, d, J=8 Hz). MS (ES+): 340.15.

### Example 68-1

### 2-Bromo-2-(4-methoxyphenyl)-1-(6-methoxy-3-pyridinyl)ethanone

To a solution of 2-(4-methoxyphenyl)-1-(6-methoxy-3-pyridinyl)ethanone (1.0 g, 3.89 mmol) in dichloromethane (10 mL) were added pyridinium tribromide (1.37 g, 4.28 mmol) and hydrogen bromide (33% solution in acetic acid, 1 mL) at room temperature under nitrogen, and the mixture was stirred at the same temperature for 40 min.

The reaction mixture was evaporated in vacuo and acetic acid was azeotropically removed with toluene. The residue was partitioned between water and ethyl acetate. The organic layer was separated, washed with water and brine, dried over magnesium sulfate, and evaporated in vacuo to give the title compound (1.32 g, 101%) as an oil.

¹H-NMR (300 MHz, CDCl₃): δ 3.80(3H, s), 3.99(3H, s), 6.29(1H, s), 6.77(1H, d, J=8 Hz), 6.90(2H, d, J=8 Hz), 7.45(2H, d, J=8 Hz), 8.16(1H, dd, J=8.2 Hz), 8.80(1H, d, J=2 Hz).

### Example 68-2

### 2-Hydroxy-2-(4-methoxyphenyl)-1-(6-methoxy-3-pyridinyl)ethanone

2-Bromo-2-(4-methoxyphenyl)-1-(6-methoxy-3-pyridinyl)ethanone obtained by Example 68-1 (1.30 g, 3.87 mmol) was dissolved in acetone (10 mL) and water (5 mL), and heated to reflux for 1 hr.

The reaction mixture was evaporated in vacuo, and the residue was partitioned between water and ethyl acetate. The organic layer was separated, washed with water and brine, dried over magnesium sulfate, and evaporated in vacuo. The residue was purified by silica gel column chromatography (n-hexane:ethyl acetate=2:1) to give the title compound (770 mg, 72.9%) as an oil.

¹H-NMR (300 MHz, CDCl₃): δ 3.77(3H, s), 3.96(3H, s), 4.46(1H, d, J=7 Hz), 5.80(1H, d, J=7 Hz), 6.74(1H, d, J=8 Hz), 6.86(2H, d, J=8 Hz), 7.25(2H, d, J=8 Hz), 8.10(1H, dd, J=8.2 Hz), 8.72(1H, d, J=2 Hz).

### Example 68-3

### 1-(4-Methoxyphenyl)-2-(6-methoxy-3-pyridinyl)-2-oxoethyl methoxyacetate

The title compound (128 mg, 101.3%) was obtained as an oil from 2-hydroxy-2-(4-methoxyphenyl)-1-(6-methoxy-3-pyridinyl)ethanone obtained by Example 68-2 (100 mg, 0.366 mmol) and methoxyacetyl chloride (47.7 mg, 0.439 mmol) in a manner similar to that of Example 64-1.

¹H-NMR (300 MHz, CDCl₃): δ 3.48(3H, s), 3.88(3H, s), 3.96(3H, s), 4.16(1H, d, J=17 Hz), 4.25(1H, d, J=17 Hz), 6.74(1H, d, J=8 Hz), 6.80(1H, s), 6.90(2H, d, J=8 Hz), 7.36(2H, d, J=8 Hz), 8.10(1H, dd, J=8.2 Hz), 8.75(1H, d, J=2 Hz).

### Example 68-4

### 2-Methoxy-5-[2-(methoxymethyl)-5-(4-methoxyphenyl)-1,3-oxazol-4-yl]pyridine

The title compound (80 mg, 66.1%) was obtained as an oil from 1-(4-methoxyphenyl)-2-(6-methoxy-3-pyridinyl)-2-oxoethyl methoxyacetate obtained by Example 68-3 (128 mg, 0.371 mmol) and ammonium acetate (286 mg, 3.71 mmol) in a manner similar to that of Example 64-2.

¹H-NMR (300 MHz, CDCl₃): δ 3.52(3H, s), 3.84(3H, s), 3.96(3H, s), 4.60(2H, s), 6.75(1H, d, J=8 Hz), 6.90(2H, d, J=8 Hz), 7.50(2H, d, J=8 Hz), 7.83(1H, dd, J=8.2 Hz), 8.42(1H, d, J=2 Hz).

### Example 69-1

### 1-(4-Methoxyphenyl)-2-(6-methoxy-3-pyridinyl)-2-oxoethyl (acetyloxy)acetate

The title compound (990 mg, 100.2%) was obtained as an oil from 2-hydroxy-2-(4-methoxyphenyl)-1-(6-methoxy-3-pyridinyl)ethanone obtained by Example 68-2 (725 mg, 2.65 mmol) and acetoxyacetyl chloride (542 mg, 3.97 mmol) in a manner similar to that of Example 64-1.

¹H-NMR (300 MHz, CDCl₃): δ 2.15(3H, s), 3.79(3H, s), 3.96(3H, s), 4.74(1H, d, J=17 Hz), 4.81(1H, d, J=17 Hz), 6.74(1H,d, J=8 Hz), 6.77(1H, s), 6.90(2H, d, J=8 Hz), 7.37(2H, d, J=8 Hz), 8.09(1H, dd, J=8.2 Hz), 8.74(1H, d, J=2 Hz).

### Example 69-2

### [5-(4-Methoxyphenyl)-4-(6-methoxy-3-pyridinyl)-1,3-oxazol-2-yl]methyl acetate

The title compound (415 mg, 48%) was obtained as an oil from 1-(4-methoxyphenyl)-2-(6-methoxy-3-pyridinyl)-2-oxoethyl (acetyloxy)acetate obtained by Example 69-1 (990 mg, 2.65 mmol) and ammonium acetate (2.04 g, 26.5 mmol) in a manner similar to that of Example 64-2.

¹H-NMR (300 MHz, CDCl₃): δ 2.18(3H, s), 3.84(3H, s), 3.96(3H, s), 5.22(2H, s), 6.75(1H, d, J=8 Hz), 6.91(2H, d, J=8 Hz), 7.50(2H, d, J=8 Hz), 7.83(1H, dd, J=8.2 Hz), 8.42(1H, d, J=2 Hz).

### Example 70

### [5-(4-Methoxyphenyl)-4-(6-methoxy-3-pyridinyl)-1,3-oxazol-2-yl]methanol

To a solution of [5-(4-methoxyphenyl)-4-(6-methoxy-3-pyridinyl)-1,3-oxazol-2-yl]methyl acetate obtained by Example 69-2 (410 mg, 1.26 mmol) in methanol (8 mL) was added potassium carbonate (208 mg, 1.51 mmol) at room temperature, and the mixture was stirred at the same temperature for 1 hr.

The reaction mixture was evaporated in vacuo, and the residue was partitioned between water and ethyl acetate. The organic layer was separated, washed with 1 mol/L hydrochloric acid, water, saturated sodium bicarbonate solution and brine, dried over magnesium sulfate, and evaporated in vacuo: The residue was purified by silica gel column chromatography (n-hexane:ethyl acetate=2:1) and triturated with isopropyl ether to give the title compound (247 mg, 63.0%) as an amorphous powder.

¹H-NMR (300 MHz, CDCl₃): δ 2.61(1H, t, J=7 Hz), 3.84(3H, s), 3.97(3H, s), 4.80(2H, d, J=7 Hz), 6.75(1H, d, J=8 Hz), 6.90(2H, d, J=8 Hz), 7.49(2H, d, J=8 Hz), 7.81(1H, dd, J=8.2 Hz), 8.42(1H, d, J=2 Hz). MS (ES+): 313.06.

### Example 71

### 5-(4-Methoxyphenyl)-4-(6-methoxy-3-pyridinyl)-1,3-oxazole-2-carbaldehyde

A mixture of [5-(4-methoxyphenyl)-4-(6-methoxy-3-pyridinyl)-1,3-oxazol-2-yl]methanol obtained by Example 70 (192 mg, 0.615 mmol) and manganese (IV) oxide (187 mg, 2.15 mmol) in chloroform (5 mL) was heated to reflux with stirring for 2 hrs.

After cooling, the reaction mixture was filtered through Celite and evaporated in vacuo. The residue was triturated with petroleum ether to give the title compound (178 mg, 93.3%) as an amorphous powder.

¹H-NMR (300 MHz, CDCl₃): δ 3.86(3H, s), 3.99(3H, s), 6.81(1H, d, J=8 Hz), 6.94(2H, d, J=8 Hz), 7.62(2H, d, J=8 Hz), 7.86(1H, dd, J=8.2 Hz), 8.48(2H, d, J=8 Hz), 9.78(1H, s).

### Example 72

### [5-(4-Methoxyphenyl)-4-(6-methoxy-3-pyridinyl)-1,3-oxazol-2-yl](phenyl)methanol

To a solution of 5-(4-methoxyphenyl)-4-(6-methoxy-3-pyridinyl)-1,3-oxazole-2-carbaldehyde obtained by Example 71 (70 mg, 0.226 mmol) in tetrahydrofuran (3 mL) was added 3N solution of phenylmagnesium bromide in diethyl ether (0.1 mL, 0.3 mmol) dropwise at 0° C. under nitrogen, and the mixture was stirred at the same temperature for 3 hrs.

The reaction mixture was poured into water and extracted with ethyl acetate. The organic layer was washed with 1 mol/L hydrochloric acid, water, saturated sodium bicarbonate solution and brine, dried over magnesium sulfate, and evaporated in vacuo. The residue was purified by preparative thin layer chromatography (n-hexane:ethyl acetate=2:1) to give the title compound (62.3 mg, 71.1%) as an oil.

¹H-NMR (300 MHz, CDCl₃): δ 3.30(1H, d, J=7 Hz), 3.82(3H, s), 3.96(3H, s), 5.93(1H, d, J=7 Hz), 6.75(1H, d, J=8 Hz), 6.87(2H, d, J=8 Hz), 7.32-7.46(5H,m), 7.55(2H, d, J=8 Hz), 7.83(1H, dd, J=8.2 Hz), 8.41(1H, d, J=2 Hz). MS (ES+): 389.10.

### Example 73

### [5-(4-Methoxyphenyl)-4-(6-methoxy-3-pyridinyl)-1,3-oxazol-2-yl](phenyl)methanone

The title compound (42 mg, 70.4%) was obtained as yellow crystals from [5-(4-methoxyphenyl)-4-(6-methoxy-3-pyridinyl)-1,3-oxazol-2-yl](phenyl)methanol obtained by Example 72 (60 mg, 0.154 mmol) in a manner similar to that of Example 71.

MP: 156-158° C. ¹H-NMR (300 MHz, CDCl₃): δ 3.87(3H, s), 3.99(3H, s), 6.82(1H, d, J=8 Hz), 6.95(2H, d, J=8 Hz), 7.50-7.58(2H, m), 7.62-7.70(3H, m), 7.90(1H, dd, J=8.2 Hz), 8.53-8.59(3H, m). MS (ES+): 387.05.

### Example 74

### 5-(4-Methoxyphenyl)-4-(6-methoxy-3-pyridinyl)-1,3-oxazole-2-carboxylic acid

To a suspension of 5-(4-methoxyphenyl)-4-(6-methoxy-3-pyridinyl)-1,3-oxazole-2-carbaldehyde obtained by Example 71 (103 mg, 0.332 mmol) in a mixture of water (0.8 mL) and tert-buthylalcohol (3 mL) were added 2-methyl-2-butene (103 mg, 1.47 mmol) and sodium dihydrogenphosphate (43.8 mg, 0.365 mmol) in water bath. To the mixture was added sodium chlorite (133 mg, 1. 47 mmol) portionwise and the resulting mixture was stirred in water bath for 1.5 hrs.

The reaction mixture was evaporated in vacuo, and the residue was dissolved in water. The solution was adjusted to pH4 with 1 mol/L hydrochloric acid and extracted with chloroform. The organic layer was dried over magnesium sulfate and evaporated in vacuo to give the title compound (110 mg, 101.6%) as an amorphous powder.

¹H-NMR (300 MHz, CDCl₃): δ 3.85(3H, s), 3.97(3H, s), 6.80(1H, d, J=8 Hz), 6.94(2H, d, J=8 Hz), 7.58(2H, d, J=8 Hz), 7.87(2H, d, J=8 Hz), 8.44(1H, s). MS (ES+): 327.03.

### Example 75

### 5-(4-Methoxyphenyl)-4-(6-methoxy-3-pyridinyl)-1,3-oxazole-2-carboxamide

A mixture of 5-(4-methoxyphenyl)-4-(6-methoxy-3-pyridinyl)-1,3-oxazole-2-carboxylic acid obtained by Example 74 (110 mg, 0.337 mmol), 1-hydroxybenzotriazole (61.5 mg, 0.455 mmol) and 1-(3-dimethylaminopropyl)-3-ethylcarbodiimide hydrochloride (84 mg, 0.438 mmol) in N,N-dimethylformamide (6 mL) was added ammonia solution (28%, 27 mg, 0.438 mmol) at 0° C., and the mixture was stirred at the same temperature for 18 hrs.

The mixture was partitioned between water and ethyl acetate. The organic layer was separated, washed with 1 mol/L hydrochloric acid, water, saturated sodium bicarbonate solution and brine, dried over magnesium sulfate, and evaporated in vacuo to give the title compound (110 mg, 100.3%) as an amorphous powder.

¹H-NMR (300 MHz, CDCl₃): δ 3.85(3H, s), 3.98(3H, s), 5.69(1H, br s), 6.79(1H, d, J=8 Hz), 6.89-7.02(3H, m), 7.59(2H, d, J=8 Hz), 7.82(1H, dd, J=8.2 Hz), 8.45(1H, d, J=2 Hz). MS (ES+): 326.06.

### Example 76

### 5-(4-Methoxyphenyl)-4-(6-methoxy-3-pyridinyl)-1,3-oxazole-2-carbonitrile

The title compound (57 mg, 54.9%) was obtained as an amorphous powder from 5-(4-methoxyphenyl)-4-(6-methoxy-3-pyridinyl)-1,3-oxazole-2-carboxamide obtained by Example 75 (110 mg, 0.338 mmol) in a manner similar to that of Example 3.

¹H-NMR (300 MHz, CDCl₃): δ 3.86(3H, s), 3.98(3H, s), 6.80(1H, d, J=8 Hz), 6.95(2H, d, J=8 Hz), 7.54(2H, d, J=8 Hz), 7.81(1H, dd, J=8.2 Hz), 8.44(1H, d, J=2 Hz). MS (ES+): 308.04.

### Example 77

### 5-[2-(Difluoromethyl)-5-(4-methoxyphenyl)-1,3-oxazol-4-yl]-2-methoxypyridine

To a solution of 5-(4-methoxyphenyl)-4-(6-methoxy-3-pyridinyl)-1,3-oxazole-2-carbaldehyde obtained by Example 71 (100 mg, 0.322 mmol) in dichloromethane (2 mL) was added diethylaminosulfur trifluoride (62.3 mg, 0.51 mmol) at 0° C. under nitrogen, and the mixture was stirred at the same temperature for 3 hrs.

The reaction mixture was partitioned between water and chloroform. The organic layer was separated, washed with 1 mol/L hydrochloric acid, water, saturated sodium bicarbonate solution and brine, dried over magnesium sulfate, and evaporated in vacuo. The residue was purified by preparative thin layer chromatography (toluene:ethyl acetate=9:1) and triturated with hexane to give the title compound (41 mg, 38.3%) as an amorphous powder.

MP: 87-89° C. ¹H-NMR (300 MHz, CDCl₃): δ 3.85(3H, s), 3.97(3H, s), 6.71(1H, t, J=52 Hz), 6.78(1H, d, J=8 Hz), 6.94(2H, d, J=8 Hz), 7.54(2H, d, J=8 Hz), 7.82(1H, dd, J=8.2 Hz), 8.44(1H, d, J=2 Hz). MS (ES+): 333.08.

### Example 78-1

### Diphenyl anilino(4-cyanophenyl)methylphosphonate

To a solution of 4-formylbenzonitrile (175 g) in isopropyl acetate (2.1 L) was added potassium fluoride (77.5 mg) followed by addition of aniline (124 g), and the mixture was heated to 60° C. with stirring. To the mixture was added dropwise diphenyl phosphonate (469 g) over 45 min, and the mixture was heated at 60° C. for additional 30 min. To the mixture was added dropwise n-heptane (2.8 L) over 2 hrs, and the mixture was cooled to 15° C.

The resulting precipitate was collected by filtration, washed successively with water, 50% isopropyl acetate in n-heptane, and dried to give the title compound as crystals (494 g, 84%).

¹H-NMR (300 MHz, DMSO-d₆): δ 5.70-6.00(1H, m), 6.61(1H, t, J=7 Hz), 6.80-7.49(15H, m), 7.79-8.00(4H, m).

### Example 78-2

### 4-[(4-Methoxyphenyl)acetyl]benzonitrile

To a mixture of diphenyl anilino(4-cyanophenyl)methylphosphonate obtained by Example 78-1 (493 g) and 4-methoxybenzaldehyde (168 g) in tetrahydrofuran (1.0 L) and 2-propanol (2.8 L) was added potassium tert-butoxide (138 g) in tetrahydrofuran (1.8 L) over 6 hrs. The mixture was stirred for additional 30 min. To the mixture was added dropwise 2N hydrochloric acid (2.0 L), and the mixture was heated at 45° C. for 1 hr.

The mixture was neutralized to pH 6 by adding 6N sodium hydroxide solution (700 mL). The mixture was cooled to 5° C., and the resulting precipitate was collected by filtration, washed successively with 50% 2-propanol in cooled water, water, and dried to give the title compound as crystals (200 g, 71%).

¹H-NMR (300 MHz, CDCl₃): δ 3.78(3H, s), 4.23(2H, s), 6.87(2H, d, J=8.4 Hz), 7.15(2H, d, J=8.4 Hz), 7.74(2H, d, J=8.2 Hz), 8.07(2H, d, J=8.2 Hz).

### Example 78-3

### 4-[Bromo(4-methoxyphenyl)acetyl]benzonitrile

To a solution of 4-[(4-methoxyphenyl)acetyl]benzonitrile obtained by Example 78-2 (3.0 g, 11.9 mmol) in tetrahydrofuran (30 mL) was added pyridinium tribromide (3.82 g, 11.9 mmol) portionwise at room temperature under nitrogen, and the mixture was stirred at the same temperature for 1.5 hrs.

The reaction mixture was partitioned between water and ethyl acetate. The organic layer was separated, washed with water and brine, dried over magnesium sulfate, and evaporated in vacuo. The residue was triturated with hexane to give the title compound (3.77 g, 95.6%) as a powder.

¹H-NMR (300 MHz, CDCl₃): δ 3.81(3H, s), 6.24(1H, s), 6.91(2H, d, J=8 Hz), 7.44(2H, d, J=8 Hz), 7.75(2H, d, J=8 Hz), 8.06(2H, d, J=8 Hz).

### Example 78-4

### 2-(4-Cyanophenyl)-1-(4-methoxyphenyl)-2-oxoethyl (acetyloxy)acetate

To a solution of 4-[bromo(4-methoxyphenyl)acetyl]benzonitrile obtained by Example 78-3 (500 mg, 1.51 mmol) in acetone were added acetoxyacetic acid (179 mg, 1.51 mmol) and cesium carbonate (493 mg, 1.51 mmol) at room temperature under nitrogen, and the mixture was stirred at the same temperature for 18 hrs.

The reaction mixture was evaporated in vacuo, and the residue was partitioned between water and ethyl acetate. The organic layer was separated, washed with 1 mol/L hydrochloric acid, water, saturated sodium bicarbonate solution and brine, dried over magnesium sulfate, and evaporated in vacuo. The residue was purified by silica gel column chromatography (n-hexane:ethyl acetate=2:1) to give the title compound (337 mg, 60.6%) as an oil.

¹H-NMR (300 MHz, CDCl₃): δ 2.15(3H, s), 3.85(3H, s), 4.74(1H, d, J=16 Hz), 4.82(1H, d, J=16 Hz), 6.87-6.96(3H, m), 7.58(2H, d, J=9 Hz), 7.68(2H, d, J=9 Hz), 7.90(2H, d, J=9 Hz). MS (ES−): 366.15.

### Example 78-5

### [4-(4-Cyanophenyl)-5-(4-methoxyphenyl)-1,3-oxazol-2-yl]methyl acetate

The title compound (250 mg, 78.8%) was obtained as an oil from 2-(4-cyanophenyl)-1-(4-methoxyphenyl)-2-oxoethyl (acetyloxy)acetate obtained by Example 78-4 (335 mg, 0.912 mmol) and ammonium acetate (562 mg, 7.3 mmol) in a manner similar to that of Example 64-2.

¹H-NMR (300 MHz, CDCl₃): δ 2.19(3H, s), 3.86(3H, s), 5.25(2H, s), 6.95(2H, d, J=8 Hz), 7.53(2H, d, J=8 Hz), 7.63(2H, d, J=8 Hz), 7.71(2H, d, J=8 Hz). MS (ES+): 349.03.

### Example 79

### 4-[2-(Hydroxymethyl)-5-(4-methoxyphenyl)-1,3-oxazol-4-yl]benzonitrile

The title compound (100 mg, 45.5%) was obtained as a powder from [4-(4-cyanophenyl)-5-(4-methoxyphenyl)-1,3-oxazol-2-yl]methyl acetate obtained by Example 78-5 (250 mg, 0.718 mmol) in a manner similar to that of Example 70.

MP: 151-153° C. ¹H-NMR (300 MHz, CDCl₃): δ 2.50(1H, t, J=5 Hz), 3.87(3H, s), 4.84(2H, d, J=5 Hz), 6.95(2H, d, J=8 Hz), 7.53(2H, d, J=8 Hz), 7.62(2H, d, J=8 Hz), 7.70(2H, d, J=8 Hz). MS (ES+): 307.03.

### Example 80-1

### 4-[l-Bromo-2-(4-methoxyphenyl)-2-oxoethyl]benzonitrile

The title compound (2.09 g, 106%) was obtained as a powder from 4-[2-(4-methoxyphenyl)-2-oxoethyl]-benzonitrile (1.5 g, 5.97 mmol) in a manner similar to that of Example 78-3.

¹H-NMR (300 MHz, CDCl₃): δ 3.88(3H, s), 6.28(1H, s), 6.96(2H, d, J=8 Hz), 7.67(4H, s), 7.98(2H, d, J=8 Hz).

### Example 80-2

### 1-(4-Cyanophenyl)-2-(4-methoxyphenyl)-2-oxoethyl methoxyacetate

The title compound (426 mg, 82.9%) was obtained as an oil from 4-[1-bromo-2-(4-methoxyphenyl)-2-oxoethyl]benzonitrile obtained by Example 80-1 (500 mg, 1.51 mmol) and methoxyacetic acid (179 mg, 1.51 mmol) in a manner similar to that of Example 78-4.

¹H-NMR (300 MHz, CDCl₃): δ 3.48(3H, s), 3.85(3H, s), 4.17(1H, d, J=15 Hz), 4.25(1H, d, J=15 Hz), 6.90(2H, d, J=8 Hz), 6.95(1H, s), 7.59(2H, d, J=8 Hz), 7.66(2H, d, J=8 Hz), 7.91(2H, d, J=8 Hz). MS (ES−): 338.18.

### Example 80-3

### 4-[2-(Methoxymethyl)-4-(4-methoxyphenyl)-1,3-oxazol-5-yl]benzonitrile

The title compound (188 mg, 47.1%) was obtained as crystals from 1-(4-cyanophenyl)-2-(4-methoxyphenyl)-2-oxoethyl methoxyacetate obtained by Example 80-2 (423 mg, 1.51 mmol) in a manner similar to that of Example 64-2.

MP: 85-86° C. ¹H-NMR (300 MHz, CDCl₃): δ 3.53(3H, s), 3.86(3H, s), 4.62(2H, s), 6.95(2H, d, J=8 Hz), 7.54(2H, d, J=8 Hz), 7.62(2H, d, J=8 Hz), 7.73(2H, d, J=8 Hz). MS (ES+): 321.08.

### Example 81-1

### 2-(4-Cyanophenyl)-1-(4-methoxyphenyl)-2-oxoethyl methoxyacetate

The title compound (229 mg, 89.1%) was obtained as an oil from 4-[bromo(4-methoxyphenyl)acetyl]-benzonitrile obtained by Example 78-3 (250 mg, 0.757 mmol) and methoxyacetic acid (89.4 mg, 0.757 mmol) in a manner similar to that of Example 78-4.

¹H-NMR (300 MHz, CDCl₃): δ 3.48(3H, s), 3.79(3H, s), 4.16(1H, d, J=15 Hz), 4.25(1H, d, J=15 Hz), 6.82(1H, s), 6.90(2H, d, J=8 Hz), 7.34(2H, d, J=8 Hz), 7.70(2H, d, J=8 Hz), 7.96(2H, d, J=8 Hz).

### Example 81-2

### 4-[2-(Methoxymethyl)-5-(4-methoxyphenyl)-1,3-oxazol-4-yl]benzonitrile

The title compound (47 mg, 21.9%) was obtained as crystals from 2-(4-cyanophenyl)-1-(4-methoxyphenyl)-2-oxoethyl methoxyacetate obtained by Example 81-1 (227 mg, 0.669 mmol) in a manner similar to that of Example 64-2.

¹H-NMR (300 MHz, CDCl₃): δ 3.52(3H, s), 3.86(3H, s), 4.60(2H, s), 6.94(2H, d, J=8 Hz), 7.50(2H, d, J=8 Hz), 7.62(2H, d, J=8 Hz), 7.80(2H, d, J=8 Hz). MS (ES+): 321.10.

### Example 82-1

### 2-[4-(Benzyloxy)phenyl]-1-(4-methoxyphenyl)-2-oxoethyl (acetyloxy)acetate

The title compound (1.26 g, 100%) was obtained as an oil from 1-[4-(benzyloxy)phenyl]-2-bromo-2-(4-methoxyphenyl)ethanone obtained by Example 9-1 (1.24 g, 2.81 mmol) and acetoxyacetic acid (332 mg, 2.81 mmol) in a manner similar to that of Example 78-4.

¹H-NMR (300 MHz, CDCl₃): δ 2.14(3H, s), 3.78(3H, s), 4.72(1H, d, J=15 Hz), 4.80(1H, d, J=15 Hz), 5.08(2H, s), 6.85(1H, s), 6.87(2H, d, J=8 Hz), 6.93(2H, d, J=8 Hz), 7.30-7.43(7H, m), 7.89(2H, d, J=8 Hz).

### Example 82-2

### [4-[4-(Benzyloxy)phenyl]-5-(4-methoxyphenyl)-1,3-oxazol-2-yl]methyl acetate

The title compound (1.2 g, 99.5%) was obtained as an oil from 2-[4-(benzyloxy)phenyl]-1-(4-methoxyphenyl)-2-oxoethyl (acetyloxy)acetate obtained by Example 82-1 (1.26 g, 2.81 mmol) and ammonium acetate (1.73 g, 22.5 mmol) in a manner similar to that of Example 64-2.

¹H-NMR (300 MHz, CDCl₃): δ 2.17(3H, s), 3.84(3H, s), 5.09(2H, s), 5.21(2H, s), 6.90(2H, d, J=8 Hz), 6.93(2H, d, J=8 Hz), 7.28-7.47(5H, m), 7.52(2H, d, J=8 Hz), 7.56(2H, d, J=8 Hz).

### Example 83

### [4-[4-(Benzyloxy)phenyl]-5-(4-methoxyphenyl)-1,3-oxazol-2-yl]methanol

The title compound (570 mg, 52.7%) was obtained as an oil from [4-[4-(benzyloxy)phenyl]-5-(4-methoxyphenyl)-1,3-oxazol-2-yl]methyl acetate obtained by Example 82-2 (1.2 g, 2.79 mmol) in a manner similar to that of Example 70.

¹H-NMR (300 MHz, CDCl₃): δ 2.70(1H, br peak), 3.84(3H, s), 4.80(2H, s), 5.09(2H, s), 6.90(2H, d, J=8 Hz), 6.98(2H, d, J=8 Hz), 7.30-7.47(5H, m), 7.51(2H, d, J=8 Hz), 7.56(2H, d, J=8 Hz). MS (ES+): 388.06.

### Example 84

### 4-[4-(Benzyloxy)phenyl]-5-(4-methoxyphenyl)-1,3-oxazole-2-carbaldehyde

The title compound (438 mg, 77.2%) was obtained as a powder from [4-[4-(benzyloxy)phenyl]-5-(4-methoxyphenyl)-1,3-oxazol-2-yl]methanol obtained by Example 83 (570 mg, 1.47 mmol) in a manner similar to that of Example 71.

¹H-NMR (300 MHz, CDCl₃): δ 3.85(3H, s), 5.12(2H, s), 6.91(2H, d, J=8 Hz), 7.02(2H, d, J=8 Hz), 7.30-7.50(5H, m), 7.60(2H, d, J=8 Hz), 7.65(2H, d, J=8 Hz), 9.76(1H, s).

### Example 85

### 4-[4-(Benzyloxy)phenyl]-2-(difluoromethyl)-5-(4-methoxyphenyl)-1,3-oxazole

The title compound (392 mg, 76.5%) was obtained as a powder from 4-[4-(benzyloxy)phenyl]-5-(4-methoxyphenyl)-1,3-oxazole-2-carbaldehyde obtained by Example 84 (485 mg, 1.26 mmol) in a manner similar to that of Example 77.

¹H-NMR (300 MHz, CDCl₃): δ 3.85(3H, S), 5.10(2H, s), 6.70(1H, t, J=53 Hz), 6.92(2H, d, J=8 Hz), 6.99(2H, d, J=8 Hz), 7.29-7.49(5H, m), 7.53-7.61(4H, m). MS (ES+): 408.03.

### Example 86

### 4-[2-(Difluoromethyl)-5-(4-methoxyphenyl)-1,3-oxazol-4-yl]phenol

The title compound (279 mg, 92.3%) was obtained as a powder from 4-[4-(benzyloxy)phenyl]-2-(difluoromethyl)-5-(4-methoxyphenyl)-1,3-oxazole obtained by Example 85 (388 mg, 0.952 mmol) in a manner similar to that of Example 65.

¹H-NMR (300 MHz, CDCl₃): δ 3.85(3H, s), 5.10(1H, br-s), 6.70(1H, t, J=53 Hz), 6.85(2H, d, J=8 Hz), 6.92(2H, d, J=8 Hz), 7.51(2H, d, J=8 Hz), 7.56(2H, d, J=8 Hz). MS (ES−): 316.25.

### Example 87

### 2-{4-[2-(Difluoromethyl)-5-(4-methoxyphenyl)-1,3-oxazol-4-yl]phenoxy}ethanol

To a solution of 4-[2-(difluoromethyl)-5-(4-methoxyphenyl)-1,3-oxazol-4-yl]phenol obtained by Example 86 (120 mg, 0.378 mmol) in N,N-dimethylformamide (2 mL) were added 2-chloroethanol (76.1 mg, 0.946 mmol), potassium iodide (157 mg, 0.946 mmol) and potassium carbonate (209 mg, 1.51 mmol) at room temperature, and the mixture was stirred at 75° C. for 18 hrs.

The reaction mixture was poured into water and extracted with ethyl acetate. The organic layer was washed with 1 mol/L hydrochloric acid, water, saturated sodium bicarbonate solution and brine, dried over magnesium sulfate, and evaporated in vacuo. The residue was purified by preparative thin layer chromatography (n-hexane:ethyl acetate=2:3) to give the title compound (52.6 mg, 38.5%) as an amorphous powder.

¹H-NMR (300 MHz, CDCl₃): δ 2.03(1H, t, J=7 Hz), 3.85(3H, s), 3.94-4.03(2H, m), 4.13(2H, t, J=5 Hz), 6.70(1H, t, J=53 Hz), 6.92(2H, d, J=8 Hz), 6.94(2H, d, J=8 Hz), 7.51-7.60(4H, m).

### Example 88

### tert-Butyl 2-{4-[2-(difluoromethyl)-5-(4-methoxyphenyl)-1,3-oxazol-4-yl]phenoxy}ethylcarbamate

To a solution of 4-[2-(difluoromethyl)-5-(4-methoxyphenyl)-1,3-oxazol-4-yl]phenol obtained by Example 86 (208 mg, 0.656 mmol), N-(tert-butoxycarbonyl)-2-aminoethanol (127 mg, 0.787 mmol) and diethyl azodicarboxylate (171 mg, 0.983 mmol) in anhydrous tetrahydrofuran (2 mL) was added dropwise a solution of triphenylphosphine (258 mg, 0.983 mmol) in anhydrous tetrahydrofuran (4 mL) at room temperature, and the mixture was stirred at the same temperature for 18 hrs.

The mixture was evaporated in vacuo and the residue was purified by preparative thin layer chromatography (n-hexane:ethyl acetate=3:1) to give the title compound (138 mg, 45.7%) as an oil.

¹H-NMR (300 MHz, CDCl₃): δ 1.46(9H, s), 3.55(2H, q, J=5 Hz), 3.85(3H, s), 4.05(2H, t, J=5 Hz), 5.00(1H, br peak), 6.70(1H, t, J=52 Hz), 6.86-6.95(4H, m), 7.51-7.60(4H, m).

### Example 89

### 2-{4-[2-(Difluoromethyl)-5-(4-methoxyphenyl)-1,3-oxazol-4-yl]phenoxy}ethanamine hydrochloride

The title compound (96 mg, 81.9%) was obtained as an amorphous powder from tert-butyl 2-{4-[2-(difluoromethyl)-5-(4-methoxyphenyl)-1,3-oxazol-4-yl]phenoxy}ethylcarbamate obtained by Example 88 (136 mg, 0.295 mmol) in a manner similar to that of Example 17.

¹H-NMR (300 MHz, DMSO-d₆): δ 3.24(2H, t, J=5 Hz), 3.81(3H, s), 4.20(2H, t, J=5 Hz), 7.01-7.10(4H, m), 7.30(1H, t, J=53 Hz), 7.50(2H, d, J=8 Hz), 7.55(2H, d, J=8 Hz), 8.06(3H, br peak). MS (ES+): 361.09.

### Example 90

### N-(2-{4-[2-(Difluoromethyl)-5-(4-methoxyphenyl)-1,3-oxazol-4-yl]phenoxy}ethyl)urea

The title compound (70 mg, 87.2%) was obtained as an amorphous powder from 2-{4-[2-(difluoromethyl)-5-(4-methoxyphenyl)-1,3-oxazol-4-yl]phenoxy}ethanamine hydrochloride obtained by Example 89 (79 mg, 0.199 mmol) in a manner similar to that of Example 18.

¹H-NMR (300 MHz, DMSO-d₆): δ 3.26-3.40(2H, m), 3.81(3H, s), 3.98(2H, t, J=5 Hz), 5.54(2H, s), 6.18(1H, t, J=5 Hz), 7.00(2H, d, J=8 Hz), 7.06(2H, d, J=8 Hz), 7.30 (1H, t, J=52 Hz), 7.46-7.55(4H, m). MS (ES+): 404.07.

### Example 91-1

### Benzyl 2-(4-bromophenyl)ethyl ether

To a slurry of sodium hydride (abt. 60% oil suspension, 4.58 g) in N,N-dimethylformamide (150 mL) was added dropwise 2-(4-bromophenyl)ethanol (20 g) in N,N-dimethylformamide (50 mL) at 0° C., and the mixture was stirred for 1 hr at room temperature. To the mixture was added dropwise benzyl bromide (19.6 g) at 0° C., and the mixture was stirred at room temperature for 6 hrs.

The resulting mixture was partitioned between saturated aqueous ammonium chloride and ethyl acetate. The organic layer was washed with brine, dried over anhydrous magnesium sulfate, and concentrated in vacuo to give the title compound as a colorless oil (29.0 g, 100%).

¹H-NMR (300 MHz, CDCl₃): δ 2.88(2H, t, J=7 Hz), 3.67(2H, t, J=7 Hz), 4.52(2H, s), 7.11(2H, d, J=8 Hz), 7.27-7.37(5H, m), 7.41(2H, d, J=8 Hz).

### Example 91-2

### 4-[2-(Benzyloxy)ethyl]benzaldehyde

To a solution of benzyl 2-(4-bromophenyl)ethyl ether obtained by Example 91-1 (29.0 g) in dry tetrahydrofuran (300 mL) was added dropwise n-butyllithium (1.57mol/L solution in hexanes, 66.5 mL) at −78° C. under nitrogen, and the mixture was stirred at −78° C. for 1 hr. To the mixture was added dropwise N,N-dimethylformamide (15.4 mL).

After being stirred for 1.5 hrs at −78° C., the mixture was warmed to room temperature, then poured into saturated aqueous ammonium chloride, and extracted with ether three times. The combined organic extracts were washed with water, brine, dried over anhydrous magnesium sulfate, and concentrated to give the title compound as a yellow oil (23.9 g, 100%).

¹H-NMR (300 MHz, CDCl₃): δ 3.02(2H, t, J=7 Hz), 3.74(2H, t, J=7 Hz), 4.53(2H, s), 7.37-7.27(5H, m), 7.41(2H, d, J=8 Hz), 7.82(2H, d, J=8H), 10.00(1H, s).

### Example 91-3

### (2E)- and (2Z)-3-{4-[2-(Benzyloxy)ethyl]phenyl}-2-(4-methoxyphenyl)-2-propenoic acid

A mixture of 4-[2-(benzyloxy)ethyl]benzaldehyde obtained by Example 91-2 (23.9 g) and 4-methoxyphenylacetic acid (16.5 g) in acetic anhydride (30 mL) and triethylamine (17 mL) was heated under reflux with stirring for 8 hrs.

After cooling, the mixture was concentrated, and partitioned between 1N sodium hydroxide solution (500 mL) and ether. The ether layer was discarded. The aqueous layer was acidified with 1 mol/L hydrochloric acid. The resulting precipitate was collected by filtration, washed with water, and dried to give the title compound as crystals (19.8 g, 51.2%).

¹H-NMR (300 MHz, DMSO-d₆, a mixture of E- and Z-isomers): δ 2.78(2H×0.76, t, J=7 Hz), 2.86(2H×0.24, t, J=7 Hz), 3.59(2H×0.76, t, J=7 Hz), 3.66(2H×0.24, t, J=7 Hz), 3.78(3H×0.76, s), 3.78(3H×0.24, s), 4.44(2H×0.76, s), 4.49(2H×0.24, s), 6.91-7.69(14H, m). MS(ESI): 389.09(M+H), 387.22(M−H).

### Example 91-4

### 2-{4-[2-(Benzyloxy)ethyl]phenyl}-1-(4-methoxyphenyl)ethanone

To a solution of (2E)- and (2Z)-3-{4-[2-(benzyloxy)ethyl]phenyl}-2-(4-methoxyphenyl)-2-propenoic acid obtained by Example 91-3 (19.4 g) in 1,4-dioxane (200 mL) was added triethylamine (7.66 mL) followed by addition of diphenylphosphoryl azide (15.1 g). The mixture was heated at 100° C. with stirring for 30 min. To the mixture was added dropwise 50% acetic acid in water (200 mL), and the mixture was heated at 100° C. for 1.5 hrs.

After cooling, the mixture was concentrated, and the residue was neutralized with sodium hydrogencarbonate and extracted with ethyl acetate. The organic layer was washed with brine, dried over anhydrous magnesium sulfate, and concentrated in vacuo. The residual oil was dissolved in ethanol with stirring to give the title compound as crystals (12.3 g, 68.3%).

¹H-NMR (300 MHz, CDCl₃): δ 2.90(2H, t, J=7 Hz), 3.67(2H, t, J=7 Hz), 3.86(3H, s), 4.20(2H, s), 4.51(2H, s), 6.92(2H, d, J=9 Hz), 7.18(4H s), 7.24-7.34(5H , m), 7.99(2H, d, J=9 Hz). MS(ESI): 361.13.

### Example 91-5

### 2-{4-[2-(Benzyloxy)ethyl]phenyl}-2-bromo-1-(4-methoxyphenyl)ethanone

The title compound (4.3 g, 100%) was obtained as an oil from 2-{4-[2-(benzyloxy)ethyl]phenyl}-1-(4-methoxyphenyl)ethanone obtained by Example 91-4 (3.5 g, 9.71 mmol) and pyridinium tribromide (3.42 g, 10.7 mmol) in a manner similar to that of Example 78-3.

¹H-NMR (300 MHz, CDCl₃): δ 2.90(2H, t, J=7 Hz), 3.66(2H, t, J=7 Hz), 3.85(3H, s), 4.50(2H, s), 6.35(1H, s), 6.90(2H, d, J=8 Hz), 7.15-7.35(7H, m), 7.44(2H, d, J=8 Hz), 7.96 (2H, d, J=8 Hz).

### Example 91-6

### 1-{4-[2-(Benzyloxy)ethyl]phenyl}-2-(4-methoxyphenyl)-2-oxoethyl (acetyloxy)acetate

The title compound (4.2 g, 90.2%) was obtained as an oil from 2-{4-[2-(benzyloxy)ethyl]phenyl}-2-bromo-1-(4-methoxyphenyl)ethanone obtained by Example 91-5 (4.3 g, 9.79 mmol) and acetoxyacetic acid (1.16 g, 9.79 mmol) in a manner similar to that of Example 78-4.

¹H-NMR (300 MHz, CDCl₃): δ 2.14(3H, s), 2.89(2H, t, J=7 Hz), 3.64(2H, t, J=7 Hz), 3.82(3H, s), 4.49(2H, s), 4.73(1H, d, J=15 Hz), 4.80(1H, d, J=15 Hz), 6.81-6.90(3H, m), 7.18-7.32(7H, m), 7.36(2H, d, J=8 Hz), 7.90(2H, d, J=8 Hz).

### Example 91-7

### [5-{4-[2-(Benzyloxy)ethyl]phenyl}-4-(4-methoxyphenyl)-1,3-oxazol-2-yl]methanol

To a solution of 1-{4-[2-(benzyloxy)ethyl]phenyl}-2-(4-methoxyphenyl)-2-oxoethyl (acetyloxy)acetate obtained by Example 91-6 (4.2 g, 8.83 mmol) in acetic acid (40 mL) was added ammonium acetate (5.44 g, 70.6 mmol) at room temperature, and the mixture was heated to reflux with stirring for 4 hrs.

After cooling, the reaction mixture was evaporated in vacuo and acetic acid was azeotropically removed with toluene. The residue was partitioned between water and ethyl acetate. The organic layer was separated, washed with water, saturated sodium bicarbonate solution and brine, successively, dried over magnesium sulfate.

After evaporation of solvent, the residue was dissolved in methanol (20 mL). To a solution was added potassium carbonate (610 mg) at room temperature, and the mixture was stirred at the same temperature for 1 hr.

The reaction mixture was evaporated in vacuo, and the residue was partitioned between water and ethyl acetate. The organic layer was separated, washed with 1 mol/L hydrochloric acid, water, saturated sodium bicarbonate solution and brine, dried over magnesium sulfate, and evaporated in vacuo. The residue was purified by silica gel column chromatography (chloroform) to give the title compound (2.67 g, 72.8%) as an oil.

¹H-NMR (300 MHz, CDCl₃): δ 2.94(2H, t, J=7 Hz), 3.70(2H, t, J=7 Hz), 3.84(3H, s), 4.53(2H, s), 4.80(2H, s), 6.90(2H, d, J=8 Hz), 7.15-7.39(7H, m), 7.50(2H, d, J=8 Hz), 7.56(2H, d, J=8 Hz).

### Example 92

### 5-{4-[2-(Benzyloxy)ethyl]phenyl}-4-(4-methoxyphenyl)-1,3-oxazole-2-carbaldehyde

The title compound (605 mg, 22.8%) was obtained as an oil from [5-{4-[2-(benzyloxy)ethyl]phenyl}-4-(4-methoxyphenyl)-1,3-oxazol-2-yl]methanol obtained by Example 91-7 (2.37 g, 6.43 mmol) in a manner similar to that of Example 71.

¹H-NMR (300 MHz, CDCl₃): δ 2.96(2H, t, J=7 Hz), 3.73(2H, t, J=7 Hz), 3.87(3H, s), 4.53(2H, s), 6.95(2H, d, J=8 Hz), 7.20-7.40(7H, m), 7.55-7.67(4H, m), 9.79(1H, s).

### Example 93

### 5-{4-[2-(Benzyloxy)ethyl]phenyl}-2-(difluoromethyl)-4-(4-methoxy phenyl)-1,3-oxazole

The title compound (483 mg, 75.8%) was obtained as an oil from 5-{4-[2-(benzyloxy)ethyl]phenyl}-4-(4-methoxyphenyl) -1,3-oxazole-2-carbaldehyde obtained by Example 92 (605 mg, 1.46 mmol) in a manner similar to that of Example 77.

¹H-NMR (300 MHz, CDCl₃): δ 2.99(2H, t, J=7 Hz), 3.71(2H, t, J=7 Hz), 3.85(3H, s), 4.54(2H, s), 6.70(1H, t, J=53 Hz), 6.91(2H, d, J=8 Hz), 7.19-7.37(7H, m), 7.50-7.63(4H, m).

### Example 94

### 2-{4-[2-(Difluoromethyl)-4-(4-methoxyphenyl)-1,3-oxazol-5-yl]phenyl}ethanol

The title compound (305 mg, 80%) was obtained as a powder from 5-{4-[2-(benzyloxy)ethyl]phenyl}-2-(difluoromethyl)-4-(4-methoxyphenyl)-1,3-oxazole obtained by Example 93 (481 mg, 1.1 mmol) in a manner similar to that of Example 31.

¹H-NMR (300 MHz, CDCl₃): δ 1.41(1H, t, J=7 Hz), 2.91(2H, t, J=7 Hz), 3.85(3H, s), 3.90(2H, q, J=7 Hz), 6.70(1H, t, J=53 Hz), 6.92(2H, d, J=8 Hz), 7.26(2H, d, J=8 Hz), 7.54-7.62(4H, m). MS (ES+): 346.14.

### Example 95

### 2-{4-[2-(Difluoromethyl)-4-(4-methoxyphenyl)-1,3-oxazol-5-yl]phenyl}ethyl methanesulfonate

The title compound (308 mg, 100%) was obtained as an oil from 2-{4-[2-(difluoromethyl)-4-(4-methoxyphenyl)-1,3-oxazol-5-yl]phenyl}ethanol obtained by Example 94 (250 mg, 0.724 mmol) in a manner similar to that of Example 34.

¹H-NMR (300 MHz, CDCl₃): δ 2.92(3H, s), 3.09(2H, t, J=7 Hz), 3.85(3H, s), 4.45(2H, t, J=7 Hz), 6.70(1H, t, J=53 Hz), 6.93(2H, d, J=8 Hz), 7.26(2H, d, J=8 Hz), 7.55(2H, d, J=8 Hz), 7.60(2H, d, J=8 Hz).

### Example 96

### 2-(2-{4-[2-(Difluoromethyl)-4-(4-methoxyphenyl)-1,3-oxazol-5-yl]phenyl}ethyl)-1H-isoindole-1,3(2H)-dione

The title compound (365 mg, 107%) was obtained as a powder from 2-{4-[2-(difluoromethyl)-4-(4-methoxyphenyl)-1,3-oxazol-5-yl]phenyl}ethyl methanesulfonate obtained by Example 95 (305 mg, 0.72 mmol) and potassium phthalimide (200 mg, 1.08 mmol) in a manner similar to that of Example 35.

¹H-NMR (300 MHz, CDCl₃): δ 3.03(2H, t, J=7 Hz), 3.85(3H, s), 3.95(2H, t, J=7Hz), 6.69(1H, t, J=53 Hz), 6.90(2H, d, J=8 Hz), 7.26(2H, d, J=8 Hz), 7.49-7.58(4H, m), 7.68-7.74(2H, m), 7.80-7.86(2H, m).

### Example 97

### 2-{4-[2-(Difluoromethyl)-4-(4-methoxyphenyl)-1,3-oxazol-5-yl]phenyl}ethylamine

The title compound (300 mg, 115%) was obtained as an oil from 2-(2-{4-[2-(difluoromethyl)-4-(4-methoxyphenyl)-1,3-oxazol-5-yl]phenyl}ethyl)-1H-isoindole-1,3(2H)-dione obtained by Example 96 (360 mg, 0.759 mmol) in a manner similar to that of Example 44.

¹H-NMR (300 MHz, CDCl₃): δ 2.68-2.90(4H, m), 3.85(3H, s), 6.70(1H, t, J=53 Hz), 6.92(2H, d, J=9 Hz), 7.15-7.30(2H, m), 7.44-7.64(4H, m).

### Example 98

### N-(2-{4-[2-(Difluoromethyl)-4-(4-methoxyphenyl)-1,3-oxazol-5-yl]phenyl}ethyl)methanesulfonamide

The title compound (78 mg, 42.4%) was obtained as an oil from 2-{4-[2-(difluoromethyl)-4-(4-methoxyphenyl)-1,3-oxazol-5-yl]phenyl}ethylamine obtained by Example 97 (150 mg, 0.434 mmol) in a manner similar to that of Example 38.

¹H-NMR (300 MHz, CDCl₃): δ 2.90(3H, s), 2.92(2H, t, J=7 Hz), 3.44(2H, t, J=7 Hz), 3.86(3H, s), 4.22(1H, t, J=6 Hz), 6.71(1H, t, J=53 Hz), 6.94(2H, d, J=8 Hz), 7.25(2H, d, J=8 Hz), 7.56(2H, d, J=8 Hz), 7.60(2H, d, J=8 Hz). MS (ES−): 421.19.

### Example 99

### N-(2-{4-[2-(Difluoromethyl)-4-(4-methoxyphenyl)-1,3-oxazol-5-yl]phenyl}ethyl)urea

The title compound (32 mg, 19%) was obtained as a powder from 2-{4-[2-(difluoromethyl)-4-(4-methoxyphenyl)-1,3-oxazol-5-yl]phenyl}ethylamine obtained by Example 97 (150 mg, 0.436 mmol) in a manner similar to that of Example 18.

¹H-NMR (300 MHz, DMSO-d₆): δ 2.73(2H, t, J=7 Hz), 3.22(2H, q, J=7 Hz), 3.80(3H, s), 5.44(2H, s), 5.95(1H, t, J=6 Hz), 7.00(2H, d, J=8 Hz), 7.31(1H, t, J=53 Hz), 7.33(2H, d, J=8 Hz), 7.46-7.56(4H, m). MS (ES+): 388.15.

### Example 100-1

### 2-[4-(Benzyloxy)phenyl]-1-(6-methoxy-3-pyridinyl)ethanone

1.56M n-Butyllithium in hexane (134 mL, 209 mmol) was added dropwise to a solution of 5-bromo-2-methoxypyridine (36.3 g, 193 mmol) in tetrahydrofuran (340 mL) at −78° C. and the suspension stirred at the same temperature for 1 hr. 2-[4-(Benzyloxy)phenyl]-N-methoxy-N-methylacetamide (55.1 g, 193 mmol) in tetrahydrofuran (340 mL) was then added and stirring continued for a further 2.5 hrs.

The mixture was allowed to 3° C. and then it was poured into NH₄Cl solution. The mixture was extracted with ethyl acetate (1000 mL) and the organic extract was washed with brine. The organic extract was dried (magnesium sulfate) and the solvent was removed to give the title compound as solid. The solid was washed with isopropyl alcohol-isopropyl ether to give the title compound as white crystals.

¹H-NMR (300 MHz, CDCl₃): δ 3.99(3H, s), 4.16(2H, s), 5.04(2H, s), 6.78(1H, d, J=8 Hz), 6.94(2H, d, J=8 Hz), 7.18(2H, d, J=8 Hz), 7.30-7.43(5H, m), 8.16(1H, dd, J=8.2 Hz), 8.85(1H, d, J=2 Hz). MS (ES+): 334.10.

### Example 100-2

### 2-[4-(Benzyloxy)phenyl]-2-bromo-1-(6-methoxy-3-pyridinyl)ethanone

The title compound as an oil (1.87 g, 100%) was obtained from 2-[4-(benzyloxy)phenyl]-1-(6-methoxy-3-pyridinyl)ethanone obtained by Example 100-1 (1.5 g, 4.5 mmol) in a manner similar to that of Example 78-3.

¹H-NMR (300 MHz, CDCl₃): 4.00(3H, s), 5.06(2H, s), 6.28(1H, s), 6.78(1H, d, J=9 Hz), 6.96(2H, d, J=9 Hz), 7.29-7.50(7H, m), 8.16(1H, dd, J=9.2 Hz), 8.81(1H, d, J=2 Hz).

### Example 100-3

### 1-[4-(Benzyloxy)phenyl]-2-(6-methoxy-3-pyridinyl)-2-oxoethyl 2-methylpropanoate

The title compound (819 mg, 43%) was obtained as an oil from 2-[4-(benzyloxy)phenyl]-2-bromo-1-(6-methoxy-3-pyridinyl)ethanone obtained by Example 100-2 (1.87 g, 4.54 mmol) and isobutyric acid (400 mg, 4.54 mmol) in a manner similar to that of Example 78-4.

¹H-NMR (300 MHz, CDCl₃): 1.19(3H, d, J=7 Hz), 1.26(3H, d, J=7 Hz), 2.63-2.78(1H, m), 3.96(3H, s), 5.03(2H, s), 6.66(1H, s), 6.72(1H, d, J=9 Hz), 6.95(2H, d, J=9 Hz), 7.26-7.43(7H, m), 8.10(1H, dd, J=8.2 Hz), 8.78(1H, d, J=2 Hz).

### Example 100-4

### 5-{5-[4-(Benzyloxy)phenyl]-2-isopropyl-1,3-oxazol-4-yl}-2-methoxypyridine

The title compound (562 mg, 71.9%) was obtained as a powder from 1-[4-(benzyloxy)phenyl]-2-(6-methoxy-3-pyridinyl)-2-oxoethyl 2-methylpropanoate obtained by Example 100-3 (819 mg, 1.95 mmol) and ammonium acetate (1.2 g, 15.6 mmol) in a manner similar to that of Example 64-2.

¹H-NMR (300 MHz, CDCl₃): δ 1.41(6H, d, J=7 Hz), 3.09-3.21(1H, m), 3.96(3H, s), 5.09(2H, s), 6.75(1H, d, J=9 Hz), 6.96(2H, d, J=9 Hz), 7.29-7.51(7H, m), 7.81(1H, dd, J=9.2 Hz), 8.40(1H, d, J=2 Hz).

### Example 101

### 4-[2-Isopropyl-4-(6-methoxy-3-pyridinyl)-1,3-oxazol-5-yl]phenol

The title compound (410 mg, 97.6%) was obtained as a powder from 5-{5-[4-(benzyloxy)phenyl]-2-isopropyl-1,3-oxazol-4-yl}-2-methoxypyridine obtained by Example 100-4 (542 mg, 1.35 mmol) in a manner similar to that of Example 31.

¹H-NMR (300 MHz, DMSO-d₆): δ 1.34(6H, d, J=7 Hz), 3.05-3.20(1H, m), 3.87(3H, s), 6.82(2H, d, J=9 Hz), 6.86(1H, d, J=9 Hz), 7.34(2H, d, J=9 Hz), 7.80(1H, dd, J=9.2 Hz), 8.32(1H, d, J=2 Hz), 9.91(1H, br peak). MS (ES+): 311.22.

### Example 102

### 2-{4-[2-Isopropyl-4-(6-methoxy-3-pyridinyl)-1,3-oxazol-5-yl]phenoxy}ethanol

The title compound (385 mg, 84.3%) was obtained as a powder from 4-[2-isopropyl-4-(6-methoxy-3-pyridinyl)-1,3-oxazol-5-yl]phenol obtained by Example 101 (400 mg, 1.29 mmol) and chloroethanol (623 mg, 7.73 mmol) in a manner similar to that of Example 87.

¹H-NMR (300 MHz, CDCl₃): δ 1.42(6H, d, J=7 Hz), 2.02(1H, t, J=6 Hz), 3.09-3.22(1H, m), 3.96(3H, s), 3.96-4.01(2H, m), 4.10(2H, t, J=5 Hz), 6.74(1H, d, J=9Hz), 6.91(2H, d, J=9 Hz), 7.48(2H, d, J=9Hz), 7.81(1H, dd, J=9.2 Hz), 8.40(1H, d, J=2 Hz). MS (ES+): 355.24.

### Example 103

### 2-{4-[2-Isopropyl-4-(6-methoxy-3-pyridinyl)-1,3-oxazol-5-yl]phenoxy}ethyl methanesulfonate

The title compound (400 mg, 99.9%) was obtained as an oil from 2-{4-[2-isopropyl-4-(6-methoxy-3-pyridinyl)-1,3-oxazol-5-yl]phenoxy}ethanol obtained by Example 102 (328 mg, 0.926 mmol) in a manner similar to that of Example 34.

¹H-NMR (300 MHz, CDCl₃): δ 1.43(6H, d, J=7 Hz), 3.11(3H, s), 3.11-3.22(1H, m), 3.96(3H, s), 4.23-4.30(2H, m), 4.54-4.61(2H, m), 6.76(1H, d, J=9 Hz), 6.90(2H, d, J=9 Hz), 7.49(2H, d, J=9 Hz), 7.82(1H, dd, J=9.2 Hz), 8.39(1H, d, J=2 Hz).

### Example 104

### 2-(2-{4-[2-Isopropyl-4-(6-methoxy-3-pyridinyl)-1,3-oxazol-5-yl]phenoxy}ethyl)-1H-isoindole-1,3(2H)-dione

The title compound (355 mg, 79.4%) was obtained as a powder from 2-{4-[2-isopropyl-4-(6-methoxy-3-pyridinyl)-1,3-oxazol-5-yl]phenoxy}ethyl methanesulfonate obtained by Example 103 (400 mg, 0. 925 mmol) and potassium phthalimide (257 mg, 1.39 mmol) in a manner similar to that of Example 35.

¹H-NMR (300 MHz, CDCl₃): δ 1.41(6H, d, J=7 Hz), 3.06-3.20(1H, m), 3.94(3H, s), 4.12(2H, t, J=5 Hz), 4.25(2H, t, J=5 Hz), 6.73(1H, d, J=9 Hz), 6.86(2H, d, J=9 Hz), 7.43(2H, d, J=9 Hz), 7.69-7.80(3H, m), 7.80-7.93(2H, m), 8.36(1H, d, J=2 Hz). MS (ES+): 484.17.

### Example 105

### 2-{4-[2-Isopropyl-4-(6-methoxy-3-pyridinyl)-1,3-oxazol-5-yl]phenoxy}ethylamine

The title compound (327 mg, 127%) was obtained as an an oil from 2-(2-{4-[2-isopropyl-4-(6-methoxy-3-pyridinyl)-1,3-oxazol-5-yl]phenoxy}ethyl)-1H-isoindole-1,3(2H)-dione obtained by Example 104 (353 mg, 0.73 mmol) in a manner similar to that of Example 36.

¹H-NMR (300 MHz, CDCl₃): δ 1.41(6H, d, J=7 Hz), 3.05-3.21(3H, m), 3.95(3H, s), 4.00(2H, t, J=5 Hz), 6.75(1H, d, J=9 Hz), 6.90(2H, d, J=9 Hz), 7.46(2H, d, J=9 Hz), 7.81(1H, dd, J=9.2 Hz), 8.40(1H, d, J=2 Hz). MS (ES+): 354.21.

### Example 106

### N-(2-{4-[2-Isopropyl-4-(6-methoxy-3-pyridinyl)-1,3-oxazol-5-yl]phenoxy}ethyl)methanesulfonamide

The title compound (67 mg, 54.9%) was obtained as a powder from 2-{4-[2-isopropyl-4-(6-methoxy-3-pyridinyl)-1,3-oxazol-5-yl]phenoxy}ethylamine obtained by Example 105 (100 mg, 0.283 mmol) in a manner similar to that of Example 38.

¹H-NMR (300 MHz, CDCl₃): δ 1.41(6H, d, J=7 Hz), 3.04(3H, s), 3.10-3.21(1H, m), 3.56(2H, q, J=5 Hz), 3.96(3H, s), 4.12(2H, t, J=5 Hz), 4.76(1H, br peak), 6.75(1H, d, J=9 Hz), 6.88(2H, d, J=9 Hz), 7.49(2H, d, J=9 Hz), 7.81(1H, dd, J=9.2 Hz), 8.39(1H, d, J=2 Hz). MS (ES+): 432.19.

### Example 107

### N-(2-{4-[2-Isopropyl-4-(6-methoxy-3-pyridinyl)-1,3-oxazol-5-yl]phenoxy}ethyl)urea

The title compound (121 mg, 61.3%) was obtained as a powder from 2-{4-[2-isopropyl-4-(6-methoxy-3-pyridinyl)-1,3-oxazol-5-yl]phenoxy}ethylamine obtained by Example 105 (176 mg, 0.498 mmol) in a manner similar to that of Example 18.

¹H-NMR (300 MHz, CDCl₃): δ 1.42(6H, d, J=7 Hz), 3.09-3.21(1H, m), 3.61(2H, q, J=5 Hz), 3.95(3H, s), 4.06(2H, t, J=5 Hz), 4.42(2H, br-s), 5.00(1H, br peak), 6.75(1H, d, J=9 Hz), 6.88(2H, d, J=9 Hz), 7.46(2H, d, J=9 Hz), 7.82(1H, dd, J=9.2 Hz), 8.38(1H, d, J=2 Hz). MS (ES+): 397.18.

### Example 108-1

### 2-[4-(Benzyloxy)phenyl]-2-bromo-1-(4-methoxyphenyl)ethanone

The title compound (10 g, 101%) was obtained as an oil from 2-[4-(benzyloxy)phenyl]-1-(4-methoxyphenyl)-ethanone (8.0 g, 24.1 mmol) in a manner similar to that of Example 78-3.

¹H-NMR (300 MHz, CDCl₃): δ 3.86(3H, s), 5.05(2H, s), 6.37(1H, s), 6.90(2H, d, J=9 Hz), 6.95(2H, d, J=9 Hz), 7.27-7.50(7H, m), 7.96(2H, d, J=9 Hz).

### Example 108-2

### 1-[4-(Benzyloxy)phenyl]-2-(4-methoxyphenyl)-2-oxoethyl cyclopropanecarboxylate

The title compound (1.68 g, 83%) was obtained as an oil from 2-[4-(benzyloxy)phenyl]-2-bromo-1-(4-methoxyphenyl)ethanone obtained by Example 108-1 (2.0 g, 4.86 mmol) and cyclopropanecarboxylic acid (419 mg, 4.86 mmol) in a manner similar to that of Example 78-4.

¹H-NMR (300 MHz, CDCl₃): δ 0.85-0.96(2H, m), 1.01-1.11(2H, m), 1.71-1.85(1H, m), 3.82(3H, s), 5.03(2H, s), 6.80(1H, s), 6.86(2H, d, J=9 Hz), 6.95(2H, d, J=9 Hz), 7.26-7.44(7H, m), 7.91(2H, d, J=9 Hz).

### Example 108-3

### 5-[4-(Benzyloxy)phenyl]-2-cyclopropyl-4-(4-methoxyphenyl)-1,3-oxazole

The title compound (1.28 g, 80.8%) was obtained as an oil from 1-[4-(benzyloxy)phenyl]-2-(4-methoxyphenyl)-2-oxoethyl cyclopropanecarboxylate obtained by Example 108-2 (1.66 g, 3.99 mmol) and ammonium acetate (2.46 g, 31.9 mmol) in a manner similar to that of Example 64-2.

¹H-NMR (300 MHz, CDCl₃): δ 1.00-1.11(2H, m), 1.11-1.19(2H, m), 2.05-2.17(1H, m), 3.83(3H, s), 5.08(2H, s), 6.87(2H, d, J=9 Hz), 6.95(2H, d, J=9 Hz), 7.30-7.49(7H, m), 7.54(2H, d, J=9 Hz) MS (ES+): 398.18

### Example 109

### 4-[2-Cyclopropyl-4-(4-methoxyphenyl)-1,3-oxazol-5-yl]phenol

The title compound (912 mg, 94.4%) was obtained as a powder from 5-[4-(benzyloxy)phenyl]-2-cyclopropyl-4-(4-methoxyphenyl)-1,3-oxazole Example 108-3 (1.25 g, 3.14 mmol) in a manner similar to that of Example 31.

¹H-NMR (300 MHz, CDCl₃): δ 1.00-1.11(2H, m), 1.11-1.19(2H, m), 2.05-2.18(1H, m), 3.82(3H, s), 5.13(1H, br-s), 6.80(2H, d, J=9 Hz), 6.88(2H, d, J=9 Hz), 7.40(2H, d, J=9 Hz), 7.53(2H, d, J=9 Hz). MS (ES+): 308.18.

### Example 110

### 2-{4-[2-Cyclopropyl-4-(4-methoxyphenyl)-1,3-oxazol-5-yl]phenoxy}ethanol

The title compound (765 mg, 74.3%) was obtained as a powder from 4-[2-cyclopropyl-4-(4-methoxyphenyl)-1,3-oxazol-5-yl]phenol obtained by Example 109 (900 mg, 2.93 mmol) and2-chloroethanol (1.41 g, 17.6 mmol) in a manner similar to that of Example 87.

¹H-NMR (300 MHz, DMSO-d₆): δ 0.97-1.13(4H, m), 2.18-2.21(1H, m), 3.71(2H, q, J=5 Hz), 3.77(3H, s), 4.00(2H, t, J=5 Hz), 4.89(1H, t, J=5.5 Hz), 6.93(2H, d, J=9 Hz), 6.98(2H, d, J=9 Hz), 7.41(2H, d, J=9 Hz), 7.95(2H, d, J=9 Hz). MS (ES+): 352.20.

### Example 111

### 2-{4-[2-Cyclopropyl-4-(4-methoxyphenyl)-1,3-oxazol-5-yl]phenoxy}ethyl methanesulfonate

The title compound (308 mg, 100%) was obtained as an oil from 2-{4-[2-cyclopropyl-4-(4-methoxyphenyl)-1,3-oxazol-5-yl]phenoxy}ethanol obtained by Example 110 (250 mg, 0.711 mmol) in a manner similar to that of Example 34.

¹H-NMR (300 MHz, CDCl₃): δ 1.00-1.12(2H, m), 1.12-1.20(2H, m), 2.06-2.19(1H, m), 3.10(3H, s), 3.83(3H, s), 4.23-4.30(2H, m), 4.55-4.61(2H, m), 6.83-6.91(4H, m), 7.46(2H, d, J=9 Hz), 7.51(2H, d, J=9 Hz).

### Example 112

### 2-(2-{4-[2-Cyclopropyl-4-(4-methoxyphenyl)-1,3-oxazol-5-yl]phenoxy}ethyl)-1H-isoindole-1,3(2H)-dione

The title compound (237 mg, 68.8%) was obtained as a powder from 2-{4-[2-cyclopropyl-4-(4-methoxyphenyl)-1,3-oxazol-5-yl]phenoxy}ethyl methanesulfonate obtained by Example 111 (308 mg, 0.717 mmol) and potassium phthalimide (199 mg, 1.08 mmol) in a manner similar to that of Example 35.

¹H-NMR (300 MHz, DMSO-d₆): δ 0.97-1.09(4H, m), 2.06-2.21(1H, m), 3.76(3H, s), 3.96(2H, t, J=6 Hz), 4.25(2H, t, J=6 Hz), 6.89-6.99(4H, m), 7.38(2H, d, J=9 Hz), 7.42(2H, d, J=9 Hz), 7.81-7.94(4H, m). MS (ES+): 481.17.

### Example 113

### 2-{4-[2-Cyclopropyl-4-(4-methoxyphenyl)-1,3-oxazol-5-yl]phenoxy}ethylamine

The title compound (201 mg, 119%) was obtained as an oil from 2-(2-{4-[2-cyclopropyl-4-(4-methoxyphenyl)-1,3-oxazol-5-yl]phenoxy}ethyl)-1H-isoindole-1,3(2H)-dione obtained by Example 112 (233 mg, 0.482 mmol) in a manner similar to that of Example 36.

¹H-NMR (300 MHz, CDCl₃): δ 1.00-1.11(2H, m), 1.11-1.20(2H, m), 2.05-2.18(1H, m), 3.09(2H, t, J=5 Hz), 3.93(3H, s), 4.01(2H, d, J=5 Hz), 6.81-6.92(4H, m), 7.45(2H, d, J=9 Hz), 7.53(2H, d, J=9 Hz).

### Example 114

### N-(2-{4-[2-Cyclopropyl-4-(4-methoxyphenyl)-1,3-oxazol-5-yl]phenoxy}ethyl)methanesulfonamide

The title compound (64 mg, 69.8%) was obtained as an oil from 2-{4-[2-cyclopropyl-4-(4-methoxyphenyl)-1,3-oxazol-5-yl]phenoxy}ethylamine obtained by Example 113 (75 mg, 0.214 mmol) in a manner similar to that of Example 38.

¹H-NMR (300 MHz, CDCl₃): δ 1.01-1.11(2H, m), 1.11-1.20(2H, m), 2.04-2.18(1H, m), 3.03(3H, s), 3.56(2H, q, J=5 Hz), 3.80(3H, s), 4.12(2H, t, J=5 Hz), 4.75(1H, br peak), 6.85(2H, d, J=9 Hz), 6.89(2H, d, J=9 Hz), 7.46(2H, d, J=9 Hz), 7.52(2H, d, J=9 Hz).

### Example 115

### N-(2-{4-[2-Cyclopropyl-4-(4-methoxyphenyl)-1,3-oxazol-5-yl]phenoxy}ethyl)urea

The title compound (94 mg, 66.4%) was obtained as a powder from 2-{4-[2-cyclopropyl-4-(4-methoxyphenyl)-1,3-oxazol-5-yl]phenoxy}ethylamine obtained by Example 113 (126 mg, 0.36 mmol) in a manner similar to that of Example 18.

¹H-NMR (300 MHz, DMSO-d₆): δ 0.96-1.11(4H, m), 2.09-2.20(1H, m), 3.26-3.36(2H, m), 3.76(3H, s), 3.96(2H, t, J=5 Hz), 5.564(2H, s), 6.66(1H, t, J=5 Hz), 6.94(2H, d, J=9 Hz), 7.00(2H, d, J=9 Hz), 7.41(2H, d, J=9 Hz), 7.45(2H, d, J=9 Hz). MS (ES+): 394.21.

### Example 116-1

### 1-[4-(Benzyloxy)phenyl]-2-(6-methoxy-3-pyridinyl)-2-oxoethyl cyclopropanecarboxylate

The title compound (1.72 g, 93.8%) was obtained as an oil from 2-[4-(benzyloxy)phenyl]-2-bromo-1-(6-methoxy-3-pyridinyl)ethanone (1.85 g, 4.39 mmol) and cyclopropanecarboxylic acid (378 mg, 4.39 mmol) in a manner similar to that of Example 78-4.

¹H-NMR (300 MHz, CDCl₃): δ 0.85-0.99(2H, m), 1.04-1.14(2H, m), 1.71-1.85(1H, m), 3.96(3H, s), 5.04(2H, s), 6.70(1H, s), 6.73(1H, d, J=9 Hz), 6.97(2H, d, J=9 Hz), 7.28-7.45(7H, m), 8.10(1H, dd, J=9.2 Hz), 8.78(1H, d, J=2 Hz). MS (ES+): 418.18.

### Example 116-2

### 5-{5-[4-(Benzyloxy)phenyl]-2-cyclopropyl-1,3-oxazol-4-yl}-2-methoxypyridine

The title compound (1.14 g, 69.4%) was obtained as a powder from 1-[4-(benzyloxy)phenyl]-2-(6-methoxy-3-pyridinyl)-2-oxoethyl cyclopropanecarboxylate obtained by Example 116-1 (1.72 g, 4.12 mmol) and ammonium acetate (2.54 g, 33 mmol) in a manner similar to that of Example 64-2.

¹H-NMR (300 MHz, CDCl₃): δ 1.03-1.11(2H, m), 1.11-1.20(2H, m), 2.06-2.19(1H, m), 3.95(3H, s), 5.08(2H, s), 6.74(1H, d, J=9 Hz), 6.95(2H, d, J=9 Hz), 7.30-7.48(7H,m), 7.80(1H,dd, J=8.2 Hz), 8.39(1H, d, J=2 Hz). MS (ES+): 399.17.

### Example 117

### 4-[2-Cyclopropyl-4-(6-methoxy-3-pyridinyl)-1,3-oxazol-5-yl]phenol

The title compound (710 mg, 83.4%) was obtained as a powder from 5-{5-[4-(benzyloxy)phenyl]-2-cyclopropyl-1,3-oxazol-4-yl}-2-methoxypyridine obtained by Example 116-2 (1.1 g, 2.76 mmol) in a manner similar to that of Example 31.

¹H-NMR (300 MHz, CDCl₃): δ 1.01-1.11(2H, m), 1.11-1.20(2H, m), 2.06-2.18(1H,m), 3.95(3H, s), 6.16(1H, br peak), 6.75(1H, d, J=9 Hz), 6.81(2H, d, J=9 Hz), 7.38 (2H, d, J=9 Hz), 7.84(1H, dd, J=9.2 Hz), 8.38(1H, d, J=2 Hz). MS (ES+): 309.14.

### Example 118

### 2-{4-[2-Cyclopropyl-4-(6-methoxy-3-pyridinyl)-1,3-oxazol-5-yl]phenoxy}ethanol

The title compound (575 mg, 71.9%) was obtained as a powder from 4-[2-cyclopropyl-4-(6-methoxy-3-pyridinyl)-1,3-oxazol-5-yl]phenol obtained by Example 117 (700 mg, 2.27 mmol) and 2-chloroethanol (1.1 g, 13.6 mmol) in a manner similar to that of Example 87.

¹H-NMR (300 MHz, CDCl₃): δ 1.02-1.11(2H, m), 1.11-1.20(2H, m), 2.02(1H, t, J=6 Hz), 2.06-2.17(1H, m), 3.95(3H, s), 3.98(2H, t, J=5 Hz), 4.10(2H, t, J=5 Hz), 6.74(1H, d, J=9 Hz), 6.90(2H, d, J=9 Hz), 7.44(2H, d, J=9 Hz), 7.79(1H, dd, J=9.2 Hz), 8.38(1H, d, J=2 Hz). MS (ES+): 353.19.

### Example 119

### 2-{4-[2-Cyclopropyl-4-(6-methoxy-3-pyridinyl)-1,3-oxazol-5-yl]phenoxy}ethyl methanesulfonate

The title compound (310 mg, 102%) was obtained as an oil from 2-{4-[2-cyclopropyl-4-(6-methoxy-3-pyridinyl)-1,3-oxazol-5-yl]phenoxy}ethanol obtained by Example 118 (250 mg, 0.709 mmol) in a manner similar to that of Example 34.

¹H-NMR (300 MHz, CDCl₃): δ 1.04-1.13(2H, m), 1.13-1.21(2H, m), 2.08-2.20(1H, m), 3.11(3H, s), 3.97(3H, s), 4.22-4.30(2H, m), 4.55-4.61(2H, m), 6.76(1H, d, J=9 Hz), 6.89(2H, d, J=9 Hz), 7.45(2H, d, J=9 Hz), 7.82(1H, dd, J=9.2 Hz), 8.39(1H, d, J=2 Hz). MS (ES+): 431.11.

### Example 120

### 2-(2-{4-[2-Cyclopropyl-4-(6-methoxy-3-pyridinyl)-1,3-oxazol-5-yl]phenoxy}ethyl)-1H-isoindole-1,3(2H)-dione

The title compound (256 mg, 73.8%) was obtained as a powder from 2-{4-[2-cyclopropyl-4-(6-methoxy-3-pyridinyl)-1,3-oxazol-5-yl]phenoxy}ethyl methanesulfonate obtained by Example 119 (310 mg, 0.72 mmol) and potassium phthalimide (200 mg, 1.08 mmol) in a manner similar to that of Example 35.

¹H-NMR (300 MHz, DMSO-d₆): δ 1.00-1.12(4H, m), 2.11-2.23(1H, m), 3.86(3H, s), 3.97(2H, t, J=5 Hz), 4.26(2H, t, J=5 Hz), 6.84(1H, d, J=9 Hz), 6.95(2H, d, J=9 Hz), 7.39(2H, d, J=9 Hz), 7.75(1H, dd, J=9.2 Hz), 7.80-7.94(4H, m), 8.28(1H, d, J=2 Hz). MS (ES+): 482.16.

### Example 121

### 2-{4-[2-Cyclopropyl-4-(6-methoxy-3-pyridinyl)-1,3-oxazol-5-yl]phenoxy}ethylamine

The title compound (220 mg, 121%) was obtained as an oil from 2-(2-{4-[2-cyclopropyl-4-(6-methoxy-3-pyridinyl)-1,3-oxazol-5-yl]phenoxy}ethyl)-1H-isoindole-1,3(2H)-dione obtained by Example 120 (250 mg, 0.519 mmol) in a manner similar to that of Example 36.

¹H-NMR (300 MHz, CDCl₃): δ 1.00-1.11(2H, m), 1.11-1.20(2H, m), 2.06-2.19(1H, m), 3.10(2H, t, J=5 Hz), 3.95(3H, s), 4.00(2H, t, J=5 Hz), 6.74(1H, d, J=9 Hz), 6.89(2H, d, J=9 Hz), 7.44(2H, d, J=9 Hz), 7.79(1H, dd, J=9.2 Hz), 8.39(1H, d, J=2 Hz). MS (ES+): 352.22.

### Example 122

### N-(2-{4-[2-Cyclopropyl-4-(6-methoxy-3-pyridinyl)-1,3-oxazol-5-yl]phenoxy}ethyl)methanesulfonamide

The title compound (57 mg, 51.8%) was obtained as a powder from 2-{4-[2-cyclopropyl-4-(6-methoxy-3-pyridinyl)-1,3-oxazol-5-yl]phenoxy}ethylamine obtained by Example 121 (90 mg, 0.256 mmol) in a manner similar to that of Example 38.

¹H-NMR (300 MHz, CDCl₃): δ 1.03-1.12(2H, m), 1.12-1.21(2H, m), 2.06-2.19(1H,m), 3.04(3H, s), 3.50-3.60(2H,m), 3.95(3H, s), 4.11(2H, t, J=5 Hz), 4.76(1H, br peak), 6.75(1H, d, J=9 Hz), 6.86(2H, d, J=9 Hz), 7.45(2H, d, J=9 Hz), 7.80(1H, dd, J=9.2 Hz), 8.38(1H, d, J=2 Hz). MS (ES+): 430.10.

### Example 123

### N-(2-{4-[2-Cyclopropyl-4-(6-methoxy-3-pyridinyl)-1,3-oxazol-5-yl]phenoxy}ethyl)urea

The title compound (63 mg, 43.2%) was obtained as a powder from 2-{4-[2-cyclopropyl-4-(6-methoxy-3-pyridinyl)-1,3-oxazol-5-yl]phenoxy}ethylamine obtained by Example 121 (130 mg, 0.37 mmol) in a manner similar to that of Example 18.

¹H-NMR (300 MHz, DMSO-d₆): δ 0.99-1.15(4H, m), 2.12-2.24(1H, m), 3.29-3.39(2H, m), 3.87(3H, s), 3.97(2H, t, J=5 Hz), 5.54(2H, br-s), 6.16(1H, t, J=5 Hz), 6.86(1H, d, J=9 Hz), 7.01(2H, d, J=9 Hz), 7.42(2H, d, J=9 Hz), 7.78(1H, dd, J=9.2 Hz), 8.31(1H, d, J=2 Hz). MS (ES+): 395.17.

### Example 124-1

### 1-[4-(Benzyloxy)phenyl]-2-(4-methoxyphenyl)-2-oxoethyl (acetyloxy)acetate

The title compound (8.75 g, 100%) was obtained as an oil from 2-[4-(benzyloxy)phenyl]-2-bromo-1-(4-methoxyphenyl)ethanone (8.3 g, 19.5 mmol) and acetoxyacetic acid (2.3 g, 19.5 mmol) in a manner similar to that of Example 78-4.

¹H-NMR (300 MHz, CDCl₃): δ 2.14(3H, s), 3.82(3H, s), 4.72(1H, d, J=16 Hz), 4.80(1H, d, J=16 Hz), 5.02(2H, s), 6.80-6.90(3H, m), 6.95(2H, d, J=9 Hz), 7.28-7.43(7H, m), 7.89(2H, d, J=9 Hz).

### Example 124-2

### [5-[4-(Benzyloxy)phenyl]-4-(4-methoxyphenyl)-1,3-oxazol-2-yl]methanol

The title compound (4.88 g, 64.6%) was obtained as a powder from 1-[4-(benzyloxy)phenyl]-2-(4-methoxyphenyl)-2-oxoethyl (acetyloxy)acetate obtained by Example 124-1 (8.75 g, 19.5 mmol) in a manner similar to that of Example 91-7.

¹H-NMR (300 MHz, CDCl₃): δ 3.84(3H, s), 4.78(2H, s), 5.08(2H, s), 6.90(2H, d, J=9 Hz), 6.96(2H, d, J=9 Hz), 7.29-7.46(5H, m), 7.50(2H, d, J=9 Hz), 7.55(2H, d, J=9 Hz).

### Example 125

### 5-[4-(Benzyloxy)phenyl]-4-(4-methoxyphenyl)-1,3-oxazole-2-carbaldehyde

The title compound (3.08 g, 63.4%) was obtained as a powder from [5-[4-(benzyloxy)phenyl]-4-(4-methoxyphenyl)-1,3-oxazol-2-yl]methanol obtained by Example 124-2 (4.88 g, 12.6 mmol) in a manner similar to that of Example 71.

H-NMR (300 MHz, CDCl₃): δ 3.87(3H, s), 5.11(2H, s), 6.95(2H, d, J=9 Hz), 7.00(2H, d, J=9 Hz), 7.30-7.50(5H, m), 7.60(2H, d, J=9 Hz), 7.65(2H, d, J=9 Hz), 9.76(1H, s).

### Example 126

### 1-[5-[4-(Benzyloxy)phenyl]-4-(4-methoxyphenyl)-1,3-oxazol-2-yl]-2-methyl-1-propanol

The title compound (150 mg, 26.9%) was obtained as an oil from [5-[4-(benzyloxy)phenyl]-4-(4-methoxyphenyl)-1,3-oxazole-2-carbaldehyde obtained by Example 125 (500 mg, 1.3 mmol) and isopropylmagnesium bromide (0.7M solution in tetrahydrofuran, 2.78 mL) in a manner similar to that of Example 72.

¹H-NMR (300 MHz, CDCl₃): δ 0.98-1.07(6H, m), 2.15-2.34(1H, m), 3.83(3H, s), 4.59(1H, br peak), 5.08(2H, s), 6.90(2H, d, J=9 Hz), 6.95(2H, d, J=9 Hz), 7.29-7.45(5H, m), 7.50(2H, d, J=9 Hz), 7.55(2H, d, J=9 Hz). MS (ES+): 430.19.

### Example 127

### 4-[2-(1-Hydroxy-2-methylpropyl)-4-(4-methoxyphenyl)-1,3-oxazol-5-yl]phenol

The title compound (231 mg, 108%) was obtained as an oil from 1-[5-[4-(benzyloxy)phenyl]-4-(4-methoxyphenyl)-1,3-oxazol-2-yl]-2-methyl-1-propanol obtained by Example 126 (270 mg, 0.629 mmol) in a manner similar to that of Example 31.

¹H-NMR (300 MHz, CDCl₃): δ 0.99-1.08(6H, m), 2.15-2.31(1H, m), 2.74(1H, d, J=7 Hz), 3.83(3H, s), 4.60(1H, t, J=7 Hz), 5.41(1H, s), 6.82(2H, d, J=9 Hz), 7.90(2H, d, J=9 Hz), 7.45(2H, d, J=9 Hz), 7.55(2H, d, J=9 Hz). MS (ES+): 340.19.

### Example 128

### 1-[5-[4-(2-Hydroxyethoxy)phenyl]-4-(4-methoxyphenyl)-1,3-oxazol-2-yl]-2-methyl-1-propanol

The title compound (126 mg, 48.9%) was obtained as an oil from 4-[2-(1-hydroxy-2-methylpropyl)-4-(4-methoxyphenyl)-1,3-oxazol-5-yl]phenol obtained by Example 127 (228 mg, 0.672 mmol) and 2-chloroethanol (325 mg, 4.03 mmol) in a manner similar to that of Example 87.

¹H-NMR (300 MHz, CDCl₃): δ 1.00-1.10(6H, m), 2.00(1H, t, J=6 Hz), 2.19-2.33(1H, m), 2.65(1H, d, J=6 Hz), 3.84(3H, s), 3.96(2H, q, J=5 Hz), 4.10(2H, t, J=5 Hz), 4.60(1H, t, J=6 Hz), 6.85-6.95(4H, m), 7.50(2H, d, J=9 Hz), 7.55(2H, d, J=9 Hz). MS (ES+): 384.18.

### Example 129

### 1-[5-[4-(2-Hydroxyethoxy)phenyl]-4-(4-methoxyphenyl)-1,3-oxazol-2-yl]-2-methyl-1-propanone

The title compound (17 mg, 13.6%) was obtained.as an oil from 1-[5-[4-(2-hydroxyethoxy)phenyl]-4-(4-methoxyphenyl)-1,3-oxazol-2-yl]-2-methyl-1-propanol obtained by Example 128 (126 mg, 0.329 mmol) in a manner similar to that of Example 71.

¹H-NMR (300 MHz, CDCl₃): δ 1.29(6H, d, J=7 Hz), 1.99(1H, t-like), 3.70-3.83(1H, m), 3.86(3H, s), 3.95-4.04(2H, m), 4.12(2H, t, J=5 Hz), 6.88-6.99(4H, m), 7.58(2H, d, J=9 Hz), 7.62(2H, d, J=9 Hz). MS (ES+): 382.13.

### Example 130

### 1-[5-[4-(Benzyloxy)phenyl]-4-(4-methoxyphenyl)-1,3-oxazol-2-yl]-3-methyl-1-butanol

The title compound (143 mg, 24.9%) was obtained as an oil from [5-[4-(benzyloxy)phenyl]-4-(4-methoxyphenyl)-1,3-oxazole-2-carbaldehyde obtained by Example 125 (500 mg, 1.3 mmol) and isobutylmagnesium bromide (2M solution in diethyl ether, 0.78 mL) in a manner similar to that of Example 72.

¹H-NMR (300 MHz, CDCl₃): δ 1.00(6H, d, J=7 Hz), 1.74-1.99(3H, m), 2.50(1H, d, J=6 Hz), 3.84(3H, s), 4.84-4.96(1H, m), 5.09(2H, s), 6.89(2H, d, J=9 Hz), 6.96(2H, d, J=9 Hz), 7.28-7.46(5H, m), 7.50(2H, d, J=9 Hz), 7.55(2H, d, J=9 Hz). MS (ES+): 444.21.

### Example 131

### 4-[2-(1-Hydroxy-3-methylbutyl)-4-(4-methoxyphenyl)-1,3-oxazol-5-yl]phenol

The title compound (112 mg, 99.7%) was obtained as an oil from 1-[5-[4-(benzyloxy)phenyl]-4-(4-methoxyphenyl)-1,3-oxazol-2-yl]-3-methyl-1-butanol obtained by Example 130 (141 mg, 0.318 mmol) in a manner similar to that of Example 31.

¹H-NMR (300 MHz, CDCl₃): δ 1.00(6H, d, J=7 Hz), 1.76-1.96(3H, m), 2.59(1H, br peak), 3.83(3H, s), 4.85-4.95(1H, m), 5.37(1H, br peak), 6.81(2H, d, J=9 Hz), 6.90(2H, d, J=9 Hz), 7.44(2H, d, J=9 Hz), 7.54(2H, d, J=9 Hz). MS (ES+): 354.19.

### Example 132

### 1-[5-[4-(2-Hydroxyethoxy)phenyl]-4-(4-methoxyphenyl)-1,3-oxazol-2-yl]-3-methyl-1-butanol

The title compound (118 mg, 95.4%) was obtained as an oil from 4-[2-(1-hydroxy-3-methylbutyl)-4-(4-methoxyphenyl)-1,3-oxazol-5-yl]phenol obtained by Example 131 (110 mg, 0.311 mmol) and 2-chloroethanol (150 mg, 1.87 mmol) in a manner similar to that of Example 87.

¹H-NMR (300 MHz, CDCl₃): δ 1.01(6H, d, J=7 Hz), 1.75-1.96(3H, m), 2.05(1H, br peak), 2.62(1H, br peak), 3.84(3H, s), 3.94-4.02(2H, m), 4.11(2H, t, J=5 Hz), 4.90(1H, br peak), 6.85-6.95(4H, m), 7.50(2H, d, J=9 Hz), 7.55(2H, d, J=9 Hz). MS (ES+): 398.20.

### Example 133

### 1-[5-[4-(2-Hydroxyethoxy)phenyl]-4-(4-methoxyphenyl)-1,3-oxazol-2-yl]-3-methyl-1-butanone

The title compound (42.5 mg, 36.8%) was obtained as an oil from 1-[5-[4-(2-hydroxyethoxy)phenyl]-4-(4-methoxyphenyl)-1,3-oxazol-2-yl]-3-methyl-1-butanol obtained by Example 132 (116 mg, 0.292 mmol) in a manner similar to that of Example 71.

¹H-NMR (300 MHz, CDCl₃): δ 1.04(6H, d, J=7 Hz), 2.00(1H, t-like, J=5 Hz), 2.30-2.46(1H, m), 3.00(2H, d, J=7 Hz), 3.86(3H, s), 3.95-4.04(2H, m), 4.12(2H, t, J=5 Hz), 6.88-6.99(4H, m), 7.59(2H, d, J=9 Hz), 7.62(2H, d, J=9 Hz). MS (ES+): 396.19.

### Example 134

### 5-[4-(Benzyloxy)phenyl]-4-(4-methoxyphenyl)-1,3-oxazole-2-carboxylic acid

The title compound (1.05 g, 100%) was obtained as an amorphous from 5-[4-(benzyloxy)phenyl]-4-(4-methoxyphenyl)-1,3-oxazole-2-carbaldehyde obtained by Example 125 (1.0 g, 2.59 mmol) in a manner similar to that of Example 74.

¹H-NMR (300 MHz, DMSO-d₆): δ 3.78(3H, s), 5.14(2H, s), 6.98(2H, d, J=9 Hz), 7.10(2H, d, J=9 Hz), 7.30-7.54(9H, m). MS (ES−): 400.19.

### Example 135

### 5-[4-(Benzyloxy)phenyl]-N,N-diethyl-4-(4-methoxyphenyl)-1,3-oxazole-2-carboxamide

The title compound (132 mg, 44.1%) was obtained as a powder from 5-[4-(benzyloxy)phenyl]-4-(4-methoxyphenyl)-1,3-oxazole-2-carboxylic acid obtained by Example 134 (263 mg, 0.655 mmol) and diethylamine (57.5 mg, 0.786 mmol) in a manner similar to that of Example 75.

¹H-NMR (300 MHz, CDCl₃): δ 1.26(3H, t, J=7 Hz), 1.35(3H, t), 3.57(2H, q, J=7 Hz), 3.85(3H, s), 3.91(2H, q, J=7 Hz), 5.09(2H, s), 6.90(2H, d, J=9 Hz), 6.96(2H, d, J=9 Hz), 7.30-7.46(5H, m), 7.54-7.64(4H, m).

### Example 136

### N,N-Diethyl-5-(4-hydroxyphenyl)-4-(4-methoxyphenyl)-1,3-oxazole-2-carboxamide

The title compound (95 mg, 91.1%) was obtained as a powder from 5-[4-(benzyloxy)phenyl]-N,N-diethyl-4-(4-methoxyphenyl)-1,3-oxazole-2-carboxamide obtained by Example 135 (130 mg, 0.285 mmol) in a manner similar to that of Example 31.

¹H-NMR (300 MHz, CDCl₃): δ 1.30(3H, t, J=7 Hz), 1.39(3H, t, J=7 Hz), 3.61(2H, q, J=7 Hz), 3.85(3H, s), 4.05(2H, q, J=7 Hz), 6.91(2H, d, J=9 Hz), 7.00(2H, d, J=9 Hz), 7.45(2H, d, J=9 Hz), 7.55-7.66(3H, m). MS (ES+): 367.20.

### Example 137

### N,N-Diethyl-5-[4-(2-hydroxyethoxy)phenyl]-4-(4-methoxyphenyl)-1,3-oxazole-2-carboxamide

The title compound (58 mg, 57.5%) was obtained as a powder from N,N-diethyl-5-(4-hydroxyphenyl)-4-(4-methoxyphenyl)-1,3-oxazole-2-carboxamide obtained by Example 136 (90 mg, 0.246 mmol) and 2-chloroethanol (119 mg, 1.47 mmol) in a manner similar to that of Example 87.

¹H-NMR (300 MHz, DMSO-d₆): δ 1.16(3H, t, J=7 Hz), 1.27(3H, t, J=7 Hz), 3.46(2H, q, J=7 Hz), 3.66-3.82(7H, m), 4.04(2H, t, J=5 Hz), 4.90(1H, t, J=5 Hz), 7.00(2H, d, J=9 Hz), 7.05(2H, d, J=9 Hz), 7.46-7.55(4H, m). MS (ES+): 411.19.

### Example 138

### 1-{[5-[4-(Benzyloxy)phenyl]-4-(4-methoxyphenyl)-1,3-oxazol-2-yl]carbonyl}piperidine

The title compound (185 mg, 49.5%) was obtained as a powder from 5-[4-(benzyloxy)phenyl]-4-(4-methoxyphenyl)-1,3-oxazole-2-carboxylic acid obtained by Example 134 (320 mg, 0.797 mmol) and piperidine (81.5 mg, 0.957 mmol) in a manner similar to that of Example 75.

¹H-NMR (300 MHz, CDCl₃): δ 1.61-1.78(6H, m), 3.69-3.79(2H, m), 3.84(3H, s), 4.04-4.13(2H, m), 5.09(2H, s), 6.91(2H, d, J=9 Hz), 6.96(2H, d, J=9 Hz), 7.30-7.48(5H, m), 7.54-7.64(4H, m). MS (ES+): 469.20.

### Example 139

### 4-[4-(4-Methoxyphenyl)-2-(1-piperidinylcarbonyl)-1,3-oxazol-5-yl]phenol

The title compound (138 mg, 94.9%) was obtained as a powder from 1-{[5-[4-(benzyloxy)phenyl]-4-(4-methoxyphenyl)-1,3-oxazol-2-yl]carbonyl}piperidine obtained by Example 138 (180 mg, 0.384 mmol) in a manner similar to that of Example 31.

¹H-NMR (300 MHz, CDCl₃): δ 1.64-1.76(6H, m), 3.72-3.82(2H, m), 3.84(3H, s), 4.16-4.26(2H, m), 6.90(2H, d, J=9 Hz), 6.96(2H, d, J=9 Hz), 7.24(1H, s), 7.45(2H, d, J=9 Hz), 7.49(2H, d, J=9 Hz). MS (ES−): 377.28.

### Example 140

### 2-{4-[4-(4-Methoxyphenyl)-2-(1-piperidinylcarbonyl)-1,3-oxazol-5-yl]phenoxy}ethanol

The title compound (96 mg, 66.1%) was obtained as a powder from 4-[4-(4-methoxyphenyl)-2-(1-piperidinylcarbonyl)-1,3-oxazol-5-yl]phenol obtained by Example 139 (130 mg, 0.344 mmol) and 2-chloroethanol (166 mg, 2.06 mmol) in a manner similar to that of Example 87.

¹H-NMR (300 MHz, DMSO-d₆): δ 1.53-1.72(6H, m), 3.63(2H, t, J=5.5 Hz), 3.72(2H, q, J=5 Hz), 3.79(3H, s), 3.94(2H, t, J=5.5 Hz), 4.04(2H, t, J=5 Hz), 4.90(1H, t, J=5.5 Hz), 7.00(2H, d, J=9 Hz), 7.05(2H, d, J=9 Hz), 7.46-7.55(4H, m). MS (ES+): 423.15.

### Example 141-1

### Ethyl {[2-[4-(benzyloxy)phenyl]-1-(6-methoxy-3-pyridinyl)-2-oxoethyl]amino}(oxo)acetate

The title compound (3.0 g, 103%) was obtained from 2-amino-1-[4-(benzyloxy)phenyl]-2-(6-methoxy-3-pyridinyl)ethanone hydrochloride obtained by Example 30-5 in a manner similar to that of Example 1-1.

¹H-NMR (300 MHz, CDCl₃): δ 1.37(3H, t, J=7 Hz), 3.88(3H, s), 4.35(2H, q, J=7 Hz), 5.10(2H, s), 6.41(1H, d, J=7 Hz), 6.67(1H, d, J=8 Hz), 6.97(2H, d, J=8 Hz), 7.31-7.40(5H, m), 7.56(1H, dd, J=8.2 Hz), 7.94(2H, d, J=8 Hz), 8.27(1H, d, J=2 Hz), 8.55(1H, d, J=7 Hz).

### Example 141-2

### Ethyl 5-[4-(benzyloxy)phenyl]-4-(6-methoxy-3-pyridinyl)-1,3-oxazole-2-carboxylate

The title compound was obtained (2.3 g, 82.6%) from ethyl {[2-[4-(benzyloxy)phenyl]-1-(6-methoxy-3-pyridinyl)-2-oxoethyl]amino}(oxo)acetate obtained by Example 141-1 in a manner similar to that of Example 9-5.

¹H-NMR (300 MHz, CDCl₃): δ 1.46(3H, t, J=7 Hz), 3.97(3H, s), 4.52(2H, q, J=7 Hz), 5.10(2H, s), 6.79(1H, d, J=8 Hz), 7.00(2H, d, J=8 Hz), 7.32-7.46(5H, m), 7.59(2H, d, J=8 Hz), 7.86(1H, dd, J=8.2 Hz), 8.44(1H, d, J=2 Hz). MS (ES+): 431.17.

### Example 142

### 5-[4-(Benzyloxy)phenyl]-4-(6-methoxy-3-pyridinyl)-1,3-oxazole-2-carboxamide

To a solution of ethyl 5-[4-(benzyloxy)phenyl]-4-(6-methoxy-3-pyridinyl)-1,3-oxazole-2-carboxylate obtained by Example 141-2 (2.18 g, 5.06 mmol) in 80 mL of 1,4-dioxane at 0° C. was added 2M NH₃ in methanol (25 mL, 50.6 mmol). The clear solution was stirred for 30 min at the same temperature and ammonia gas was bubbled for 5 min. The reaction mixture was allowed to warm to room temperature and stirred for 3 hrs.

The solution was evaporated to give the title compound (2.1 g, quant.) as white crystals.

¹H-NMR (300 MHz, CDCl₃): δ 3.98(3H, s), 5.10(2H, s), 5.75(1H, br-s), 6.79(1H, d, J=8 Hz), 6.97(1H, br-s), 7.00(2H, d, J=8 Hz), 7.34-7.45(5H, m), 7.59(2H, d, J=8 Hz), 7.82(1H, dd, J=8.2 Hz), 8.45(1H, d, J=2 Hz). MS (ES+): 402.13.

### Example 143

### 5-(4-Hydroxyphenyl)-4-(6-methoxy-3-pyridinyl)-1,3-oxazole-2-carboxamide

The title compound was obtained (1.7 g, 99.6%) from 5-[4-(benzyloxy)phenyl]-4-(6-methoxy-3-pyridinyl)-1,3-oxazole-2-carboxamide obtained by Example 142 in a manner similar to that of Example 65.

¹H-NMR (300 MHz, DMSO-d₆): δ 3.89(3H, s), 6.87(2H, d, J=8 Hz), 6.92(1H, d, J=8 Hz), 7.44(2H, d, J=8 Hz), 7.86(1H, dd, J=8.2 Hz), 7.94(1H, br-s), 8.31(1H, br-s), 8.38(1H, d, J=2 Hz). MS (ES+): 312.15.

### Example 144

### tert-Butyl 2-{4-[2-(aminocarbonyl)-4-(6-methoxy-3-pyridinyl)-1,3-oxazol-5-yl]phenoxy}ethylcarbamate

The title compound was obtained (2.1 g, 98.5%) from 5-(4-hydroxyphenyl)-4-(6-methoxy-3-pyridinyl)-1,3-oxazole-2-carboxamide obtained by Example 143 in a manner similar to that of Example 13.

¹H-NMR (300 MHz, CDCl₃): δ 1.46(9H, s), 3.55(2H, m), 3.98(3H, s), 4.05(2H, t, J=5 Hz), 5.02(1H, br), 5.83(1H, br-s), 6.79(1H, d, J=8 Hz), 6.91(2H, d, J=8 Hz), 6.99(1H, br-s), 7.58(2H, d, J=8 Hz), 7.81(1H, dd, J=8.2 Hz), 8.43(1H, d, J=2 Hz). MS (ES+): 455.08.

### Example 145

### tert-Butyl 2-{4-[2-cyano-4-(6-methoxy-3-pyridinyl)-1,3-oxazol-5-yl]phenoxy}ethylcarbamate

The title compound was obtained (1.4 g, 69.4%) from tert-butyl 2-{4-[2-(aminocarbonyl)-4-(6-methoxy-3-pyridinyl)-1,3-oxazol-5-yl]phenoxy}ethylcarbamate obtained by Example 144 in a manner similar to that of Example 23.

¹H-NMR (300 MHz, CDCl₃): δ 1.46(9H, s), 3.56(2H, m), 3.98(3H, s), 4.07(2H, t, J=5 Hz), 4.98(1H, br), 6.80(1H, d, J=8 Hz), 6.94(2H, d, J=8 Hz), 7.54(2H, d, J=8 Hz), 7.80(1H, dd, J=8.2 Hz), 8.42(1H, d, J=2 Hz). MS (ES+): 437.09.

### Example 146

### 5-[4-(2-Aminoethoxy)phenyl]-4-(6-methoxy-3-pyridinyl)-1,3-oxazole-2-carbonitrile

The title compound was obtained (1.3 g, 108%) from tert-butyl 2-{4-[2-cyano-4-(6-methoxy-3-pyridinyl)-1,3-oxazol-5-yl]phenoxy}ethylcarbamate obtained by Example 145 in a manner similar to that of Example 17.

¹H-NMR (300 MHz, CDCl₃): δ 3.10-3.14(2H, m), 3.89(1H, br-s), 3.97(3H, s), 4.03(2H, m), 4.28(1H, br), 6.78(1H, m), 6.98(2H, m), 7.54(2H, dd, J=8.2 Hz), 7.80(1H, m), 8.43(1H, s). MS (ES+): 337.13.

### Example 147

### N-(2-{4-[2-Cyano-4-(6-methoxy-3-pyridinyl)-1,3-oxazol-5-yl]phenoxy}ethyl)methanesulfonamide

The title compound was obtained (20 mg, 9.2%) from 5-[4-(2-aminoethoxy)phenyl]-4-(6-methoxy-3-pyridinyl)-1,3-oxazole-2-carbonitrile obtained by Example 146 in a manner similar to that of Example 38.

¹H-NMR (300 MHz, CDCl₃): δ 3.04(3H, s), 3.55-3.61(2H, m), 3.98(3H, s), 4.16(2H, t, J=5 Hz), 4.83(1H, br-t, J=5 Hz), 6.81(1H, d, J=8 Hz), 6.94(2H, d, J=8 Hz), 7.56(2H, d, J=8 Hz), 7.81(1H, dd, J=8.2 Hz), 8.42(1H, d, J=2 Hz). MS (ES+): 415.01.

### Example 148

### N-(2-{4-[2-Cyano-4-(6-methoxy-3-pyridinyl)-1,3-oxazol-5-yl]phenoxy}ethyl)urea

The title compound was obtained as crystals (55 mg, 79.7%) from 5-[4-(2-aminoethoxy)phenyl]-4-(6-methoxy-3-pyridinyl)-1,3-oxazole-2-carbonitrile obtained by Example 146 in a manner similar to that of Example 18.

¹H-NMR (300 MHz, DMSO-d₆): δ 3.30-3.39(2H, m), 3.90(3H, s), 4.01(2H, t, J=5 Hz), 5.55(2H, s), 6.18(1H, br-t, J=5 Hz), 6.94(1H, d, J=8 Hz), 7.10(2H, d, J=8 Hz), 7.56(2H, d, J=8 Hz), 7.85(1H, dd, J=8.2 Hz), 8.38(1H, d, J=2 Hz). MS (ES+): 380.09.

### Example 149-1

### 1-(4-Methoxyphenyl)-2-(6-methoxy-3-pyridinyl)-2-oxoethyl 2-hydroxy-2-methylpropanoate

The title compound (1.32 g, 51.7%) was obtained from 2-(4-methoxyphenyl)-2-bromo-1-(6-methoxy-3-pyridinyl)ethanone and 2-hydroxy-2-methylpropionic acid in a manner similar to that of Example 78-4.

¹H-NMR (300 MHz, CDCl₃): δ 1.48(3H, s), 1.59(3H, s), 1.67(1H, br-s), 3.79(3H, s), 3.96(3H, s), 6.72(1H, s), 6.74(1H, d, J=8.8 Hz), 6.91(2H, d, J=8.8 Hz), 7.37(2H, d, J=8.8 Hz), 8.09(1H, dd, J=8.8, 2.6 Hz), 8.77(1H, d, J=2.6 Hz). MS (ES+): 360.20.

### Example 149-2

### 2-[5-(4-Methoxyphenyl)-4-(6-methoxy-3-pyridinyl)-1,3-oxazol-2-yl]-2-propanol

The title compound (175 mg, 14%) was obtained from 1-(4-methoxyphenyl)-2-(6-methoxy-3-pyridinyl)-2-oxoethyl 2-hydroxy-2-methylpropanoate obtained by Example 149-1 and ammonium acetate in a manner similar to that of Example 64-2.

¹H-NMR (300 MHz, CDCl₃): δ 1.72(6H, s), 2.48(1H, br-s), 3.84(3H, s), 3.96(3H, s), 6.77(1H, d, J=8.4 Hz), 6.92(2H, d, J=8.8 Hz), 7.48(2H, d, J=8.8 Hz), 7.84(1H, dd, J=8.4, 2.6 Hz), 8.43(1H, d, J=2.6 Hz). MS (ES+): 341.18 (M+1).

### Example 150-1

### 4,5-Bis(4-methoxyphenyl)-1,3-oxazol-2(3H)-one

A mixture of 2-hydroxy-1,2-bis(4-methoxyphenyl)ethanone (25 g) and urethane (24.5 g) was heated at 190° C. overnight.

The mixture was poured into a mixture of water (150 mL) and acetone (150 mL). The resulting precipitates were collected, washed with 50% acetone aqueous solution, coevaporated with toluene twice, and triturated with ethyl acetate. The resulting powder was collected, washed with ethyl acetate, and dried in vacuo. This crude product was used for the next step without further purification.

MS (ESI): 296.2 (M−1).

### Example 150-2

### 4,5-Bis(4-methoxyphenyl)-2-chloro-1,3-oxazole

A mixture of 4,5-bis(4-methoxyphenyl)-1,3-oxazol-2(3H)-one obtained by Example 150-1 (18.73 g), phosphoryl chloride (58.7 mL) and triethylamine (8.78 mL) was stirred under reflux at 120° C. for 5 hrs.

The mixture was cooled, concentrated, coevaporated with toluene twice, dissolved in ethyl acetate (150 mL), washed with water twice, dried over magnesium sulfate, and concentrated in vacuo. The residue was chromatographed on silica gel (n-hexane/ethyl acetate=9/1) to give the crude product, which was purified by recrystallization from methanol (5.5 g).

¹H NMR (CDCl₃): δ 3.83(3H, s), 3.84(3H, s), 6.80-7.70(8H, m). MS (ESI): 338.2 (M+Na)⁺.

### Example 151-1

### 2-Bromo-1-(4-methoxyphenyl)-2-(6-methoxy-3-pyridinyl)ethanone

Under a nitrogen atmosphere, pyridinium tribromide (4.62 g) was added to a suspension of 1-(4-methoxyphenyl)-2-(6-methoxy-3-pyridinyl)ethanone (3.72 g) in a mixture of 30% hydrogen bromide in acetic acid (3 mL) and dichloromethane (30 mL).

The mixture was stirred for 30 min, and poured into a mixture of cold water and ethyl acetate. The solution was adjusted pH 5 with 10% aqueous potassium dicarbonate and the aqueous layer was separated. The organic layer was washed with 5% aqueous sodium thiosulfate, saturated aqueous sodium hydrogencarbonate and brine, and dried over magnesium sulfate. Evaporation of the solvent afforded the title compound (4.88 g).

¹H-NMR (DMSO-d₆): δ 3.84 (3H, s), 3.85 (3H, s), 6.83 (1H, d, J=10.1 Hz), 7.08 (2H, d, J=9 Hz), 7.88 (1H, dd, J=2.6, 8.6 Hz), 8.37 (2H, d, J=2.4 Hz).

### Example 151-2

### 2-Amino-1-(4-methoxyphenyl)-2-(6-methoxy-3-pyridinyl)ethanone hydrochloride

2-Bromo-1-(4-methoxyphenyl)-2-(6-methoxy-3-pyridinyl)ethanone obtained by Example 151-1 (1.82 g) was dissolved in dimethylformamide (18 mL) and the solution was cooled at 0° C. Ammonium gas was bubbled into the solution for 30 min. Ammonium gas was ceased and nitrogen was passed through the solution for 15 min at the same temperature.

The solution was poured into a mixture of cold water and ethyl acetate, and the aqueous layer was separated. The organic layer was washed with water and brine, and dried over magnesium sulfate. The solution was conecntrated to about 20 mL and 4N hydrochloric acid in ethyl acetate (0.6 mL) was added. The resulting precipitate was collected by filtration, washed with ethyl acetate, and dried in vacuo to give the title compound (1.44 g).

¹H-NMR (DMSO-d₆): δ 3.85(3H, s), 3.88(3H, s), 6.89(1H, d, J=8.6 Hz), 7.03(2H, d, J=8.8 Hz), 7.88(1H, dd, J=2.2, 8.6 Hz), 8.03(2H, d, J=8.8 Hz), 8.92(1H, d, J=2 Hz).

### Example 151-3

### 2-{[2-(4-Methoxyphenyl)-1-(6-methoxy-3-pyridinyl)-2-oxoethyl]amino}-2-oxoethyl acetate

Under a nitrogen atmosphere, acetoxyacetyl chloride (0.75 mL) and triethylamine (2.6 mL) was added successively to a soluton of 2-amino-1-(4-methoxyphenyl)-2-(6-methoxy-3-pyridinyl)ethanone hydrochloride obtained by Example 151-2 (1.43 g) in dichloromethane (15 mL) at 0° C.

The mixture was stirred for 2 hrs at the same temperature, and poured into a mixture of cold water and ethyl acetate. The aqueous layer was separated And the organic layer was washed with diluted aqueous hydrochloric acid, water and brine, and dried over magnesium sulfate. Evaporation of the solvent afforded the title compound (1.51 g).

¹H-NMR (DMSO-d₆): δ 2.11(3H, s), 3.81(3H, s), 3.83(3H, s), 4.53(2H, s), 6.8(1H, d, J=8.6 Hz), 7.01(2H, d, J=8.8 Hz), 7.68(1H, dd, J=2.3, 8.6 Hz), 7.96(2H, d, J=8.8 Hz), 8.26(1H, d, J=2.3 Hz), 8.88(1H, d, J=7 Hz). MS (ESI): 395.2 (M+Na)⁺.

### Example 151-4

### [5-(4-Methoxyphenyl)-4-(6-methoxy-3-pyridinyl)-1,3-oxazol-2-yl]methyl acetate

To a mixture of triphenylphosphine (3.17 g), iodine (3.07 g) and triethylamine (3.4 ml) in dichloromethane (30 mL), a solution of 2-{[2-(4-methoxyphenyl)-1-(6-methoxy-3-pyridinyl)-2-oxoethyl]amino}-2-oxoethyl acetate obtained by Example 151-3 (1.5 g) in dichloromethane (15 mL) was added at 0° C. under nitrogen, and the mixture was stirred overnight at same temperature.

The reaction mixture was poured into cold water and dichloromethane. The organic layer was separated, washed with 1 N aqueous hydrochloric acid, water, saturated sodium bicarbonate solution and brine, successively, dried over magnesium sulfate. After evaporation of solvent, the residue was purified by column chromatography on silica-gel eluting with dichloromethane and acetone to give the title compound (255 mg).

¹H-NMR (DMSO-d₆): δ 2.12(3H, s), 3.83(3H, s), 3.87(3H, s), 5.23(2H, s), 6.89(1H, d, J=8.6 Hz), 7.05(2H, d, J=8.9 Hz), 7.47(2H, d, J=8.9 Hz), 7.82(1H, dd, J=2.5, 8.6 Hz), 8.34(1H, d, J=2.5 Hz). MS (ESI): 377.2 (M+Na)⁺.

### Example 152

### 5-[4-(Benzyloxy)phenyl]-4-(4-methoxyphenyl)-1,3-oxazole-2-carboxylic acid

1N aqueous sodium hydroxide solution (2.33 mL) solution was added to a solution of ethyl 5-[4-(benzyloxy)phenyl]-4-(4-methoxyphenyl)-1,3-oxazole-2-carboxylate (100 mg) in methanol (0.5 mL) and tetrahydrofuran (0.5 mL) at 0° C.

After stirring for 10 hrs at room temperature, the pH of the solution was justified to 1 with 1N hydrochloric acid. The precipitate was produced, which was collected by filtration to give the title compound (94.0 mg).

MS (ESI): 402 (M+H)⁺.

### Example 153

### 1-{[5-[4-(Benzyloxy)phenyl]-4-(4-methoxyphenyl)-1,3-oxazol-2-yl]carbonyl}piperidine

1-(3-Dimethylaminopropyl)-3-ethylcarbodiimide hydrochloride (EDCI.HCl) (44.9 mg) was added to a solution of 5-[4-(benzyloxy)phenyl]-4-(4-methoxyphenyl)-1,3-oxazole-2-carboxylic acid obtained bye Example 152 (94.0 mg) in dimethylformamide (1.0 mL) at room temperature. After stirring for 5 min, 1-hydroxybenzotriazole hydrate (HOBT) was added to the mixture at room temperature. After stirring for 5 min, to the mixture was added piperidine. The mixture was stirred for 3 days.

The products were extracted with ethyl acetate. The combined extracts were washed with brine, drid over magnesium sulfate, and evaporated. The residue was purified by preparative thin layer chromatography to give the title compound (90.1 mg).

¹H-NMR (200 MHz, CDCl₃): δ 1.7(6H, br-s), 3.7-3.78(2H, m), 3.81(3H, s), 4-4.09(2H, m), 5.08(2H, s), 6.92(2H, d, J=8.5 Hz), 6.97(2H, d, J=9 Hz), 7.29-7.5(5H, m), 7.52-7.66(4H, m). MS (ESI): 469 (M+H)⁺.

### Example 154

### 4-[4-(4-Methoxyphenyl)-2-(1-piperidinylcarbonyl)-1,3-oxazol-5-yl]phenol

The title compound was obtained from 1-{[5-[4-(benzyloxy)phenyl]-4-(4-methoxyphenyl)-1,3-oxazol-2-yl]carbonyl}piperidine obtained by Example 153 in a manner similar to Example 163 described later.

¹H-NMR (200 MHz, CDCl₃): δ 1.49 (6H, br-s), 3.72-3.87(2H, m), 3.84(3H, s), 4.19-4.35(2H, m), 6.91(2H, d, J=9 Hz), 7.01(2H, d, J=9 Hz), 7.42(2H, d, J=9 Hz), 7.6(2H, d, J=9 Hz).

### Example 155

### 4,5-Bis(4-methoxyphenyl)-2-methoxy-1,3-oxazole

To a solution of 4,5-bis(4-methoxyphenyl)-2-chloro-1,3-oxazole obtained by Example 150-2 (102 mg) in methanol (10 ml), 28% sodium methoxide in methanol (1 ml) was added dropwise, and the mixture was stirred at 60° C. for 1 hr.

The mixture was concentrated, diluted with water, and extracted with dichloromethane three times. The combined extracts were concentrated. The residue was chromatographed on silica gel (n-hexane/ethyl acetate=4/1) to give the title compound (83 mg).

¹H-NMR (CDCl₃): δ 3.82(3H, s), 3.83(3H, s), 4.14(3H, s), 6.70-7.70(8H, m). MS (ESI): 312.2 (M+H)⁺.

### Example 156

### 7-[4,5-Bis(4-methoxyphenyl)-1,3-oxazol-2-yl]-5,6,7,8-tetrahydroimidazo[1,2-a]pyrazine dihydrochloride

A mixture of 4,5-bis(4-methoxyphenyl)-2-chloro-1,3-oxazole obtained by Example 150-2 (100 mg), 5,6,7,8-tetrahydroimidazo[1,2-a]pyrazine dihydrochloride (92.2 mg), potassium carbonate (438 mg) in dimethylsulfoxied (10 ml) was stirred at 120° C. overnight.

The mixture was cooled, diluted with ethyl acetate, washed with water three times, dried over magnesium sulfate, and concentrated. The residue was purified by preparative thin layer chromatography using 10% methanol in dichloromethane as an eluent to give the title compound, which was converted to the corresponding hydrochloride salt (47 mg).

¹H-NMR (DMSO-d₆): δ 2.00-5.00(19H, m), 6.80-7.70(8H, m). MS (ESI): 403.3 (M+H)⁺ (free).

### Example 157

### 4,5-Bis(4-methoxyphenyl)-2-(methylthio)-1,3-oxazole

A mixture of 4,5-bis(4-methoxyphenyl)-2-chloro-1,3-oxazole obtained by Example 150-2 (3 g) and sodium thiomethoxide (1.33 g) in ethanol was stirred at 85° C. for 1.2 hrs.

The mixture was cooled, diluted with ethyl acetate, washed with water, dried over magnesium sulfate, and concentrated to give the title compound (3.12 g).

¹H-NMR (CDCl₃): δ 2.71(3H, s), 3.83(6H, s), 6.80-7.80(8H, m). MS (ESI): 328.1 (M+H)⁺.

### Example 158

### 4,5-Bis(4-methoxyphenyl)-2-(methylsulfonyl)-1,3-oxazole

A mixture of 4,5-bis(4-methoxyphenyl)-2-(methylthio)-1,3-oxazole obtained by Example 157 (3.07 g), m-chloroperbenzoic acid (4.85 g) in dichloromethane was stirred at room temperature overnight.

The mixture was concentrated, diluted with ethyl acetate, washed with sodium thiosulfate (Na₂S₂O₃) aqueous solution, sodium hydrogencarbonate aqueous solution and brine. The combined extracts were dried over magnesium sulfate and concentrated. The residue was chromatographed on silica gel (dichloromethane) to afford a solid, which was triturated with diisopyopylether to give the titele compound (1.9 g).

¹H-NMR (CDCl₃): δ 3.41(3H, s), 3.85(3H, s), 3.86(3H, s), 6.80-7.80(8H, m). MS (ESI): 382.1 (M+Na)⁺.

### Example 159

### N-[4,5-Bis(4-methoxyphenyl)-1,3-oxazol-2-yl]-N,N′,N′-trimethyl-1,2-ethanediamine dihydrochloride

A mixture of 4,5-bis(4-methoxyphenyl)-2-chloro-1,3-oxazole obtained by Example 150-2 (200 mg) and N,N,N′-trimethyl-1,2-ethanediamine (324 mg) in dioxane was stirred at 85° C. overnight.

The mixture was cooled, diluted with ethyl acetate, washed with water three times, dried over magnesium sulfate, and concentrated. The residue was purified by thin layer chromatography (dichloromethane/methanol=9/1) to give the title compound, which was converted to the corresponding dihydrochloride (192 mg).

¹H-NMR (DMSO-d₆): δ 2.00-5.00(19H, m), 6.80-7.70(8H, m). MS (ESI): 382.3 (M+H)⁺ (free).

### Example 160-1

### Ethyl {[1-[4-(benzyloxy)phenyl]-2-(4-methoxyphenyl)-2-oxoethyl]amino}(oxo)acetate

Ethyl chlorooxoacetate (427 mg) was added to a solution of 2-amino-2-[4-(benzyloxy)phenyl]-1-(4-methoxyphenyl)ethanone hydrochloride (1.00 g) in benzene (20 mL) at room temperature. The mixture was refluxed for 1 hr.

The products were extracted with ethyl acetate. The combined extracts were washed with 1N hydrochloric acid, saturated sodium hydrrogencarbonate aquetous solution and brine, dried over magnesium sulfate, and evaporated in vacuo to afford the title compound (1.20 g).

¹H-NMR (200 MHz, CDCl₃): δ 1.37(3H, t, J=7 Hz), 3.83(3H, s), 4.34(2H, q, J=7 Hz), 4.99(2H, s), 6.42(1H, d, J=7.5 Hz), 6.87(2H, d, J=6 Hz), 6.91(2H, d, J=6 Hz), 7.27-7.45(6H, m), 7.95(2H, d, J=9 Hz), 8.49(1H, d, J=7.5 Hz). MS (ESI): 470 (M+Na)⁺.

### Example 160-2

### Ethyl 4-[4-(benzyloxy)phenyl]-5-(4-methoxyphenyl)-1,3-oxazole-2-carboxylate

Phosphorus oxychloride (1.00 mL) was added to a solution of ethyl {[1-[4-(benzyloxy)phenyl]-2-(4-methoxyphenyl)-2-oxoethyl]amino}(oxo)acetate obtained by Example 160-1 (1.20 g) in toluene (12 mL) at 0° C. The mixture was refluxed for 15 hrs.

The products were extracted with ethyl acetate. The combined extracts were washed with 1N hydrochloric acid, saturated sodium hydrogencarbonate aqueous solution and brine, dried over magnesium sulfate, and evaporated in vacuo. The residue was purified by silica gel column chromatography to give the title compound (752 mg).

¹H-NMR (200 MHz, CDCl₃): δ 1.45(3H, t, J=7.1 Hz), 3.84(3H, s), 4.51(2H, q, J=7.1 Hz), 5.1(2H, s), 6.91(2H, d, J=8.5 Hz), 6.99(2H, d, J=8.5 Hz), 7.3-7.5(5H, m), 7.57-7.63(4H, m). MS (ESI): 452 (M+N)⁺.

### Example 161

### 4-[4-(Benzyloxy)phenyl]-N-methoxy-5-(4-methoxyphenyl)-N-methyl-1,3-oxazole-2-carboxamide

Under a nitrogen atmosphere, trimethylaluminium (0.98M in hexane, 2.48 mL) was added to a solution of N,O-dimethylhydroxylamine hydrochloride (504 mg) in tetrahydrofuran (10 mL) at 0° C. After stirring for 1 hr at room temperature, a solution of ethyl 4-[4-(benzyloxy)phenyl]-5-(4-methoxyphenyl)-1,3-oxazole-2-carboxylate obtained by Example 160-2 (740 mg) in tetrahydrofuran (14 mL) was added to the reaction mixture.

After stirring for 12 hrs at 43° C., the reaction mixture was stopped by adding 1N hydrochloric acid at 0° C. The products were extracted with ethyl acetate. The combined extracts were washed with saturated sodium hydrogencarbonate aqueous solution and brine, dried over magnesium sulfate, and evaporated in vacuo. The residue was purified by silica gel column chromatography to give the title compound (625 mg).

¹H-NMR (200 MHz): δ 3.33(3H, s), 3.81(3H, s), 3.87(3H, s), 5.14(2H, s), 7.05(2H, d, J=6.5 Hz), 7.1(2H, d, J=6.4 Hz), 7.32-7.57 (9H, m) MS (ESI): 467 (M+Na)⁺.

### Example 162

### 1-[4-[4-(Benzyloxy)phenyl]-5-(4-methoxyphenyl)-1,3-oxazol-2-yl]-2-methyl-1-propanone

Under a nitrogen atmosphere, isopropylmagnesium chloride (2.0M in diethyl ether, 0.77 mL) was added to a solution of 4-[4-(benzyloxy)phenyl]-N-methoxy-5-(4-methoxyphenyl)-N-methyl-1,3-oxazole-2-carboxamide obtained by Example 161 (320 mg) in diethyl ether (6.5 mL) at −78° C., and the mixture was stirred at 0° C. for 1.5 hrs.

The mixture was poured into saturated ammonium chloride aqueous solution and the products were extracted with ethyl acetate. The combined extracts were washed with brine, dried over magnesium sulfate, and evaporated in vacuo. The residue was purified by silica gel column chromatography to give the title compound (195 mg).

¹H-NMR (200 MHz, CDCl₃): δ 1.3(6H, d, J=7 Hz), 3.69-3.84(1H, m), 3.85(3H, s), 5.11(2H, s), 6.91(2H, d, J=8.5 Hz), 7.01(2H, d, J=8.5 Hz), 7.3-7.51(5H, m), 7.59(2H, d, J=6 Hz), 7.63(2H, d, J=6 Hz).

### Example 163

### 1-[4-(4-Hydroxyphenyl)-5-(4-methoxyphenyl)-1,3-oxazol-2-yl]-2-methyl-1-propanone

10% Pd/C (44 mg) was added to 1-[4-[4-(benzyloxy)phenyl]-5-(4-methoxyphenyl)-1,3-oxazol-2-yl]-2-methyl-1-propanone obtained by Example 162 (181 mg) in methanol (2.1 mL) and dioxane (2.1 mL) at room temperature.

After stirring for 10 hrs under a hydrogen atmosphere, the mixture was filtered through celite pad and the filtrate was evaporated in vacuo to give the title compound (163 mg).

¹H-NMR (200 MHz, CDCl₃): δ 1.3(6H, d, J=7 Hz), 3.74-3.85(1H, m), 3.85(3H, s), 5.21(1H, br-s), 6.86(2H, d, J=6.5 Hz), 6.91(2H, d, J=6.5 Hz), 7.54(2H, d, J=8.5 Hz), 7.62(2H, d, J=9 Hz). MS (ESI): 360 (M+Na)⁺.

### Example 164

### 1-[4-[4-(2-{[tert-Butyl(dimethyl)silyl]oxy}ethoxy)phenyl]-5-(4-methoxyphenyl)-1,3-oxazol-2-yl]-2-methyl-1-propanone

NaH (60% in mineral oil, 14.8 mg)was added to a solution of 1-[4-(4-hydroxyphenyl)-5-(4-methoxyphenyl)-1,3-oxazol-2-yl]-2-methyl-1-propanone obtained by Example 163 (160 mg) in dimethylformamide (2.3 mL) at 0° C. After stirring for 10 min, a solution of (2-bromoethoxy)(tert-butyl)dimethylsilane (139 mg) in dimethylformamide (2.0 mL) was added. The mixture was stirred for 4 hrs at room temperature.

The mixture was poured into ice-cooling water and the products were extracted with ethyl acetate. The combined extracts were washed with brine, drid over magnesium sulfate, and evaporated. The residue was purified by silica gel column chromatography to give the title compound (105 mg).

¹H-NMR (200 MHz, CDCl₃): δ 0.12(6H, s), 0.92(9H, s), 1.3(6H, d, J=7 Hz), 3.74-3.86(1H, m), 3.85(3H, s), 3.93-4.10(m, 4H), 6.9(2H, d, J=8.5 Hz), 6.94(2H, d, J=9.0 Hz), 7.58(2H, d, J=8.5 Hz), 7.62(2H, d, J=9 Hz). MS (ESI): 496 (M+H)⁺.

### Example 165

### 5-[4-(Benzyloxy)phenyl]-N-methoxy-4-(4-methoxyphenyl)-N-methyl-1,3-oxazole-2-carboxamide

The title compound was obtained from ethyl 5-[4-(benzyloxy)phenyl]-4-(4-methoxyphenyl)-1,3-oxazole-2-carboxylate in a manner similar to Example 161.

¹H-NMR (200 MHz, DMSO-d₆): δ 3.34(3H, s), 3.8(3H, s), 3.87(3H, s), 5.16(2H, s), 7.01(2H, d, J=8.7 Hz), 7.14(2H, d, J=8.8 Hz), 7.31-7.56(9H, m). MS (ESI): 445 (M+H)⁺.

### Example 166

### 1-[4-[4-(2-Hydroxyethoxy)phenyl]-5-(4-methoxyphenyl)-1,3-oxazol-2-yl]-2-methyl-1-propanone

Tetrabutylammonium fluoride (1N in tetrahydrofuran 0.424 mL) was added to a solution of 1-[4-[4-(2-{[tert-butyl(dimethyl)silyl]oxy}ethoxy)phenyl]-5-(4-methoxyphenyl)-1,3-oxazol-2-yl]-2-methyl-1-propanone obtained by Example 164 (105 mg) in tetrahydrofuran (1.2 mL) at 0° C.

After stirring for 1 hr, the products were extracted with ethyl acetate. The combined extracts were washed with brine, drid over magnesium sulfate, and evaporated. The residue was purified by preparative thin layer chromatography to give the title compound (36.5 mg).

¹H-NMR (200 MHz, CDCl₃): δ 1.3(6H, d, J=3.5 Hz), 3.75-3.84(1H, m), 3.85(3H, s), 4(2H, d, J=2.2 Hz), 6.91(2H, d, J=4.4 Hz), 7.6(2H, d, J=3.3 Hz), 7.62(2H, d, J=3.3 Hz). MS (ESI): 382 (M+H)⁺.

### Example 167-1

### 2-[4-(Benzyloxy)phenyl]-2-hydroxy-1-(4-methoxyphenyl)ethanone

A mixture of 2-[4-(benzyloxy)phenyl]-2-bromo-1-(4-methoxyphenyl)ethanone (2.83 g) in acetone (30 mL) and water (15 mL) was stirred under reflux at 70° C. for 1 hr.

The mixture was concentrated, diluted with water, and extracted with ethyl acetate twice. The combined extracts were dried over magnesium sulfate and concentrated. The residue was chromatographed on silica gel (n-hexane/ethyl acetate=4/1) to give the title compound (1.98 g).

¹H-NMR (CDCl₃): δ 3.83(3H, s), 4.58(1H, d, J=6.0 Hz), 5.01(2H, s), 5.85(1H, d, J=6.0 Hz), 6.70-8.10(13H, m). MS (ESI): 371.2 (M+Na)⁺.

### Example 167-2

### 5-[4-(Benzyloxy)phenyl]-4-(4-methoxyphenyl)-1,3-oxazol-2(3H)-one

To a warmed (80° C.) solution of 2-[4-(benzyloxy)phenyl]-2-hydroxy-1-(4-methoxyphenyl)ethanone obtained by Example 167-1 (4.1 g) in dimethylformamide (8 mL) were added potassium cyanate (1.91 g) and acetic acid (1.48 mL) in sequence.

After stirring at this temperature under a nitrogen atmosphere for 2 hrs, the mixture was poured into water (30 mL). The resulting powder was collected, washed with water, coevaporated with toluene, and dried in vacuo to give the crude product (4.87 g), which was used for the next step without further purification.

MS (ESI): 372.3 (M−1)⁻.

### Example 167-3

### 5-[4-(Benzyloxy)phenyl]-2-chloro-4-(4-methoxyphenyl)-1,3-oxazole

The title compound was obtained from 5-[4-(benzyloxy)phenyl]-4-(4-methoxyphenyl)-1,3-oxazol-2(3H)-one obtained by Example 167-2 in a manner similar to Example 150-2.

¹H-NMR (CDCl₃): δ 3.84(3H, s), 5.09(2H, s), 6.80-7.80(13H, m). MS (ESI): 392.2 (M+H)⁺.

### Example 168

### 5-[4-(Benzyloxy)phenyl]-2-methoxy-4-(4-methoxyphenyl)-1,3-oxazole

To a suspension of 5-[4-(benzyloxy)phenyl]-2-chloro-4-(4-methoxyphenyl)-1,3-oxazole obtained by Example 167-3 (1 g) in methanol (20 mL) was added dropwise 28% methanol solution of sodium methoxide (5.2 mL).

After stirring at 60° C. overnight, the mixture was concentrated, diluted with ethyl acetate, and washed with water and brine. The organic layer was dried over magnesium sulfate and concentrated. The residue was triturated with methanol, and the resulting powder was collected, washed with methanol, and dried in vacuo (50° C.) to give the title compound (0.72 g).

¹H-NMR (CDCl₃): δ 3.82(3H, s), 4.14(3H, s), 5.07(2H, s), 6.70-7.70(13H, m). MS (ESI): 388.3 (M+H)⁺.

### Example 169

### 5-(4-Hydroxyphenyl)-2-methoxy-4-(4-methoxyphenyl)-1,3-oxazole

A mixture of 5-[4-(benzyloxy)phenyl]-2-methoxy-4-(4-methoxyphenyl)-1,3-oxazole obtained by Example 168 (0.72 g) and 20% palladium hydroxide (dry base) on carbon (wet; 0.22 g) in ethanol (10 mL) and cyclohexene (5 mL) was stirred under reflux at 95° C. for 2 hrs.

The mixture was filtered and concentrated to give the title compound (490 mg).

¹H-NMR (CDCl₃): δ 3.83(3H, s), 4.14(3H, s), 5.11(1H, s), 6.70-7.70(8H, m). MS (ESI): 298.1 (M+H)⁺.

### Example 170

### 2-{4-[2-Methoxy-4-(4-methoxyphenyl)-1,3-oxazol-5-yl]phenoxy}ethanol

A mixture of 5-(4-hydroxyphenyl)-2-methoxy-4-(4-methoxyphenyl)-1,3-oxazole obtained by Example 169 (486 mg), (2-bromoethoxy)(tert-butyl)dimethylsilane (1.17 g), potassium carbonate (1.13 g) and potassium iodide (814 mg) in dimethylformamide was stirred at 75° C. for 3 hrs.

The mixture was diluted with ethyl acetate, washed with water three times, dried over magnesium sulfate, and concentrated. To a solution of the residue in tetrahydrofuran was added 1M tetrahydrofuran solution of tetrabutylammoniumfluoride (7 mL), and the mixture was stirred at room temperature under a nitrogen atmosphere for 1.5 hrs.

The reaction mixture was quenched with water and extracted with ethyl acetate twice. The combined extracts were washed with water twice and brine, dried over magnesium sulfate, and concentrated. The residue was chromatographed on silica gel (n-hexane/ethyl acetate=1/1) to give the title compound (346 mg).

¹H-NMR (CDCl₃): δ 2.01(1H, t, J=6.0 Hz), 3.82(3H, s), 3.85-4.30(7H, m), 6.70-7.70(8H, m). MS (ESI): 364.1 (M+Na)⁺.

### Example 171

### 2-{4-[2-Methoxy-4-(4-methoxyphenyl)-1,3-oxazol-5-yl]phenoxy}ethyl methanesulfonate

To a solution of 2-{[4-[2-methoxy-4-(4-methoxyphenyl)-1,3-oxazol-5-yl]phenoxy}ethanol obtained by Example 170 (334 mg) and triethylamine (0.409 mL) in ethyl acetate, methanesulfonylchloride (0.114 ml) was added dropwsie. And the mixture was stirred at room temperature for 1 hr.

The reaction mixture was quenched with water and extracted with ethyl acetate twice. The combined extracts were dried over magnesium sulfate and concentrated to give the crude procuct (0.44 g), which was used for the next step without further purification.

### Example 172

### 2-(2-{4-[2-Methoxy-4-(4-methoxyphenyl)-1,3-oxazol-5-yl]phenoxy}ethyl)-1H-isoindole-1,3(2H)-dione

A mixture of crude 2-{4-[2-methoxy-4-(4-methoxyphenyl)-1,3-oxazol-5-yl]phenoxy}ethyl methanesulfonate obtained by Example 171 (0.44 g) and potassium phthalimide (272 mg) in dimethylformamide was stirred at 60° C. overnight.

The mixture was cooled, diluted with water, and extracted with ethyl acetate twice. The conbined extracts were dried over magnesium sulfate and concentrated to give the crude product (0.57 g), which, was used for the next step without further pirification.

MS (ESI): 493.1 (M+Na)⁺.

### Example 173

### (2-{4-[2-Methoxy-4-(4-methoxyphenyl)-1,3-oxazol-5-yl]phenoxy}ethyl)amine

A mixture of the crude 2-(2-{4-[2-methoxy-4-(4-methoxyphenyl)-1,3-oxazol-5-yl]phenoxy}ethyl)-1H-isoindole-1,3(2H)-dione obtained by Example 172 (0.57 g) and hydrazine monohydrate (0.142 ml) in ethanol was stirred at 70° C. for 2 hrs.

The mixture was cooled, diluted with water, extracted with dichloromethane three times. The combined extracts were dried over magnesium sulfate and concentrated. The residue was chromatographed on silica gel (dichloromethane/methanol=9/1) to give the title compound (246 mg) as an oil.

¹H-NMR (CDCl₃): δ 1.00-4.30(12H, m), 6.60-7.70(8H, m). MS (ESI): 341.2 (M+H)⁺.

### Example 174

### N-(2-{4-[2-Methoxy-4-(4-methoxyphenyl)-1,3-oxazol-5-yl]phenoxy}ethyl)urea

A mixture of (2-{4-[2-methoxy-4-(4-methoxyphenyl)-1,3-oxazol-5-yl]phenoxy}ethyl)amine obtained by Example 173 (80 mg), trimethylsilylisocyanate (0.16 mL) and triethylamine (0.16 mL) in dichloromethane was stirred at room temperature overnight.

The reaction mixture was quenched with water and extracted with ethyl acetate twice. The combined extracts were washed with water three times, dried over magnesium sulfate, and concentrated. The residue was purified by preparative thin-layer chromatography (dichloromethane/methanol=9/1) to give the title compound (54 mg).

¹H-NMR (CDCl₃): δ 2.80-5.60(13H, m), 6.40-8.30(8H, m). MS (ESI): 441.20 (M+Na)⁺.

### Example 175

### 5-[4-(Benzyloxy)phenyl]-4-(4-methoxyphenyl)-1,3-oxazole

1N NaOH aqueous solution (51.7 mL) was added to a solution of ethyl 5-[4-(benzyloxy)phenyl]-4-(4-methoxyphenyl)-1,3-oxazole-2-carboxylate (1.11 g) in methanol (9.0 mL) and tetrahydrofuran (25.0 mL) at 0° C.

After stirring for 1 hr at room temperature, the pH of the mixture was justified to 1 with 1N hydrochloric acid followed by extraction with ethyl acetate. The combined extracts were washed with brine, dried over magnesium sulfate, and evaporated in vacuo to give the title compound (800 mg).

¹H-NMR (200 MHz, DMSO-d₆): δ 3.78(3H, s), 5.14(2H, s), 6.98(2H, d, J=4.4 Hz), 7.11(2H, d, J=4.4 Hz), 7.32-7.52(9H, m), 8.43(1H, s). MS (ESI): 358 (M+H)⁺.

### Example 176

### 4-[4-(4-Methoxyphenyl)-1,3-oxazol-5-yl]phenol

The title compound was obtained from 5-[4-(benzyloxy)phenyl]-4-(4-methoxyphenyl)-1,3-oxazole obtained by Example 175 in a manner similar to Example 163.

### Example 177

### 5-[4-(2-{[tert-Butyl(dimethyl)silyl]oxy}ethoxy)phenyl]-4-(4-methoxyphenyl)-1,3-oxazole

The title compound was obtained from 4-[4-(4-methoxyphenyl)-1,3-oxazol-5-yl]phenol obtained by Example 176 and (2-bromoethoxy)(tert-butyl)dimethylsilane in a manner similar to Example 164.

¹H-NMR (200 MHz, CDCl₃): δ 0.01(6H, s), 0.81(9H, s), 3.73(3H, s), 3.9-3.99(4H, m), 6.8(4H, d, J=8.8 Hz), 7.41(2H, d, J=8.9 Hz), 7.47(2H, d, J=8.9 Hz), 7.79(1H, s). MS (ESI): 426 (M+H)⁺.

### Example 178

### 2-{4-[4-(4-Methoxyphenyl)-1,3-oxazol-5-yl]phenoxy}ethanol

The title compound was obtained from 5-[4-(2-{[tert-butyl(dimethyl)silyl]oxy}ethoxy)phenyl]-4-(4-methoxyphenyl)-1,3-oxazole obtained by Example 177 in a manner similar to Example 166.

¹H-NMR (200 MHz, CDCl₃): δ 2.12(1H, br-s), 3.81(3H, s), 3.97-4.02(2H, m), 4.1-4.14(2H, m), 6.86-6.97(4H, m), 7.53(2H, d, J=9 Hz), 7.58(2H, d, J=9 Hz), 7.9(1H, s). MS (ESI): 312 (M+H)⁺.

### Example 179

### 5-[5-[4-(Benzyloxy)phenyl]-2-(1-piperidinylcarbonyl)-1,3-oxazol-4-yl]-2-methoxypyridine

To a solution of N,O-dimethylhydroxyamine hydrochloride (509 mg) in dry benzene (4.2 mL), 2.3 mL of triethylaluminum (2M solution in toluene) was added dropwise at 0° C. under nitrogen atmosphere. And the mixture was stirred at room temperature for 2 hrs. A solution of ethyl 5-[4-(benzyloxy)phenyl]-4-(6-methoxy-3-pyridinyl)-1,3-oxazole-2-carboxylate (720 mg) in dry benzene (16.7 mL) was added dropwise to the mixture at room temperature and the reaction mixture was refluxed for 2 hrs.

The reaction mixture was cooled to room temperature and quenched with 5% aqueous hydrochloric acid. The mixture was poured into 1M aqueous hydrochloric acid and extracted with ethyl acetate. The organic layer was washed with water and brine, dried over magnesium sulfate, and evaporated in vacuo. The residue was purified by column chromatography on silica-gel eluting with n-hexane and ethyl acetate to give the title compound (550 mg).

¹H-NMR (DMSO-d₆): δ 1.5-1.75(6H, br-s), 3.6-3.7(2H, m), 3.89(3H, s), 3.9-4.0(2H, m), 5.16(2H, s), 6.91(1H, d, J=9.0 Hz), 7.14(2H, d, J=8.9 Hz), 7.3-7.6(7H, m), 7.86(1H, dd, J=9.0, 2.3 Hz), 8.37(1H, d, J=2.3 Hz). MS (ESI): 492.2 (M+Na)⁺.

### Example 180

### 4-[4-(6-Methoxy-3-pyridinyl)-2-(1-piperidinylcarbonyl)-1,3-oxazol-5-yl]phenol

10% Palladium on carbon (50% wet, 50 mg) and ammonium formate (210 mg) was added to a solution of 5-[5-[4-(benzyloxy)phenyl]-2-(1-piperidinylcarbonyl)-1,3-oxazol-4-yl]-2-methoxypyridine obtained by Example 179 (520 mg) in ehtanol(10 mL), tetrahydrofuran (4 mL) and water (3 mL). The mixture was stirred at reflux condition for 4 hrs and cooled to room temperature.

After filtration through celite, the filtrate was concentared in vacuo. The residue was dissolved in a mixture of water and ethyl acetate. The aqueous layer was separated, the organic layer was washed with brine, and dried over magnesium sulfate. After evaporation of the solvent, the residue was purified by column chromatography on silica-gel eluting with dichloromethane and acetone to give the title compound (330 mg).

¹H-NMR (DMSO-d₆) d: 1.5-1.75(6H, br-s), 3.6-3.7(2H, m), 3.89(3H, s), 3.9-4.0(2H, m), 5.16(2H, s), 6.91(1H, d, J=9.0 Hz), 7.14(2H, d, J=8.9 Hz), 7.3-7.6(7H, m), 7.86(1H, dd, J=9.0, 2.3 Hz), 8.37(1H, d, J=2.3 Hz). MS (ESI): 492.2 (M+Na)⁺.

### Example 181

### 2-{4-[4-(6-Methoxy-3-pyridinyl)-2-(1-piperidinylcarbonyl)-1,3-oxazol-5-yl]phenoxy}ethanol

Under a nitrogen atmosphere, sodium hydride(12.7 mg) was added to a solution of 4-[4-(6-methoxy-3-pyridinyl)-2-(1-piperidinylcarbonyl)-1,3-oxazol -5-yl]phenol obtained by Example 180 (100 mg) in dimethylformamide (5 mL) at 0° C. After 10 min, a solution of (2-bromoethoxy)trimethylsilane (104 mg) in dimethylformamide (1 mL) was added. The whole mixture was stirred overnight at room temperature.

The mixture was poured into a mixture of water and ethyl acetate, and the aqueous layer was separated. The organic layer was washed with water, brine, and dried over magnesium sulfate. After evaporation of the solvent, the residue was dissolved in tetrahydrofuran (5 mL). Tetrabutylammonium fluoride (1M in tetrahydrofuran, 0.52 mL) was added to this solution.

The mixture was stirred at room temperature for 3 hrs, and poured into into a mixture of water and ethyl acetate. The aqueous layer was separated, the organic layer was washed with water and brine, and dried over magnesium sulfate. After evaporation of the solvent, the residue was purified by column chromatography on silica-gel eluting with dichloromethane and acetone to give the title compound (86 mg).

¹H-NMR (DMSO-d₆): δ 1.5-1.8(6H, br-s), 3.55-3.8(4H, m), 3.89(3H, s), 3.85-3.95(2H, m), 4.05(2H, t), 4.92(1H, t, J=5.5 Hz), 6.91(1H, d, J=8.6 Hz), 7.07(2H, d, J=8.7 Hz), 7.51(2H, d, J=8.7 Hz), 7.85(1H, dd, J=8.6, 2.3 Hz), 8.38(1H, J=2.3 Hz). MS (ESI): 466.0 (M+CH₃CN)⁺.

### Example 182

### tert-Butyl (2-{4-[4-(6-methoxy-3-pyridinyl)-2-(1-piperidinylcarbonyl)-1,3-oxazol-5-yl]phenoxy}ethyl)carbamate

Under a nitrogen atmosphere, sodium hydride(59 mg) was added to a solution of 4-[4-(6-methoxy-3-pyridinyl)-2-(1-piperidinylcarbonyl)-1,3-oxazol-5-yl]phenol obtained by Example 180 (280 mg) in dimethylformamide (5 mL) at 0° C. After 10 min, a solution of tert-butyl (2-bromoethyl)carbamate (496 mg) in dimethylformamide (1 mL) was added. The whole mixture was stirred overnight at room temperature.

The mixture was poured into a mixture of water and ethyl acetate. The aqueous layer was separated, the organic layer was washed with water and brine, and dried over magnesium sulfate. After evaporation of the solvent, the residue was purified by column chromatography on silica-gel eluting with dichloromethane and acetone to give the title compound (361 mg).

¹H-NMR (DMSO-d₆): δ 1.38(9H, s), 1.6-1.8(6H, br-s), 3.25-3.4(4H, m), 3.6-3.7(2H, b), 3.88(3H, s), 3.8-4.1(4H, m), 6.91(1H, d, J=8.7 Hz), 7.05(2H, d, J=8.8 Hz), 7.51(2H, d, J=8.8 Hz), 7.86(1H, dd, J=8.7, 2.2 Hz), 8.38(1H, J=2.2). MS (ESI): 545.0 (M+Na)⁺.

### Example 183

### 5-[4-(Benzyloxy)phenyl]-N-methoxy-4-(6-methoxy-3-pyridinyl)-N-methyl-1,3-oxazole-2-carboxamide

The title compound (480 mg) was obtained from ethyl 5-[4-(benzyloxy)phenyl]-4-(6-methoxy-3-pyridinyl)-1,3-oxazole-2-carboxylate (680 mg) and N,O-dimethylhydroxylamine hydrochloride (385 mg) in a manner similar to Example 179.

¹H-NMR (DMSO-d₆): δ 3.87(3H, s), 3.89(6H, s), 5.16(2H, s), 6.92(1H, d, J=8.5 Hz), 7.15(2H, d, J=8.8 Hz), 7.4-7.6(7H, m), 7.88(1H, dd, J=8.5, 2.3 Hz), 8.40(1H, d, J=2.3 Hz). MS (ESI): 468.0 (M+Na)⁺.

### Example 184

### 4,5-Bis(4-methoxyphenyl)-2-[(1-methyl-3-pyrrolidinyl)oxy]-1,3-oxazole

To a suspension of sodium hydride (40 mg, 60% in mineral oil) and 1-methyl-3-pyrrolidinol (101 mg), 4,5-bis(4-methoxyphenyl)-2-(methylsulfonyl)-1,3-oxazole obtained by Example 158 (120 mg) was added in portions. And the mixture was stirred at room temperature overnight.

The mixture was diluted with water and extracted with ethyl acetate twice. The combined extracts were dried over magnesium sulfate and concentrated. The residue was purified by thin layer chromatography (dichloromethane/methanol=9/1) to give the title compound as an oil (121 mg)

¹H-NMR (CDCl₃): δ 0.70-3.10(9H, m), 3.82(3H, s), 3.83(3H, s), 5.41(1H, m). 6.80-7.70(8H, m). MS (ESI): 403.13 (M+Na)⁺.

### Example 185

### 4-[4-(4-Methoxyphenyl)-2-(methylthio)-1,3-oxazol-5-yl]phenol

To a solution of 5-[4-(benzyloxy)phenyl]-4-(4-methoxyphenyl)-2-(methylthio)-1,3-oxazole (0.88 g) in chloroform was added dropwise trimethylsilyliodide (1.45 mL) at 0° C., and the mixture was stirred at room temperature overnight. The reaction mixture was quenched with methanol (1 mL), stirred for 15 min, diluted with water, and extracted with ethyl acetate.

The organic phase was washed with water, 10% sodium hydrogencarbonate aqueous solution and brine, dried over magnesium sulfate, and concentrated. The residue was chromatographed on silica gel (n-hexane/ethyl acetate=7/3) to give the title compound (0.63 g).

¹H NMR(CDCl₃): δ 2.71(3H, s), 3.83(3H, s), 5.28(2H, s), 6.70-7.70(8H, m). Mass (ESI): 314.2 (M+H)⁺.

### Example 186

### 2-{4-[4-(4-Methoxyphenyl)-2-(methylthio)-1,3-oxazol-5-yl]phenoxy}ethanol

A mixture of 4-[4-(4-methoxyphenyl)-2-(methylthio)-1,3-oxazol-5-yl]phenol obtained by Example 185 (0.63 g), (2-bromoethoxy)(tert-butyl)dimethylsilane (721 mg), potassium carbonate (1.39 g) and potassium iodide (1 g) in dimethylformamide was stirred at 75° C. for 2 hrs.

The mixture was diluted with water, and extracted with ethyl acetate twice. The combined extracts were washed with water three times, dried over magnesium sulfate, and concentrated.

To a solution of the residue in tetrahydrofuran, 1M tetrahydrofuran solution of tetrabutylammonium fluoride (6 mL) was added dropwise at 0° C., and the mixture was stirred at room temperature for 30 min. The reaction mixture was quenched with water and extracted with ethyl acetate. The organic layer was washed with water twice and brine, dried over magnesium sulfate, and concentrated. The residue was chromatographed on silica gel (n-hexane/ethyl acetate=1/1) to give the title compound (0.71 g).

¹H-NMR (CDCl₃): δ 2.03(1H, t, J=6.2 Hz), 2.71(3H, s), 3.83(3H, s), 3.90-4.20(4H, m), 6.70-7.70(8H, m). MS (ESI): 358.20 (M+H)⁺.

### Example 187

### 2-{4-[4-(4-Methoxyphenyl)-2-(methylthio)-1,3-oxazol-5-yl]phenoxy}ethyl methanesulfonate

The title compound was obtained from 2-{4-[4-(4-methoxyphenyl)-2-(methylthio)-1,3-oxazol-5-yl]phenoxy}ethanol obtained by Example 186 in a manner similar to Example 171.

### Example 188

### 2-(2-{4-[4-(4-Methoxyphenyl)-2-(methylthio)-1,3-oxazol-5-yl]phenoxy}ethyl)-1H-isoindole-1,3(2H)-dione

The title compound was obtained from 2-{4-[4-(4-methoxyphenyl)-2-(methylthio)-1,3-oxazol-5-yl]phenoxy}ethyl methanesulfonate obtained by Example 187 in a manner similar to Example 172.

¹H-NMR (CDCl₃): δ 2.70(3H, s), 3.82(3H, s), 4.00-4.30(4H, m), 6.70-8.00(12H, m). MS (ESI): 509.27 (M+Na)⁺.

### Example 189

### (2-{4-[4-(4-Methoxyphenyl)-2-(methylthio)-1,3-oxazol-5-yl]phenoxy}ethyl)amine

The title compound was obtained from 2-(2-{4-[4-(4-methoxyphenyl)-2-(methylthio)-1,3-oxazol-5-yl]phenoxy}ethyl)-1H-isoindole-1,3(2H)-dione obtained by Example 188 in a manner similar to Example 173.

¹H-NMR (CDCl₃): δ 2.71(3H, s), 3.11(2H, m), 3.83(3H, s), 4.02(2H, t, J=5.1 Hz), 6.70-7.80(8H, m). MS (ESI): 357.20 (M+H)⁺.

### Example 190

### N-(2-{4-[4-(4-Methoxyphenyl)-2-(methylthio)-1,3-oxazol-5-yl]phenoxy}ethyl)methanesulfonamide

The title compound was obtained from obtained by Example 189 in a manner similar to Example 221 described later.

¹H-NMR (CDCl₃): δ 2.71(3H, s), 3.03(3H, s), 3.56(2H, m), 3.84(3H, s), 4.13(2H, t, J=5.0 Hz), 4.76(1H, br-s), 6.80-7.80(8H, m). MS (ESI): 457.27 (M+Na)⁺.

### Example 191

### N-(2-{4-[4-(4-Methoxyphenyl)-2-(methylsulfinyl)-1,3-oxazol-5-yl]phenoxy}ethyl)methanesulfonamide

The title compound was obtained from N-(2-{4-[4-(4-methoxyphenyl)-2-(methylthio)-1,3-oxazol-5-yl]phenoxy}ethyl)methanesulfonamide obtained by Example 190 in a manner similar to Example 193 described later.

¹H-NMR (CDCl₃): δ 3.04(3H, s), 3.19(3H, s), 3.58(2H, m), 3.85(3H, s), 4.15(2H, t, J=5.0 Hz), 4.78(1H, br-s), 6.80-7.70(8H, m). MS (ESI): 472.87 (M+Na)⁺.

### Example 192

### N-(2-{4-[4-(4-Methoxyphenyl)-2-(methylsulfonyl)-1,3-oxazol-5-yl]phenoxy}ethyl)methanesulfonamide

A mixture of N-(2-{4-[4-(4-methoxyphenyl)-2-(methylsulfinyl)-1,3-oxazol-5-yl]phenoxy}ethyl)methanesulfonamide obtained by Example 191 (38 mg) and m-chloroperbenzoic acid (44 mg) in dichloromethane was stirred at room temperature overnight. The mixture was diluted with AcOEt, washed with 10% NaHSO3 aqueous solution, saturated NaHCO3 aqueous solution and brine, dried over magnesium sulfate and concentrated to give the title compound (34 mg).

¹H-NMR (CDCl₃): δ 3.04(3H, s), 3.41(3H, s), 3.58(2H, m), 3.85(3H, s), 4.13(2H, t, J=5.0 Hz), 4.77(1H, t, J=6.0 Hz), 6.80-7.80(8H, m). MS (ESI): 488.87 (M+Na)⁺.

### Example 193

### 2-{4-[4-(4-Methoxyphenyl)-2-(methylsulfinyl)-1,3-oxazol-5-yl]phenoxy}ethanol

A mixture of 2-(4-[4-{4-methoxyphenyl)-2-(methylthio)-1,3-oxazol-5-yl]phenoxy}ethanol obtained by Example 186 (63 mg) and oxone (325 mg) in tetrahydrofuran (15 mL) and water (15 mL) was stirred at room temperature for 2 hrs.

The mixture was diluted with ethyl acetate, washed with water and brine, dried over magnesium sulfate, and concentrated. The residue was purified by preparative thin-layer chromatography (ethyl acetate) to give the title compound (28 mg).

¹H-NMR (CDCl₃): δ 2.00(1H, t, J=6.1 Hz), 3.18(3H, s), 3.85(3H, s), 3.90-4.20(4H, m), 6.80-7.70(8H, m). MS (ESI): 396.20 (M+H)⁺.

### Example 194

### tert-Butyl (2-{4-[4-(4-methoxyphenyl)-2-(methylthio)-1,3-oxazol-5-yl]phenoxy}ethyl)carbamate

A mixture of 4-[4-(4-methoxyphenyl)-2-(methylthio)-1,3-oxazol-5-yl]phenol obtained by Example 185 (186 mg), tert-butyl (2-bromoethyl)carbamate (399 mg), potassium carbonate (410 mg) and potassium iodide (493 mg) in dimethylformamide was stirred at 80° C. for 2 hrs.

The reaction mixture was cooled, dilute with water, and extracted with ethyl acetate twice. The combined extracts were washed with water three times, dried over magnesium sulfate, and concentrated. The residue was chromatographed on silica gel (n-hexane/ethyl acetate=4/1) to give the title compound (252 mg).

¹H-NMR (CDCl₃): δ 1.00-5.40(19H, m), 6.60-7.70(8H, m). MS (ESI): 479.1 (M+Na)⁺.

### Example 195

### (2-{4-[4-(4-Methoxyphenyl)-2-(methylthio)-1,3-oxazol-5-yl]phenoxy}ethyl)amine hydrochloride

To a solution of tert-butyl (2-{4-[4-(4-methoxyphenyl)-2-(methylthio)-1,3-oxazol-5-yl]phenoxy}ethyl)carbamate obtained by Example 194 (249 mg) in ethyl acetate (5 mL) was added 4N hydrogen chloride in ethyl acetate (5 mL), and the mixture was stirred at room temperature for 3 hrs.

The resulting powder was collected, washed with ethyl acetate, and dried in vacuo to give the title compound (194 mg).

¹H-NMR (CDCl₃): δ 2.71(3H, s), 3.22(2H, m), 3.78(3H, s), 4.22(2H, t, J=5.0 Hz), 6.80-7.70(8H, m), 8.23(3H, br-s). MS (ESI): 357.1 (M+H)⁺ (free).

### Example 196

### N-(2-{4-[4-(4-Methoxyphenyl)-2-(methylthio)-1,3-oxazol-5-yl]phenoxy}ethyl)urea

To a mixture of (2-{4-[4-(4-methoxyphenyl)-2-(methylthio)-1,3-oxazol-5-yl]phenoxy}ethyl)amine hydrochloride obtained by Example 195 (191 mg) and sodium acetate (80 mg) in dimethylformamide (3 mL) and water (2 mL) was added potassium cyanate (79 mg), and the mixture was stirred at room temperature overnight.

The mixture was diluted with ethyl acetate, washed with water three times, dried over magnesium sulfate, and concentrated. The residue was chromatographed on silica gel (dichloromethane/methanol=9/1) to give the title compound (126 mg).

¹H-NMR (CDCl₃): δ 2.71(3H, s), 3.62(2H, m), 3.82(3H, s), 4.06(2H, t, J=5.0 Hz), 4.51(2H, br-s), 5.03(1H, br-s), 6.70-7.60(8H, m). MS (ESI): 422.2 (M+Na)⁺.

### Example 197

### N-(2-{4-[4-(4-Methoxyphenyl)-2-(methylsulfonyl)-1,3-oxazol-5-yl]phenoxy}ethyl)urea

A mixture of N-(2-{4-[4-(4-methoxyphenyl)-2-(methylthio)-1,3-oxazol-5-yl]phenoxy}ethyl)urea obtained by Example 196 (123 mg) and m-chloroperbenzoic acid (213 mg) in dichloromethane was stirred at room temperature overnight.

The mixture was diluted with ethyl acetate, washed with 10% sodium hydrogencarbonate aqueous solution, saturated hydrogencarbonate aqueous solution and brine, dried over magnesium sulfate, and concentrated. The residue was triturated with ethanol, and the resulting powder was collected, washed with ethanol, and dried in vacuo to give the title compound (90 mg).

¹H-NMR (CDCl₃): δ 3.41(3H, s), 3.63(2H, m), 3.85(3H, s), 4.09(2H, t, J=5.0 Hz), 4.37(2H, br-s), 4.90(1H, br-s), 6.80-7.80(8H, m). MS (ESI): 454.1 (M+Na)⁺.

### Example 198

### (2-{4-[4-(6-Methoxy-3-pyridinyl)-2-(1-piperidinylcarbonyl)-1,3-oxazol-5-yl]phenoxy}ethyl)amine hydrochloride

4N hydrogen chloride solution in ethyl acetate (0.67 mL) was added to a solution of tert-butyl (2-{4-[4-(6-methoxy-3-pyridinyl)-2-(1-piperidinylcarbonyl)-1,3-oxazol-5-yl]phenoxy}ethyl)carbamate obtained by Example 182 (350 mg) in ethyl acetate (5 mL) at 0° C. The mixture was stirred overnight at room temperature.

The product was collected by filtration, washed with ethyl acetate, and dried under reduced pressure to give the title compound (259 mg).

¹H-NMR (DMSO-d₆): δ 1.5-1.8(6H, m), 3.1-3.3(2H, m), 3.6-3.7(2H, m), 3.89(3H, s), 3.8-4.0(2H, m), 4.26(2H, t, J=4.9 Hz), 6.93(1H, d, J=8.6 Hz), 7.12(2H, d, J=8.9 Hz), 7.56(2H, d, J=8.9 Hz), 7.85(1H, dd, J=8.6, 1.9 Hz), 8.2-8.3(2H, br-s), 8.37(1H, d, J=1.9 Hz). MS (ESI): 423.0 (M+H)⁺.

### Example 199

### N-(2-{4-[4-(6-Methoxy-3-pyridinyl)-2-(1-piperidinylcarbonyl)-1,3-oxazol-5-yl]phenoxy}ethyl)methanesulfonamide

Under a nitrogen atmosphere, methanesulfonyl chloride (42.7 mg) was added to a solution of (2-{4-[4-(6-methoxy-3-pyridinyl)-2-(1-piperidinylcarbonyl)-1,3-oxazol-5-yl]phenoxy}ethyl)amine hydrochloride obtained by Example 198 (114 mg) and triethylamine (101 mg) in dichloromethane (1.5 mL) at 0° C.

The mixture was poured into a mixture of cold water and ethyl acetate, and stirred for 20 min. The aqueous layer was separated and the organic layer was washed with diluted hydrochloric acid, water and brine, and dried over magnesium sulfate. After evaporation of the solvent, the residue was purified by column chromatography on silica-gel eluting with dichloromethane and acetone to give the title compound (60 mg).

¹H-NMR (DMSO-d₆): δ 1.5-1.7(6H, m), 2.96(3H, s), 3.3-3.4(2H, m), 3.6-3.7(2H, m), 3.89(3H, s), 3.9-4.0(2H, m), 4.0-4.1(2H, m), 6.92(1H, d, J=8.6 Hz), 7.08(2H, d, J=8.7 Hz), 7.53(2H, d, J=8.7 Hz), 7.86(1H, dd, J=8.6, 2.2 Hz), 8.37(1H, d, J=2.2 Hz). MS (ESI): 522.9 (M+Na)⁺.

### Example 200

### N-(2-{4-[4-(6-Methoxy-3-pyridinyl)-2-(1-piperidinylcarbonyl)-1,3-oxazol-5-yl]phenoxy}ethyl)urea

A solution of potassium cyanate (49.1 mg) in water (1 mL) was added to a mixture of (2-{4-[4-(6-methoxy-3-pyridinyl)-2-(1-piperidinylcarbonyl)-1,3-oxazol-5-yl]phenoxy}ethyl)amine hydrochloride obtained by Example 198 (139 mg) and sodium acetate (49.7 mg) in a mixture of dimethylformamide (2 mL) and water (0.5 mL) at room temperature.

The mixture was stirred overnight at 50° C., and was poured into a mixture of water and ethyl acetate. The aqueous layer was separated, the organic layer was washed with water and brine, and dried over magnesium sulfate. After evaporation of the solvent, the residue was purified by column chromatography on silica-gel eluting with dichloromethane and acetone to give the title compound (77 mg).

¹H-NMR (DMSO-d₆): δ 1.5-1.8(6H, m), 3.3-3.4(2H, m), 3.6-3.7(2H, m), 3.89(3H, s), 3.8-4.1(4H, m), 5.55(2H, s), 6.19(1H, t, J=5.6 Hz), 6.91(1H, d, J=8.6 Hz), 7.07(2H, d, J=8.7 Hz), 7.53(2H, d, J=8.7 Hz), 7.86(1H, dd, J=8.6, 2.2 Hz), 8.38(1H, d, J=2.2 Hz). MS (ESI): 488.0 (M+Na)⁺.

### Example 201

### [5-[4-(Benzyloxy)phenyl]-4-(6-methoxy-3-pyridinyl)-1,3-oxazol-2-yl]methanol

Under a nitrogen atmoshere, potassium carbonate (383 mg) was added to a solution of

### [5-[4-(benzyloxy)phenyl]-4-(6-methoxy-3-pyridinyl)-1,3-oxazol-2-yl]methyl acetate (995 mg) in methanol (20 mL) at room tempetarue.

The mixture was stirred overnight at the same temperature and poured into a mixture of water and ethyl acetate. The aqueous layer was separated, the organic layer was washed with water and brine, and dried over magnesium sulfate. After evaporation of the solvent, the residue was purified by column chromatography on silica-gel eluting with dichloromethane and acetone to give the title compound (790 mg).

¹H-NMR (DMSO-d₆): δ 3.87(3H, s), 4.58(2H, d, J=6.2 Hz), 2.57(1H, t, J=−515.2 Hz), 6.88(1H, d, J=8.6 Hz), 7.12(2H, d, J=8.8 Hz), 7.3-7.6(7H, m), 7.82(1H, dd, J=2.4, 8.6 Hz), 8.35(1H, d, J=2.3 Hz). MS (ESI): 389.0 (M+H)⁺.

### Example 202

### 1-[5-[4-(Benzyloxy)phenyl]-4-(6-methoxy-3-pyridinyl)-1,3-oxazol-2-yl]-3-methyl-1-butanone

Under a nitrogen atmosphere, isobutylmagnesium bromide (2M solution in tetrahydrofuran, 1.5 mL) was added to a solution of 5-[4-(benzyloxy)phenyl]-N-methoxy-4-(6-methoxy-3-pyridinyl)-N-methyl-1,3-oxazole-2-carboxamide obtained by Example 183 (632 mg) in tetrahydrofuran (10 mL) at −78° C. The mixture was warmed to 0° C. and stirred for 3 hrs at the same temperature.

The reaction mixture was quenched with saturated aqueous ammonium chloride, and the mixture was poured into a mixture of water and ethyl acetate. The aqueous layer was separated, the organic layer was washed with water and brine, and dried over magnesium sulfate. After evaporation of the solvent, the residue was purified by column chromatography on silica-gel eluting with dichloromethane and acetone to give the title compound (361 mg).

¹H-NMR (DMSO-d₆): δ 0.97(6H, d, J=6.6 Hz), 2.1-2.3(1H, m), 2.97(2H, d, J=6.9 Hz), 3.90(3H, s), 5.16(2H, s), 6.93(1H, d, J=8.6 Hz), 7.15(2H, d, J=8.9 Hz), 7.3-7.6(7H, m), 7.88(1H, dd, J=8.6, 1.8 Hz), 8.37(1H, d, J=1.8 Hz). MS (ESI): 465.0 (M+Na)⁺.

### Example 203

### [5-[4-(Benzyloxy)phenyl]-4-(4-methoxyphenyl)-1,3-oxazol-2-yl](cyclopropyl)methanone

The title compound was obtained from 5-[4-(benzyloxy)phenyl]-N-methoxy-4-(4-methoxyphenyl)-N-methyl-1,3-oxazole-2-carboxamide obtained by Example 165 in a manner similar to Example 162.

¹H-NMR (200 MHz, CDCl₃): δ 1.05-1.2(2H, m), 1.28-1.4(2H, m), 3.07-3.26(1H, m), 3.85(3H, s), 5.1(2H, s), 6.94(2H, d, J=6.5 Hz), 6.98(2H, d, J=6.4 Hz), 7.3-7.5(5H, m), 7.55-7.67(4H, m). MS (ESI): 426 (M+H)⁺.

### Example 204

### 4-[2-(1-Hydroxybutyl)-4-(4-methoxyphenyl)-1,3-oxazol-5-yl]phenol

The title compound was obtained from [5-[4-(benzyloxy)phenyl]-4-(4-methoxyphenyl)-1,3-oxazol-2-yl](cyclopropyl)methanone obtained by Example 203 in a manner similar to Example 163.

¹H-NMR (200 MHz, CDCl₃): δ 0.97(3H, t, J=7.3 Hz), 1.34-1.64 (2H, m), 1.8-2.08(2H, m), 2.94(1H, br-s), 3.82(3H, s), 4.85(1H, t, J=6.5 Hz), 5.86(1H, br-s), 6.81(2H, d, J=9 Hz), 6.89(2H, d, J=9 Hz), 7.43(2H, d, J=8.5 Hz), 7.54(2H, d, J=8.5 Hz). MS (ESI): 340 (M+H)⁺.

### Example 205

### 1-[5-[4-(2-{[tert-Butyl(dimethyl)silyl]oxy}ethoxy)phenyl]-4-(4-methoxyphenyl)-1,3-oxazol-2-yl]-1-butanol

The title compound was obtained from 4-[2-(1-hydroxybutyl)-4-(4-methoxyphenyl)-1,3-oxazol-5-yl]phenol obtained by Example 204 in a manner similar to Example 164.

¹H-NMR (200 MHz, CDCl₃): δ 0.11(5H, s), 0.91(9H, s), 0.98(3H, t, J=7.3 Hz), 1.37-1.64(2H, m), 1.84-2.07(2H, m), 3.02(1H, br-s), 3.83(3H, s), 3.91-4.08(4H, m), 4.84(1H, t, J=6.5 Hz), 6.89(2H, d, J=8 Hz), 6.89(2H, d, J=8 Hz), 7.48(2H, d, J =8 Hz), 7.55(2H, d, J=8 Hz). MS (ESI): 498 (M+H)⁺.

### Example 206

### 1-[5-[4-(2-Hydroxyethoxy)phenyl]-4-(4-methoxyphenyl)-1,3-oxazol-2-yl]-1-butanol

The title compound was obtained from 1-[5-[4-(2-([tert-butyl(dimethyl)silyl]oxy}ethoxy)phenyl]-4-(4-methoxyphenyl)-1,3-oxazol-2-yl]-1-butanol obtained by Example 205 in a manner similar to Example 166.

¹H-NMR (200 MHz): δ 0.98(3H, t, J=7.3 Hz), 1.36-1.7(2H, m), 1.76-2.12(2H, m), 3.83(3H, s), 3.93-4.04(2H, m), 4.05-4.15(2H, m), 4.85(1H, t, J=6.5 Hz), 6.9(4H, d, J=8 Hz), 7.5(2H, d, J=9.5 Hz), 7.55(2H, d, J=9.5 Hz). MS (ESI): 384 (M+H)⁺.

### Example 207

### 1-[5-[4-(Benzyloxy)phenyl]-4-(4-methoxyphenyl)-1,3-oxazol-2-yl]-3-methyl-1-butanone

The title compound was obtained from 5-[4-(benzyloxy)phenyl]-N-methoxy-4-(4-methoxyphenyl)-N-methyl-1,3-oxazole-2-carboxamide obtained by Example 165 in a manner similar to Example 162.

¹H-NMR (200 MHz, CDCl₃): δ 1.01(3H, s), 1.05(3H, s), 2.26-2.5(1H, m), 3(2H, d, J=7 Hz), 3.85(3H, s), 5.1(2H, s), 6.94(2H, d, J=7.5 Hz), 6.98(2H, d, J=7.5 Hz), 7.36-7.48(5H, m), 7.58(2H, d, J=6 Hz), 7.63(2H, d, J=6 Hz).

### Example 208

### 1-[5-(4-Hydroxyphenyl)-4-(4-methoxyphenyl)-1,3-oxazol-2-yl]-3-methyl-1-butanone

The title compound was obtained from 1-[5-[4-(benzyloxy)phenyl]-4-(4-methoxyphenyl)-1,3-oxazol-2-yl]-3-methyl-1-butanone obtained by Example 207 in a manner similar to Example 163.

### Example 209

### tert-Butyl(2-{4-[4-(4-methoxyphenyl)-2-(3-methylbutanoyl)-1,3-oxazol-5-yl]phenoxy}ethyl)carbamate

The title compound was obtained from 1-[5-(4-hydroxyphenyl)-4-(4-methoxyphenyl)-1,3-oxazol-2-yl]-3-methyl-1-butanone obtained by Example 208 in a manner similar to Example 215 described later.

### Example 210

### 1-[5-[4-(2-Aminoethoxy)phenyl]-4-(4-methoxyphenyl)-1,3-oxazol-2-yl]-3-methyl-1-butanone hydrochloride

The title compound was obtained from tert-butyl(2-{4-[4-(4-methoxyphenyl)-2-(3-methylbutanoyl)-1,3-oxazol-5-yl]phenoxy}ethyl)carbamate obtained by Example 209 in a manner similar to Example 216 described later.

### Example 211

### N-(2-{4-[4-(4-Methoxyphenyl)-2-(3-methylbutanoyl)-1,3-oxazol-5-yl]phenoxy}ethyl)urea

The title compound was obtained from 1-[5-[4-(2-aminoethoxy)phenyl]-4-(4-methoxyphenyl)-1,3-oxazol-2-yl]-3-methyl-1-butanone hydrochloride obtained by Example 210 in a manner similar to Example 217 described later.

¹H-NMR (200 MHz, CDCl₃): δ 1.01(3H, s), 1.05(3H, s), 2.25-2.51(1H, m), 3(2H, d, J=7 Hz), 3.56-3.7(2H, m), 3.85(3H, s), 4-4.12(2H, m), 4.49(2H, br-s), 5.08(1H, t, J=5.7 Hz), 6.88(2H, d, J=9 Hz), 6.94(2H, d, J=9 Hz), 7.57(2H, d, J=6.5 Hz), 7.61(2H, d, J=6.5 Hz). MS (ESI): 438 (M+H)⁺, 481 (M+HCO₂)⁻.

### Example 212

### N-(2-{4-[4-(4-Methoxyphenyl)-2-(3-methylbutanoyl)-1,3-oxazol-5-yl]phenoxy}ethyl)methanesulfonamide

The title compound was obtained from 1-[5-[4-(2-aminoethoxy)phenyl]-4-(4-methoxyphenyl)-1,3-oxazol-2-yl]-3-methyl-1-butanone hydrochloride obtained by Example 210 in a manner similar to Example 218 described later.

¹H-NMR (200 MHz, CDCl₃): δ 1.01(3H, s), 1.05(3H, s), 2.28-2.48(1H, m), 2.99(2H, s), 3.03(3H, s), 3.5-3.64(2H, m), 3.85(3H, s), 4.14(2H, t, J=5 Hz), 4.92(1H, t, J=6 Hz), 6.88(2H, d, J=9 Hz), 6.94(2H, d, J=9 Hz), 7.57(2H, d, J=9 Hz), 7.62(2H, d, J=9 Hz). MS (ESI): 473 (M+H)⁺, 516 (M+HCO₂)⁻.

### Example 213

### 1-[5-(4-Hydroxyphenyl)-4-(6-methoxy-3-pyridinyl)-1,3-oxazol-2-yl]-3-methyl-1-butaone

The title compound (190 mg) was obtained from 1-[5-[4-(benzyloxy)phenyl]-4-(6-methoxy-3-pyridinyl)-1,3-oxazol-2-yl]-3-methyl-1-butanone obtained by Example 202 (340 mg) in a manner similar to Example 180.

¹H-NMR (DMSO-d₆): δ 1.5-1.8(6H, br-s), 3.6-3.7(2H, m), 3.8-3.9(2H, m), 3.88(3H, s), 6.8-6.95(3H, m), 7.40(2H, d, J=8.6 Hz), 7.85(1H, dd, J=8.7,2.4 Hz), 8.37(1H, d, J=2.4 Hz). MS (ESI): 353.0 (M+H)⁺.

### Example 214

### 1-[5-[4-(2-Hydroxyethoxy)phenyl]-4-(6-methoxy-3-pyridinyl)-1,3-oxazol-2-yl]-3-methyl-1-butanone

The title compound (55 mg) was obtained from 1-[5-(4-hydroxyphenyl)-4-(6-methoxy-3-pyridinyl)-1,3-oxazol-2-yl]-3-methyl-1-butaone obtained by Example 213 (120 mg) in a manner similar to Example 181.

¹H-NMR (DMSO-d₆): δ 0.96(6H, d, J=6.8 Hz), 2.1-2.3(1H, m), 2.97(1H, d, J=6.9 Hz), 3.6-3.8(2H, m), 3.90(3H, s), 4.0-4.1(2H, m), 4.92(1H, t, J=5.5 Hz), 6.9-7.2(3H, m), 7.55(2H, d, J=8.7 Hz), 7.87(1H, dd, J=8.5,2.4 Hz), 8.38(1H, d, J=2.4 Hz). MS (ESI): 419.2 (M+Na)⁺.

### Example 215

### tert-Butyl(2-{4-[4-(4-methoxyphenyl)-2-(1-piperidinylcarbonyl)-1,3-oxazol-5-yl]phenoxy}ethyl)carbamate

NaH (60% in mineral oil, 64.1 mg) was added to a solution of 4-[4-(4-methoxyphenyl)-2-(1-piperidinylcarbonyl)-1,3-oxazol-5-yl]phenol obtained by Example 154 (303 mg) in dimethylformamide (2.0 mL) at 0° C. After stirring for 15 min, a solution of tert-butyl(2-bromoethyl)carbamate (449 mg) in dimethylformamide (2.0 mL) was added. The mixture was stirred for 10 hrs at 45° C.

The mixture was poured into saturated ammonium chloride aqueous solution at 0° C. and the products were extracted with ethyl acetate. The combined extracts were washed with brine, drid over magnesium sulfate, and evaporated. The residue was purified by silica gel column chromatography to give the title compound (485 mg).

¹H-NMR (200 MHz, CDCl₃): δ 1.46(9H, s), 1.71(6H, br-s), 3.43-3.63(2H, m), 3.68-3.81(2H, m), 3.85(3H, s), 4-4.15(4H, m), 5(1H, br-s), 6.88(2H, d, J=6.5 Hz), 6.92(2H, d, J=6.5 Hz), 7.56(2H, d, J=3 Hz), 7.61(2H, d, J=3 Hz). MS (ESI): 521 (M+H)⁺.

### Example 216

### (2-{4-[4-(4-Methoxyphenyl)-2-(1-piperidinylcarbonyl)-1,3-oxazol-5-yl]phenoxy}ethyl)amine hydrochloride

4N HCl-dioxane (2.50 mL) was added to a solution of tert-butyl (2-{4-[4-(4-methoxyphenyl)-2-(1-piperidinylcarbonyl)-1,3-oxazol-5-yl]phenoxy)ethyl)carbamate obtained by Example 215 (485 mg) in dichloromethane (2.5 mL) at 0° C.

After stirring for 2 hrs at room temperature, the mixture was evaporated in vacuo to give the title compound (616 mg). MS (LC): 422 (M+H)⁺ (free).

### Example 217

### N-(2-{4-[4-(4-Methoxyphenyl)-2-(1-piperidinylcarbonyl)-1,3-oxazol-5-yl]phenoxy}ethyl)urea

Triethylamine (141 mg) and trimethylsilyl isocyanate (80.4 mg) were added to a solution of (2-{4-[4-(4-methoxyphenyl)-2-(1-piperidinylcarbonyl)-1,3-oxazol-5-yl]phenoxy}ethyl)amine hydrochloride obtained by Example 216 (213 mg) in dichlorimethane (2.2 mL) at 0° C.

After stirring for 10 hrs at room temperature, the product was extracted with ethyl acetate. The combined extracts were washed with 1N hydrochloric acid, saturated sodium hydrogencarbonate aqueous solution and brine, dried over magnesium sulfate, and evaporated under reduced presure. The residue was triturated in isopropylether to give the title compound (92.0 mg).

¹H-NMR (200 MHz, CDCl₃): δ 1.71(6H, br-s), 3.56-3.66(2H, m), 3.71-3.78(2H, m), 3.84(3H, s), 4.05(2H, t, J=2.5 Hz), 4.08-4.17(2H, m), 4.55(2H, br-s), 5.11-5.23(1H, m), 6.86(2H, d, J=4.4 Hz), 6.91(2H, d, J=4.4 Hz), 7.5-7.63(4H, m). MS (ESI): 465(M+H)⁺.

### Example 218

### N-(2-{4-[4-(4-Methoxyphenyl)-2-(1-piperidinylcarbonyl)-1,3-oxazol-5-yl]phenoxy}ethyl)methanesulfonamide

Triethylamine (141 mg) and methanesulfonyl chloride (79.9 mg) were added to a solution of (2-{4-[4-(4-methoxyphenyl)-2-(1-piperidinylcarbonyl)-1,3-oxazol-5-yl]phenoxy}ethyl)amine hydrochloride obtained by Example 216 (213 mg) in dichloromethane (2.2 mL) at 0° C.

After stirring for 10 hrs at room temperature, the product was extracted with ethyl acetate. The combined extracts were washed with 1N hydrochloric acid, saturated sodium hydrogencarbonate aqueous solution and brine, dried over magnesium sulfate, and evaporated under reduced presure. The residue was purified by preparative thin layer chromatography to give the title compound (114 mg).

¹H-NMR (200 MHz): δ 1.71(6H, br-s), 3.02(3H, s), 3.43-3.8(4H, m), 3.84(3H, s), 4-4.14(4H, m), 5.15(1H, t, J=5.9 Hz), 6.86(2H, d, J=8.9 Hz), 6.92(2H, d, J=8.9 Hz), 7.53-7.6(4H, m). MS (ESI): 500 (M+H)⁺.

### Example 219

### N-(2-{4-[4-(4-Methoxyphenyl)-2-(2,2,2-trifluoroethoxy)-1,3-oxazol-5-yl]phenoxy}ethyl)urea

To a solution of 2,2,2-trifluoroethanol (102 mg) and sodium hydride (60% in mineral oil; 41 mg) in dioxane, N-(2-{4-[4-(4-methoxyphenyl)-2-(methylsulfonyl)-1,3-oxazol-5-yl]phenoxy}ethyl)urea obtained by Example 197 (88 mg) was added. And the mixture was stirred at room temperature overnight under a nitrogen atmosphere.

The reaction mixture was quenched with water, extracted with dichloromethane three times. The combined extracts were dried over magnesium sulfate and concentrated. The residue was purified by preparative thin-layer chromatography (dichloromethane/methanol=9/1) to give the title compound (70 mg).

¹H NMR (CDCl₃): δ 3.61(2H, m), 3.83(3H, s), 4.07(2H, t, J=4.9 Hz), 4.39(1H, br-s), 4.84(2H, q, J=8.0 Hz), 4.94(1H, br-s), 6.80-7.70(8H, m). MS (ESI): 474.1 (M+Na)⁺.

### Example 220

### N-[4,5-Bis(4-methoxyphenyl)-1,3-oxazol-2-yl]-2-pyridinamine

A mixture of 4,5-bis(4-methoxyphenyl)-2-(methylsulfonyl)-1,3-oxazole obtained by Example 158 (132 mg), 2-aminopyridine (104 mg) and sodium hydride (60% in mineral oil; 44 mg) in dioxane was stirred at 85° C. under a nitrogen atmosphere for 3 hrs.

The reaction mixture was cooled, quenched with water, and extracted with ethyl acetate twice. The combined extracts were washed with water three times, dried over magnesium sulfate, and concentrated. The residue was chromatographed on silica gel (n-hexane/ethyl acetate=1/1) to give the title compound (34 mg).

¹H-NMR (CDCl₃): δ 3.85(3H, s), 3.86(3H, s), 6.70-8.40(13H, m). MS (ESI): 374.2 (M+H)⁺.

### Example 221

### N-(2-{4-[2-Methoxy-4-(4-methoxyphenyl)-1,3-oxazol-5-yl]phenoxy}ethyl)methanesulfonamide

To a solution of (2-{4-[2-methoxy-4-(4-methoxyphenyl)-1,3-oxazol-5-yl]phenoxy}ethyl)amine obtained by Example 173 (73 mg) and triethylamine (90 μL) in dichloromethane, methanesulfonylchloride (25 μL) was added dropwsie. And the mixture was stirred at room temperature for 2 hrs. The reaction mixture was quenched with water and extracted with ethyl acetate twice. The combined extracts were dried over magnesium sulfate and concentrated. The residue was purified by preparative thin-layer chromatography (dichloromethane/methanol=9/1) to give the title compound (47 mg).

¹H-NMR (CDCl₃): δ 2.90-5.00(14H, m), 6.60-7.70(8H, m). MS (ESI): 441.20 (M+Na)⁺.

### Example 222

### N-(2-{4-[2-Ethoxy-4-(4-methoxyphenyl)-1,3-oxazol-5-yl]phenoxy}ethyl)urea

The title compound was obtained from N-(2-{4-[4-(4-methoxyphenyl)-2-(methylsulfonyl)-1,3-oxazol-5-yl]phenoxy}ethyl)urea obtained by Example 197 in a manner similar to

### Example 219.

¹H-NMR (CDCl₃): δ 1.48(3H, t, J=7.1 Hz), 3.50-4.20(7H, m), 4.40(2H, br-s), 4.53(2H, q, J=7.1 Hz), 5.01(1H, br-s), 6.70-7.70(8H, m). MS (ESI): 398.2 (M+H)⁺.

### Example 223

### N-(2-{4-[2-Isopropoxy-4-(4-methoxyphenyl)-1,3-oxazol-5-yl]phenoxy}ethyl)urea

The title compound was obtained from N-(2-{4-[4-(4-methoxyphenyl)-2-(methylsulfonyl)-1,3-oxazol-5-yl]phenoxy}ethyl)urea obtained by Example 197 in a manner similar to Example 219.

¹H-NMR (CDCl₃): δ 1.47(2H, d, J=6.1 Hz), 3.60(2H, m), 3.83(3H, s), 4.05(2H, t, J=4.9 Hz), 4.40(2H, br-s), 4.95(1H, br-s), 5.17(1H, heptet, J=6.1 Hz), 6.70-7.70(8H, m). MS (ESI): 434.2 (M+Na)⁺.

### Example 224

### N-(2-{4-[2-(Isopropylthio)-4-(4-methoxyphenyl)-1,3-oxazol-5-yl]phenoxy}ethyl)urea

The title compound was obtained from N-(2-{4-[4-(4-methoxyphenyl)-2-(methylsulfonyl)-1,3-oxazol-5-yl]phenoxy}ethyl)urea obtained by Example 197 in a manner similar to Example 219.

¹H-NMR (CDCl₃): δ 1.49(6H, d, J=6.9 Hz), 3.60-4.20(8H, m), 4.42(2H, br-s), 4.96(1H, br-s), 6.70-7.70(8H, m). MS (ESI): 428.2 (M+H)⁺.

### Example 225

### N-(2-{4-[2-(Isopropylsulfonyl)-4-(4-methoxyphenyl)-1,3-oxazol-5-yl]phenoxy}ethyl)urea

The title compound was obtained from N-(2-{4-[2-(isopropylthio)-4-(4-methoxyphenyl)-1,3-oxazol-5-yl]phenoxy}ethyl)urea obtained by Example 224 in a manner similar to Example 197.

¹H-NMR (CDCl₃): δ 1.50(6H, d, J=6.9 Hz), 3.40-3.70(3H, m), 3.85(3H, s), 4.09(2H, t, J=5.0 Hz), 4.45(2H, br-s), 5.00(1H, br-s), 6.80-7.80(8H, m). MS (ESI): 482.0 (M+Na)⁺.

### Example 226

### N-(2-{4-[2-(2-Ethoxyethoxy)-4-(4-methoxyphenyl)-1,3-oxazol-5-yl]phenoxy}ethyl)urea

The title compound was obtained from N-(2-{4-[4-(4-methoxyphenyl)-2-(methylsulfonyl)-1,3-oxazol-5-yl]phenoxy}ethyl)urea obtained by Example 197 in a manner similar to Example 219.

¹H-NMR (CDCl₃): δ 3.40-4.20(11H, m), 4.44(2H, br-s), 4.61(2H, m), 4.99(1H, br-s), 6.70-7.70(8H, m). MS (ESI): 442.3 (M+H)⁺.

### Example 227

### 2-(Isopropylthio)-4,5-bis(4-methoxyphenyl)-1,3-oxazole

To a solution of 2-propanethiol (127 mg) and sodium hydride (60% in mineral oil; 67 mg) in dioxane, 4,5-bis(4-methoxyphenyl)-2-(methylsulfonyl)-1,3-oxazole obtained by Example 158 (120 mg) was added. And the mixture was stirred at room temperature overnight under a nitrogen atmosphere.

The reaction mixture was quenched with water, and extracted with dichloromethane twice. The combined extracts were dried over magnesium sulfate and concentrated to give the title compound (134 mg).

¹H-NMR (CDCl₃): δ 1.49(6H, d, J=6.9 Hz), 3.70-4.00(7H, m), 6.70-7.80(8H, m). MS (ESI): 356.2 (M+H)⁺.

### Example 228

### N-(2-{4-[2-(Dimethylamino)-4-(4-methoxyphenyl)-1,3-oxazol-5-yl]phenoxy}ethyl)urea

A mixture of N-(2-{4-[4-(4-methoxyphenyl)-2-(methylsulfonyl)-1,3-oxazol-5-yl]phenoxy}ethyl)urea obtained by Example 197 (100 mg) in 50% dimethylamine aqueous solution (5 mL) and dioxane (5 mL) was stirred at 60° C. for 3 hrs.

The mixture was diluted with ethyl acetate, washed with water three times, dried over magnesium sulfate, and concentrated. The residue was triturated with ethanol, the resulting powder was collected, washed with ethanol, and dried in vacuo to give the title compound (46 mg).

¹H-NMR (CDCl₃): δ 3.12(6H, s), 3.60(2H, m), 3.83(3H, s), 4.04(2H, t, J=5.0 Hz), 4.40(2H, br-s), 4.96(1H, br-s), 6.70-7.70(8H, m). MS (ESI): 397.1 (M+H)⁺.

### Example 229

### N-(2-{4-[2-(Cyclopentyloxy)-4-(4-methoxyphenyl)-1,3-oxazol-5-yl]phenoxy}ethyl)urea

The title compound was obtained from N-(2-{4-[4-(4-methoxyphenyl)-2-(methylsulfonyl)-1,3-oxazol-5-yl]phenoxy}ethyl)urea obtained by Example 197 in a manner similar to Example 219.

¹H-NMR (CDCl₃): δ 0.80-4.20(15H, m), 4.43(2H, br-s), 4.98(1H, br-s), 5.38(1H, m), 6.70-7.70(8H, m). MS (ESI): 460.2 (M+Na)⁺.

### Example 230

### N-(2-{4-[2-(2-Fluoroethoxy)-4-(4-methoxyphenyl)-1,3-oxazol-5-yl]phenoxy}ethyl)urea

The title compound was obtained from N-(2-{4-[4-(4-methoxyphenyl)-2-(methylsulfonyl)-1,3-oxazol-5-yl]phenoxy}ethyl)urea obtained by Example 197 in a manner similar to Example 219.

¹H-NMR (CDCl₃): δ 3.50(2H, m), 3.83(3H, s), 4.06(2H, t, J=4.9 Hz), 4.45(2H, br-s), 4.60-5.00(4H, m), 5.00(1H, br-s), 6.70-7.70(8H, m). MS (ESI): 416.4 (M+H)⁺.

### Example 231

### N-(2-{4-[2-(2,2-Difluoroethoxy)-4-(4-methoxyphenyl)-1,3-oxazol-5-yl]phenoxy}ethyl)urea

The title compound was obtained from N-(2-{4-[4-(4-methoxyphenyl)-2-(methylsulfonyl)-1,3-oxazol-5-yl]phenoxy}ethyl)urea obtained by Example 197 in a manner similar to Example 219.

¹H-NMR (CDCl₃): δ 3.40-6.60(13H, m), 6.70-7.70(8H, m). MS (ESI): 456.2 (M+Na)⁺.

### Example 232-1

### (1,3-Dioxo-1,3-dihydro-2H-isoindol-2-yl)acetic acid

2-Benzofuran-1,3-dione was added to a solution of aminoacetic acid (10.0 g) in dioxane (40 mL) at room temperature. The mixture was refluxed for 2 hrs.

The mixture was evaporated under reduced presure. The residue was triturated in water to give the title compound (28.5 g).

¹H-NMR (200 MHz, DMSO-d₆): δ 3.42(1H, br-s), 4.33(2H, s), 7.81-8.02(4H, m).

### Example 232-2

### 1-[4-(Benzyloxy)phenyl]-2-(4-methoxyphenyl)-2-oxoethyl(1,3-dioxo-1,3-dihydro-2H-isoindol-2-yl)acetate

(1,3-Dioxo-1,3-dihydro-2H-isoindol-2-yl)acetic acid obtained by Example 232-1 (670 mg) and cesium carbonate (1.06 g) were added to a solution of 2-[4-(benzyloxy)phenyl]-2-bromo-1-(4-methoxyphenyl)ethanone (1.28g) in acetone(13.0 mL) at 0° C.

After stirring for 10 hrs at room temperature, the mixture was evaporated under reduced presure. The residue was triturated in isopropylether to give the title compound (566 mg).

¹H-NMR (200 MHz, CDCl₃): δ 3.8(3H, s), 4.51(1H, d, J=17.4 Hz), 4.72(1H, d, J=17.5 Hz), 5.03(2H, s), 6.75-6.98(5H, m), 7.33-7.42(7H, m), 7.66-7.79(2H, m), 7.81-7.95(4H, m). MS (ESI): 558 (M+Na)⁺.

### Example 232-3

### 2-{[5-[4-(Benzyloxy)phenyl]-4-(4-methoxyphenyl)-1,3-oxazol-2-yl]methyl}-1H-isoindole-1,3(2H)-dione

Ammonium acetate (432 mg) was added to a solution of 1-[4-(benzyloxy)phenyl]-2-(4-methoxyphenyl)-2-oxoethyl (1,3-dioxo-1,3-dihydro-2H-isoindol-2-yl)acetate obtained by Example 232-2 (300 mg) in acetic acid (5.60 mL) at room temperature.

The mixture was refluxed for 1.5 hrs, and evaporated under reduced presure. The residue was washed with saturated sodium hydrogencarbonate aqueous solution and water to give the title compound (181 mg).

¹H-NMR (200 MHz, DMSO-d₆): δ 3.76(3H, s), 5(2H, s), 5.13(2H, s), 6.94(2H, d, J=8.9 Hz), 7.08(2H, d, J=8.9 Hz), 7.36-7.57(9H, m), 7.78-8.03(4H, m).

### Example 233

### 1-[5-[4-(Benzyloxy)phenyl]-4-(4-methoxyphenyl)-1,3-oxazol-2-yl]methanamine

Hydrazine monohydrate (4.47 g) was added to 2-{[5-[4-(benzyloxy)phenyl]-4-(4-methoxyphenyl)-1,3-oxazol-2-yl]methyl}-1H-isoindole-1,3(2H)-dione obtained by Example 232-3 (5.77 g) in tetrahydrofuran (58.0 mL) at room temperature.

After a stirring for 1 hr at 80° C., the mixture was washed with 0.1N hydrochloric acid and brine, dried over magnesium sulfate, and evaporated under reduced pressure to give the title compound (5.29 g).

¹H-NMR (200 MHz, CDCl₃): δ 3.83(3H, s), 4.01(2H, s), 5.08(2H, s), 6.9(2H, d, J=9 Hz), 6.96(2H, d, J=9 Hz), 7.3-7.59(9H, m). MS (ESI): 387 (M+H)⁺.

### Example 234

### 5-[4-(Benzyloxy)phenyl]-N,N-diethyl-4-(6-methoxy-3-pyridinyl)-1,3-oxazole-2-carboxamide

The title compound (900 mg) was obtained from ethyl 5-[4-(benzyloxy)phenyl]-4-(6-methoxy-3-pyridinyl)-1,3-oxazole-2-carboxylate (1.0 g) in a manner similar to Example 179.

¹H-NMR (DMSO-d₆): δ 1.17(3H, t, J=7 Hz), 1.27(3H, t, J=6.9 Hz), 3.48(2H, q, J=7.1 Hz), 3.76(2H, q, J=6.9 Hz), 3.89(3H, s), 5.16(2H, s), 6.92(1H, d, J=9.1 Hz), 7.15(2H, d, J=8.9 Hz), 7.3-7.6(7H, m), 7.86(1H, dd, J=2.4, 8.7 Hz), 8.4(1H, d, J=2.4 Hz). MS (ESI): 458.2 (M+H)⁺.

### Example 235

### N,N-Diethyl-5-(4-hydroxyphenyl)-4-(6-methoxy-3-pyridinyl)-1,3-oxazole-2-carboxamide

20% Palladium hydroxide on carbon (50% wet, 272 mg) was added to a solution of 5-[4-(benzyloxy)phenyl]-N,N-diethyl-4-(6-methoxy-3-pyridinyl)-1,3-oxazole-2-carboxamide obtained by Example 234 (890 mg) in ethanol (15 mL) and cyclohexene (5 mL). The mixture was stirred at reflux condition for 10 hour and cooled to room temperature.

After filtration through celite, the reaction mixture was evaporated. The residue was purified by column chromatography on silica-gel eluting with dichloromethane and acetone to give the title compound (621 mg).

¹H-NMR (DMSO-d₆): δ 1.16(3H, t, J=7 Hz), 1.27(3H, t, J=6.9 Hz), 3.34(2H, br-s), 3.47(2H, q, J=7 Hz), 3.77(2H, q, J=7 Hz), 3.88(3H, s), 6.8-7(3H, m), 7.41(2H, d, J=9.5 Hz), 7.85(1H, dd, J=2.4,8.7 Hz). MS (ESI): 390.2 (M+Na)⁺.

### Example 236

### N,N-Diethyl-5-[4-(2-hydroxyethoxy)phenyl]-4-(6-methoxy-3-pyridinyl)-1,3-oxazole-2-carboxamide

The title compound (135 mg) was obtained from N,N-diethyl-5-(4-hydroxyphenyl)-4-(6-methoxy-3-pyridinyl)-1,3-oxazole-2-carboxamide obtained by Example 235 (200 mg) in a manner similar to Example 181.

¹H-NMR (DMSO-d₆): δ 1.17(3H, t, J=7 Hz), 1.28(3H, t, J=6.9 Hz), 3.48(2H, q, J=7 Hz), 3.7-3.8(4H, m), 3.89(3H, s), 4.05(2H, t, J=4.8 Hz), 4.92(1H, t, J=5.5 Hz), 6.92(1H, d, J=8.7 Hz), 7.07(2H, d, J=8.8 Hz), 7.53(2H, d, J=8.8 Hz), 7.86(1H, dd, J=2.4,8.6 Hz), 8.4(1H, d, J=2.2 Hz). MS (ESI): 434.2 (M+Na)⁺.

### Example 237

### N-(2-{4-[2-(Ethylthio)-4-(4-methoxyphenyl)-1,3-oxazol-5-yl]phenoxy}ethyl)urea

The title compound was obtained from N-(2-{4-[4-(4-methoxyphenyl)-2-(methylsulfonyl)-1,3-oxazol-5-yl]phenoxy}ethyl)urea obtained by Example 197 in a manner similar to Example 219.

¹H-NMR (CDCl₃): δ 1.50(3H, t, J=7.4 Hz), 3.24(2H, q, J=7.4 Hz), 3.50-4.20(7H, m), 4.40(2H, br-s), 4.97(1H, br-s), 6.70-7.70(8H, m). MS (ESI): 436.3 (M+Na)⁺.

### Example 238

### N-(2-{4-[2-(Ethylsulfonyl)-4-(4-methoxyphenyl)-1,3-oxazol-5-yl]phenoxy}ethyl)urea

The title compound was obtained from N-(2-{4-[2-(ethylthio)-4-(4-methoxyphenyl)-1,3-oxazol-5-yl]phenoxy}ethyl)urea obtained by Example 237 in a manner similar to Example 197.

¹H-NMR (CDCl₃): δ 1.50(3H, t, J=7.4 Hz), 3.50(2H, q, J=7.4 Hz), 3.60-4.20(7H, m), 4.46(2H, br-s), 4.98(1H, br-s), 6.80-7.80(8H, m). MS (ESI): 468.2 (M+Na)⁺.

### Example 239

### N-[4,5-Bis(4-methoxyphenyl)-1,3-oxazol-2-yl]acetamide

A mixture of 4,5-bis(4-methoxyphenyl)-2-(methylsulfonyl)-1,3-oxazole obtained by Example 158 (150 mg),acetamide (123 mg) and sodium hydride (60% in mineral oil; 84 mg) in dioxane was stirred at 70° C. for 3 hrs.

The mixture was diluted with ethyl acetate, washed with water three times, dried over magnesium sulfate, and concentrated. The residue was purified by preparative thin-layer chromatography (hexane/ethyl acetate=1/1) to give the title compound (91 mg).

¹H-NMR (CDCl₃): δ 1.58(3H, s), 3.82(3H, s), 3.83(3H, s), 6.70-7.70(8H, m). MS (ESI): 339.2 (M+H)⁺.

### Example 240

### 2-{[4,5-Bis(4-methoxyphenyl)-1,3-oxazol-2-yl]thio}ethanol

The title compound was obtained from 4,5-bis(4-methoxyphenyl)-2-(methylsulfonyl)-1,3-oxazole obtained by Example 158 in a manner similar to Example 227.

¹H-NMR (CDCl₃): δ 3.30-4.20(10H, m), 6.80-7.70(8H, m). MS (ESI): 380.3 (M+Na)⁺.

### Example 241

### N-(2-(4-[2-{[2-(Dimethylamino)ethyl]thio}-4-(4-methoxyphenyl)-1,3-oxazol-5-yl]phenoxy}ethyl)urea

The title compound was obtained from N-(2-{4-[4-(4-methoxyphenyl)-2-(methylsulfonyl)-1,3-oxazol-5-yl]phenoxy}ethyl)urea obtained by Example 197 in a manner similar to Example 219.

¹H-NMR (CDCl₃): δ 2.32(6H, s), 2.74(2H, t, J=7.0 Hz), 3.78(2H, t, J=7.0 Hz), 3.50-4.20(7H, m), 4.46(2H, br-s), 5.03(1H, br-s), 6.70-7.80 (8H, m). MS (ESI): 457.3 (M+H)⁺.

### Example 242

### tert-Butyl(2-{4-[2-[(diethylamino)carbonyl]-4-(6-methoxy-3-pyridinyl)-1,3-oxazol-5-yl]phenoxy}ethyl)carbamate

The title compound (601 mg) was obtained from N,N-diethyl-5-(4-hydroxyphenyl)-4-(6-methoxy-3-pyridinyl)-1,3-oxazole-2-carboxamide obtained by Example 235 (444 mg) in a manner similar to Example 182.

¹H-NMR (DMSO-d₆): δ 1.17(3H, t, J=7 Hz), 1.28(3H, t, J=6.9 Hz), 1.38(9H, s), 3.2-3.3(2H, m), 3.48(2H, q, J=7 Hz), 3.77(2H, q, J=6.9 Hz), 3.89(3H, s), 4.02(2H, t, J=5.6 Hz), 6.92(1H, d, J=8.5 Hz), 7.06(2H, d, J=8.8 Hz), 7.52(2H, d, J=8.8 Hz), 7.86(1H, dd, J=2.4,8.6 Hz), 8.4(1H, d, J=1.9 Hz). MS (ESI): 511.3 (M+H)⁺.

### Example 243

### 5-[4-(2-Aminoethoxy)phenyl]-N,N-diethyl-4-(6-methoxy-3-pyridinyl)-1,3-oxazole-2-carboxamide hydrochloride

The title compound (430 mg) was obtained from tert-butyl N,N-diethyl-5-(4-hydroxyphenyl)-4-(6-methoxy-3-pyridinyl)-1,3-oxazole-2-carboxamide obtained by Example 242 (580 mg) in a manner similar to Example 198.

¹H-NMR (DMSO-d₆): δ 1.16(3H, t, J=7.0 Hz), 1.27(3H, t, J=7.0 Hz), 3.1-3.3(2H, m), 3.48(2H, q, J=7.0 Hz), 3.77(2H, q, J=7.0 Hz), 3.89(3H, s), 4.27(2H, t, J=4.9 Hz), 6.93(2H, d, J=8.5 Hz), 7.12(2H, d, J=8.8 Hz), 7.57(2H, d, J=8.8 Hz), 7.86(1H, dd, J=8.5,1.9 Hz), 8.36(2H, br-s), 8.39(1H, dd, J=1.9 Hz).

### Example 244

### N,N-Diethyl-4-(6-methoxy-3-pyridinyl)-5-(4-{2-[(methylsulfonyl)amino]ethoxy}phenyl)-1,3-oxazole-2-carboxamide

The title compound (144 mg) was obtained from 5-[4-(2-aminoethoxy)phenyl]-N,N-diethyl-4-(6-methoxy-3-pyridinyl)-1,3-oxazole-2-carboxamide hydrochloride obtained by Example 243 (200 mg) in a manner similar to Example 199.

¹H-NMR (DMSO-d₆): δ 1.17(3H, t, J=6.9 Hz), 1.28(3H, t, J=6.9 Hz), 2.97(3H, s), 3.3-3.5(4H, m), 3.77(2H, q, J=6.9 Hz), 3.89(3H, s), 4.10(2H, t, J=5.4 Hz), 6.92(1H, d, J=8.6 Hz), 7.09(2H, d, J=8.7 Hz), 7.33(1H, t, J=5.8 Hz), 7.54(2H, d, J=8.7 Hz), 7.86(1H, dd, J=8.6,2.1 Hz), 8.39(1H, d, J=2.1 Hz). MS (ESI): 489.2 (M+H)⁺.

### Example 245

### 4-Nitrophenyl(2-(4-[4-(6-methoxy-3-pyridinyl)-2-(trifluoromethyl)-1,3-oxazol-5-yl]phenoxy}ethyl)carbamate

Under a nitrogen atmosphere, 4-nitrophenyl chloroformate (202 mg) was added to a suspension of (2-{4-[4-(6-methoxy-3-pyridinyl)-2-trifluoromethyl-1,3-oxazol-5-yl]phenoxy}ethyl)amine hydrochloride (416 mg) and triethylamine (253 mg) in dichloromethane (10 ml) at 0° C.

The mixture was stirred at the same temperature for 2 hrs, and poured into a mixture of cold water and ethyl acetate. The mixture was adjusted pH 1 with 1N aqueous hydrochloric acid and the aquesous layer was separeated. The organic layer was washed with saturated aqueous sodium hydrogencarbonate and brine, and dried over magnesium sulfate. Evaporation of the solvent afforded the title compound (511 mg).

¹H-NMR (DMSO-d₆): δ 3.3-3.6(2H, m), 3.89(3H, s), 4.1-4.3(2H, m), 6.93(1H, d, J=9.0 Hz), 7.12(2H, d, J=8.8 Hz), 7.57(2H, d, J=8.8 Hz), 7.86(1H, dd, J=9.0,1.8 Hz), 8.1-8.4(5H, m).

### Example 246

### N-(2-Hydroxyethyl)-N′-(2-{4-[4-(6-methoxy-3-pyridinyl)-2-(trifluoromethyl)-1,3-oxazol-5-yl]phenoxy}ethyl)urea

Under a nitrogen atmopshere, hydroxyethylamine (44.9 mg) was added to a solution of 4-nitrophenyl(2-{4-[4-(6-methoxy-3-pyridinyl)-2-(trifluoromethyl)-1,3-oxazol-5-yl]phenoxy}ethyl)carbamate obtained by Example 245 (200 mg) in dimethylformamide (5 mL) at 0° C. Ice bath was removed after 5 min and the mixture was stirred at room temperature for 2 hrs.

The mixture was poured into a mixture of cold water and ethyl acetate. The aquesous layer was separeated, the organic layer was washed with water and brine, and dried over magnesium sulfate. After evaporation of the solvent, the residue was purified by column chromatography on silica-gel eluting with dichloromethane and acetone to afford the title compound (90.1 mg).

¹H-NMR (DMSO-d₆): δ 3.67(2H, q, J=5.5 Hz), 3.3-3.5(4H, m), 3.89(3H, s), 4.02(2H, t, J=5.5 Hz), 6.05(1H, t, J=5.6 Hz), 6.24(1H, t, J=5.6 Hz), 6.93(1H, d, J=8.6 Hz), 7.10(2H, d, J=8.8 Hz), 7.55(2H, d, J=8.8 Hz), 7.86(1H, dd, J=8.6,2.3 Hz), 8.37(1H, d, J=2.3 Hz). MS (ESI): 488.9 (M+Na)⁺.

### Example 247

### 5-(4-{2-[(Aminocarbonyl)amino]ethoxy}phenyl)-N,N-diethyl-4-(6-methoxy-3-pyridinyl)-1,3-oxazole-2-carboxamide

The title compound (120 mg) was obtained from 5-[4-(2-aminoethoxy)phenyl]-N,N-diethyl-4-(6-methoxy-3-pyridinyl)-1,3-oxazole-2-carboxamide hydrochloride obtained by Example 243 (203 mg) in a manner similar to Example 200.

¹H-NMR (DMSO-d₆): δ 1.20(3H, t, J=7.9 Hz), 1.31(3H, t, J=7.9 Hz), 3.3-3.6(4H, m), 3.77(2H, q, J=7.9 Hz), 3.89(3H, s), 4.02(2H, t, J=5.4 Hz), 5.58(2H, s), 6.22(1H, t, J=5.6 Hz), 6.91(1H, d, J=8.6 Hz), 7.08(2H, d, J=8.7 Hz),7.53(2H, d, J=8.7 Hz),7.86(1H, dd, J=8.6,2.2 Hz), 8.39(1H, d, J=2.2 Hz). MS (ESI): 476.2 (M+Na)⁺.

### Example 248

### 2-({[(2-{4-[4-(6-Methoxy-3-pyridinyl)-2-(trifluoromethyl)-1,3-oxazol-5-yl]phenoxy}ethyl)amino]carbonyl}amino)acetamide

The title compound (108 mg) was obtained from 4-nitrophenyl(2-{4-[4-(6-methoxy-3-pyridinyl)-2-(trifluoromethyl)-1,3-oxazol-5-yl]phenoxy}ethyl)carbamate obtained by Example 245 (200 mg) and glycinamide hydrochloride (81.2 mg) in a manner similar to Example 246.

¹H-NMR (DMSO-d₆): δ 3.3-3.5(2H, m), 3.61(2H, d, J=5.4 Hz), 3.89(3H, s), 4.02(2H, t, J=5.4 Hz), 6.18(1H, t, J=5.4 Hz), 6.44(1H, t, J=5.4 Hz), 6.93(1H, d, J=8.7 Hz), 6.98(1H, br-s), 7.10(2H, d, J=8.8 Hz), 7.29(1H, br-s), 7.55(2H, d, J=8.8 Hz), 7.86(1H, dd, J=8.7,2.2 Hz), 8.37(1H, d, J=2.2 Hz). MS (ESI): 502.1 (M+Na)⁺.

### Example 249

### N-(2-Methoxyethyl)-N′-(2-{4-[4-(6-methoxy-3-pyridinyl)-2-(trifluoromethyl)-1,3-oxazol-5-yl]phenoxy}ethyl)urea

The title compound (112 mg) was obtained from 4-nitrophenyl(2-{4-[4-(6-methoxy-3-pyridinyl)-2-(trifluoromethyl)-1,3-oxazol-5-yl]phenoxy}ethyl)carbamate obtained by Example 245 (150 mg) and 2-methoxyethylamine (62.1 mg) in a manner similar to Example 246.

¹H-NMR (DMSO-d₆): δ 3.1-3.6(6H, m), 3.24(3H, s), 3.89(3H, s), 4-4.1(2H, m), 6.06(1H, br-s), 6.2(1H, br-s), 6.93(1H, d, J=8.6 Hz), 7.1(2H, d, J=8.4 Hz), 7.55(2H, d, J=8.4 Hz), 7.86(1H, d, J=8.6,2.3 Hz), 8.38(1H, d, J=2.3 Hz). MS (ESI): 503.2 (M+Na)⁺.

### Example 250

### N-{[5-[4-(Benzyloxy)phenyl]-4-(4-methoxyphenyl)-1,3-oxazol-2-yl]methyl}propanamide

Propanoyl chloride (503 mg) and pyridine (1.47 mL) was added to a solution of 1-[5-[4-(benzyloxy)phenyl]-4-(4-methoxyphenyl)-1,3-oxazol-2-yl]methanamine obtained by Example 233 (1.40 g) in dimethylformamide (14.0 mL) at 0° C.

After stirring for 1.5 hrs at room temperature, the product was extracted with diethylether, washed with brine, dried over magnesium sulfate, and evaporated. The residue was purified by silica gel column chromatography to give the title compound (1.18 g).

¹H-NMR (200 MHz, CDCl₃): δ 1.21(3H, t, J=7.6 Hz), 2.32(2H, q, J=7.5 Hz), 3.83(3H, s), 4.63(2H, d, J=5.5 Hz), 5.08(2H, s), 6.25(1H, br-s), 6.9(2H, d, J=9 Hz), 6.96(2H, d, J=9 Hz), 7.32-7.56(9H, m). MS (ESI): 443 (M+H)⁺, 465 (M+Na)⁺.

### Example 251

### N-{[5-(4-Hydroxyphenyl)-4-(4-methoxyphenyl)-1,3-oxazol-2-yl]methyl}propanamide

The title compound was obtained from N-{[5-[4-(benzyloxy)phenyl]-4-(4-methoxyphenyl)-1,3-oxazol-2-yl]methyl)propanamide obtained by Example 250 in a manner similar to Example 163.

MS (ESI): 353 (M+H)⁺.

### Example 252

### N′-{[5-[4-(benzyloxy)phenyl]-4-(4-methoxyphenyl)-1,3-oxazol-2-yl]methyl}-N,N-dimethylurea

The title compound was obtained from 1-[5-[4-(benzyloxy)phenyl]-4-(4-methoxyphenyl)-1,3-oxazol-2-yl]methanamine obtained by Example 233 and dimethylcarbamic chloride in a manner similar to Example 250.

¹H-NMR (200 MHz, CDCl₃): δ 2.95(6H, s), 3.83(3H, s), 4.6(2H, d, J=5.3 Hz), 5.08(2H, s), 5.29(1H, br-s), 6.89(2H, d, J=9 Hz), 6.95(2H, d, J=9 Hz), 7.32-7.6(9H, m). MS (ESI): 480 (M+Na)⁺, 458(M+H)⁺.

### Example 253

### N′-{[5-(4-Hydroxyphenyl)-4-(4-methoxyphenyl)-1,3-oxazol-2-yl]methyl}-N,N-dimethylurea

The title compound was obtained from N′-{[5-[4-(benzyloxy)phenyl]-4-(4-methoxyphenyl)-1,3-oxazol-2-yl]methyl}-N,N-dimethylurea obtained by Example 252 in a manner similar to Example 255 described later. MS (ESI): 368 (M+H)⁺.

### Example 254

### Methyl{[5-[4-(benzyloxy)phenyl]-4-(4-methoxyphenyl)-1,3-oxazol-2-yl]methyl}carbamate

The title compound was obtained from 1-[5-[4-(benzyloxy)phenyl]-4-(4-methoxyphenyl)-1,3-oxazol-2-yl]methanamine obtained by Example 233 and methyl chloridocarbonate in a manner similar to Example 250.

¹H-NMR (200 MHz, CDCl₃): δ 3.74(3H, s), 3.84(3H, s), 4.57(2H, d, J=5.6 Hz), 5.09(2H, s), 5.37(1H, br-s), 6.9(2H, d, J=9 Hz), 6.96(2H, d, J=9 Hz), 7.33-7.58(9H, m). MS (ESI): 467 (M+Na)⁺.

### Example 255

### Methyl{[5-(4-hydroxyphenyl)-4-(4-methoxyphenyl)-1,3-oxazol-2-yl]methyl}carbamate

Thioanisole (1.06 mL) was added to a solution of methyl {[5-[4-(benzyloxy)phenyl]-4-(4-methoxyphenyl)-1,3-oxazol-2-yl]methyl}carbamate obtained by Example 254 (1.00 g) in trifluoroacetic acid (10.0 mL) at 0° C.

After stirring for 10 hrs at room temperature, the mixture was poured into ice-cooling water. The pH of the mixture was justified to 10 with sodium hydroxide followed by extraction with ethyl acetate. The combined extracts were washed with brine, dried over magnesium sulfate, and evaporated in vacuo. The residue was triturated in isopropylether to give the title compound (799 mg). MS (ESI): 353 (M−H)⁻.

### Example 256

### N-{[5-[4-(2-([tert-Butyl(dimethyl)silyl]oxy}ethoxy)phenyl]-4-(4-methoxyphenyl)-1,3-oxazol-2-yl]methyl}propanamide

The title compound was obtained from N-{[5-(4-hydroxyphenyl)-4-(4-methoxyphenyl)-1,3-oxazol-2-yl]methyl}propanamide obtained by Example 251 and (2-bromoethoxy)(tert-butyl)dimethylsilane in a manner similar to Example 164.

MS (ESI): 511 (M+H)⁺.

### Example 257

### N-{[5-[4-(2-Hydroxyethoxy)phenyl]-4-(4-methoxyphenyl)-1,3-oxazol-2-yl]methyl}propanamide

The title compound was obtained from N-{[5-[4-(2-{[tert-butyl(dimethyl)silyl]oxy}ethoxy)phenyl]-4-(4-methoxyphenyl)-1,3-oxazol-2-yl]methyl}propanamide obtained by Example 256 in a manner similar to Example 166.

¹HNMR (200 MHz, CDCl₃): δ 1.21(3H, t, J=7.5 Hz), 2.33(2H, q, J=7.5 Hz), 3.84(3H, s), 3.92-4.04(2H, m), 4.05-4.15(2H, m), 4.64(2H, d, J=5 Hz), 6.22(1H, br-s), 6.9(4H, d, J=8.5 Hz), 7.49(2H, d, J=8.5 Hz), 7.53(2H, d, J=8.5 Hz). MS (ESI): 419 (M+Na)⁺, 397 (M+H)⁺.

### Example 258

### tert-Btyl[2-(4-{4-(4-methoxyphenyl)-2-(propionylamino)methyl]-1,3-oxazol-5-yl)phenoxy}ethyl]carbamate

The title compound was obtained from N-{[5-(4-hydroxyphenyl)-4-(4-methoxyphenyl)-1,3-oxazol-2-yl]methyl}propanamide obtained by Example 251 and tert-butyl (2-bromoethyl)carbamate in a manner similar to Example 215.

MS (ESI): 496 (M+H)⁺.

### Example 259

### Methyl{[5-[4-(2-{[tert-butyl(dimethyl)silyl]oxy}-ethoxy)phenyl]-4-(4-methoxyphenyl)-1,3-oxazol-2-yl]methyl}carbamate

The title compound was obtained from methyl {[5-(4-hydroxyphenyl)-4-(4-methoxyphenyl)-1,3-oxazol-2-yl]methyl}carbamate obtained by Example 255 and (2-bromoethoxy)(tert-butyl)dimethylsilane in a manner similar to Example 164.

MS (ESI): 513 (M+H)⁺.

### Example 260

### Methyl{[5-[4-(2-hydroxyethoxy)phenyl]-4-(4-methoxyphenyl)-1,3-oxazol-2-yl]methyl}carbamate

The title compound was obtained from methyl {[5-[4-(2-{[tert-butyl(dimethyl)silyl]oxy}ethoxy)phenyl]-4-(4-methoxyphenyl)-1,3-oxazol-2-yl]methyl}carbamate obtained by Example 259 in a manner similar to Example 166.

¹H-NMR (200 MHz, CDCl₃): δ 3.74(3H, s), 3.83(3H, s), 3.92-4.04(2H, m), 4.09-4.14(2H, m), 4.57(2H, d, J=5.5 Hz), 5.4(1H, br-s), 6.9(2H, d, J=8.5 Hz), 6.9(2H, d, J=9 Hz), 7.49(2H, d, J=8.5 Hz), 7.53(2H, d, J=9 Hz). MS (ESI): 421 (M+Na)⁺, 399 (M+H)⁺.

### Example 261

### Methyl{[5-(4-{2-[(tert-butoxycarbonyl)amino]ethoxy}-phenyl)-4-(4-methoxyphenyl)-1,3-oxazol-2-yl]methyl}carbamate

The title compound was obtained from methyl {[5-(4-hydroxyphenyl)-4-(4-methoxyphenyl)-1,3-oxazol-2-yl]methyl}carbamate obtained by Example 255 and tert-butyl (2-bromoethyl)carbamate in a manner similar to Example 215.

### Example 262

### N-{[5-[4-(2-Aminoethoxy)phenyl]-4-(4-methoxyphenyl)-1,3-oxazol-2-yl]methyl}propanamide

4N HCl in dioxane (6.0 mL) was added to a solution of tert-butyl[2-(4-{4-(4-methoxyphenyl)-2-[(propionylamino)methyl]-1,3-oxazol-5-yl}phenoxy)ethyl]carbamate obtained by Example 258 (766 mg) in dichloromethane (4.0 mL) at 0° C.

After stirring for 1 hr at 0° C., the product was washed with saturated sodium hydrogencarbonate aqueous solution and brine, dried over magnesium sulfate, and evaporated in vacuo to give the title compound (336 mg).

### Example 263

### N-{[5-(4-{2-[(Aminocarbonyl)amino]ethoxy}phenyl)-4-(4-methoxyphenyl)-1,3-oxazol-2-yl]methyl}propanamide

Triethylamine (0.182 mL) and trimethylsilyl isocyanate (75.2 mg) were added to a solution of N-{[5-[4-(2-aminoethoxy)phenyl]-4-(4-methoxyphenyl)-1,3-oxazol-2-yl]methyl}propanamide obtained by Example 262 (172 mg) in dichloromethane (2.20 mL) at 0° C.

After stirring for 10 hrs at room temperature, the product was extracted with ethyl acetate. The combined extracts were washed with 1N hydrochloric acid, saturated sodium hydrogencarbonate aqueous solution and brine, dried over magnesium sulfate, and evaporated under reduced presure. The residue was triturated in isopropylether, hexane and dichloromethane to give the title compound (62.6 mg).

¹H-NMR (200 MHz, CDCl₃): δ 1.21(3H, t, J=7.5 Hz), 2.33(2H, q, J=7.7 Hz), 3.53-3.68(2H, m), 3.83(3H, s), 4-4.09(2H, m), 4.43(2H, br-s), 4.63(2H, d, J=5 Hz), 5.02(1H, br-s), 6.24(1H, br-s), 6.86(2H, d, J=8.5 Hz), 6.9(2H, d, J=8.5 Hz), 7.47(2H, d, J=9 Hz), 7.52(2H, d, J=9 Hz). MS (ESI): 461 (M+Na)⁺.

### Example 264

### N-{[4-(4-Methoxyphenyl)-5-(4-{2-[(methylsulfonyl)amino]ethoxy}phenyl)-1,3-oxazol-2-yl]methyl}propanamide

Methanesulfonyl chloride (72.1 mg) and triethylamine (0.176 mL) were added to a solution of N-{[5-[4-(2-aminoethoxy)phenyl]-4-(4-methoxyphenyl)-1,3-oxazol-2-yl]methyl}propanamide obtained by Example 262 (166 mg) in dichloromethane (2.10 mL) at 0° C.

After stirring for 10 hrs at room temperature, the product was extracted with ethyl acetate. The combined extracts were washed with 1N hydrochloric acid, saturated sodium hydrogencarbonate aqueous solution and brine, dried over magnesium sulfate, and evaporated in vacuo. The residue was purified by silica gel column chromatography to give the title compound (200 mg).

¹H-NMR (200 MHz, CDCl₃): δ 1.21(3H, t, J=7.5 Hz), 2.33(2H, q, J=7.5 Hz), 3.03(3H, s), 3.51-3.6(2H, m), 3.84(3H, s), 4.1-4.15(2H, m), 4.63(2H, d, J=5 Hz), 4.87(1H, br-s), 6.24(1H, br-s), 6.86(2H, d, J=9.5 Hz), 6.91(2H, d, J=9.5 Hz), 7.49(2H, d, J=6.5 Hz), 7.53(2H, d, J=6.5 Hz). MS (ESI): 496 (M+Na)⁺.

### Example 265

### N′-{[5-[4-(2-{[tert-Butyl(dimethyl)silyl]oxy}ethoxy)phenyl]-4-(4-methoxyphenyl)-1,3-oxazol-2-yl]methyl)-N,N-dimethylurea

The title compound was obtained from N′-{[5-(4-hydroxyphenyl)-4-(4-methoxyphenyl)-1,3-oxazol-2-yl]methyl}-N,N-dimethylurea obtained by Example 253 and (2-bromoethoxy)(tert-butyl)dimethylsilane in a manner similar to Example 164.

### Example 266

### tert-Butyl(2-{4-[2-({[(dimethylamino)carbonyl]-amino}methyl)-4-(4-methoxyphenyl)-1,3-oxazol-5-yl]phenoxy}ethyl)carbamate

The title compound was obtained from N′-{[5-(4-hydroxyphenyl)-4-(4-methoxyphenyl)-1,3-oxazol-2-yl]methyl}-N,N-dimethylurea obtained by Example 253 and tert-butyl (2-bromoethyl)carbamate in a manner similar to Example 215.

### Example 267

### N′-{[5-[4-(2-Hydroxyethoxy)phenyl]-4-(4-methoxyphenyl)-1,3-oxazol-2-yl]methyl}-N,N-dimethylurea

The title compound was obtained from N′-{[5-[4-(2-{[tert-butyl(dimethyl)silyl]oxy}ethoxy)phenyl]-4-(4-methoxyphenyl)-1,3-oxazol-2-yl]methyl}-N,N-dimethylurea obtained by Example 265 in a manner similar to Example 166.

¹H-NMR (200 MHz, CDCl₃): δ 2.16(1H, br-s), 2.97(6H, s), 3.83(3H, s), 3.91-4.04(2H, m), 4.04-4.16(2H, m), 4.61(2H, d, J=5.5 Hz), 5.18(1H, br-s), 6.83-6.96(4H, m), 7.49(2H, d, J=9 Hz), 7.54(2H, d, J=9 Hz). MS (ESI): 410 (M−H)⁻.

### Example 268-1

### 5-[4-(Benzyloxy)phenyl]-4-(6-methoxy-3-pyridinyl)-1,3-oxazole-2(3H)-thione

To a mixture of 2-amino-1-[4-(benzyloxy)phenyl]-2-(6-methoxy-3-pyridinyl)ethanone hydrochloride (2 g) and carbon disulfide (CS₂) (870 mg) in ethanol, triethylamine (0.91 mL) under a nitrogen atmosphere was added dropwise, and the mixture was stirred at room temperature for 1 hrs. Triethylamine (0.91 mL) was further added, and the mixture was stirred at room temperature for 10 min. After water (15 mL) was added, the mixture was refluxed at 95° C. for 3 hrs.

After the mixture was cooled, the resulting precipitates were removed, and the mother liquor was extracted with dichloromethane twice. The combined extracts were dried over magnesium sulfate and concentrated to give the crude product (2.21 g), which was used for the next step without further purification.

### Example 268-2

### 5-[5-[4-(Benzyloxy)phenyl]-2-(methylthio)-1,3-oxazol-4-yl]-2-methoxypyridine

To a solution of 5-[4-(benzyloxy)phenyl]-4-(6-methoxy-3-pyridinyl)-1,3-oxazole-2(3H)-thione obtained by Example 268-1 (1.99 g) in dimethylformamide (20 mL), sodium hydride (60% in mineral oil; 306 mg) was added at 0° C. under a nitrogen atmosphere, and the mixture was stirred for 5 min. Methyl iodide (0.48 mL) was added dropwise, and the mixture was stirred at this temperature for 1.5 hrs.

The reaction mixture was quenched with water and extracted with ethyl acetate. The organic layer was washed with water three times, dried over magnesium sulfate, and concentrated. The residue was triturated with methanol, and the resulting powder was collected, washed with methanol, and dried in vacuo to give the title compound (0.99 g).

¹H-NMR (CDCl₃): δ 2.71(3H, s), 3.96(3H, s), 5.09(2H, s), 6.60-8.50(12H, m). MS (ESI): 405.00 (M+H)⁺.

### Example 269

### 4-[4-(6-Methoxy-3-pyridinyl)-2-(methylthio)-1,3-oxazol-5-yl]phenol

A mixture of 5-[5-[4-(benzyloxy)phenyl]-2-(methylthio)-1,3-oxazol-4-yl]-2-methoxypyridine obtained by Example 268-2 (0.99 g) and thioanisole (1.15 mL) in trifluoroacetic acid (10 mL) was stirred at room temperature overnight.

The mixture was concentrated, basified with saturated sodium hydrogencarbonate aqueous solution, and extracted with dichloromethane twice. The combined extracts were dried over magnesium sulfate and concentrated to give the title compound (0.86 g)

¹H-NMR (CDCl₃): δ 2.71(3H, s), 4.01(3H, s), 6.70-8.70(8H, m). MS (ESI): 315.1 (M+H)⁺.

### Example 270

### tert-Butyl(2-{4-[4-(6-methoxy-3-pyridinyl)-2-(methylthio)-1,3-oxazol-5-yl]phenoxy}ethyl)carbamate

The title compound was obtained from 4-[4-(6-methoxy-3-pyridinyl)-2-(methylthio)-1,3-oxazol-5-yl]phenol obtained by Example 269 in a manner similar to Example 194.

MS (ESI): 480.2 (M+Na)⁺.

### Example 271

### (2-{4-[4-(6-Methoxy-3-pyridinyl)-2-(methylthio)-1,3-oxazol-5-yl]phenoxy}ethyl)amine

To a solution of crude tert-butyl(2-{4-[4-(6-methoxy-3-pyridinyl)-2-(methylthio)-1,3-oxazol-5-yl]phenoxy}ethyl)carbamate obtained by Example 270 (1.38 g) in dichloromethane (15 mL), trifluoroacetic acid (8 mL) was added at 0° C., and the mixture was stirred at this temperature for 1 hr.

The mixture was concentrated, basified with 1N sodium hydroxide, and extracted with dichloromethane five times. The combined extracts were dried over magnesium sulfate and concentrated. The residue was chromatographed on silica gel (dichloromethane/methanol=9/1) to give the title compound (625 mg).

¹H-NMR (CDCl₃): δ 2.71(3H, s), 3.11(2H, t, J=5.1 Hz), 3.96(2H, t, J=5.1 Hz), 6.60-8.60(7H, m). MS (ESI): 358.1 (M+H)⁺.

### Example 272

### N-(2-{4-[4-(6-Methoxy-3-pyridinyl)-2-(methylthio)-1,3-oxazol-5-yl]phenoxy}ethyl)urea

The title compound was obtained from (2-{4-[4-(6-methoxy-3-pyridinyl)-2-(methylthio)-1,3-oxazol-5-yl]phenoxy}ethyl)amine obtained by Example 271 in a manner similar to Example 196.

¹H-NMR (CDCl₃): δ 2.71(3H, s), 3.61(2H, m), 4.00(3H, s), 4.06(2H, t, J=4.9 Hz), 4.48(2H, br-s), 5.12(1H, br-s), 6.70-8.60(7H, m). MS (ESI): 423.1 (M+Na)⁺.

### Example 273

### N-(2-{4-[4-(6-Methoxy-3-pyridinyl)-2-(methylsulfonyl)-1,3-oxazol-5-yl]phenoxy}ethyl)urea

The title compound was obtained from N-(2-{4-[4-(6-methoxy-3-pyridinyl)-2-(methylthio)-1,3-oxazol-5-yl]phenoxy}ethyl)urea obtained by Example 272 in a manner similar to Example 197.

¹H-NMR (CDCl₃): δ 3.42(3H, s), 3.63(2H, m), 3.97(3H, s), 4.09(2H, t, J=5.0 Hz), 4.46(2H, br-s), 5.00(1H, br-s), 6.80-8.50(7H, m). MS (ESI): 432.45 (M+Na)⁺.

### Example 274

### N-(2-{4-[2-(2-Ethoxyethoxy)-4-(6-methoxy-3-pyridinyl)-1,3-oxazol-5-yl]phenoxy}ethyl)urea

The title compound was obtained from N-(2-{4-[4-(6-methoxy-3-pyridinyl)-2-(methylsulfonyl)-1,3-oxazol-5-yl]phenoxy}ethyl)urea obtained by Example 273 in a manner similar to Example 219.

¹H-NMR (CDCl₃): δ 1.25(3H, t, J=6.9 Hz), 3.40-3.90(6H, m), 3.95(3H, s), 4.06(2H, t, J=4.9 Hz), 4.40(2H, br-s), 4.61(2H, m), 4.97(1H, br-s), 6.70-8.70(7H, m). MS (ESI): 465.2 (M+Na)⁺.

### Example 275

### N′-{[5-[4-(2-Aminoethoxy)phenyl]-4-(4-methoxyphenyl)-1,3-oxazol-2-yl]methyl}-N,N-dimethylurea

The title compound was obtained from tert-butyl (2-{4-[2-({[(dimethylamino)carbonyl]amino}methyl)-4-(4-methoxyphenyl)-1,3-oxazol-5-yl]phenoxy}ethyl)carbamate obtained by Example 266 in a manner similar to Example 262.

### Example 276

### Methyl{[5-[4-(2-aminoethoxy)phenyl]-4-(4-methoxyphenyl)-1,3-oxazol-2-yl]methyl}carbamate

The title compound was obtained from methyl {[5-(4-{2-[(tert-butoxycarbonyl)amino]ethoxy}phenyl)-4-(4-methoxyphenyl)-1,3-oxazol-2-yl]methyl}carbamate obtained by Example 261 in a manner similar to Example 262.

### Example 277

### N-(2-{4-[2-({[(Dimethylamino)carbonyl]amino}methyl)-4-(4-methoxyphenyl)-1,3-oxazol-5-yl]phenoxy}ethyl)methanesulfonamide

The title compound was obtained from N′-{[5-[4-(2-aminoethoxy)phenyl]-4-(4-methoxyphenyl)-1,3-oxazol-2-yl]methyl}-N,N-dimethylurea obtained by Example 275 in a manner similar to Example 264.

¹H-NMR (200 MHz, CDCl₃): δ 2.97(6H, s), 3.02(3H, s), 3.48-3.65(2H, m), 3.83(3H, s), 4.07-4.14(2H, m), 4.6(2H, d, J=5.5 Hz), 4.97(1H, br-s), 5.23(1H, br-s), 6.85(2H, d, J=9 Hz), 6.9(2H, d, J=9 Hz), 7.49(2H, d, J=6.5 Hz), 7.53(2H, d, J=6.5 Hz). MS (ESI): 511 (M+Na)⁺.

### Example 278

### N′-{[5-(4-{2-[(Aminocarbonyl)amino]ethoxy}phenyl)-4-(4-methoxyphenyl)-1,3-oxazol-2-yl]methyl}-N,N-dimethylurea

The title compound was obtained from N′-{[5-[4-(2-aminoethoxy)phenyl]-4-(4-methoxyphenyl)-1,3-oxazol-2-yl]methyl}-N,N-dimethylurea obtained by Example 275 in a manner similar to Example 263.

¹H-NMR (200 MHz, CDCl₃): δ 2.96(6H, s), 3.49-3.7(2H, m), 3.83(3H, s), 3.94-4.09(2H, m), 4.58(2H, d, J=5 Hz), 4.65(2H, br-s), 5.27(1H, br-s), 5.43(1H, br-s), 6.82(2H, d, J=9 Hz), 6.88(2H, d, J=9 Hz), 7.43(2H, d, J=9 Hz), 7.5(2H, d, J=9 Hz). MS (ESI): 454 (M+H)⁺.

### Example 279

### Methyl{[4-(4-methoxyphenyl)-5-(4-{2-[(methylsulfonyl)amino]ethoxy}phenyl)-1,3-oxazol-2-yl]methyl}carbamate

The title compound was obtained from methyl {[5-[4-(2-aminoethoxy)phenyl]-4-(4-methoxyphenyl)-1,3-oxazol-2-yl]methyl}carbamate obtained by Example 276 in a manner similar to Example 264.

¹H-NMR (200 MHz, CDCl₃): δ 3.03(3H, s), 3.45-3.63(2H, m), 3.74(3H, s), 3.83(3H, s), 4.05-4.18(2H, m), 4.56(2H, d, J=6 Hz), 4.85(1H, br-s), 5.43(1H, br-s), 6.86(2H, d, J=6.5 Hz), 6.9(2H, d, J=7 Hz), 7.49(2H, d, J=6.5 Hz), 7.53(2H, d, J=7 Hz). MS (ESI): 498 (M+Na)⁺.

### Example 280

### Methyl{[5-(4-{2-[(aminocarbonyl)amino]ethoxy}-phenyl)-4-(4-methoxyphenyl)-1,3-oxazol-2-yl]methyl}carbamate

The title compound was obtained from methyl{[5-[4-(2-aminoethoxy)phenyl]-4-(4-methoxyphenyl)-1,3-oxazol-2-yl]methyl}carbamate obtained by Example 276 in a manner similar to Example 263.

¹H-NMR (200 MHz, CDCl₃): δ 3.49-3.68(2H, m), 3.74(3H, s), 3.83(3H, s), 3.95-4.15(2H, m), 4.4-4.7(4H, m), 5.09(1H, br-s), 5.44(1H, br-s), 6.85(2H, d, J=6.5 Hz), 6.9(2H, d, J=6.5 Hz), 7.47(2H, d, J=9 Hz), 7.52(2H, d, J=9 Hz).

### Example 281

### N-(2-{4-[4-(6-Methoxy-3-pyridinyl)-2-(trifluoromethyl)-1,3-oxazol-5-yl]phenoxy}ethyl)-2,2-dimethylhydrazinecarboxamide

The title compound (115 mg) was obtained from 4-nitrophenyl(2-{4-[4-(6-methoxy-3-pyridinyl)-2-(trifluoromethyl)-1,3-oxazol-5-yl]phenoxy}ethyl)carbamate obtained by Example 245 (300 mg) and N,N-dimethylhydrazine (166 mg) in a manner similar to Example 246.

¹H-NMR (DMSO-d₆): d 2.4(6H, s), 3.3-3.5(2H, m), 3.89(3H, s), 4.06(2H, t, J=6 Hz), 6.67(1H, t, J=5.9 Hz), 6.93(1H, d, J=8.9 Hz), 7-7.15(3H, m), 7.54(2H, d, J=8.7 Hz), 7.86(1H, dd, J=2.2,8.9 Hz), 8.38(1H, d, J=2.2 Hz). MS (ESI): 488.2 (M+Na)⁺.

### Example 282

### 5-(4-Hydroxyphenyl)-N-methoxy-4-(6-methoxy-3-pyridinyl)-N-methyl-1,3-oxazole-2-carboxamide

The title compound (1.29 g) was obtained from 5-[4-(benzyloxy)phenyl]-N-methoxy-4-(6-methoxy-3-pyridinyl)-N-methyl-1,3-oxazole-2-carboxamide (2.0 g) in a manner similar to Example 235.

¹H-NMR (DMSO-d₆): δ 3.34(3H, s), 3.86(3H, s), 3.89(3H, s), 6.88(2H, d, J=8.6 Hz), 6.91(1H, d, J=7.3 Hz), 7.43(2H, d, J=8.6 Hz), 7.87(1H, dd, J=2.5,8.6 Hz), 8.4(1H, d, J=2.3 Hz). MS (ESI): 378.3 (M+Na)⁺.

### Example 283

### 5-[4-(2-{[tert-Butyl(dimethyl)silyl]oxy}ethoxy)phenyl]-N-methoxy-4-(6-methoxy-3-pyridinyl)-N-methyl-1,3-oxazole-2-carboxamide

Under a nitrogen atmosphere, sodium hydride(197 mg) was added to a solution of 5-(4-hydroxyphenyl)-N-methoxy-4-(6-methoxy-3-pyridinyl)-N-methyl-1,3-oxazole-2-carboxamide obtained by Example 282 (1.46 g) in dimethylformamide (15 mL) at 0° C. After 10 min, a solution of (2-bromoethoxy)trimethylsilane (104 mg) in dimethylformambide (1 mL) was added. The whole mixture was stirred at room temperature for 30 min and at 40° C. for 2 hrs.

The mixture was poured into a mixture of cold water and ethyl acetate, and the aqueous layer was separated. The organic layer was washed with water and brine, and dried over magnesium sulfate. Evaporation of the solvent afforded the title compound (1.38 g).

¹H-NMR (DMSO-d₆): δ 0.04(6H, s), 0.86(9H, s), 3.33(3H, s), 3.87(3H, s), 3.89(3H, s), 3.8-3.9(2H, m), 4-4.1(2H, m), 6.91(1H, d, J=9.1 Hz), 7.07(2H, d, J=8.9 Hz), 7.53(2H, d, J=8.8 Hz), 7.87(1H, dd, J=2.4,8.7 Hz), 8.4(1H, d, J=2.3 Hz). MS (ESI): 536.2 (M+Na)⁺.

### Example 284

### Cyclopropyl[5-[4-(2-hydroxyethoxy)phenyl]-4-(6-methoxy-3-pyridinyl)-1,3-oxazol-2-yl]methanone

Under a nitrogen atmosphere, 0.5M solution of cyclopropylmagnesium bromide in tetrahydrofuran (1.5 mL) was added to a solution of 5-[4-(2-{[tert-butyl(dimethyl)silyl]oxy}ethoxy)phenyl]-N-methoxy-4-(6-methoxy-3-pyridinyl)-N-methyl-1,3-oxazole-2-carboxamide obtained by Example 283 (400 mg) in tetrahydrofuran (4.2 mL) at −78° C.

The mixture was stirred for 3 hrs at the same temperature and the reaction mixture was quenched with saturated ammonium chloride aqueous solution. The mixture was poured into a mixture of water and ethyl acetate, and the aqueous layer was separated. The organic layer was washed with water and brine, and dried over magnesium sulfate. After evaporation of the solvent, the residue was dissolved in tetrahydrofuran (5 mL).

1M solution of tetrabutylammonium fluoride (0.41 mL) was added to the solution. The mixture was stirred at room temperature for 1 hr, and poured into into a mixture of water and ethyl acetate. The aqueous layer was separated, and the organic layer was washed with brine and dried over magnesium sulfate. After evaporation of the solvent, the residue was purified by column chromatography on silica-gel eluting with dichloromethane and acetone to give the title compound (98 mg).

¹H-NMR (DMSO-d₆): δ 1.1-1.3(4H, m), 3.0-3.1(1H, m), 3.7-3.8(2H, m), 3.90(3H, s), 4.0-4.1(2H, m), 4.90(1H, t, J=5.5 Hz), 6.94(1H, d, J=8.6 Hz), 7.07(2H, d, J=8.8 Hz), 7.55(2H, d, J=8.8 Hz), 7.89(1H, dd, J=8.6,2.3 Hz), 8.40(1H, d, J=2.3 Hz). MS (ESI): 403.1 (M+Na)⁺.

### Example 285

### [5-[4-(Benzyloxy)phenyl]-4-(6-methoxy-3-pyridinyl)-1,3-oxazol-2-yl]methyl methanesulfonate

Under a nitrogen atmosphere, methanesulfonyl chloride (0.21 mL) was added to a solution of [5-[4-(benzyloxy)phenyl]-4-(6-methoxy-3-pyridinyl)-1,3-oxazol-2-yl]methanol obtained by Example 201 (700 mg) and triethylamine (0.75 mL) in dichloromethane (14 mL) at −10° C.

The mixture was stirred for 1 hr at the same temperature, and poured into a mixture of cold water and ethyl acetate. The aqueous layer was separated, and the organic layer was washed with diluted hydrochloric acid, water and brine, and dried over magnesium sulfate. Evaporation of the solvent afforded the title compound (720 mg).

¹H-NMR (DMSO-d₆): δ 3.36(3H, s), 3.88(3H, s), 4.98(2H, s), 5.15(2H, s), 6.9(1H, d, J=8.5 Hz), 7.14(1H, d, J=10 Hz), 7.3-7.5(7H, m), 7.84(1H, dd, J=2.4,8.7 Hz), 8.37(1H, d, J=1.9 Hz).

### Example 286

### 5-{5-[4-(Benzyloxy)phenyl]-2-[(4-pyridinylthio)methyl]-1,3-oxazol-4-yl}-2-methoxypyridine

Under a nitrogen atmosphere, 4-mercaptopyridine (250 mg) and N,N-diisoproylethylamine (0.39 mL) was added successively to a solution of [5-[4-(benzyloxy)phenyl]-4-(6-methoxy-3-pyridinyl)-1,3-oxazol-2-yl]methyl methanesulfonate obtained by Example 285 (700 mg) in dimethylformamide (7 mL) at 0 ° C. The mixture was stirred at the same temperature for 2 hrs, and poured into a mixture of cold water and ethyl acetate. The aqueous layer was separated, the organic layer was washed with water and brine, and dried over magnesium sulfate. After evaporation of the solvent, the residue was purified by column chromatography on silica-gel eluting with dichloromethane and acetone to afford the title compound (670 mg).

¹H-NMR (DMSO-d₆): δ 3.87(3H, s), 4.67(2H, s), 5.14(2H, s), 6.88(1H, d, J=8.5 Hz), 7.1(2H, d, J=8.9 Hz), 7.36-7.49(9H, m), 7.79(1H, dd, J=2.5,8.6 Hz), 8.32(1H, d, J=2.3 Hz), 8.44(2H, dd, J=1.6,4.6 Hz). MS (ESI): 482.2 (M+H)⁺.

### Example 287

### 4-(4-(6-Methoxy-3-pyridinyl)-2-[(4-pyridinylthio)methyl]-1,3-oxazol-5-yl}phenol

Under a nitrogen atmosphere, thioanisole was added to a solution of 5-{5-[4-(benzyloxy)phenyl]-2-[(4-pyridinylthio)methyl]-1,3-oxazol-4-yl}-2-methoxypyridine obtained by Example 286 (660 mg) in trifluoroacetic acid (7 mL) at 0° C.

After 30 min, ice bath was removed and the mixture was stirred overnight at room temperature. The mixture was poured into a mixture of cold saturated sodium hydrogencarbonate aqueous solution and ethyl acetate. The aqueous layer was separated, the organic layer was washed with saturated sodium hydrogencarbonate aqueous solution, water and brine, and dried over magnesium sulfate. After evaporation of the solvent, the residue was purified by column chromatography on silica-gel eluting with dichloromethane and acetone to afford the title compound (420 mg).

¹H-NMR (DMSO-d₆): δ 3.87(3H, s), 4.66(2H, s), 6.8(2H, d, J=4.7 Hz), 6.87(1H, d, J=8.4 Hz), 7.29(2H, dd, J=1.9,6.8 Hz), 7.48(1H, dd, J=1.6, 4.6 Hz), 7.78(1H, dd, J=2.5, 8.6 Hz), 8.32(1H, d, J=2.4 Hz), 8.44(2H, dd, J=1.5, 4.6 Hz), 9.93(1H, s).

### Example 288

### 5-{5-[4-(Benzyloxy)phenyl]-2-[(2-pyridinylthio)methyl]-1,3-oxazol-4-yl}-2-methoxypyridine

The title compound (580 mg) was obtained from [5-[4-(benzyloxy)phenyl]-4-(6-methoxy-3-pyridinyl)-1,3-oxazol-2-yl]methyl methanesulfonate obtained by Example 285 (660 mg) and 2-mercaptopyridine (236 mg) in a manner similar to Example 286.

¹H-NMR (DMSO-d₆): δ 3.86(3H, s), 4.69(2H, s), 5.13(2H, s), 6.86(1H, d, J=8.6 Hz), 7.09(2H, d, J=8.8 Hz), 7.15-7.5(9H, m), 7.6-7.85(2H, m), 8.31(1H, d, J=2.3 Hz), 8.5(1H, dd, J=2.3, 8.6 Hz). MS (ESI): 504.1 (M+Na)⁺.

### Example 289

### (E)-Cyclopropyl[5-[4-(2-hydroxyethoxy)phenyl]-4-(6-methoxy-3-pyridinyl)-1,3-oxazol-2-yl]methanone oxime

Hydroxylamine hydrochloride (63.9 mg) was added to a solution of cyclopropyl[5-[4-(2-hydroxyethoxy)phenyl]-4-(6-methoxy-3-pyridinyl)-1,3-oxazol-2-yl]methanone obtained by Example 284 (70 mg) in pyridine (3 mL) at room temperature. The mixture was stirred at 80° C. for 8 hrs and cooled to room temperature.

The solvent was evaporated, and the residue was dissolved in a mixture of water and ethyl acetate. The aqueous layer was separated, and the organic layer was washed with diluted aqueous hydrochloric acid, water and brine, and dried over magnesium sulfate. After evaporation of the solvent, the residue was purified by preparative thin layer chromatography on silica-gel eluting with dichloromethane and acetone to afford the title compound (41 mg).

¹H-NMR (DMSO-d₆): δ 0.8-1.0(2H, m), 1.4-2.5(2H, m), 2.4-2.5(1H, m), 3.65-3.75(2H, m), 3.88(3H, s), 4.0-4.1(2H, m), 4.90(1H, t, J=5.5 Hz), 6.90(1H, d, J=8.6 Hz), 7.04(2H, d, J=8.8 Hz), 7.46(2H, d, J=8.8 Hz), 7.83(1H, dd, J=8.6,2.3 Hz), 8.35(1H, d, J=8.6 Hz), 12.03(1H, s). MS (ESI): 394.1 (M−H)⁻.

### Example 290-1

### 5-(4-Methoxyphenyl)-4-(6-methoxy-3-pyridinyl)-1,3-oxazole-2(3H)-thione

Triethylamine (5.60 mL) and carbon disulfide (1.64 mL) was added to a solution of 2-amino-1-(4-methoxyphenyl)-2-(6-methoxy-3-pyridinyl)ethanone hydrochloride (4.00 g) in ethanol (40.0 mL) at 0° C.

After stirring for 1.5 hrs at 55° C., the mixture was poured into ice cooling water at room temperature. The product was extracted with ethyl acetate. The combined extracts were washed with brine dried over magnesium sulfate, and evaporated in vacuo to give the title compound (7.92 g).

### Example 290-2

2-Methoxy-5-[5-(4-methoxyphenyl)-2-(methylthio)-1,3-oxazol-4-yl]pyridine

A solution of 5-(4-methoxyphenyl)-4-(6-methoxy-3-pyridinyl)-1,3-oxazole-2(3H)-thione obtained by Example 290-1 (8.79 g) in dimethylformamide (25.0 mL) and methyl iodide (3.13 mL) in dimethylformamide (23.0 mL) was added to a solution of sodium hydride (2.01 g) in dimethylformamide (45.0 mL) at 0° C.

After stirring for 20 min, the reaction mixture was quenched with water at 0° C. The precipitate was produced, which as collected by filtration with isopropylether and it was purified by silica gel column chromatography to give the title compound (4.90 g).

¹H-NMR (200 MHz, CDCl₃): δ 2.71(3H, s), 3.8(3H, s), 3.94(3H, s), 6.75(1H, d, J=8.5 Hz), 6.89(2H, d, J=9 Hz), 7.45(2H, d, J=9 Hz), 7.82(1H, dd, J=2.5,8.5 Hz), 8.43(1H, d, J=2.5 Hz). MS (ESI): 329 (M+H)⁺, 351 (M+Na)⁺.

### Example 291

### tert-Butyl[2-(4-{4-(6-methoxy-3-pyridinyl)-2-[(4-pyridinylthio)methyl]-1,3-oxazol-5-yl}phenoxy)ethyl]carbamate

The title compound (310 mg) was obtained from 4-{4-(6-methoxy-3-pyridinyl)-2-[(4-pyridinylthio)methyl]-1,3-oxazol-5-yl}phenol obtained by Example 287 (280 mg) in a manner similar to Example 182.

¹H-NMR (DMSO-d₆): δ 1.37(9H, s), 3.3-3.4(2H, m), 3.87(3H, s), 3.9-4.0(2H, m), 4.66(2H, s), 6.87(1H, d, J=8.6 Hz), 7.01(2H, d, J=8.8 Hz), 7.39(2H, d, J=8.8 Hz), 7.48(2H, d, J=4.6 Hz), 7.78(1H, dd, J=8.6,2.3 Hz), 8.31(1H, d, J=2.3 Hz), 8.43(2H, d, J=4.6 Hz). MS (ESI): 535.2 (M+H)⁺.

### Example 292

### [2-(4-{4-(6-Methoxy-3-pyridinyl)-2-[(4-pyridinylthio)methyl]-1,3-oxazol-5-yl}phenoxy)ethyl]amine

The title compound (218 mg) was obtained from tert-butyl [2-(4-{4-(6-methoxy-3-pyridinyl)-2-[(4-pyridinylthio)methyl]-1,3-oxazol-5-yl}phenoxy)ethyl]carbamate obtained by Example 291 (300 mg) in a manner similar to Example 198.

¹H-NMR (DMSO-d₆): δ 2.87(2H, t, J=5.6 Hz), 3.87(3H, s), 3.95(2H, t, J=5.6 Hz), 4.66(2H, s), 6.87(1H, d, J=8.6 Hz), 7.03(2H, d, J=8.8 Hz), 7.29(2H, d, J=8.8 Hz), 7.4-7.5(4H, m), 7.79(1H, dd, J=8.6,2.5 Hz), 8.31(1H, d, J=2.3 Hz), 8.44(2H, d, J=4.9 Hz). MS (ESI): 435.2 (M+H)⁺.

### Example 293

### N-[2-(4-{4-(6-Methoxy-3-pyridinyl)-2-[(4-pyridinylthio)methyl]-1,3-oxazol-5-yl}phenoxy)ethyl]methanesulfonamide

The title compound (69 mg) was obtained from [2-(4-{4-(6-methoxy-3-pyridinyl)-2-[(4-pyridinylthio)methyl]-1,3-oxazol-5-yl)phenoxy)ethyl]amine obtained by Example 292 (80 mg) in a manner similar to Example 199.

¹H-NMR (DMSO-d₆): δ 2.96(3H, s), 3.3-3.47(2H, m), 3.87(3H, s), 3.9-4.0(2H, m), 4.67(2H, s), 6.88(1H, d, J=8.6 Hz), 7.04(2H, d, J=8.8 Hz), 7.3-7.5(5H, m), 7.79(1H, dd, J=8.6,2.3 Hz), 8.33(1H, d, J=2.3 Hz), 8.44(1H, d, J=4.9 Hz). MS (ESI): 513.1 (M+H)⁺.

### Example 294

### N-[2-(4-{4-(6-Methoxy-3-pyridinyl)-2-[(4-pyridinylthio)methyl]-1,3-oxazol-5-yl}phenoxy)ethyl]urea

The title compound (104 mg) was obtained from [2-(4-{4-(6-methoxy-3-pyridinyl)-2-[(4-pyridinylthio)methyl]-1,3-oxazol-5-yl}phenoxy)ethyl]amine obtained by Example 292 (140 mg) in a manner similar to Example 200.

¹H-NMR (DMSO-d₆): δ 3.3-3.4(2H, m), 3.87(3H, s), 3.9-4.0(2H, m), 4.67(2H, s), 5.54(2H, s), 6.17(1H, t, J=5.6 Hz), 6.87(1H, d, J=8.6 Hz), 7.03(2H, d, J=8.8 Hz), 7.40(2H, d, J=8.8 Hz), 7.48(2H, d, J=6.2 Hz), 7.78(2H, dd, J=8.8,2.4 Hz), 8.32(1H, d, J=2.4 Hz), 8.44(1H, d, J=3.1 Hz). MS (ESI): 478.1 (M+H)⁺.

### Example 295

### [5-[4-(2-Azidoethoxy)phenyl]-4-(6-methoxy-3-pyridinyl)-1,3-oxazol-2-yl](cyclopropyl)methanone

Under a nitrogen atmosphere, methanesulfonyl chloride (90 mg) was added to a solution of cyclopropyl[5-[4-(2-hydroxyethoxy)phenyl]-4-(6-methoxy-3-pyridinyl)-1,3-oxazol-2-yl]methanone obtained by Example 284 (199 mg) and triethylamine (212 mg) in dichloromethane (6 mL) at −10° C.

The mixture was stirred for 1 hr at the same temperature, and poured into a mixture of cold water and ethyl acetate. The aqueous layer was separated, and the organic layer was washed with water and brine, and dried over magnesium sulfate. After evaporation of the solvent, the residue was dissolved in dimethylformamice (6 mL) and sodium azide (68 mg) was added to this solution. The mixture was stirred overnight at 50° C., and poured into a mixture of water and ethyl acetate. The aqueous layer was separated, the organic layer was washed with water and brine, and dried over magnesium sulfate. After evaporation of the solvent, the residue was purified by column chromatography on silica-gel eluting with dichloromethane and acetone to give the title compound (223 mg).

MS (ESI): 406.1 (M+H)⁺.

### Example 296

### N-(2-{4-[2-(Cyclopropylcarbonyl)-4-(6-methoxy-3-pyridinyl)-1,3-oxazol-5-yl]phenoxy}ethyl)methanesulfonamide

Triphenylphosphine (59.5 mg) and water (100 μL) were added to a solution of [5-[4-(2-azidoethoxy)phenyl]-4-(6-methoxy-3-pyridinyl)-1,3-oxazol-2-yl](cyclopropyl)methanone obtained by Example 295 (92 mg) in ethyl acetate (2 mL). The mixture was stirred overnight at room temperature and dried over magnesium sulfate.

After evaporation of the solvent, the residue was dissolved in dichloromethane (4 mL) and cooled at −20° C. under a nitrogen atmosphere. Triethylamine (91.8 mg) and methanesulfonyl chloride (39 mg) were added to this solution.

The mixture was stirred for 45 min at the same temperature, and poured into a mixture of cold water and ethyl acetate. The aqueous layer was separated, and the organic layer was washed with diluted hydrochloric acid, water and brine, and dried over magnesium sulfate. After evapoation of the solvent, the residue was purified by column chromatography on silica-gel eluting with dichloromethane and acetone to afford the title compound (22.5 mg).

¹H-NMR (DMSO-d₆): δ 1.15-1.25(4H, m), 2.95(3H, s), 3.02-3.11(1H, m), 3.32-3.39(2H, m), 3.9(3H, s), 4.1(2H, t, J=5.5 Hz), 6.94(1H, d, J=8.5 Hz), 7.09(2H, d, J=8.8 Hz), 7.31(1H, t, J=5.8 Hz), 7.57(2H, d, J=8.8 Hz), 7.9(1H, dd, J=2.5,8.6 Hz), 8.41(1H, d, J=2.3 Hz). MS (ESI): 458.0 (M+H)⁺.

### Example 297

### 1-[5-[4-(2-Hydroxyethoxy)phenyl]-4-(6-methoxy-3-pyridinyl)-1,3-oxazol-2-yl]-2-methyl-1-propanone

The title compound (23.0 mg) was obtained from 5-[4-(2-{[tert-butyl(dimethyl)silyl]oxy}ethoxy)phenyl]-N-methoxy-4-(6-methoxy-3-pyridinyl)-N-methyl-1,3-oxazole-2-carboxamide obtained by Example 283 (150 mg) and isopropylmagnesium bromide (1.29 mL) in a manner similar to Example 284.

¹H-NMR (DMSO-d₆): δ 0.9-1.2(1H, m), 1.21(6H, d, J=6.9 Hz), 3.5-3.8(2H, m), 3.9(3H, s), 3.95-4.1(2H, m), 4.91(1H, t, J=5.4 Hz), 6.94(1H, d, J=8.9 Hz), 7.08(2H, d, J=8.8 Hz), 7.56(2H, d, J=7 Hz), 7.89(1H, dd, J=2.4,8.6 Hz), 8.39(1H, d, J=2.4 Hz). MS (ESI): 405.2 (M+Na)⁺.

### Example 298

### N-(2-{4-[2-(Cyclopropylcarbonyl)-4-(6-methoxy-3-pyridinyl)-1,3-oxazol-5-yl]phenoxy}ethyl)urea

Triphenylphosphine (76.3 mg) and water (100 μL) were added to a solution of [5-[4-(2-azidoethoxy)phenyl]-4-(6-methoxy-3-pyridinyl)-1,3-oxazol-2-yl](cyclopropyl)methanone obtained by Example 295 (118 mg) in ethyl acetate (5 mL). The mixture was stirred overnight at room temperature and dried over magnesium sulfate.

After evaporation of the solvent, the residue was dissolved in a mixture of dimethylformamide (3 mL) and water (0.75 mL). To this solution, sodium acetate (143 mg) and a solution of potassium cyanate (142 mg) in water(1 mL) were added successively at room temperature.

The mixture was stirred overnight at 60° C., and was poured into a mixture of water and ethyl acetate. Aqueous layer was separated, the organic layer was washed with water and brine, and dried over magnesium sulfate. After evaporation of the solvent, the residue was purified by column chromatography on silica-gel eluting with dichloromethane and acetone to give the title compound (61.2 mg).

¹H-NMR (DMSO-d₆): δ 1.0-1.5(4H, m), 3.0-3.2(1H, m), 3.3-3.4(2H, m), 3.9(3H, s), 3.92-4.15(2H, m), 5.54(2H, s), 6.18(1H, t, J=5.6 Hz), 6.94(1H, d, J=8.6 Hz), 7.09(2H, d, J=8.9 Hz), 7.56(2H, d, J=8.8 Hz), 7.9(1H, dd, J=2.5,8.7 Hz), 8.41(1H, d, J=2.3 Hz). MS (ESI): 445.1 (M+Na)⁺.

### Example 299

### N-(2-{4-[2-[(E)-Cyclopropyl(hydroxyimino)methyl]-4-(6-methoxy-3-pyridinyl)-1,3-oxazol-5-yl]phenoxy}ethyl)methanesulfonamide

The title compound (51.1 mg) was obtained from N-(2-{4-[2-(cyclopropylcarbonyl)-4-(6-methoxy-3-pyridinyl)-1,3-oxazol-5-yl]phenoxy}ethyl)methanesulfonamide obtained by Example 296 (60 mg) in a manner similar to Example 289.

¹H-NMR (DMSO-d₆): δ 0.8-1.1(2H, m), 1.4-1.5(2H, m), 2.4-2.5(1H, m), 2.95(3H, s), 3.3-3.4(2H, m), 3.88(3H, s), 4.08(1H, t, J=5.4 Hz), 6.9(1H, d, J=8.6 Hz), 7.06(2H, d, J=8.8 Hz), 7.31(1H, t, J=5.8 Hz), 7.48(2H, d, J=8.8 Hz), 7.84(1H, dd, J=2.4,8.6 Hz), 8.36(1H, d, J=2.4 Hz). MS (ESI): 495.1 (M+Na)⁺.

### Example 300

### N-(2-{4-[2-[(E)-Cyclopropyl(hydroxyimino)methyl]-4-(6-methoxy-3-pyridinyl)-1,3-oxazol-5-yl]phenoxy}ethyl)urea

The title compound (21.1 mg) was obtained from N-(2-{4-[2-(cyclopropylcarbonyl)-4-(6-methoxy-3-pyridinyl)-1,3-oxazol-5-yl]phenoxy}ethyl)urea obtained by Example 298 (45 mg) in a manner similar to Example 289.

¹H-NMR (DMSO-d₆): δ 0.8-1.1(2H, m), 1.3-1.5(2H, m), 2.4-2.5(1H, m), 3.88(3H, s), 3.9-4(2H, m), 5.54(2H, s), 6.18(1H, br-s), 6.9(1H, d, J=8.6 Hz), 7.05(2H, d, J=8.5 Hz), 7.47(2H, d, J=8.5 Hz), 7.83(1H, dd, J=2.2,8.6 Hz), 8.36(1H, d, J=2.2 Hz). MS (ESI): 460.1 (M+Na)⁺.

### Example 301

### S-1H-Tetrazol-5-yl(2-{4-[4-(6-methoxy-3-pyridinyl)-2-(trifluoromethyl)-1,3-oxazol-5-yl]phenoxy}ethyl)thiocarbamate

The title compound (51.1 mg) was obtained from 4-nitrophenyl(2-{4-[4-(6-methoxy-3-pyridinyl)-2-(trifluoromethyl)-1,3-oxazol-5-yl]phenoxy}ethyl)carbamate obtained by Example 245 (200 mg) in a manner similar to Example 246.

### Example 302

### 1-[5-[4-(2-Hydroxyethoxy)phenyl]-4-(6-methoxy-3-pyridinyl)-1,3-oxazol-2-yl]ethanone

The title compound (104 mg) was obtained from 5-[4-(2-{[tert-butyl(dimethyl)silyl]oxy}ethoxy)phenyl]-N-methoxy-4-(6-methoxy-3-pyridinyl)-N-methyl-1,3-oxazole-2-carboxamide obtained by Example 283 (300 mg) and methyllithium (1.46 mL) in a manner similar to Example 284.

¹H-NMR (DMSO-d₆): δ 2.63(3H, s), 3.6-3.8(2H, m), 3.9(3H, s), 4-4.1(2H, m), 4.91(1H, t, J=5.5 Hz), 6.94(1H, d, J=8.6 Hz), 7.08(2H, d, J=8.8 Hz), 7.53(2H, d, J=9.7 Hz), 7.88(1H, dd, J=2.5,8.6 Hz), 8.38(1H, d, J=2.4 Hz). MS (ESI): 377.2 (M+Na)⁺.

### Example 303

### 4-{4-(6-Methoxy-3-pyridinyl)-2-[(2-pyridinylthio)methyl]-1,3-oxazol-5-yl}phenol

The title compound (311 mg) was obtained from 5-{5-[4-(benzyloxy)phenyl]-2-[(2-pyridinylthio)methyl]-1,3-oxazol-4-yl}-2-methoxypyridine obtained by Example 288 (570 mg) in a manner similar to Example 287.

¹H-NMR (DMSO-d₆): δ 3.86(3H, s), 4.68(2H, s), 6.7-6.9(3H, m), 7.1-7.2(1H, m), 7.28(2H, d, J=8.6 Hz), 7.46(1H, d, J=8.1 Hz), 7.6-7.8(2H, m), 8.31(1H, d, J=2.4 Hz), 8.49(1H, dd, J=1,6.3 Hz), 9.9(1H, br-s). MS (ESI): 414.1(M+Na)⁺.

### Example 304

### tert-Butyl[2-(4-{4-(6-methoxy-3-pyridinyl)-2-[(2-pyridinylthio)methyl]-1,3-oxazol-5-yl}phenoxy}ethyl]carbamate

The title compound (355 mg) was obtained from 4-{4-(6-methoxy-3-pyridinyl)-2-[(2-pyridinylthio)methyl]-1,3-oxazol-5-yl}phenol obtained by Example 303 (298 mg) in a manner similar to Example 182.

¹H-NMR (DMSO-d₆): δ 1.37(9H, s), 3.2-3.4(2H, m), 3.86(3H, s), 3.99(1H, t, J=5.7 Hz), 4.69(2H, s), 6.87(1H, d, J=8.5 Hz), 7(2H, d, J=8.7 Hz), 7.1-7.3(1H, m), 7.4-7.5(3H, m), 7.7-7.85(2H, m), 8.31(1H, d, J=2.4 Hz), 8.4-8.5(1H, m). MS (ESI): 557.2 (M+Na)⁺.

### Example 305

### [2-(4-{4-(6-methoxy-3-pyridinyl)-2-[(2-pyridinylthio)methyl]-1,3-oxazol-5-yl)phenoxy}ethyl]amine

The title compound (261 mg) was obtained from tert-butyl [2-(4-{4-(6-methoxy-3-pyridinyl)-2-[(2-pyridinylthio)methyl]-1,3-oxazol-5-yl}phenoxy)ethyl]carbamate obtained by Example 304 (345 mg) in a manner similar to Example 198.

¹H-NMR (DMSO-d₆): δ 2.9(2H, t, J=5.6 Hz), 3.86(3H, s), 3.97(2H, t, J=5.6 Hz), 4.69(2H, s), 6.87(1H, d, J=8.7 Hz), 7.01(2H, d, J=8.7 Hz), 7.1-7.3(1H, m), 7.38(2H, d, J=8.6 Hz), 7.46(1H, d, J=8 Hz), 7.6-7.8(2H, m), 8.31(1H, d, J=2.2 Hz), 8.49(1H, d, J=4.3 Hz). MS (ESI): 435.1 (M+Na)⁺.

### Example 306

### N-[2-(4-{4-(6-Methoxy-3-pyridinyl)-2-[(2-pyridinylthio)methyl]-1,3-oxazol-5-yl}phenoxy)ethyl]methanesulfonamide

The title compound (68.1 mg) was obtained from [2-(4-{4-(6-methoxy-3-pyridinyl)-2-[(2-pyridinylthio)methyl]-1,3-oxazol-5-yl}phenoxy)ethyl]amine obtained by Example 305 (100 mg) in a manner similar to Example 199.

MS (ESI): 513.1 (M+H)⁺.

### Example 307

### N-[2-(4-{4-(6-Methoxy-3-pyridinyl)-2-[(2-pyridinylthio)methyl]-1,3-oxazol-5-yl}phenoxy)ethyl]methanesulfonamide methanesulfonate

N-[2-(4-{4-(6-Methoxy-3-pyridinyl)-2-[(2-pyridinylthio)methyl]-1,3-oxazol-5-yl}phenoxy)ethyl]methanesulfonamide obtained by Example 306 (80 mg) was dissolved in ethyl acetate (1 mL) and cooled with ice. 0.1M methanesulfonic acid in ethyl acetate (1.57 mL) was added to this solution.

The resulting precipitate was collected by filtraion, washed with ethyl acetate under a nitrogen stream, and dried in vacuo to give the title compound (48 mg).

¹H-NMR (DMSO-d₆): δ 2.37(3H, s), 2.95(3H, s), 3.33(2H, br-s), 3.87(3H, s), 4-4.1(2H, m), 4.7(2H, s), 6.87(1H, d, J=8.6 Hz), 7.03(2H, d, J=8.8 Hz), 7.1-7.2(1H, m), 7.4(2H, d, J=8.7 Hz), 7.48(1H, d, J=8.1 Hz), 7.6-7.8(2H, m), 8.31(1H, d, J=2.1 Hz), 8.5(1H, d, J=4.2 Hz).

### Example 308

### N-[2-(4-{4-(6-Methoxy-3-pyridinyl)-2-[(2-pyridinylthio)methyl]-1,3-oxazol-5-yl}phenoxy)ethyl]urea

The title compound (105 mg) obtained from (2-(4-{4-(6-methoxy-3-pyridinyl)-2-[(2-pyridinylthio)methyl]-1,3-oxazol-5-yl}phenoxy)ethyl]amine obtained by Example 305 (140 mg) in a manner similar to Example 200.

¹H-NMR (DMSO-d₆): δ 3.86(3H, s), 3.98(2H, t, J=5.5 Hz), 4.69(2H, s), 5.53(2H, s), 6.17(1H, t, J=5.6 Hz), 6.87(1H, d, J=8.8 Hz), 7.02(2H, d, J=8.8 Hz), 7.1-7.2(1H, m), 7.39(2H, d, J=8.7 Hz), 7.46(1H, d, J=8.1 Hz), 7.6-7.8(2H, m), 8.31(1H, d, J=2.3 Hz), 8.49(1H, dd, J=1,6.2 Hz).

### Example 309

### 2-Methoxy-5-[5-(4-methoxyphenyl)-2-(methylsulfonyl)-1,3-oxazol-4-yl]pyridine

To a solution of 2-methoxy-5-[5-(4-methoxyphenyl)-2-(methylthio)-1,3-oxazol-4-yl]pyridine obtained by Example290-2(505 mg) in methanol (10 mL)-tetrahydrofuran(3.0 mL), a solution of oxone (2.84 g) in water (13.0 mL) was added at 0° C.

After stirring for 10 hrs at room temperature, the mixture was poured into ice cooling water. The product was extracted with ethyl acetate. The combined extracts were washed with saturated sodium hydrogencarbonate aqueous solution and brine, dried over magnesium sulfate, and evaporated in vacuo to give the title compound (547 mg).

¹H-NMR (200 MHz, CDCl₃): δ 3.41(3H, s), 3.86(3H, s), 3.97(3H, s), 6.79(1H, d, J=8 Hz), 6.94(2H, d, J=9 Hz), 7.56(2H, d, J=9 Hz), 7.84(1H, dd, J=2.5,8.5 Hz), 8.44(1H, d, J=2.5 Hz) MS (ESI): 383 (M+Na)⁺.

### Example 310

### 5-[2-Isopropoxy-5-(4-methoxyphenyl)-1,3-oxazol-4-yl]-2-methoxypyridine

To a solution of 2-propanol (63.7 μL in dioxane (1.3 mL), NaH and a solution of 2-methoxy-5-[5-(4-methoxyphenyl)-2-(methylsulfonyl)-1,3-oxazol-4-yl]pyridine obtained by Example 309 (100 mg) in dioxane (1.5 mL) were added at 0° C.

The mixture was refluxed for 10 min, and poured into saturated ammonium chloride aqueous solution at 0° C. The product was extracted with ethyl acetate. The combined extracts were washed with brine, dried over magnesium sulfate, and evaporated in vacuo. The residue was purified by preparative thin layer chromatography to give the title compound (72.5 mg).

¹H-NMR (200 MHz, CDCl₃): δ 1.47(6H, d, J=6.5 Hz), 3.82(3H, s), 3.97(3H, s), 5.09-5.23(1H, m), 6.74(1H, d, J=8.5 Hz), 6.87(2H, d, J=9 Hz), 7.42(2H, d, J=9 Hz), 7.82(1H, dd, J=2.3,9 Hz), 8.44(1H, d, J=2.3 Hz). MS (ESI): 341 (M+H)⁺, 363 (M+Na)⁺.

### Example 311

### 2-Methoxy-5-[5-(4-methoxyphenyl)-2-(2,2,2-trifluoroethoxy)-1,3-oxazol-4-yl]pyridine

The title compound was obtained from 2-methoxy-5-[5-(4-methoxyphenyl)-2-(methylsulfonyl)-1,3-oxazol-4-yl]pyridine obtained by Example 309 and 2,2,2-trifluoroethanol in a manner similar to Example 310.

¹H-NMR (200 MHz, CDCl₃): δ 3.83(3H, s), 3.97(3H, s), 4.84(2H, q, J=8 Hz), 6.75(1H, d, J=8 Hz), 6.9(2H, d, J=6.5 Hz), 7.44(2H, d, J=9 Hz), 7.79(1H, dd, J=2.3,9 Hz), 8.42(1H, d, J=2 Hz) MS (ESI): 381 (M+H)⁺, 403 (M+Na)⁺.

### Example 312

### 5-[2-(Cyclohexyloxy)-5-(4-methoxyphenyl)-1,3-oxazol-4-yl]-2-methoxypyridine

The title compound was obtained from 2-methoxy-5-[5-(4-methoxyphenyl)-2-(methylsulfonyl)-1,3-oxazol-4-yl]pyridine obtained by Example 309 and cyclohexanol in a manner similar to Example 310.

¹H-NMR (200 MHz, CDCl₃): δ 1.47-2.09(10H, m), 3.82(3H, s), 3.97(3H, s), 4.8-5(1H, m), 6.74(1H, d, J=9 Hz), 6.87(2H, d, J=8.5 Hz), 7.42(2H, d, J=9 Hz), 7.82(1H, dd, J=2.5,8.5 Hz), 8.43(1H, d, J=2 Hz). MS (ESI): 403 (M+Na)⁺.

### Example 313

### 5-[2-(Cyclopentyloxy)-5-(4-methoxyphenyl)-1,3-oxazol-4-yl]-2-methoxypyridine

The title compound was obtained from 2-methoxy-5-[5-(4-methoxyphenyl)-2-(methylsulfonyl)-1,3-oxazol-4-yl]pyridine obtained by Example 309 and cyclopentanol in a manner similar to Example 310.

¹H-NMR (200 MHz, CDCl₃): δ 1.5-2.2(8H, m), 3.82(3H, s), 3.96(3H, s), 6.74(1H, d, J=9.5 Hz), 6.87(2H, d, J=9 Hz), 7.42(2H, d, J=9 Hz), 7.82(1H, dd, J=2.3,8.5 Hz), 8.43(1H, d, J=2.3 Hz). MS (ESI): 367 (M+H)⁺, 389(M+N)⁺.

### Example 314

### 5-[2-sec-Butoxy-5-(4-methoxyphenyl)-1,3-oxazol-4-yl]-2-methoxypyridine

The title compound was obtained from 2-methoxy-5-[5-(4-methoxyphenyl)-2-(methylsulfonyl)-1,3-oxazol-4-yl]pyridine obtained by Example 309 and 2-butanol in a manner similar to Example 310.

¹H-NMR (200 MHz, CDCl₃): δ 1.02(3H, t, J=7.5 Hz), 1.44(3H, d, J=6 Hz), 1.6-2(2H, m), 3.82(3H, s), 3.96(3H, s), 4.92-5.03(1H, m), 6.74(1H, d, J=8.5 Hz), 6.87(2H, d, J=8.5 Hz), 7.43(2H, d, J=8.5 Hz), 7.82(1H, dd, J=2.5,8.5 Hz), 8.43(1H, d, J=2.5 Hz). MS (ESI): 355 (M+H)⁺, 377 (M+Na)⁺.

### Example 315

### 2-(4-{4-(6-Methoxy-3-pyridinyl)-2-[(2-pyridinylthio)methyl]-1,3-oxazol-5-yl}phenoxy)ethanol

The title compound (19.8 mg) was obtained from 4-{4-(6-methoxy-3-pyridinyl)-2-[(2-pyridinylthio)methyl]-1,3-oxazol-5-yl}phenol (90 mg) obtained by Example 303 in a manner similar to Example 181.

¹H-NMR (DMSO-d₆): δ 3.6-3.8(2H, m), 3.86(3H, s), 3.9-4.1(2H, m), 4.69(2H, s), 4.88(1H, br-s), 6.86(1H, d, J=8.6 Hz), 7.01(2H, d, J=8.7 Hz), 7.1-7.2(1H, m), 7.3-7.5(3H, m), 7.6-7.8(2H, m), 8.31(1H, d, J=2 Hz), 8.5(1H, br-s). MS (ESI): 458.2 (M+Na)⁺.

### Example 316

### [5-(4-Methoxyphenyl)-4-(6-methoxy-3-pyridinyl)-1,3-oxazol-2-yl]methyl methanesulfonate

The title compound (241 mg) was obtained from [5-(4-methoxyphenyl)-4-(6-methoxy-3-pyridinyl)-1,3-oxazol-2-yl]methanol (200 mg) in a manner similar to Example 285.

¹H-NMR (DMSO-d₆): δ 2.38(3H, s), 3.8(3H, s), 3.88(3H, s), 4.2-4.4(2H, m), 6.89(1H, d, J=9.1 Hz), 7.04(2H, d, J=8.9 Hz), 7.4-7.8(3H, m), 8.35(1H, d, J=2.2 Hz).

### Example 317

### 2-Methoxy-5-{5-(4-methoxyphenyl)-2-[(4-pyridinylthio)methyl]-1,3-oxazol-4-yl}pyridine methanesulfonate

The title compound (37 mg) was obtained from [5-(4-methoxyphenyl)-4-(6-methoxy-3-pyridinyl)-1,3-oxazol-2-yl]methyl methanesulfonate obtained by Example 316 (51 mg) and 4-mercaptopyridine (29.1 mg) in manners similar to Examples 286 and 307.

¹H-NMR (DMSO-d₆): δ 2.33(3H, s), 3.79(3H, s), 3.87(3H, s), 4.92(2H, s), 6.88(1H, d, J=8.6 Hz), 7.03(2H, d, J=8.8 Hz), 7.44(2H, d, J=8.8 Hz), 7.79(1H, dd, J=2.2,8.6 Hz), 8.06(2H, d, J=6.7 Hz), 8.34(1H, d, J=2.2 Hz), 8.72(2H, d, J=6.7 Hz). MS (ESI): 406.3 (M+H)⁺.

### Example 318

### 2-Methoxy-5-{5-(4-methoxyphenyl)-2-[(2-pyridinylthio)methyl]-1,3-oxazol-4-yl}pyridine methanesulfonate

The title compound (29.5 mg) was obtained from [5-(4-methoxyphenyl)-4-(6-methoxy-3-pyridinyl)-1,3-oxazol-2-yl]methyl methanesulfonate obtained by Example 316 (51 mg) and 2-mercaptopyridine (29.1 mg) in manners similar to Examples 286 and 307.

¹H-NMR (DMSO-d₆): δ 2.43(3H, s), 3.78(3H, s), 3.87(3H, s), 4.7(2H, s), 6.09(1H, br-s), 6.88(1H, d, J=8.5 Hz), 7.01(2H, d, J=8.8 Hz), 7.1-7.3(1H, m), 7.39(2H, d, J=8.9 Hz), 7.5(1H, d, J=8.2 Hz), 7.7-7.8(2H, m), 8.31(1H, d, J=2.3 Hz), 8.51(1H, d, J=4.1 Hz). MS (ESI): 428.2 (M+Na)⁺.

### Example 319

### 2-Methoxy-5-[5-(4-methoxyphenyl)-2-(2-propyn-1-yloxy)-1,3-oxazol-4-yl]pyridine

The title compound was obtained from 2-methoxy-5-[5-(4-methoxyphenyl)-2-(methylsulfonyl)-1,3-oxazol-4-yl]pyridine obtained by Example 309 and 2-propyn-1-ol in a manner similar to Example 310.

¹H-NMR (200 MHz, CDCl₃): δ 2.62(1H, t, J=2.3 Hz), 3.83(3H, s), 3.97(3H, s), 5.07(2H, d, J=2.3 Hz), 6.75(1H, d, J=8.5 Hz), 6.89(2H, d, J=9 Hz), 7.43(2H, d, J=9 Hz), 7.8(1H, d, J=2.5 Hz), 7.44(1H, d, J=2.5 Hz). MS (ESI): 337 (M+H)⁺, 359 (M+Na)⁺.

### Example 320

### 5-[2-(Cyclobutyloxy)-5-(4-methoxyphenyl)-1,3-oxazol-4-yl]-2-methoxypyridine

The title compound was obtained from 2-methoxy-5-[5-(4-methoxyphenyl)-2-(methylsulfonyl)-1,3-oxazol-4-yl]pyridine obtained by Example 309 and cyclobutanol in a manner similar to Example 310.

¹H-NMR (200 MHz, CDCl₃): δ 1.6-2.5(6H, m), 3.82(3H, s), 3.99(3H, s), 5.1-5.22(1H, m), 6.73(1H, d, J=8.5 Hz), 6.87(2H, d, J=9 Hz), 7.41(2H, d, J=9 Hz), 7.79(1H, dd, J=2,8.5 Hz), 8.41(1H, d, J=2 Hz). MS (ESI): 353 (M+H)⁺; 375(M+Na)⁺.

### Example 321

### 5-[2-(Cyclopentylmethoxy)-5-(4-methoxyphenyl)-1,3-oxazol-4-yl]-2-methoxypyridine

The title compound was obtained from 2-methoxy-5-[5-(4-methoxyphenyl)-2-(methylsulfonyl)-1,3-oxazol-4-yl]pyridine obtained by Example 309 and cyclopentylmethanol in a manner similar to Example 310.

¹H-NMR (200 MHz, CDCl₃): δ 1.19-1.98(8H, m), 2.27-2.52(1H, m), 3.82(3H, s), 3.95(3H, s), 4.33(2H, d, J=7 Hz), 6.74(1H, d, J=8.5 Hz), 6.87(2H, d, J=9 Hz), 7.42(2H, d, J=9 Hz), 7.8(1H, dd, J=2.5,8.5 Hz), 8.41(1H, d, J=2.5 Hz). MS (ESI): 403 (M+Na)⁺, 381 (M+H)⁺.

### Example 322

### 2-Methoxy-5-[2-(2-methoxyethoxy)-5-(4-methoxyphenyl)-1,3-oxazol-4-yl]pyridine

The title compound was obtained from 2-methoxy-5-[5-(4-methoxyphenyl)-2-(methylsulfonyl)-1,3-oxazol-4-yl]pyridine obtained by Example 309 and 2-methoxyethanol in a manner similar to Example 310.

¹H-NMR (200 MHz, CDCl₃): δ 3.45(3H, s), 3.75-3.83(2H, m), 3.82(3H, s), 3.98(3H, s), 4.57-4.64(2H, m), 6.76(1H, d, J=8.5 Hz), 6.88(2H, d, J=9 Hz), 7.42(2H, d, J=9 Hz), 7.83(1H, dd, J=2.5,8.5 Hz), 8.44(1H, d, J=2.5 Hz). MS (ESI): 357 (M+H)⁺, 379 (M+Na)⁺.

### Example 323

### 5-[2-(2-Fluoroethoxy)-5-(4-methoxyphenyl)-1,3-oxazol-4-yl]-2-methoxypyridine

The title compound was obtained from 2-methoxy-5-[5-(4-methoxyphenyl)-2-(methylsulfonyl)-1,3-oxazol-4-yl]pyridine obtained by Example 309 and 2-fluoroethanol in a manner similar to Example 310.

¹H-NMR (200 MHz, CDCl₃): δ 3.83(3H, s), 3.96(3H, s), 4.62-4.69(2H, m), 4.75-4.8(1H, m), 4.89-4.94(1H, m), 6.74(1H, d, J=8 Hz), 6.89(2H, d, J=8.5 Hz), 7.43(2H, d, J=8.5 Hz), 7.79(1H, dd, J=2.3,8 Hz), 8.41(1H, d, J=2.3 Hz). MS (ESI): 345 (M+H)⁺, 367 (M+Na)⁺.

### Example 324

### 5-[2-(ethylthio)-5-(4-methoxyphenyl)-1,3-oxazol-4-yl]-2-methoxypyridine

The title compound was obtained from 2-methoxy-5-[5-(4-methoxyphenyl)-2-(methylsulfonyl)-1,3-oxazol-4-yl]pyridine obtained by Example 309 in a manner similar to Example 310.

¹H-NMR (200 MHz, CDCl₃): δ 1.49(3H, t, J=7.4 Hz), 3.24(2H, q, J=7.4 Hz), 3.83(3H, s), 3.96(3H, s), 6.75(1H, d, J=8.5 Hz), 6.9(2H, d, J=9 Hz), 7.46(2H, d, J=9 Hz), 7.82(1H, dd, J=2, 8.5 Hz), 8.44(1H, d, J=2 Hz). MS (ESI): 343 (M+H)⁺, 365 (M+Na)⁺.

### Example 325

### 5-[2-(Cyclopropylmethoxy)-5-(4-methoxyphenyl)-1,3-oxazol-4-yl]-2-methoxypyridine

The title compound was obtained from 2-methoxy-5-[5-(4-methoxyphenyl)-2-(methylsulfonyl)-1,3-oxazol-4-yl]pyridine obtained by Example 309 and cyclopropylmethanol in a manner similar to Example 310.

¹H-NMR (200 MHz, CDCl₃): δ 0.36-0.47(2H, m), 0.63-0.73(2H, m), 1.26-1.48(1H, m), 3.82(3H, s), 3.95(3H, s), 4.29(2H, d, J=7 Hz), 6.73(1H, d, J=8.5 Hz), 6.87(2H, d, J=6.5 Hz), 7.43(2H, d, J=9 Hz), 7.79(1H, dd, J=2.3,8.5 Hz), 8.41(1H, d, J=2.5 Hz). MS (ESI): 353 (M+H)⁺.

### Example 326

### 2-Methoxy-5-{5-(4-methoxyphenyl)-2-[(1H-tetrazol-5-ylthio)methyl]-1,3-oxazol-4-yl}pyridine

The title compound (21.2 mg) was obtained from [5-(4-methoxyphenyl)-4-(6-methoxy-3-pyridinyl)-1,3-oxazol-2-yl]methyl methanesulfonate obtained by Example 316 (51 mg) and 2-mercaptotetrazole (26.7 mg) in a manner similar to Example 286.

¹H-NMR (DMSO-d₆): δ 3.79(3H, s), 3.87(3H, s), 4.41(2H, s), 6.86(1H, d, J=8.6 Hz), 7.01(2H, d, J=8.8 Hz), 7.4(2H, d, J=8.8 Hz), 7.8(1H, dd, J=1.8,9.9 Hz), 8.31(1H, d, J=2.1 Hz). MS (ESI): 395.2 (M−H)⁻.

### Example 327

### 5-[2-(2-Ethoxyethoxy)-5-(4-methoxyphenyl)-1,3-oxazol-4-yl]-2-methoxypyridine

The title compound was obtained from 2-methoxy-5-[5-(4-methoxyphenyl)-2-(methylsulfonyl)-1,3-oxazol-4-yl]pyridine obtained by Example 309 and 2-ethoxyethanol in a manner similar to Example 310.

¹H-NMR (200 MHz, CDCl₃): δ 1.25(3H, t, J=7 Hz), 3.6(2H, q, J=7 Hz), 3.78-3.85(2H, m), 3.82(3H, s), 3.95(3H, s), 4.58-4.62(2H, m), 6.74(1H, d, J=8.5 Hz), 6.87(2H, d, J=9 Hz), 7.42(2H, d, J=8.5 Hz), 7.79(1H, dd, J=2.3,9 Hz), 8.41(1H, d, J=2.3 Hz). MS (ESI): 393(M+Na)⁺.

### Example 328

### 5-[4-(Benzyloxy)phenyl]-4-(4-methoxyphenyl)-2-(methylthio)-1,3-oxazole

The title compound was obtained from 5-[4-(benzyloxy)phenyl]-2-chloro-4-(4-methoxyphenyl)-1,3-oxazole obtained by Example 167-3 in a manner similar to Example 157.

¹H-NMR (CDCl₃): δ 2.71(3H, s), 3.83(3H, s), 5.08(2H, s), 6.70-7.70(13H, m). MS (ESI): 404.2 (M+H)⁺.

In order to illustrate the usefulness of the object compounds (I), the pharmacological test data of the compounds (I) are shown in the following.

[A] Analgesic Activity:

Effect on Adjuvant Arthritis in Rats:

(i) Test Method:

Analgesic activity of a single dose of agents in arthritic rats was studied.

Arthritis was induced by injection of 0.5 mg of Mycobacterium tuberculosis (Difco Laboratories, Detroit, Mich.) in 50 μL of liquid paraffin into the right hind footpad of Lewis rats aged 7 weeks. Arthritic rats were randomized and grouped (n=10) for drug treatment based on pain threshold of left hind paws and body weight on day 22.

Drugs (Test compounds) were administered and the pain threshold was measured 2 hrs after drug administration. The intensity of hyperalgesia was assessed by the method of Randall-Selitto. The mechanical pain threshold of the left hind paw (uninjected hind paw) was determined by compressing the ankle joint with a balance pressure apparatus (Ugo Basile Co. Ltd., Varese, Italy). The threshold pressure of rats squeaking or struggling was expressed in grams. The threshold pressure of rats treated with drugs was compared with that of non-treated rats. A dose showing the ratio of 1.5 is considered to be the effective dose.

(ii) Test Results:

| Test compound | Dose | |
|---|---|---|
| (Example No.) | (mg/kg) | The coefficient of analgesic |
| 12 | 3.2 | >1.5 |
| 33 | 3.2 | >1.5 |
| 54 | 3.2 | >1.5 |
| 55 | 3.2 | >1.5 |
| 118 | 3.2 | >1.5 |
| 122 | 3.2 | >1.5 |

[B] Inhibiting Activity Against COX-I and COX-II

(Whole Blood Assay):

(i) Test Method:

Whole blood assay for COX-I

Fresh blood was collected by syringe without anticoagulants from volunteers with consent. The subjects had no apparent inflammatory conditions and had not taken any medication for at least 7 days prior to blood collection.

500 μL Aliquots of human whole blood were immediately incubated with 2 μL of either dimethyl sulfoxide vehicle or a test compound at final concentrations for 1 hr at 37° C. to allow the blood to clot. Appropriate treatments (no incubation) were used as blanks. At the end of the incubation, 5 μL of 250 mM Indomethacin was added to stop the reaction. The blood was centrifuged at 6000×g for 5 min at 4° C. to obtain serum. A 100 μL aliquot of serum was mixed with 400 μL methanol for protein precipitation. The supernatant was obtained by centrifuging at 6000×g for 5 min at 4° C. and was assayed for TXB₂ using an enzyme immunoassay kit according to the manufacturer's procedure. For a test compound, the results were expressed as percent inhibition of thromboxane B₂(TXB₂) production relative to control incubations containing dimethyl sulfoxide vehicle.

The data were analyzed by that a test compound at the indicated concentrations was changed log value and was applied simple linear regression. IC₅₀ value was calculated by least squares method.

Whole Blood Assay for COX-II

Fresh blood was collected in heparinized tubes by syringe from volunteers with consent. The subjects had no apparent inflammatory conditions and had not taken any medication for at least 7 days prior to blood collection.

500 μL aliquots of human whole blood were incubated with either 2 μL dimethyl sulfoxide vehicle or 2 μL of a test compound at final concentrations for 15 min at 37° C. This was followed by incubation of the blood with 10 μL of 5 mg/mL lipopolysaccharide for 24 hrs at 37° C. for induction of COX-II. Appropriate PBS treatments (no LPS) were used as blanks. At the end of the incubation, the blood was centrifuged at 6000×g for 5 min at 4° C. to obtain plasma. A 100 μL aliquot of plasma was mixed with 400 μL methanol for protein precipitation. The supernatant was obtained by centrifuging at 6000×g for 5 min at 4° C. and was assayed for prostaglandin E₂ (PGE₂) using a radioimmunoassay kit after conversion of PGE₂ to its methyl oximate derivative according to the manufacturer's procedure.

For a test compound, the results were expressed as percent inhibition of PGE₂ production relative to control incubations containing dimethyl sulfoxide vehicle. The data were analyzed by that a test compound at the indicated concentrations was changed log value and was applied simple linear regression. IC₅₀ value was calculated by least squares method.

(ii) Test Results:

| Test Compound | COX-I | COX-II |
|---|---|---|
| (Example No.) | IC50 (μM) | IC50 (μM) |
| 12 | <0.01 | >0.1 |
| 33 | <0.01 | >0.1 |
| 54 | <0.01 | >0.1 |
| 55 | <0.01 | >0.1 |
| 118 | <0.01 | >0.1 |
| 122 | <0.01 | >0.1 |

It appeared, from the above-mentioned Test Results, that the compound (I) or pharmaceutically acceptable salts thereof of the present invention have an inhibiting activity against COX, particularly a selective inhibiting activity against COX-I.

[C] Inhibiting Activity on Aggregation of Platelet

(i) Methods

Preparation of Platelet-Rich Plasma

Blood from healthy human volunteers was collected into plastic vessels containing 3.8% sodium citrate (1/10 volume). The subject had no taken any compounds for at least 7 days prior to blood collection. Platelet-rich plasma was obtained from the supernatant fraction of blood after centrifugation at 1200 rpm. for 10 min. Platelet-poor plasma was obtained by centrifugation of the remaining blood at 3000 rpm for 10 min.

Measurement of Platelet Aggregation

Platelet aggregation was measured according to the turbidimetric method with an aggregometer (Hema Tracer). In the cuvette, platelet-rich plasma was pre-incubated for 2 min at 37° C. after the addition of compounds or vehicle. In order to quantify the inhibitory effects of each compound, the maximum increase in light transmission was determined from the aggregation curve for 7 min after the addition of agonist. We used collagen as agonist of platelet aggregation in this study. The final concentration of collagen was 0.5 μg/mL. The effect of each compound was expressed as percentage inhibition agonist-induced platelet aggregation compared with vehicle treatment. Data are presented as the mean±S.E.M. for six experiments. The IC₅₀ value was obtained by linear regression, and is expressed as the compound concentration required to produce 50% inhibition of agonist-induced platelet aggregation in comparison to vehicle treatment.

It appeared, from the above-mentioned Test Result, that the compound (I) or pharmaceutically acceptable salts thereof of the present invention have an inhibiting activity against platelet aggregation. Therefore, the compound (I) or pharmaceutically acceptable salts thereof are useful for preventing or treating disorders induced by platelet aggregation, such as thrombosis.

Additionally, it was further confirmed that the compounds (I) of the present invention lack undesired side-effects of non-selective NSAIDs, such as gastrointestinal disorders, bleeding, renal toxicity, cardiovascular affection, etc.

As shown above, the object compound (I) or pharmaceutically acceptable salts thereof of this invention possesses COX inhibiting activity, especially COX-I inhibiting activity, and possesses strong anti-inflammatory, antipyretic, analgesic, antithrombotic, anti-cancer activities, and so on.

The object compound (I) and pharmaceutically acceptable salt thereof, therefore, are useful for treating and/or preventing COX mediated diseases, inflammatory conditions, various pains, collagen diseases, autoimmune diseases, various immunological diseases, thrombosis, cancer and neurodegenerative diseases in human beings or animals by using administered systemically or topically.

More particularly, the object compound (I) and pharmaceutically acceptable salts thereof are useful for treating and/or preventing inflammation and acute or chronic pain in joint and muscle [e.g. rheumatoid arthritis, rheumatoidspondylitis, osteoarthritis, gouty arthritis, juvenile arthritis, scapulohumeral periarthritis, cervical syndrome, etc.]; lumbago; inflammatory skin condition [e.g. sunburn, burns, eczema, dermatitis, etc.]; inflammatory eye condition [e.g. conjunctivitis, etc.]; lung disorder in which inflammation is involved [e.g. asthma, bronchitis, pigeon fancier's disease, farmer's lung, etc.]; condition of the gastrointestinal tract associated with inflammation [e.g. aphthous ulcer, Chrohn's disease, atopic gastritis, gastritis varioloid, ulcerative colitis, coeliac disease, regional ileitis, irritable bowel syndrome, etc.]; gingivitis; menorrhalgia; inflammation, pain and tumescence after operation or injury [pain after odontectomy, etc.]; pyrexia, pain and other conditions associated with inflammation, particularly those in which lipoxygenase and cyclooxygenase products are a factor, systemic lupus erythematosus, scleroderma, polymyositis, tendinitis, bursitis, periarteritis nodose, rheumatic fever, Sjogren's syndrome, Behcet disease, thyroiditis, type I diabetes, nephrotic syndrome, aplastic anemia, myasthenia gravis, uveitis contact dermatitis, psoriasis, Kawasaki disease, sarcoidosis, Hodgkin's disease, Alzheimers disease, or the like.

Additionally, the object compound (I) or a salt thereof is expected to be useful as therapeutical and/or preventive agents for cardiovascular or cerebrovascular diseases, the diseases caused by hyperglycemia and hyperlipemia.

The object compound (I) and a salt thereof can be used for prophylactic and therapeutic treatment of arterial thrombosis, arterial sclerosis, ischemic heart diseases [e.g. angina pectoris (e.g. stable angina pectoris, unstable angina pectoris including imminent infarction, etc.), myocardial infarction (e.g. acute myocardial infarction, etc.), coronary thrombosis, etc.], ischemic brain diseases [e.g. cerebral infarction (e.g. acute cerebral thrombosis, etc.), cerebral thrombosis (e.g. cerebral embolism, etc.), transient cerebral ischemia (e.g. transient ischemic attack, etc.), cerebrovascular spasm after cerebral hemorrhage(e.g. cerebrovascular spasm after subarachnoid hemorrhage, etc.), etc.], pulmonary vascular diseases (e.g. pulmonary thrombosis, pulmonary embolism etc.), peripheral circulatory disorder [e.g. arteriosclerosis obliterans, thromboangiitis obliterans (i.e. Buerger's disease), Raynaud's disease, complication of diabetes mellitus (e.g. diabetic angiopathy, diabetic neuropathy, etc.), phiebothrombosis (e.g. deep vein thrombosis, etc.), etc.], complication of tumors (e.g. compressionthrombosis), abortion [e.g. placental thrombosis, etc.], restenosis and reocclusion [e.g. restenosis and/or reocclusion after percutaneous transluminal coronary angioplasty (PTCA), restenosis and reocclusion after the administration of thrombolytic drug (e.g. tissue plasminogen activator (TPA), etc.)], thrombus formation in case of vascular surgery, valve replacement, extracorporeal circulation [e.g. surgery (e.g. open heart surgery, pump-oxygenator, etc.) hemodialysis, etc.] or transplantation, disseminated intravascular coagulation (DIC), thrombotic thrombocytopenia, essential thrombocytosis, inflammation (e.g. nephritis, etc.), immune diseases, atrophic thrombosis, creeping thrombosis, dilation thrombosis, jumping thrombosis, mural thrombosis, etc.

The object compound (I) and a salt thereof can be used for the adjuvant therapy with thrombolytic drug (e.g. TPA, etc.) or anticoagulant (e.g. heparin, etc.).

And, the compound (I) is also useful for inhibition of thrombosis during extra corporeal circulation such as dialysis.

Particularly, the following diseases are exemplified: pains caused by or associated with rheumatoid arthritis, osteoarthritis, lumbar rheumatism, rheumatoid spondylitis, gouty arthritis, juvenile arthritis, etc; lumbago; cervico-omo-brachial syndrome; scapulohumeral periarthritis; pain and tumescence after operation or injury; etc.

The Compound (I) or pharmaceutically acceptable salts thereof of the present invention have an inhibiting cyclooxygenase, especially cyclooxygenase I. Therefore, the Compound (I) or pharmaceutically acceptable salts thereof are useful for treating and/or preventing diseases, more particularly useful for treating and/or preventing inflammatory conditions, various pains, collagen diseases, autoimmune diseases, various immunity diseases, thrombosis, cancer or neurodegerative diseases in human beings or animals.

This application is based on Australian Provisional Patent Application No. 2003900207 filed on Jan. 17, 2003 and No.2003901873 filed on Mar. 31, 2003, the contents of which are hereby incorporated by references.

Although the present invention has been fully described by way of example, it is to be understood that various changes and modifications will be apparent to those skilled in the art. Therefore, unless otherwise such changes and modifications depart from the scope of the present invention hereinafter defined, they should be construed as being included therein.

What is claimed is:

1. A compound of the formula (I): 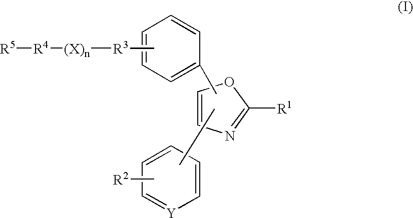
wherein
R¹ is hydrogen, (lower)alkyl, (lower)alkyl substituted with substituent(s) (i) described later, (lower)alkenyl, (lower)alkynyl, cycloalkyl, aryl, saturated heterocyclyl, heteroaryl, (lower)alkoxy, (lower)alkoxy substituted with substituent(s) (i) described later, (lower)alkenyloxy, (lower)alkynyloxy, cycloalkyloxy, aryloxy, heteroaryloxy, (saturated heterocyclyl)oxy, amino, [(lower)alkyl]amino, di[(lower)alkyl]amino, di[(lower)alkyl]amino substituted with substituent(s) (i) described later on (lower)alkyl, [(lower)acyl]amino, cycloalkylamino, arylamino, (saturated heterocyclyl)amino, heteroarylamino, carbamoyl, carbamoyl substituted with substituent(s) (ii) described later, (lower)acyl, cycloalkylcarbonyl, arylcarbonyl, (saturated heterocyclyl)carbonyl, heteroarylcarbonyl, ((lower)alkoxy]carbonyl, [(lower)alkyl]thio, [(lower)alkyl]thio substituted with substituent(s) (i) described later, [(lower)alkyl]sulfinyl, [(lower)alkyl]sulf onyl, cyano, carboxy, hydroxy, mercapto or halogen;
R² is (lower)alkyl, saturated heterocyclyl, (lower)alkoxy or cyano;
R³ is (lower)alkylene, (lower)alkenylene, or covalent bond;
R⁴ is (lower)alkylene, (lower)alkenylene, or covalent bond;
R⁵ is hydrogen, (lower)alkyl, aryl, heteroaryl, (lower)alkoxy, [(lower)acyl]oxy, [(lower)alkyl]sulfonyloxy, [tri(lower)alkyl]silyloxy, amino, [(lower)alkyl]amino, di[(lower)alkyl]amino, [(lower)acyl]amino, [(lower)alkoxy]carbonylamino, [(lower)alkyl]sulfonylamino, heteroarylthiocarbonylamino, carbamoylamino, carbamoylamino substituted with substituent(s) (ii) described later on carbamoyl, aryloxycarbonylamino (which may be substituted with substituent(s) (iii) described later on aryl), [(lower)alkoxy]carbonyl, hydroxy, cyano or azido;
X is “O”, “S”, “SO”, or “SO₂”;
Y is “CH” or “N”;
n is 0 or 1;
substituent(s) (i) is(are) selected from the group consisting of (lower)alkyl, cycloalkyl, aryl, heteroaryl, (lower)alkoxy, [(lower)acyl]oxy, aryl[(lower)alkyl]oxy, [(lower)alkyl]sulfonyloxy, amino, [(lower)alkyl]amino, di[(lower)alkyl]amino, [(lower)acyl]amino, carbamoylamino, [(lower)alkylcarbamoyl]amino, [di(lower)alkylcarbamoyl]amino, [(lower)alkoxycarbonyl]amino, [(lower)alkoxy]carbonyl, [(lower)alkyl]thio, arylthio, heteroarylthio, carboxy, hydroxy, hydroxyimino and halogen;
substituent(s) (ii) is(are) selected from the group consisting of (lower)alkyl, (lower)alkyl substituted with hydroxy, (lower)alkyl substituted with carbamoyl, (lower)alkyl substituted with (lower)alkoxy, (lower)alkoxy, amino, [(lower)alkyl]amino and di[(lower)alkyl]amino;
substituent(s) (iii) is(are) selected from the group consisting of (lower)alkyl, (lower)alkoxy, nitro and cyano;
or pharmaceutically acceptable salts thereof.

2. A compound of the formula (Ia): 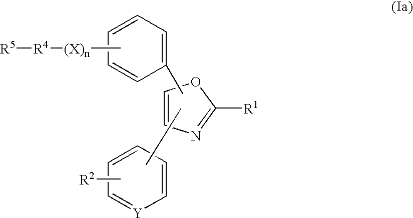
wherein
R¹ is hydrogen, (lower)alkyl, (lower)alkyl substituted with substituent(s) (i) described later, (lower)alkenyl, (lower) alkynyl, cycloalkyl, aryl, saturated heterocyclyl, heteroaryl, (lower)alkoxy, (lower)alkoxy substituted with substituent(s) (i) described later, (lower)alkenyloxy, (lower)alkynyloxy, cycloalkyloxy, aryloxy, heteroaryloxy, (saturated heterocyclyl)oxy, amino, [(lower)alkyl]amino, di[(lower)alkyl]amino, di[(lower)alkyl]amino substituted with substituent(s) (i) described later on (lower)alkyl, [(lower)acyl]amino, cycloalkylamino, arylamino, (saturated heterocyclyl)amino, heteroarylamino, carbamoyl, carbamoyl substituted with substituent(s) (ii) described later, (lower)acyl, cycloalkylcarbonyl, arylcarbonyl, (saturated heterocyclyl)carbonyl, heteroarylcarbonyl, [(lower)alkoxy]carbonyl, [(lower)alkyl]thio, [(lower)alkyl]thio substituted with substituent(s) (i) described later, [(lower)alkyl]sulfinyl, [(lower)alkyl]sulfonyl, cyano, carboxy, hydroxy, mercapto or halogen;
R² is (lower)alkyl, saturated heterocyclyl, (lower)alkoxy or cyano;
R⁴ is (lower)alkylene, (lower)alkenylene, or covalent bond;
R⁵ is hydrogen, (lower)alkyl, aryl, heteroaryl, (lower)alkoxy, [(lower)acyl]oxy, [(lower)alkyl]sulfonyloxy, [tri(lower)alkyl]silyloxy, amino, [(lower)alkyl]amino, di[(lower)alkyl]amino, [(lower)acyl]amino, [(lower)alkoxy]carbonylamino, [(lower)alkyl]sulfonylamino, heteroarylthiocarbonylamino, carbamoylamino, carbamoylamino substituted with substituent(s) (ii) described later on carbamoyl, aryloxycarbonylamino (which may be substituted with substituent(s) (iii) described later on aryl), [(lower)alkoxy]carbonyl, hydroxy, cyano or azido;
X is “O”, “S”, “SO”, or “SO₂”;
Y is “CH” or “N”;
n is 0 or 1;
substituent(s) (i) is(are) selected from the group consisting of (lower)alkyl, cycloalkyl, aryl, heteroaryl, (lower)alkoxy, [(lower)acyl]oxy, aryl[(lower)alkyl]oxy, [(lower)alkyl]sulfonyloxy, amino, [(lower)alkyl]amino, di[(lower)alkyl]amino, [(lower)acyl]amino, carbamoylamino, [(lower)alkylcarbamoyl]amino, [di(lower)alkylcarbamoyl]amino, [(lower)alkoxycarbonyl]amino, [(lower)alkoxy]carbonyl, [(lower)alkyl]thio, arylthio, heteroarylthio, carboxy, hydroxy, hydroxyimino and halogen;
substituent(s) (ii) is(are) selected from the group consisting of (lower)alkyl, (lower)alkyl substituted with hydroxy, (lower)alkyl substituted with carbamoyl, (lower)alkyl substituted with (lower)alkoxy, (lower)alkoxy, amino, [(lower)alkyl]amino and di[(lower)alkyl]amino;
substituent(s) (iii) is(are) selected from the group consisting of (lower)alkyl, (lower)alkoxy, nitro and cyano;
or pharmaceutically acceptable salts thereof.

3. The compound or pharmaceutically acceptable salts thereof according to claim 1 or 2, wherein R¹ is (lower)alkyl substituted with halogen(s), or cycloalkyl.

4. The compound or pharmaceutically acceptable salts thereof according to claim 1 or 2, wherein R² is (lower)alkoxy.

5. The compound or pharmaceutically acceptable salts thereof according to claim 1, wherein R³ is covalent bond.

6. The compound or pharmaceutically acceptable salts thereof according to claim 1 or 2, wherein R⁴ is (lower)alkylene.

7. The compound or pharmaceutically acceptable salts thereof according to claim 1 or 2, wherein R⁵ is [(lower)alkyl]sulfonylamino, carbamoylamino or hydroxy.

8. The compound or pharmaceutically acceptable salts thereof according to claim 1 or 2, wherein X is 0; and n is 1.

9. A compound selected from
2-{4-[2-(Difluoromethyl)-4-(4-methoxyphenyl)-1,3-oxazol-5-yl]phenoxy}ethanol,
2-{4-[2-(Difluoromethyl)-4-(6-methoxy-3-pyridinyl)-1,3-oxazol-5-yl]phenoxy}ethanol,
N-(2-{4-[4-(6-Methoxy-3-pyridinyl)-2-(trifluoromethyl)-1,3-oxazol-5-yl]phenoxy}ethyl)methanesulfonamide,
N-(2-{4-[4-(6-Methoxy-3-pyridinyl)-2-(trifluoromethyl)-1,3-oxazol-5-yl]phenoxy}ethyl)urea,
2-{4-[2-Cyclopropyl-4-(6-methoxy-3-pyridinyl)-1,3-oxazol-5-yl]phenoxy}ethanol and
N-(2-{4-[2-Cyclopropyl-4-(6-methoxy-3-pyridinyl)-1,3-oxazol-5-yl]phenoxy}ethyl)methanesulfonamide.

10. A method for producing the compound or pharmaceutically acceptable salts thereof according to claim 1, which comprises reacting compound (II) with phosphorus oxychloride or triphenylphosphine. 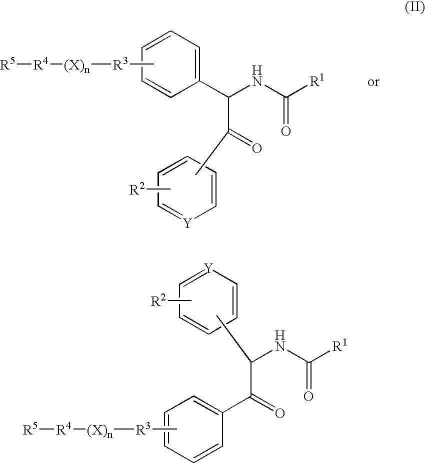
wherein R¹ to R⁵, X, Y and n represent the same meanings.

11. A method for producing the compound or pharmaceutically acceptable salts thereof according to claim 1, which comprises reacting compound (III) with ammonium. 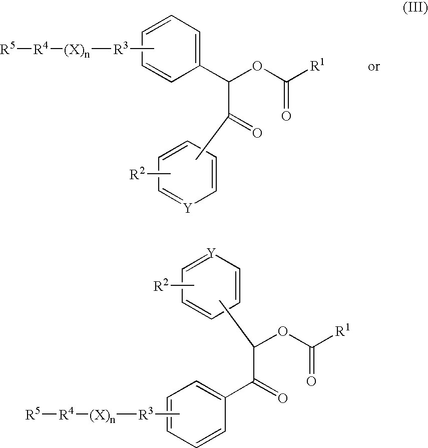
wherein R¹ to R⁵, X, Y and n represent the same meanings.

12. A compound of claim 1, 2 or 9 for use as a medicament.

13. The compound of claim 12 for use in the treatment and/or prevention of inflammatory conditions, various pains, collagen diseases, autoimmune diseases, various immunity diseases, thrombosis, cancer or neurodegerative diseases in human beings or animals.

14. A medicament comprising the compound of claim 1, 2 or 9 as an active ingredient.

15. A pharmaceutical composition comprising the compound of claim 1, 2 or 9 as an active ingredient, in association with a pharmaceutically acceptable carrier or excipient.

16. A method for treatment and/or prevention of inflammatory conditions, various pains, collagen diseases, autoimmune diseases, various immunity diseases, analgesic, thrombosis, cancer or neurodegerative diseases which comprises administering an effective amount of the compound of claim 1, 2 or 9 to human beings or animals.

17. Use of the compound of claim 1, 2 or 9 for treatment and/or prevention of inflammatory conditions, various pains, collagen diseases, autoimmune diseases, various immunity diseases, analgesic, thrombosis, cancer or neurodegerative diseases in human beings or animals.

18. An analgesic agent comprising the compound of claim 1, 2 or 9, which is usable for treating and/or preventing pains caused by or associated with acute or chronic inflammations.

19. The analgesic agent of claim 18, which is usable for treating or preventing pains caused by or associated with rheumatoid arthritis, osteoarthritis, lumbar rheumatism, rheumatoid spondylitis, gouty arthritis, juvenile arthritis; lumbago; cervico-omo-brachial syndrome; scapulohumeral periarthritis; pain and tumescence after operation or injury.

20. A commercial package comprising the pharmaceutical composition containing the compound (I) identified in claim 1, 2 or 9 and a written matter associated therewith, wherein the written matter states that the compound (I) can or should be used for preventing and/or treating inflammatory conditions, various pains, collagen diseases, autoimmune diseases, various immunity diseases, analgesic, thrombosis, cancer or neurodegerative diseases.
